SEQUENCE LISTING (209 sequences)

SEQ ID NO: 1 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSS

SEQ ID NO: 2 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPITFGGGTKVEIK

SEQ ID NO: 3 - protein, 9 aa, synthetic construct
GSFSGYYWS

SEQ ID NO: 4 - protein, 16 aa, synthetic construct
EIDHSGSTNYNPSLKS

SEQ ID NO: 5 - protein, 11 aa, synthetic construct
ARARGPWSFDP

SEQ ID NO: 6 - protein, 117 aa, synthetic construct; identical to SEQ ID NO: 1

SEQ ID NO: 7 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPITFGGGTKVEIK

SEQ ID NO: 8 - protein, 117 aa, synthetic construct; identical to SEQ ID NO: 1

SEQ ID NO: 9 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFYTFGGGTKVEIK

SEQ ID NO: 10 - protein, 117 aa, synthetic construct; identical to SEQ ID NO: 1

SEQ ID NO: 11 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQSNSYYTFGGGTKVEIK

SEQ ID NO: 12 - protein, 117 aa, synthetic construct; identical to SEQ ID NO: 1

SEQ ID NO: 13 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSYPTFGGGTKVEIK

SEQ ID NO: 14 - protein, 117 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWGFDPWGQGTLVTVSS

SEQ ID NO: 15 - protein, 107 aa, synthetic construct
ELQMTQSPSSLSASVGDRVTITCRTSQSISSYLNWYQQKPGQPPKLLIYWASTRESGVPDRFSGSGSGTDFTLTISSLQPEDSATYYCQQSYDIPYTFGQGTKLEIK

SEQ ID NO: 16 - protein, 117 aa, synthetic construct; identical to SEQ ID NO: 1

SEQ ID NO: 17 - protein, 107 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIK

SEQ ID NO: 18 - protein, 117 aa, synthetic construct; identical to SEQ ID NO: 1

SEQ ID NO: 19 - protein, 107 aa, synthetic construct
DIQMTQSPSTLSASVGDRVTITCRASQSISSWLAWYQQKPGKAPKLLIYKASSLESGVPSRFSGSGSGTDFTLTISSLQPDDFATYYCQQSKEVPWTFGQGTKVEIK

SEQ ID NO: 20 - protein, 117 aa, synthetic construct; identical to SEQ ID NO: 1

SEQ ID NO: 21 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYNSFPTFGGGTKVEIK

SEQ ID NO: 22 - protein, 117 aa, synthetic construct; identical to SEQ ID NO: 1

SEQ ID NO: 23 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYDIYPTFGGGTKVEIK

SEQ ID NO: 24 - protein, 117 aa, synthetic construct; identical to SEQ ID NO: 1

SEQ ID NO: 25 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSYPTFGGGTKVEIK

SEQ ID NO: 26 - protein, 117 aa, synthetic construct; identical to SEQ ID NO: 1

SEQ ID NO: 27 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPTFGGGTKVEIK

SEQ ID NO: 28 - protein, 117 aa, synthetic construct; identical to SEQ ID NO: 1

SEQ ID NO: 29 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSFPTFGGGTKVEIK

SEQ ID NO: 30 - protein, 117 aa, synthetic construct; identical to SEQ ID NO: 1

SEQ ID NO: 31 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYSSFSTFGGGTKVEIK

SEQ ID NO: 32 - protein, 117 aa, synthetic construct; identical to SEQ ID NO: 1

SEQ ID NO: 33 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYESYSTFGGGTKVEIK

SEQ ID NO: 34 - protein, 117 aa, synthetic construct; identical to SEQ ID NO: 1

SEQ ID NO: 35 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDSFITFGGGTKVEIK

SEQ ID NO: 36 - protein, 117 aa, synthetic construct; identical to SEQ ID NO: 1

SEQ ID NO: 37 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYQSYPTFGGGTKVEIK

SEQ ID NO: 38 - protein, 117 aa, synthetic construct; identical to SEQ ID NO: 1

SEQ ID NO: 39 - protein, 106 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYHSFPTFGGGTKVEIK

SEQ ID NO: 40 - protein, 117 aa, synthetic construct; identical to SEQ ID NO: 1

SEQ ID NO: 41 - protein, 107 aa, synthetic construct
DIQMTQSPSTLSASVGDRVTITCRASQSIGSWLAWYQQKPGKAPKLLIYKASSLESGVPSRFSGSGSGTEFTLTISSLQPDDFATYYCQQYELYSYTFGGGTKVEIK

SEQ ID NO: 42 - protein, 117 aa, synthetic construct; identical to SEQ ID NO: 1

SEQ ID NO: 43 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYDTFITFGGGTKVEIK

SEQ ID NO: 44 - protein, 125 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARGDSSIRHAYYYYGMDVWGQGTTVTVSS

SEQ ID NO: 45 - protein, 9 aa, synthetic construct
GTFSSYAIS

SEQ ID NO: 46 - protein, 17 aa, synthetic construct
GIIPIFGTANYAQKFQG

SEQ ID NO: 47 - protein, 18 aa, synthetic construct
ARGDSSIRHAYYYYGMDV

SEQ ID NO: 48 - protein, 113 aa, synthetic construct
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPITFGGGTKVEIK

SEQ ID NO: 49 - protein, 17 aa, synthetic construct
KSSQSVLYSSNNKNYLA

SEQ ID NO: 50 - protein, 7 aa, synthetic construct
WASTRES

SEQ ID NO: 51 - protein, 9 aa, synthetic construct
QQYYSTPIT

SEQ ID NO: 52 - protein, 121 aa, synthetic construct
QLQLQESGPGLVKPSETLSLTCTVSGGSISSSSYYWGWIRQPPGKGLEWIGSIYYSGSTYYNPSLKSRVTISVDTSKNQFSLKLSSVTAADTAVYYCARGSDRFHPYFDYWGQGTLVTVSS

SEQ ID NO: 53 - protein, 11 aa, synthetic construct
GSISSSSYYWG

SEQ ID NO: 54 - protein, 16 aa, synthetic construct
SIYYSGSTYYNPSLKS

SEQ ID NO: 55 - protein, 13 aa, synthetic construct
ARGSDRFHPYFDY

SEQ ID NO: 56 - protein, 107 aa, synthetic construct
EIVLTQSPATLSLSPGERATLSCRASQSVSRYLAWYQQKPGQAPRLLIYDASNRATGIPARFSGSGSGTDFTLTISSLEPEDFAVYYCQQFDTWPPTFGGGTKVEIK

SEQ ID NO: 57 - protein, 11 aa, synthetic construct
RASQSVSRYLA

SEQ ID NO: 58 - protein, 7 aa, synthetic construct
DASNRAT

SEQ ID NO: 59 - protein, 9 aa, synthetic construct
QQFDTWPPT

SEQ ID NO: 60 - protein, 117 aa, synthetic construct; identical to SEQ ID NO: 1

SEQ ID NO: 61 - protein, 106 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCEQYDSYPTFGGGTKVEIK

SEQ ID NO: 62 - protein, 126 aa, synthetic construct
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCARRGRKASGSFYYYYGMDVWGQGTTVTVSS

SEQ ID NO: 63 - protein, 9 aa, synthetic construct
GTFSSYAIS

SEQ ID NO: 64 - protein, 17 aa, synthetic construct
GIIPIFGTANYAQKFQG

SEQ ID NO: 65 - protein, 19 aa, synthetic construct
ARRGRKASGSFYYYYGMDV

SEQ ID NO: 66 - protein, 113 aa, synthetic construct [sequence omitted]

SEQ ID NO: 67 - protein, 17 aa, synthetic construct
ESSQSLLNSGNQKNYLT

SEQ ID NO: 68 - protein, 7 aa, synthetic construct
WASTRES

SEQ ID NO: 69 - protein, 9 aa, synthetic construct
QNDYSYPYT

SEQ ID NO: 70 - protein, 126 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGAPNYGDTTHDYYYMDVWGKGTTVTVSS

SEQ ID NO: 71 - protein, 9 aa, synthetic construct
YTFTSYYMH

SEQ ID NO: 72 - protein, 17 aa, synthetic construct
IINPSGGSTSYAQKFQG

SEQ ID NO: 73 - protein, 19 aa, synthetic construct
ARGAPNYGDTTHDYYYMDV

SEQ ID NO: 74 - protein, 107 aa, synthetic construct
EIVMTQSPATLSVSPGERATLSCRASQSVSSNLAWYQQKPGQAPRLLIYGASTRATGIPARFSGSGSGTEFTLTISSLQSEDFAVYYCQQYDDWPFTFGGGTKVEIK

SEQ ID NO: 75 - protein, 11 aa, synthetic construct
RASQSVSSNLA

SEQ ID NO: 76 - protein, 7 aa, synthetic construct
GASTRAT

SEQ ID NO: 77 - protein, 9 aa, synthetic construct
QQYDDWPFT

SEQ ID NO: 78 - protein, 124 aa, synthetic construct
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARDTGEYYDTDDHGMDVWGQGTTVTVSS

SEQ ID NO: 79 - protein, 9 aa, synthetic construct
YTFTGYYMH

SEQ ID NO: 80 - protein, 17 aa, synthetic construct
WINPNSGGTNYAQKFQG

SEQ ID NO: 81 - protein, 17 aa, synthetic construct
ARDTGEYYDTDDHGMDV

SEQ ID NO: 82 - protein, 107 aa, synthetic construct
EIVLTQSPGTLSLSPGERATLSCRASQSVSSNLAWYQQKPGQAPRLLIYGASTRATGIPARFSGSGSGTEFTLTISSLQSEDFAVYYCQQDDYWPPTFGGGTKVEIK

SEQ ID NO: 83 - protein, 50 aa, synthetic construct
GGSGGGGSGGGGSGGGGSGGGGSGGGGSGGGGSGGGGSGGGGSGGGGSGG

SEQ ID NO: 84 - protein, 50 aa, synthetic construct
GGGGSGGGGSGGGGSGGGGSGGGGSGGGGSGGGGSGGGGSGGGGSGGGGS

SEQ ID NO: 85 - protein, 9 aa, synthetic construct
QQDDYWPPT

SEQ ID NO: 86 - protein, 121 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSS

SEQ ID NO: 87 - protein, 9 aa, synthetic construct
FTFSSYAMS

SEQ ID NO: 88 - protein, 17 aa, synthetic construct
AISGSGGSTYYADSVKG

SEQ ID NO: 89 - protein, 14 aa, synthetic construct
AKDGGYYDSGAGDY

SEQ ID NO: 90 - protein, 107 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIK

SEQ ID NO: 91 - protein, 11 aa, synthetic construct
RASQGIDSWLA

SEQ ID NO: 92 - protein, 7 aa, synthetic construct
AASSLQS

SEQ ID NO: 93 - protein, 9 aa, synthetic construct
QQGVSYPRT

SEQ ID NO: 94 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSS

SEQ ID NO: 95 - protein, 9 aa, synthetic construct
FTFSSYSMN

SEQ ID NO: 96 - protein, 17 aa, synthetic construct
SISSSSSYIYYADSVKG

SEQ ID NO: 97 - protein, 15 aa, synthetic construct
ARGAPMGAAAGWFDP

SEQ ID NO: 98 - protein, 107 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIK

SEQ ID NO: 99 - protein, 11 aa, synthetic construct
RASQGISSWLA

SEQ ID NO: 100 - protein, 7 aa, synthetic construct
AASSLQS

SEQ ID NO: 101 - protein, 9 aa, synthetic construct
QQGVSFPRT

SEQ ID NO: 102 - protein, 125 aa, synthetic construct
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCAREGAGFAYGMDYYYMDVWGKGTTVTVSS

SEQ ID NO: 103 - protein, 40 aa, synthetic construct
GGGSGGGSGGGSGGGSGGGSGGGSGGGSGGGSGGGSGGGS

SEQ ID NO: 104 - protein, 40 aa, synthetic construct
GGSGGGSGGGSGGGSGGGSGGGSGGGSGGGSGGGSGGGSG

SEQ ID NO: 105 - protein, 18 aa, synthetic construct
AREGAGFAYGMDYYYMDV

SEQ ID NO: 106 - protein, 107 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQSDNWPFTFGGGTKVEIK

SEQ ID NO: 107 - protein, 11 aa, synthetic construct
RASQSVSSYLA

SEQ ID NO: 108 - protein, 7 aa, synthetic construct
DASNRAT

SEQ ID NO: 109 - protein, 9 aa, synthetic construct
QQSDNWPFT

SEQ ID NO: 110 - protein, 121 aa, synthetic construct
QVQLVESGGGLVKPGGSLRLSCAASGFTFSSYGMHWVRQAPGKGLEWVAFIRYDGSNKYYADSVKGRFTISRDNSKNTLYLQMNSLRAEDTAVYYCAKDRGLGDGTYFDYWGQGTTVTVSS

SEQ ID NO: 111 - protein, 110 aa, synthetic construct
QSALTQPASVSGSPGQSITISCSGSSSNIGNNAVNWYQQLPGKAPKLLIYYDDLLPSGVSDRFSGSKSGTSAFLAISGLQSEDEADYYCAAWDDSLNGPVFGGGTKLTVL

SEQ ID NO: 112 - protein, 115 aa, synthetic construct
QVHLQESGPGLVKPSETLSLTCTVSDDSISSYYWSWIRQPPGKGLEWIGHISYSGSANYNPSLKSRVTISVDTSKNQFSLKLSSVTAADTAVYYCANWDDAFNIWGQGTMVTVSS

SEQ ID NO: 113 - protein, 108 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIK

SEQ ID NO: 114 - protein, 120 aa, synthetic construct [sequence omitted]

SEQ ID NO: 115 - protein, 7 aa, synthetic construct
GFNIKDT

SEQ ID NO: 116 - protein, 6 aa, synthetic construct
YPTNGY

SEQ ID NO: 117 - protein, 11 aa, synthetic construct
WGGDGFYAMDY

SEQ ID NO: 118 - protein, 107 aa, synthetic construct [sequence omitted]

SEQ ID NO: 119 - protein, 8 aa, synthetic construct
QDVNTAVA

SEQ ID NO: 120 - protein, 7 aa, synthetic construct
SASFLYS

SEQ ID NO: 121 - protein, 9 aa, synthetic construct
QQHYTTPPT

SEQ ID NO: 122 - protein, 120 aa, synthetic construct
EVQLVESGGGLVQPGGSLRLSCAASGFTFTDYTMDWVRQAPGKGLEWVADVNPNSGGSIYNQRFKGRFTLSVDRSKNTLYLQMNSLRAEDTAVYYCARNLGPSFYFDYWGQGTLVTVSSA

SEQ ID NO: 123 - protein, 7 aa, synthetic construct
GFTFTDY

SEQ ID NO: 124 - protein, 6 aa, synthetic construct
NPNSGG

SEQ ID NO: 125 - protein, 10 aa, synthetic construct
NLGPSFYFDY

SEQ ID NO: 126 - protein, 108 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGQGTKVEIKR

SEQ ID NO: 127 - protein, 8 aa, synthetic construct
QDVSIGVA

SEQ ID NO: 128 - protein, 7 aa, synthetic construct
SASYRYT

SEQ ID NO: 129 - protein, 9 aa, synthetic construct
QQYYIYPYT

SEQ ID NO: 130 - protein, 120 aa, synthetic construct
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSS

SEQ ID NO: 131 - protein, 7 aa, synthetic construct
GFNIKDT

SEQ ID NO: 132 - protein, 6 aa, synthetic construct
YPTNGY

SEQ ID NO: 133 - protein, 11 aa, synthetic construct
WGGDGFYAMDY

SEQ ID NO: 134 - protein, 107 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIK

SEQ ID NO: 135 - protein, 8 aa, synthetic construct
QDVNTAVA

SEQ ID NO: 136 - protein, 7 aa, synthetic construct
SASFRYT

SEQ ID NO: 137 - protein, 9 aa, synthetic construct
QQHYTTPPT

SEQ ID NO: 138 - protein, 1255 aa, Homo sapiens [sequence omitted]

SEQ ID NO: 139 - protein, 247 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 140 - protein, 475 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 141 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 142 - protein, 210 aa, synthetic construct
DIQMTQSPSSVSASVGDRVTITCRASQGISSWLAWYQQKPGKAPKLLIYAASSLQSGVPSRFSGSGSGTDFTLTISSLQPEDFATYYCQQGVSFPRTFGGGTKVEIKRTVAAPSPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 143 - protein, 20 aa, synthetic construct
GGGGSGGGGSGGGGSGGGGS

SEQ ID NO: 144 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSS

SEQ ID NO: 145 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 146 - protein, 475 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIKGGGGSGGGGSGGGGSGGGGSEVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSSASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 147 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPMGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 148 - protein, 450 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 149 - protein, 214 aa, synthetic construct
DIQMTQSPSSVSASVGDRVTITCRASQGIDSWLAWYQQKPGKAPKLLIYAASSLQSGVPSRFSGSGSGTDFTLTISSLQPEDFATYYCQQGVSYPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 150 - protein, 15 aa, synthetic construct
GGGGSGGGGSGGGGS

SEQ ID NO: 151 - protein, 80 aa, synthetic construct
  VARIANT 5..80: note = GGGS repeats may be deleted
GGGSGGGSGGGSGGGSGGGSGGGSGGGSGGGSGGGSGGGSGGGSGGGSGGGSGGGSGGGSGGGSGGGSGGGSGGGSGGGS

SEQ ID NO: 152 - protein, 100 aa, synthetic construct
GGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGS

SEQ ID NO: 153 - protein, 80 aa, synthetic construct
  VARIANT 5..80: note = GGSG repeats may be deleted
GGSGGGSGGGSGGGSGGGSGGGSGGGSGGGSGGGSGGGSGGGSGGGSGGGSGGGSGGGSGGGSGGGSGGGSGGGSGGGSG

SEQ ID NO: 154 - protein, 100 aa, synthetic construct
GGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGGGGSGG

SEQ ID NO: 155 - protein, 450 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDGGYYDSGAGDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTENQVSLTCLVKGFYPSDIAVEWESNGQPENNYKTTPPVLDSDGSFFLYSWLTVDKSRWQQGNVFSCSVMHEALHNHYTQKSLSLSPG

SEQ ID NO: 156 - protein, 100 aa, synthetic construct; identical to SEQ ID NO: 154
  VARIANT 6..100: note = GGSGG repeats may be deleted

SEQ ID NO: 157 - protein, 100 aa, synthetic construct; identical to SEQ ID NO: 152
  VARIANT 6..100: note = GGGGS repeats may be deleted

SEQ ID NO: 158 - protein, 5 aa, synthetic construct
SYAIS

SEQ ID NO: 159 - protein, 16 aa, synthetic construct
GDSSIRHAYYYYGMDV

SEQ ID NO: 160 - protein, 7 aa, synthetic construct
SSSYYWG

SEQ ID NO: 161 - protein, 11 aa, synthetic construct
GSDRFHPYFDY

SEQ ID NO: 162 - protein, 5 aa, synthetic construct
SYYMH

SEQ ID NO: 163 - protein, 17 aa, synthetic construct
GAPNYGDTTHDYYYMDV

SEQ ID NO: 164 - protein, 5 aa, synthetic construct
GYYMH

SEQ ID NO: 165 - protein, 15 aa, synthetic construct
DTGEYYDTDDHGMDV

SEQ ID NO: 166 - protein, 5 aa, synthetic construct
SYAMS

SEQ ID NO: 167 - protein, 12 aa, synthetic construct
DGGYYDSGAGDY

SEQ ID NO: 168 - protein, 5 aa, synthetic construct
SYSMN

SEQ ID NO: 169 - protein, 13 aa, synthetic construct
GAPMGAAAGWFDP

SEQ ID NO: 170 - protein, 16 aa, synthetic construct
EGAGFAYGMDYYYMDV

SEQ ID NO: 171 - protein, 249 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSA

SEQ ID NO: 172 - protein, 15 aa, synthetic construct
ARGAPIGAAAGWFDP

SEQ ID NO: 173 - protein, 13 aa, synthetic construct
GAPIGAAAGWFDP

SEQ ID NO: 174 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPQGAAAGWFDPWGQGTLVTVSS

SEQ ID NO: 175 - protein, 15 aa, synthetic construct
ARGAPQGAAAGWFDP

SEQ ID NO: 176 - protein, 13 aa, synthetic construct
GAPQGAAAGWFDP

SEQ ID NO: 177 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPLGAAAGWFDPWGQGTLVTVSS

SEQ ID NO: 178 - protein, 15 aa, synthetic construct
ARGAPLGAAAGWFDP

SEQ ID NO: 179 - protein, 13 aa, synthetic construct
GAPLGAAAGWFDP

SEQ ID NO: 180 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPFGAAAGWFDPWGQGTLVTVSS

SEQ ID NO: 181 - protein, 15 aa, synthetic construct
ARGAPFGAAAGWFDP

SEQ ID NO: 182 - protein, 13 aa, synthetic construct
GAPFGAAAGWFDP

SEQ ID NO: 183 - protein, 122 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPVGAAAGWFDPWGQGTLVTVSS

SEQ ID NO: 184 - protein, 15 aa, synthetic construct
ARGAPVGAAAGWFDP

SEQ ID NO: 185 - protein, 13 aa, synthetic construct
GAPVGAAAGWFDP

SEQ ID NO: 186 - protein, 122 aa, synthetic construct
  VARIANT 102: note = M, L, I, V, Q or F
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPXGAAAGWFDPWGQGTLVTVSS

SEQ ID NO: 187 - protein, 15 aa, synthetic construct
  VARIANT 6: note = M, L, I, V, Q or F
ARGAPXGAAAGWFDP

SEQ ID NO: 188 - protein, 13 aa, synthetic construct
  VARIANT 4: note = M, L, I, V, Q or F
GAPXGAAAGWFDP

SEQ ID NO: 189 - protein, 248 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSA

SEQ ID NO: 190 - protein, 476 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 191 - protein, 476 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKRGGGGSGGGGSGGGGSGGGGSEVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 192 - protein, 477 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPRVYTLPPCRDELTKNQVSLTCLVKGFYPSDIAVEWESNGQPENNYKTTPPVLVSDGSFTLYSKLTVDKSRWQQGNVFSCSVMHEALHNHYTQKSLSLSPG

SEQ ID NO: 193 - protein, 477 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKRGGGGSGGGGSGGGGSGGGGSQVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSAASDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 194 - protein, 451 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARGAPIGAAAGWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 195 - protein, 120 aa, synthetic construct
EVQLVESGGGLVQPGGSLRLSCAASGFNIKDTYIHWVRQAPGKCLEWVARIYPTNGYTRYADSVKGRFTISADTSKNTAYLQMNSLRAEDTAVYYCSRWGGDGFYAMDYWGQGTLVTVSS

SEQ ID NO: 196 - protein, 107 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGCGTKVEIK

SEQ ID NO: 197 - protein, 120 aa, synthetic construct
EVQLVESGGGLVQPGGSLRLSCAASGFTFTDYTMDWVRQAPGKCLEWVADVNPNSGGSIYNQRFKGRFTLSVDRSKNTLYLQMNSLRAEDTAVYYCARNLGPSFYFDYWGQGTLVTVSSA

SEQ ID NO: 198 - protein, 108 aa, synthetic construct
DIQMTQSPSSLSASVGDRVTITCKASQDVSIGVAWYQQKPGKAPKLLIYSASYRYTGVPSRFSGSGSGTDFTLTISSLQPEDFATYYCQQYYIYPYTFGCGTKVEIKR

SEQ ID NO: 199 - protein, 121 aa, synthetic construct
QVQLQQSGPELVKPGASLKLSCTASGFNIKDTYIHWVKQRPEQCLEWIGRIYPTNGYTRYDPKFQDKATITADTSSNTAYLQVSRLTSEDTAVYYCSRWGGDGFYAMDYWGQGASVTVSSA

SEQ ID NO: 200 - protein, 108 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGCGTKVEIKR

SEQ ID NO: 201 - protein, 20 aa, synthetic construct
GSGSGSGSGSGSGSGSGSGS

SEQ ID NO: 202 - protein, 30 aa, synthetic construct
GGSGGSGGSGGSGGSGGSGGSGGSGGSGGS

SEQ ID NO: 203 - skipped

SEQ ID NO: 204 - protein, 40 aa, synthetic construct
  SITE 3..40: note = GS repeats may be deleted
GSGSGSGSGSGSGSGSGSGSGSGSGSGSGSGSGSGSGSGS

SEQ ID NO: 205 - protein, 60 aa, synthetic construct
  SITE 4..60: note = GGS repeats may be deleted
GGSGGSGGSGGSGGSGGSGGSGGSGGSGGSGGSGGSGGSGGSGGSGGSGGSGGSGGSGGS

SEQ ID NO: 206 - protein, 214 aa, synthetic construct
DIQMTQSPSTLSASVGDRVTITCRASQSISSWLAWYQQKPGKAPKLLIYKASSLESGVPSRFSGSGSGTEFTLTISSLQPDDFATYYCQQYGSFPITFGGGTKVEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 207 - protein, 452 aa, synthetic construct
QVQLQQWGAGLLKPSETLSLTCAVYGGSFSGYYWSWIRQPPGKGLEWIGEIDHSGSTNYNPSLKSRVTISVDTSKNQFSLKLSSVTAADTAVYYCARARGPWSFDPWGQGTLVTVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMIKGFLPAEIAVDWTSNGRTEQNYKNTATVLDSDGSYFMYSRLRVQKSTWERGSLFACSVVHEGLHNHLTTKTISRSLGK

SEQ ID NO: 208 - protein, 214 aa, synthetic construct
DIQMSQSPASLSASVGETVTITCRASGNIYNYLAWYQQKQGKSPHLLVYDAKTLADGVPSRFSGSGSGTQYSLKISSLQTEDSGNYYCQHFWSLPFTFGSGTKLEIKRADAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 209 - protein, 456 aa, synthetic construct
EVQLQQSGAELVRPGALVKLSCKTSGFNIKDYFLHWVRQRPDQGLEWIGWINPDNGNTVYDPKFQGTASLTADTSSNTVYLQLSGLTSEDTAVYFCTRRDYTYEKAALDYWGQGASVIVSSAKTTAPSVYPLAPVCGGTTGSSVTLGCLVKGYFPEPVTLTWNSGSLSSGVHTFPALLQSGLYTLSSSVTVTSNTWPSQTITCNVAHPASSTKVDKKIEPRVPITQNPCPPLKECPPCAAPDLLGGPSVFIFPPKIKDVLMISLSPMVTCVVVDVSEDDPDVQISWFVNNVEVHTAQTQTHREDYNSTLRVVSALPIQHQDWMSGKEFKCKVNNRALPSPIEKTISKPRGPVRAPQVYVLPPPAEEMTKKEFSLTCMITGFLPAEIAVDWTSNGRTEQNYKNTATVLDSDGSYLMYSKLTVQKSTWERGSLFACSVVHEGLHNHLTTKTISRSLGK

(19) United States
(12) Patent Application Publication
(10) Pub. No.: US 2023/0416402 A1
(43) Pub. Date: Dec. 28, 2023

(54) METHODS OF TREATING CANCER USING MULTI-SPECIFIC BINDING PROTEINS THAT BIND NKG2D, CD16, AND HER2

(71) Applicant: Dragonfly Therapeutics, Inc., Waltham, MA (US)

(72) Inventors: Jean-Marie Cuillerot, Somerville, MA (US); Mark DeRose, Wilmington, MA (US); Christopher Ryan Morgan, Southborough, MA (US); Michael C. Naill, Stow, MA (US); Avni Shah, Framingham, MA (US)

(21) Appl. No.: 18/177,847

(22) Filed: Mar. 3, 2023

Related U.S. Application Data
(60) Provisional application No. 63/404,349, filed on Sep. 7, 2022.
(60) Provisional application No. 63/316,304, filed on Mar. 3, 2022.

(51) Int. Cl. C07K 16/32; C07K 16/28; A61K 31/337; A61K 39/395; A61P 35/00

(52) U.S. Cl. CPC C07K 16/32; C07K 16/2851; C07K 16/283; C07K 16/2818; A61K 31/337; A61K 39/3955; A61P 35/00; C07K 2317/622

(57) ABSTRACT

This disclosure relates to methods of treating cancer using multi-specific binding proteins that bind NKG2D, CD16 and a tumor-associated antigen such as HER2, and pharmaceutical compositions thereof. Also provided are uses of the multi-specific binding protein in combination with an anti-PD-1 therapeutic and/or cytoskeletal disrupting chemotherapeutic agent.

### CROSS-REFERENCE TO RELATED APPLICATIONS

This application claims the benefit of and priority to U.S. Provisional Patent Application No. 63/316,304, filed Mar. 3, 2022, and U.S. Provisional Patent Application No. 63/404,349, filed Sep. 7, 2022, the entire contents of each of which are incorporated by reference herein for all purposes.

### SEQUENCE LISTING

The instant application contains a Sequence Listing which has been submitted electronically in XML format and is hereby incorporated by reference in its entirety. Said XML file copy, created on Feb. 10, 2023, is named DFY-126US SL.xml and is 203,473 bytes in size.

### FIELD OF THE DISCLOSURE

The present disclosure relates to methods of treating cancer using multi-specific binding proteins that bind NKG2D, CD16 and a tumor-associated antigen such as HER2.

### BACKGROUND

Cancer continues to be a significant health problem despite the substantial research efforts and scientific advances reported in the literature for treating this disease. Cancer immunotherapies are being developed to facilitate destruction of cancer cells using the patient's own immune system. The immune cells activated by cancer immunotherapies include T cells and natural killer (NK) cells. For example, bispecific T-cell engagers are designed to direct T cells against tumor cells, thereby rendering cytotoxicity against the tumor cells. Bispecific antibodies that bind NK cells and a tumor-associated antigen (TAA) have also been created for cancer treatment (see, e.g., WO 2016/134371).

HER2 is a transmembrane glycoprotein in the epidermal growth factor receptor family. It is a receptor tyrosine kinase and regulates cell survival, proliferation, and growth. HER2 plays an important role in human malignancies. The ERBB2 gene is amplified or overexpressed in approximately 30% of human breast cancers. Patients with HER2-overexpressing breast cancer have substantially lower overall survival rates and shorter disease-free intervals than patients whose cancer does not overexpress HER2. Moreover, overexpression of HER2 leads to increased breast cancer metastasis. Over-expression of HER2 is also known to occur in many other cancer types, including ovarian, esophageal, bladder and gastric cancer, salivary duct carcinoma, adenocarcinoma of the lung, and aggressive forms of uterine cancer, such as uterine serous endometrial carcinoma.

Multi-specific binding proteins that bind HER2 and one or more immune cell surface proteins have been studied. For example, WO 2018/152518 describes multi-specific binding proteins that bind HER2, NKG2D, and CD16. The present disclosure adds to these developments and provides clinical methods, including dosage regimens, to treat patients with specific HER2-targeting cancer immunotherapies with desired safety and efficacy. Furthermore, the present disclosure adds to the earlier developments in the field by providing formulations containing such cancer immunotherapies that are sufficiently stable and suitable for administration to patients.

### SUMMARY OF THE DISCLOSURE

The present disclosure provides various methods of treating cancer using a multi-specific binding protein having an antigen-binding site that binds HER2, an antigen-binding site that binds NKG2D, and an antibody Fc domain or a portion thereof sufficient to bind CD16, or a third antigen-binding site that binds CD16.

Where the cancer is a gastric cancer, in certain embodiments, the method includes administering to a subject in need thereof an effective amount of a multi-specific binding protein disclosed herein, wherein the subject has received (a) a platinum salt and a fluoropyridine and/or (b) trastuzumab or a biosimilar to trastuzumab as one or more previous therapies, e.g., (a) and (b) in combination as a first line of therapy. In certain embodiments, the subject has received (a). In certain embodiments, the subject has received (b). Previous treatment with both (a) and (b) is also contemplated. In certain embodiments, the gastric cancer has progressed after the one or more previous therapies (e.g., the first line of therapy). In certain embodiments, the gastric cancer is advanced gastric cancer or cancer of the gastro-esophageal junction under the 7th AJCC classification, optionally wherein the advanced cancer is unresectable, recurrent, or metastatic. In certain embodiments, the gastric cancer is HER2-overexpressing, e.g., having (i) a HER2 expression level scored as 3+ by immunohistochemistry or (ii) a HER2 expression level scored as 2+ by immunohistochemistry and ERBB2 gene amplification. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating a gastric cancer, the method including administering to the subject an effective amount of a multi-specific binding protein disclosed herein, wherein the subject has received (a) a platinum salt and a fluoropyridine and/or (b) a previously administered PD-1 inhibitor (e.g., anti-PD-1 antibody) as one or more previous therapies, e.g., (a) and (b) in combination as a first line of therapy. In certain embodiments, the subject has received (a). In certain embodiments, the subject has received (b). Previous treatment with both (a) and (b) is also contemplated. In certain embodiments, the gastric cancer has progressed after the one or more previous therapies (e.g., the first line of therapy). In certain embodiments, the gastric cancer is advanced under the 7th AJCC classification, optionally wherein the advanced cancer is unresectable, recurrent, or metastatic. In certain embodiments, the gastric cancer is HER2-low, e.g., having (i) absence of ERBB2 gene amplification or (ii) a HER2 expression level scored as 0, 1+, or 2+ by immunohistochemistry, wherein if the score is 0, HER2 expression is detected in at least 1% of tumor cells. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating a gastric cancer, the method including administering to a subject in need thereof an effective amount of a multi-specific binding protein disclosed herein, wherein the subject has received a previously administered PD-1 inhibitor (e.g., an anti-PD-1 antibody) as a previous therapy (e.g., a previous line of therapy) or has experienced a drug-related toxicity associated with the previous therapy (e.g., a Grade 3 or 4 drug-related toxicity during the treatment with the PD-1 inhibitor or a Grade 2 drug-related toxicity related to the PD-1 inhibitor that impacted either the lungs or the neurological system). In certain embodiments, the gastric cancer has progressed after the previous therapy. In certain embodiments, the gastric cancer is advanced gastric cancer or cancer of the gastro-esophageal junction under the 7th AJCC classification, optionally wherein the advanced cancer is unresectable, recurrent, or metastatic. In certain embodiments, the gastric cancer is HER2-overexpressing, e.g., having (i) a HER2 expression level scored as 3+ by immunohistochemistry or (ii) a HER2 expression level scored as 2+ by immunohistochemistry and ERBB2 gene amplification. In certain embodiments, the gastric cancer is HER2-low, e.g., having (i) absence of ERBB2 gene amplification or (ii) a HER2 expression level scored as 0, 1+, or 2+ by immunohistochemistry, wherein if the score is 0, HER2 expression is detected in at least 1% of tumor cells. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating a HER2-low gastric cancer, the method comprising administering to a subject in need thereof effective amounts of a PD-1 inhibitor and a multi-specific binding protein disclosed herein. The HER2-low gastric cancer can have (i) absence of ERBB2 gene amplification or (ii) a HER2 expression level scored as 0, 1+, or 2+ by immunohistochemistry, wherein if the score is 0, HER2 expression is detected in at least 1% of tumor cells. In certain embodiments, the gastric cancer is advanced gastric cancer or cancer of the gastro-esophageal junction under the 7th AJCC classification, optionally wherein the advanced cancer is unresectable, recurrent, or metastatic. In certain embodiments, the subject has received (a) a platinum salt and a fluoropyridine and/or (b) a previously administered PD-1 inhibitor (e.g., an anti-PD-1 antibody) as one or more previous therapies, e.g., (a) and (b) in combination as a first line of therapy. In certain embodiments, the gastric cancer has progressed after the one or more previous therapies (e.g., the first line of therapy). In certain embodiments, the PD-1 inhibitor administered in combination with the multi-specific binding protein includes an anti-PD-1 antibody, optionally nivolumab.

Where the cancer is an esophageal cancer, in certain embodiments, the method is used to treat an adenocarcinoma of the esophagus, the method including administering to a subject in need thereof an effective amount of a multi-specific binding protein disclosed herein. In certain embodiments, the adenocarcinoma of the esophagus is advanced under the 7th AJCC classification, optionally wherein the advanced cancer is unresectable, recurrent, or metastatic. In certain embodiments, the adenocarcinoma of the esophagus is HER2-overexpressing, e.g., having (i) a HER2 expression level scored as 3+ by immunohistochemistry or (ii) a HER2 expression level scored as 2+ by immunohistochemistry and HER2 gene amplification. In certain embodiments, the subject has received (a) a platinum salt and a fluoropyridine and/or (b) trastuzumab or a biosimilar to trastuzumab as one or more previous therapies, e.g., (a) and (b) in combination as a first line of therapy. In certain embodiments, the adenocarcinoma of the esophagus has progressed after the one or more previous therapies (e.g., the first line of therapy). In certain embodiments, the adenocarcinoma of the esophagus is HER2-low, e.g., having (i) absence of ERBB2 gene amplification or (ii) a HER2 expression level scored as 0, 1+, or 2+ by immunohistochemistry, wherein if the score is 0, HER2 expression is detected in at least 1% of tumor cells. In certain embodiments, the subject has received (a) a platinum salt and a fluoropyridine and/or (b) a previously administered PD-1 inhibitor (e.g., an anti-PD-1 antibody) as one or more previous therapies, e.g., (a) and (b) in combination as a first line of therapy. In certain embodiments, the adenocarcinoma of the esophagus has progressed after the one or more previous therapies (e.g., the first line of therapy). In certain embodiments, the subject has received a previously administered PD-1 inhibitor (e.g., an anti-PD-1 antibody) as a previous (e.g., a previous line of therapy) or has experienced a drug-related toxicity associated with the previous therapy (e.g., a Grade 3 or 4 drug-related toxicity during the treatment with the PD-1 inhibitor or a Grade 2 drug-related toxicity related to the PD-1 inhibitor that impacted either the lungs or the neurological system). In certain embodiments, the adenocarcinoma of the esophagus has progressed after the previous therapy. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating an esophageal cancer (e.g., an adenocarcinoma of the esophagus), the method including administering to a subject in need thereof an effective amount of a multi-specific binding protein disclosed herein, wherein the subject has received (a) a platinum salt and a fluoropyridine and/or (b) trastuzumab or a biosimilar to trastuzumab as one or more previous therapies, e.g., (a) and (b) in combination as a first line of therapy. In certain embodiments, the esophageal cancer has progressed after the one or more previous therapies (e.g., the first line of therapy). In certain embodiments, the esophageal cancer is advanced under the 7th AJCC classification, optionally wherein the advanced cancer is unresectable, recurrent, or metastatic. In certain embodiments, the gastric cancer is HER2-overexpressing, e.g., having (i) a HER2 expression level scored as 3+ by immunohistochemistry or (ii) a HER2 expression level scored as 2+ by immunohistochemistry and ERBB2 gene amplification. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating an esophageal cancer (e.g., an adenocarcinoma of the esophagus), the method including administering to a subject in need thereof an effective amount of a multi-specific binding protein disclosed herein, wherein the subject has received (a) a platinum salt and a fluoropyridine and/or (b) a previously administered PD-1 inhibitor (e.g., anti-PD-1 antibody) as one or more previous therapies, e.g., (a) and (b) in combination as a first line of therapy. In certain embodiments, the esophageal cancer has progressed after the one or more previous therapies (e.g., the first line of therapy). In certain embodiments, the esophageal cancer is advanced under the 7th AJCC classification, optionally wherein the advanced cancer is unresectable, recurrent, or metastatic. In certain embodiments, the esophageal cancer is HER2-low, e.g., having (i) absence of ERBB2 gene amplification or (ii) a HER2 expression level scored as 0, 1+, or 2+ by immunohistochemistry, wherein if the score is 0, HER2 expression is detected in at least 1% of tumor cells. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating an esophageal cancer (e.g., an adenocarcinoma of the esophagus), the method including administering to a subject in need thereof an effective amount of a multi-specific binding protein disclosed herein, wherein the subject has received a previously administered PD-1 inhibitor (e.g., an anti-PD-1 antibody) as a previous therapy (e.g., a previous line of therapy) or has experienced a drug-related toxicity associated with the previous therapy (e.g., a Grade 3 or 4 drug-related toxicity during the treatment with the PD-1 inhibitor or a Grade 2 drug-related toxicity related to the PD-1 inhibitor that impacted either the lungs or the neurological system). In certain embodiments, the esophageal cancer has progressed after the previous therapy. In certain embodiments, the esophageal cancer is advanced under the 7th AJCC classification, optionally wherein the advanced cancer is unresectable, recurrent, or metastatic. In certain embodiments, the esophageal cancer is HER2-overexpressing, e.g., having (i) a HER2 expression level scored as 3+ by immunohistochemistry or (ii) a HER2 expression level scored as 2+ by immunohistochemistry and ERBB2 gene amplification. In certain embodiments, the esophageal cancer is HER2-low, e.g., having (i) absence of ERBB2 gene amplification or (ii) a HER2 expression level scored as 0, 1+, or 2+ by immunohistochemistry, wherein if the score is 0, HER2 expression is detected in at least 1% of tumor cells. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating a HER2-low esophageal cancer (e.g., an adenocarcinoma of the esophagus), the method including administering to a subject in need thereof effective amounts of a PD-1 inhibitor and a multi-specific binding protein disclosed herein. The HER2-low esophageal cancer can have (i) absence of ERBB2 gene amplification or (ii) a HER2 expression level scored as 0, 1+, or 2+ by immunohistochemistry, wherein if the score is 0, HER2 expression is detected in at least 1% of tumor cells. In certain embodiments, the esophageal cancer is advanced under the 7th AJCC classification, optionally wherein the advanced cancer is unresectable, recurrent, or metastatic. In certain embodiments, the subject has received (a) a platinum salt and a fluoropyridine and/or (b) a previously administered PD-1 inhibitor (e.g., an anti-PD-1 antibody) as one or more previous therapies, e.g., (a) and (b) in combination as a first line of therapy. In certain embodiments, the esophageal cancer has progressed after the one or more previous therapies (e.g., the first line of therapy). In certain embodiments, the PD-1 inhibitor administered in combination with the multi-specific binding protein includes an anti-PD-1 antibody, optionally nivolumab.

Where the cancer is a breast cancer, in certain embodiments, the method includes administering to a subject in need thereof an effective amount of a multi-specific binding protein disclosed herein, wherein the subject has received (a) one or more anti-HER2 antibodies and/or (b) a HER2-targeting antibody-drug conjugate (ADC) as one or more previous therapies. Where the subject has received (a) as a previous therapy (e.g., a previous line of therapy), the one or more anti-HER2 antibodies can include trastuzumab, pertuzumab, or a combination of trastuzumab and pertuzumab. Where the subject has received (b) as a previous therapy (e.g., a previous line of therapy), the HER2-targeting ADC can include trastuzumab deruxtecan. Previous treatment with both (a) and (b) is also contemplated. In certain embodiments, the breast cancer has progressed after either or both previous therapies. In addition, in certain embodiments, the breast cancer has progressed after a previous line of systemic chemotherapy. In certain embodiments, the breast cancer is metastatic or locally advanced. In certain embodiments, the breast cancer is HER2-overexpressing, e.g., having (i) a HER2 expression level scored as 3+ by immunohistochemistry or (ii) a HER2 expression level scored as 2+ by immunohistochemistry and ERBB2 gene amplification. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating a breast cancer, the method comprising administering to a subject in need thereof an effective amount of a multi-specific binding protein disclosed herein, wherein the subject has received (a) a systemic chemotherapy or (b) a trastuzumab-containing antibody-drug conjugate as a previous therapy (e.g., a first line of therapy). In certain embodiments, the breast cancer has progressed after the previous therapy. In certain embodiments, the breast cancer is metastatic or locally advanced. In certain embodiments, the breast cancer is HER2-low, e.g., having (i) absence of ERBB2 gene amplification or (ii) a HER2 expression level scored as 0, 1+, or 2+ by immunohistochemistry, wherein if the score is 0, HER2 expression is detected in at least 1% of tumor cells. The multi-specific binding protein can be provided as a monotherapy. Alternatively, the method can further include administering to the subject a PD-1 inhibitor, e.g., an anti-PD-1 antibody, e.g., nivolumab.

Where the cancer is a triple-negative breast cancer, in certain embodiments, the method includes administering to a subject in need thereof a multi-specific binding protein disclosed herein. In certain embodiments, the subject is not eligible for treatment in combination with an anti-PD-L1 therapy or the cancer in the subject is PD-L1 negative. In certain embodiments, the subject is eligible for treatment with nab-paclitaxel after failure of combination chemotherapy for metastatic disease or relapse within 6 months of adjuvant chemotherapy. In certain embodiments, the cancer has no standard therapy or a standard therapy for the cancer has failed. In certain embodiments, the triple-negative breast cancer is advanced, optionally unresectable, recurrent, or metastatic. In certain embodiments, the triple-negative breast cancer is metastatic or locally advanced. In certain embodiments, the triple-negative breast cancer has a PD-L1 score (CPS) less than 10 as measured by immunohistochemistry. In certain embodiments, the subject has not previously received a chemotherapy or targeted systemic therapy. In certain embodiments, the method of treating triple-negative breast cancer in a subject in need thereof further includes administering a cytoskeletal-disrupting chemotherapeutic agent (e.g., nab-paclitaxel) to the subject.

Where the cancer is a urothelial bladder cancer, in certain embodiments, the method includes administering to a subject in need thereof an effective amount of a multi-specific binding protein disclosed herein, wherein the subject has received (a) a platinum containing chemotherapy and/or (b) a previously administered PD-1 inhibitor (e.g., an anti-PD-1 antibody) as one or more previous therapies (e.g., one or more previous lines of therapies). Where the previous treatment(s) include (a), (b), or a combination of (a) and (b), in certain embodiments, the urothelial bladder cancer has radiographic disease progression after a last line of therapy. Where the subject has received at least (b), in certain embodiments, the urothelial bladder cancer has radiographic disease progression during treatment with the previously administered PD-1 inhibitor. In certain embodiments, the urothelial bladder cancer is a locally advanced or metastatic transitional cell carcinoma of the urothelium. In certain embodiments, the urothelial bladder cancer has a HER2 level scored 1+ or higher by immunohistochemistry. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating a urothelial bladder cancer, the method including administering to a subject in need thereof effective amounts of a PD-1 inhibitor and a multi-specific binding protein disclosed herein. In certain embodiments, the urothelial bladder cancer has a HER2 level scored 1+ or higher by immunohistochemistry. the subject has received (a) a platinum containing chemotherapy and/or (b) a previously administered PD-1 inhibitor (e.g., an anti-PD-1 antibody) as one or more previous therapies (e.g., one or more previous lines of therapies). Where the previous treatment(s) include (a), (b), or a combination of (a) and (b), in certain embodiments, the urothelial bladder cancer has radiographic disease progression after a last line of therapy. Where the subject has received at least (b), in certain embodiments, the urothelial bladder cancer has radiographic disease progression during treatment with the previously administered PD-1 inhibitor. In certain embodiments, the urothelial bladder cancer is a locally advanced or metastatic transitional cell carcinoma of the urothelium. In certain embodiments, the PD-1 inhibitor administered in combination with the multi-specific binding protein includes an anti-PD-1 antibody, optionally nivolumab.

Where the cancer is a non-small-cell lung cancer (NSCLC), in certain embodiments, the method includes administering to a subject in need thereof an effective amount of a multi-specific binding protein disclosed herein, wherein the subject has received (a) a platinum doublet-based chemotherapy and/or (b) a PD-1 inhibitor as one or more previous therapies. Where the subject has received at least (a) (e.g., as a previous line of therapy), in certain embodiments, the NSCLC is recurrent or progressive during or after the chemotherapy. Where the subject has received at least (b) (e.g., as a previous line of therapy), in certain embodiments, the NSCLC has progressed during or after the previously administered PD-1 inhibitor therapy. Previous treatment with both (a) and (b) is also contemplated. In certain embodiments, the NSCLC is stage IIIB, stage IV, or recurrent. In certain embodiments, the NSCLC has a HER2 level scored 2+ or higher by immunohistochemistry and has ERBB2 gene amplification. In certain embodiments, the NSCLC has a HER2 level scored 1+ or higher by immunohistochemistry and has no ERBB2 gene amplification. The multi-specific binding protein can be provided as a monotherapy. Alternatively, the method can further include administering to the subject a PD-1 inhibitor, e.g., an anti-PD-1 antibody, e.g., nivolumab.

Also provided is a method of treating a NSCLC, the method including administering to a subject in need thereof an effective amount of a multi-specific binding protein disclosed herein, wherein the NSCLC has a HER2 level scored 1+ or higher by immunohistochemistry and has no ERBB2 gene amplification. In certain embodiments, the NSCLC is stage IIIB, stage IV, or recurrent. In certain embodiments, the subject has received (a) a platinum doublet-based chemotherapy and/or (b) a PD-1 inhibitor as one or more previous therapies. Where the subject has received at least (a) (e.g., as a previous line of therapy), in certain embodiments, the NSCLC is recurrent or progressive during or after the chemotherapy. Where the subject has received at least (b) (e.g., as a previous line of therapy), in certain embodiments, the NSCLC has progressed during or after the previously administered PD-1 inhibitor therapy. Previous treatment with both (a) and (b) is also contemplated. The multi-specific binding protein can be provided as a monotherapy. Alternatively, the method can further include administering to the subject a PD-1 inhibitor, e.g., an anti-PD-1 antibody, e.g., nivolumab.

It is also contemplated that the multi-specific binding protein disclosed herein may be used to treat cancer that have low HER2 expression levels, e.g., HER2 levels scored as 0 by immunohistochemistry. Accordingly, the present disclosure provides a method of treating a cancer having a HER2 level scored as 0 by immunohistochemistry, the method including administering to a subject in need thereof an effective amount of a multi-specific binding protein disclosed herein. It is understood that the cancer can nevertheless include HER2-expressing tumor cells, and in certain embodiments, the cancer has detectable HER2 expression in at least 1% (e.g., at least 2%, at least 3%, at least 4%, at least 5%, at least 6%, at least 7%, at least 8%, at least 9%) of cancer cells. In certain embodiments, the cancer does not have ERBB2 gene amplification.

Also provided is a method of treating cancer, the method including administering to a subject in need thereof, without first assessing HER2 expression level of the cancer, an effective amount of a multi-specific binding protein disclosed herein. In certain embodiments, the method does not include assessing ERBB2 gene amplification status of the cancer prior to the administration. In certain embodiments, the method does not include obtaining a biopsy of the cancer prior to the administration.

The two methods disclosed above can be used to treat solid tumors, e.g., breast cancer, gastric cancer, or esophageal cancer (e.g., an adenocarcinoma of the esophagus). The multi-specific binding protein can be provided as a monotherapy. Alternatively, the method can further include administering to the subject a PD-1 inhibitor, e.g., an anti-PD-1 antibody, e.g., nivolumab.

In some embodiments, the multi-specific binding protein is administered to the subject in an initial four-week treatment cycle on Day 1, Day 8, and Day 15. In some embodiments, the multi-specific binding protein is not administered on Day 22 of the initial treatment cycle. In some embodiments, after the initial treatment cycle, the multi-specific binding protein is administered on Day 1 and Day 15 of one or more subsequent four-week treatment cycles.

In some embodiments, the multi-specific binding protein is administered, in each dose, at an amount selected from 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1.0 mg/kg, 1.6 mg/kg, 5.2 mg/kg, 10 mg/kg, 15 mg/kg, 20 mg/kg, and 50 mg/kg.

In some embodiments, the multi-specific binding protein is administered, on Day 1 of the initial treatment cycle, at the amount of 5 mg/kg. The multi-specific binding protein can be administered, in each subsequent dose, at the amount of 15 mg/kg, 20 mg/kg, 10 mg/kg, or 5 mg/kg. For example, once a subsequent dose is selected from 15 mg/kg, 20 mg/kg, 10 mg/kg, and 5 mg/kg, this dose is used in all subsequent administration after Day 1 of the initial treatment cycle. This dosage regimen, with a priming dose at 5 mg/kg, is designed to improve tolerance to adverse effects and is contemplated for methods of treating various cancers (e.g., solid tumors), including but not limited to the specific types of cancer disclosed above.

In some embodiments, the multi-specific binding protein is administered by intravenous infusion. In some embodiments, the multi-specific binding protein is administered by intravenous infusion over a period of 3-4 hours in the initial treatment cycle, and over a period of 1-2 hours in each subsequent treatment cycle. The slower infusion in early doses is also designed to improve tolerance to adverse effects and contemplated for methods of treating various cancers (e.g., solid tumors), including but not limited to the specific types of cancer disclosed above.

In some embodiments, methods for treating cancer disclosed herein include administering a PD-1 inhibitor (e.g., an anti-PD-1 antibody) to the subject. In some embodiments, the anti-PD-1 antibody is nivolumab. In some embodiments, nivolumab is administered to the subject at a dose of 120 to 600 mg (e.g., 480 mg). In some embodiments, nivolumab is administered once every four weeks. In some embodiments, the multi-specific binding protein is administered in one or more four-week treatment cycles, and nivolumab is administered on day 1 of the same treatment cycles. In some embodiments nivolumab is administered to the subject by intravenous infusion.

In some embodiments, methods for treating cancer disclosed herein include administering a cytoskeletal-disrupting chemotherapeutic agent to the subject. In some embodiments, the cytoskeletal-disrupting chemotherapeutic agent is nab-paclitaxel. In some embodiments, nab-paclitaxel is administered to the subject at a dose of 50 to 300 mg/m 2. (e.g., 100 mg/m 2). In some embodiments, nab-paclitaxel is administered three times every four weeks. In some embodiments, the multi-specific binding protein is administered in one or more four-week treatment cycles, and nab-paclitaxel is administered on Day 1, Day 8, and Day 15 of the same treatment cycles. In some embodiments, nab-paclitaxel is not administered on Day 22 of the treatment cycles. In some embodiments, nab-paclitaxel is administered to the subject by intravenous infusion.

In some embodiments, multi-specific binding proteins administered in the methods disclosed herein incorporate a first antigen-binding site that includes a Fab having a heavy chain variable domain (VH) and a light chain variable domain (VL). In some embodiments, the VH of the Fab has complementarity-determining region 1 (CDR1), complementarity-determining region 2 (CDR2), and complementarity-determining region 3 (CDR3) sequences of SEQ ID NOs: 168, 96, and 188, respectively, and the VL of the Fab has CDR1, CDR2, and CDR3 sequences of SEQ ID NOs: 99, 100, and 101, respectively. In some embodiments, the VH of the Fab has CDR1, CDR2, and CDR3 sequences of SEQ ID NOs: 168, 96, and 169, respectively, and the VL of the Fab has CDR1, CDR2, and CDR3 sequences of SEQ ID NOs: 99, 100, and 101, respectively. In some embodiments, the VH of the Fab has an amino acid sequence at least 90% identical to SEQ ID NO:94, and the VL of the Fab has an amino acid sequence at least 90% identical to SEQ ID NO:98. In some embodiments the VH of the Fab has the amino acid sequence of SEQ ID NO:94, and the VL of the Fab has the amino acid sequence of SEQ ID NO:98.

In some embodiments, multi-specific binding proteins administered in the methods disclosed herein incorporate a second antigen-binding site that includes a single chain variable fragment (scFv) having a VH and a VL. In some embodiments, the VH of the scFv has CDR1, CDR2, and CDR3 sequences of SEQ ID NOs: 115, 116, and 117, respectively, and the VL of the scFv has CDR1, CDR2, and CDR3 sequences of SEQ ID NOs: 119, 120, and 121, respectively. In some embodiments, the VH of the scFv has an amino acid sequence at least 90% identical to SEQ ID NO:195, and the VL of the scFv has an amino acid sequence at least 90% identical to SEQ ID NO:196. In some embodiments, the VH of the scFv has the amino acid sequence of SEQ ID NO:195, and the VL of the scFv has the amino acid sequence of SEQ ID NO:196. In some embodiments, the VL of the scFv is linked to the VH of the scFv via a flexible linker. In some embodiments, the flexible linker comprises or consists of the amino acid sequence of SEQ ID NO:143. In some embodiments, the VL of the scFv is positioned to the N-terminus of the VH of the scFv. In some embodiments, the VH of the scFv forms a disulfide bridge with the VL of the scFv. In some embodiments, the disulfide bridge is formed between C44 of the VH and C100 of the VL. In some embodiments, the scFv has the amino acid sequence of SEQ ID NO:139.

In some embodiments, multi-specific binding proteins administered in the methods disclosed herein incorporate an antibody Fc domain including a first antibody Fc sequence linked to the Fab and a second antibody Fc sequence linked to the scFv. In some embodiments, the first antibody Fc sequence is linked to the heavy chain portion of the Fab. In some embodiments, the scFv is linked to the second antibody Fc sequence via a hinge comprising Ala-Ser. In some embodiments, the first and second antibody Fc sequences each comprise a hinge and a CH2 domain of a human IgG1 antibody. In some embodiments, the first and second antibody Fc sequences each have an amino acid sequence at least 90% identical to amino acids 234-332 of a wild-type human IgG1 antibody. In some embodiments, the first and second antibody Fc sequences incorporate different mutations that promote heterodimerization. In some embodiments, the first antibody Fc sequence is a human IgG1 Fc sequence incorporating K360E and K409W substitutions. In some embodiments, the second antibody Fc sequence is a human IgG1 Fc sequence incorporating Q347R, D399V, and F405T substitutions.

In some embodiments, a multi-specific binding protein administered in the methods disclosed herein include: a first polypeptide having the amino acid sequence of SEQ ID NO:141, a second polypeptide having the amino acid sequence of SEQ ID NO:140, and a third polypeptide having the amino acid sequence of SEQ ID NO:142.

Also disclosed herein, in various embodiments, are pharmaceutical compositions formulated prior to administration to a subject in need thereof. Some pharmaceutical compositions of the present disclosure contain at least 10 mg/mL, 10 mg/mL to equal to or greater than 50 mg/mL, 10 mg/mL to 25 mg/mL, or about 15 mg/mL of the multi-specific binding protein.

In some embodiments, pharmaceutical compositions of the present disclosure have a pH in the range of 5.5 to 6.5, or about 6.0. In some embodiments, pharmaceutical compositions of the present disclosure contain 5 to 50 mM, 10 to 25 mM histidine, or about 20 mM histidine. In some embodiments, pharmaceutical compositions of the present disclosure contain 50 to 300 mM, 150 to 300 mM, or about 250 mM sugar or sugar alcohol. In some embodiments, the sugar is sucrose. In some embodiments, pharmaceutical compositions of the present disclosure contain to 0.05% mM (w/v) polysorbate, 0.005% to 0.02% mM (w/v), or 0.01% (w/v) polysorbate. In some embodiments, the polysorbate is polysorbate-80.

Some pharmaceutical formulations of the resent disclosure contain: (i) 10 mg/mL to mg/mL of the multi-specific binding protein; (ii) 5 mM to 50 mM histidine; (iii) 50 mM to 300 mM sucrose; and (iv) 0.005% to 0.05% (w/v) polysorbate 80, at pH 5.5 to 6.5. Some pharmaceutical formulations of the resent disclosure contain: (i) 10 mg/mL to 25 mg/mL of the multi-specific binding protein; (ii) 10 mM to 25 mM histidine; (iii) 150 mM to 300 mM sucrose; and (iv) 0.005% to 0.02% (w/v) polysorbate 80, at pH 5.5 to 6.5. Some pharmaceutical formulations of the resent disclosure contain: (i) about 15 mg/mL of the multi-specific binding protein; (ii) about 20 mM histidine; (iii) about 250 mM sucrose; and (iv) about 0.01% (w/v) polysorbate 80, at about pH 6.0.

Other embodiments and details of the disclosure are presented herein below.

### BRIEF DESCRIPTION OF THE DRAWINGS

FIG. 1 is a representation of a heterodimeric, multi-specific antibody, e.g., a trispecific binding protein (TriNKET). Each arm can represent either an NKG2D binding domain or a tumor-associated antigen binding domain. In some embodiments, the NKG2D binding domain and the tumor-associated antigen binding domain can share a common light chain.

FIGS. 2A-2E illustrate five exemplary formats of a multi-specific binding protein, e.g., a trispecific binding protein (TriNKET). As shown in FIG. 2A, either the NKG2D-binding domain or the tumor-associated antigen binding domain can take the scFv format (left arm). An antibody that contains an NKG2D targeting scFv, a tumor-associated antigen targeting Fab fragment, and a heterodimerized antibody constant region is referred herein as the F3-TriNKET. An antibody that contains a tumor-associated antigen targeting scFv, an NKG2D targeting Fab fragment, and a heterodimerized antibody constant region/domain that binds CD16 is referred herein as the F3′-TriNKET (FIG. 2E). As shown in FIG. 2B, both the NKG2D binding domain and a tumor-associated antigen binding domain can take the scFv format. FIGS. 2C to 2D are illustrations of an antibody with three antigen-binding sites, including two antigen-binding sites that bind a tumor-associated antigen, and the NKG2D-binding site fused to the heterodimerized antibody constant region. These antibody formats are referred herein as F4-TriNKET. FIG. 2C illustrates that the two tumor-associated antigen binding sites are in the Fab fragment format, and the NKG2D binding site in the scFv format. FIG. 2D illustrates that the tumor-associated antigen binding sites are in the scFv format, and the NKG2D binding site is in the scFv format. FIG. 2E represents a trispecific antibody (TriNKET) that contains a tumor-associated antigen targeting scFv, an NKG2D targeting Fab fragment, and a heterodimerized antibody constant region/domain (“CD domain”) that binds CD16. The antibody format is referred herein as F3′-TriNKET. In certain exemplary multi-specific binding proteins, heterodimerization mutations on the antibody constant region include K360E and K409W on one constant domain; and Q347R, D399V and F405T on the opposite constant domain (shown as a triangular lock-and-key shape in the CD domains). The bold bar between the heavy and the light chain variable domains of the Fab fragments represents a disulfide bond.

FIG. 3 is a representation of a TriNKET in the Triomab form, which is a trifunctional, bispecific antibody that maintains an IgG-like shape. This chimera consists of two half antibodies, each with one light and one heavy chain, that originate from two parental antibodies. Triomab form may be a heterodimeric construct containing ½ of rat antibody and ½ of mouse antibody.

FIG. 4 is a representation of a TriNKET in the KiH Common Light Chain form, which involves the knobs-into-holes (KIHs) technology. KiH is a heterodimer containing 2 Fab fragments binding to target 1 and 2, and an Fc domain stabilized by heterodimerization mutations. TriNKET in the KiH format may be a heterodimeric construct with 2 Fab fragments binding to target 1 and target 2, containing two different heavy chains and a common light chain that pairs with both heavy chains.

FIG. 5 is a representation of a TriNKET in the dual-variable domain immunoglobulin (DVD-Ig™) form, which combines the target-binding domains of two monoclonal antibodies via flexible naturally occurring linkers, and yields a tetravalent IgG-like molecule. DVD-Ig™ is a homodimeric construct where variable domain targeting antigen 2 is fused to the N-terminus of a variable domain of a Fab fragment targeting antigen 1. DVD-Ig™ form contains a normal Fc domain.

FIG. 6 is a representation of a TriNKET in the Orthogonal Fab fragment interface (Ortho-Fab) form, which is a heterodimeric construct that contains 2 Fab fragments binding to target 1 and target 2 fused to Fc. Light chain (LC)-heavy chain (HC) pairing is ensured by orthogonal interface. Heterodimerization is ensured by mutations in the Fc domain.

FIG. 7 is a representation of a TriNKET in the 2-in-1 Ig format.

FIG. 8 is a representation of a TriNKET in the ES form, which is a heterodimeric construct containing two different Fab fragments binding to target 1 and target 2 fused to the Fc domain. Heterodimerization is ensured by electrostatic steering mutations in the Fc domain.

FIG. 9 is a representation of a TriNKET in the Fab Arm Exchange form: antibodies that exchange Fab fragment arms by swapping a heavy chain and attached light chain (half-molecule) with a heavy-light chain pair from another molecule, resulting in bispecific antibodies. Fab Arm Exchange form (cFae) is a heterodimer containing 2 Fab fragments binding to target 1 and 2, and an Fc domain stabilized by heterodimerization mutations.

FIG. 10 is a representation of a TriNKET in the SEED Body form, which is a heterodimer containing 2 Fab fragments binding to target 1 and 2, and an Fc domain stabilized by heterodimerization mutations.

FIG. 11 is a representation of a TriNKET in the LuZ-Y form, in which a leucine zipper is used to induce heterodimerization of two different HCs. The LuZ-Y form is a heterodimer containing two different scFabs binding to target 1 and 2, fused to an Fc domain. Heterodimerization is ensured through leucine zipper motifs fused to the C-terminus of the Fc domain.

FIG. 12 is a representation of a TriNKET in the Cov-X-Body form.

FIGS. 13A-13B are representations of TriNKETs in the κλ-Body forms, which are heterodimeric constructs with two different Fab fragments fused to a Fc domain stabilized by heterodimerization mutations: one Fab fragment targeting antigen 1 contains kappa LC, and the second Fab fragment targeting antigen 2 contains lambda LC. FIG. 13A is an exemplary representation of one form of a κλ-Body; FIG. 13B is an exemplary representation of another κλ-Body.

FIG. 14 is a representation of an Oasc-Fab heterodimeric construct that includes a Fab fragment binding to target 1 and an scFab binding to target 2, both of which are fused to the Fc domain. Heterodimerization is ensured by mutations in the Fc domain.

FIG. 15 is a representation of a DuetMab, which is a heterodimeric construct containing two different Fab fragments binding to antigens 1 and 2, and an Fc domain that is stabilized by heterodimerization mutations. Fab fragments 1 and 2 contain differential S-S bridges that ensure correct light chain and heavy chain pairing.

FIG. 16 is a representation of a CrossmAb, which is a heterodimeric construct with two different Fab fragments binding to targets 1 and 2, and an Fc domain stabilized by heterodimerization mutations. CL and CH1 domains, and VH and VL domains are switched, e.g., CH1 is fused in-line with VL, and CL is fused in-line with VH.

FIG. 17 is a representation of a Fit-Ig, which is a homodimeric construct where a Fab fragment binding to antigen 2 is fused to the N-terminus of HC of a Fab fragment that binds to antigen 1. The construct contains a wild-type Fc domain.

FIG. 18 is a graph of concentration curves of A49-F3′-TriNKET-Trastuzumab over time in formulations stored at 2-8° C. or 25° C.

FIG. 19A is an image of vials containing formulations of A49-F3′-TriNKET-Trastuzumab in varying concentrations of polysorbate-80. FIG. 19B is a graph of % Monomer species of A49-F3′-TriNKET-Trastuzumab in formulations stored at varying temperatures at specific time points as measured by size-exclusion high-performance liquid chromatography (SE-HPLC).

FIGS. 20A-20C are line graphs showing the cytotoxicity of A49-F3′-TriNKET-Trastuzumab and trastuzumab on SKBR-3 (FIG. 20A), H661 (FIG. 20B), and 786-0 (FIG. 20C) cancer cells in the presence of NK cells.

FIGS. 21A-21B are line graphs showing the cytotoxicity of A49-F3′-TriNKET-Trastuzumab and trastuzumab on ZR-75-1 (FIG. 21A) and MCF-7 (FIG. 21B) cancer cells in the presence of NK cells.

FIGS. 22A-22C are line graphs showing the cytotoxicity of A49-F3′-TriNKET-Trastuzumab and trastuzumab on BT-20 (FIG. 22A), Hs578T (FIG. 22B) and DU4475 (FIG. 22C) cancer cells in the presence of KHYG-1-CD16V cells.

FIGS. 23A-23B are diagrams detailing murine tumor models used in Example 4. FIG. 23A diagrams a murine tumor model of B16F10 tumors in B6 mice treated with Tryp1-TriNKET. FIG. 23B diagrams a murine tumor model of CT26-Tyrp1 tumors in B6 mice treated with Tyrp1-TriNKET.

FIGS. 24A-24D detail TriNKET anti-tumor efficacy in the B16F10 tumor model.

FIG. 24A shows graphs detailing average and individual tumor volumes are shown with treatment days and frequencies as indicated. FIG. 24B is a graph showing the weights of tumor tissue harvested at endpoint (Day 14 post-inoculation). FIG. 24C is a graph showing quantification (# of cells/gram tumor) of tumor-infiltrating NK cells, CD8+ T cells, and CD4+ T cells in mice treated with control or TriNKET. FIG. 24D is a graph showing quantification (# of cells/gram tumor) of tumor-infiltrating immune cells (from top to bottom, TAMs, MDSCs, NK cells, CD8 T cells, CD4 T cells, and other) in B16F10 tissues from mice treated 7 days with TriNKET or control (right panel) and the frequencies of indicated immune cells with in the total CD45+ leukocyte population (left panel).

FIGS. 25A-25B show infiltration of immune cells in B16F10 tumor tisues in mice treated with control or TriNKET. FIG. 25A shows quantification of tumor-infiltrating NK cells, CD8a+ T cells and CD4+ T cells that express the indicated activation markers in B16F10 tumor tissues. FIG. 25B shows quantification of tumor-infiltrating NK cells, CD8a+ T cells and CD4+ T cells that express the indicated immune checkpoint markers.

FIG. 26 shows quantification of tumor-infiltrating memory CD8a+ CTLs and CD4+ T cells in subcutaneous B16F10 tumors from mice treated 7 days with TriNKET or control. Graphs show the mean cell numbers of CD8+ CTLs and CD4+ T cells marked by CD44HiCD62LHi (Central memory; Tcm) or CD44HiCD62LLow (Effector memory; T_{eff}).

FIGS. 27A-27C show immune infiltrate changes in tumor draining and non-draining lymph nodes after TriNKET treatment. FIG. 27A shows absolute numbers of NK cells, CD8+ T cells and CD4+ T cells in the tumor-draining lymph node (dLN). FIG. 27B shows absolute numbers of NK cells, CD8+ T cells and CD4+ T cells in the tumor-draining lymph node (ndLN).

FIG. 27C shows the frequency of NK cells within the total cells analyzed in the ndLN at Day 5 post-treatment.

FIGS. 28A-28C show activated NK cells and CD8+ T cells that express immune-checkpoint markers in dLN of B16F10 tumor-bearing mice. FIG. 28A shows quantification (#cells) of NK cells, CD8a+ T cells and CD4+ T cells that express the indicated activation markers. FIG. 28B shows the frequency of subset of NK cells expressing 41BB, LAG3 or PD1 in dLN. FIG. 28C shows quantification (#cells) of NK cells, CD8a+ T cells and CD4+ T cells that express the indicated immune checkpoint markers.

FIGS. 29A-29B show changes in activation or immune-checkpoint marker expression on immune cells in ndLN of B16F10 TBM after TriNKET treatment. FIG. 29A shows quantification (#cells) of NK cells, CD8a+ T cells and CD4+ T cells that express the indicated activation markers in ndLN of B16F10 TBM treated for 7 days with TriNKET or control. FIG. 29A shows quantification (#cells) of NK cells, CD8a+ T cells and CD4+ T cells that express the indicated immune checkpoint markers in ndLN of B16F10 TBM treated for 7 days with TriNKET or control.

FIGS. 30A-30B show quantification of memory CD8+ CTL and CD4+ T cells in tumor draining and non-draining lymph nodes after TriNKET treatment. FIG. 30A shows absolute cell number of memory CD8+ CTLs and CD4+ T cells in the tumor-draining lymph nodes harvested from B16F10 tumor bearing mice after 7 days of treatment with TriNKET or control. FIG. 30B shows absolute cell number of memory CD8+ CTLs and CD4+ T cells in the non-draining lymph nodes harvested from B16F10 tumor bearing mice after 7 days of treatment with TriNKET or control.

FIG. 31 is graph showing the percentage of tumor-infiltrating immune cells expressing PD-1 from B16F10 tumor bearing mice after 7 days of treatment with TriNKET or control.

FIGS. 32A-32D show TriNKET anti-tumor efficacy in the CT26-Tyrp1 tumor model. FIG. 32A shows average and individual tumor volumes with treatment days and frequencies as indicated. FIG. 32B is a graph indicating the weights of tumor tissue harvested at endpoint (Day 13 post-inoculation) from mice treated 5 days with TriNKET or control. FIG. 32C is a graph showing quantification (# of cells/gram tumor) of tumor-infiltrating NK cells, CD8+ T cells, and CD4+ T cells in mice treated with control or TriNKET. FIG. 32D is a graph showing quantification (# of cells/gram tumor) of tumor-infiltrating immune cells (from top to bottom, TAMs, MDSCs, NK cells, CD8 T cells, CD4 T cells, and other) in CT26-Tyrp1 tissues from mice treated 5 days with TriNKET or mIgG2a (right panel) and the frequencies of indicated immune cells with in the total CD45+ leukocyte population (left panel).

FIG. 33 shows quantification of tumor-infiltrating NK cells, CD8a+ T cells and CD4+ T cells that express the indicated activation markers (top panels) or immune checkpoint markers (bottom panels) in CT26-Tyrp1 tumor tissues.

FIG. 34 shows quantification of tumor-infiltrating memory CD8a+ CTLs and CD4+ T cells in CT26-Tyrp1 tumors from mice treated 5 days with TriNKET or mIgG2a. Graphs show the mean cell numbers of CD8+ CTLs and CD4+ T cells marked by CD44HiCD62LHi (Central memory; Tcm) or CD44HiCD62LLow (Effector memory; T_{eff}).

FIGS. 35A-35C show immune infiltrate changes in tumor draining and non-draining lymph nodes after TriNKET treatment. FIG. 35A shows the absolute cell number of NK cells, CD8+ T cells and CD4+ T cells in the tumor-draining lymph nodes harvested from CT26-Tyrp1 tumor bearing mice after 5 days of treatment with TriNKET or control. FIG. 35B shows the absolute cell number of NK cells, CD8+ T cells and CD4+ T cells in the non-draining lymph nodes harvested from CT26-Tyrp1 tumor bearing mice after 5 days of treatment with TriNKET or control. FIG. 35C shows the frequency of NK cells in the non-draining lymph nodes harvested from CT26-Tyrp1 tumor bearing mice after 5 days of treatment with TriNKET or control.

FIGS. 36A-36C show activated NK cells and CD8+ T cells that express immune-checkpoint markers in dLN of CT26-Tyrp1 tumor-bearing mice. FIG. 36A shows quantification (#cells) of NK cells, CD8a+ T cells and CD4+ T cells that express the indicated activation markers. FIG. 36B shows the frequency of subset of NK cells expressing 41BB, LAG3 or PD1 in dLN. FIG. 36C shows quantification (#cells) of NK cells, CD8a+ T cells and CD4+ T cells that express the indicated immune checkpoint markers.

FIGS. 37A-37B show changes in activation or immune-checkpoint marker expression on immune cells in ndLN of CT26-Tyrp1 tumor-bearing mice after TriNKET treatment. FIG. 37A shows quantification (#cells) of NK cells, CD8a+ T cells and CD4+ T cells that express the indicated activation markers in ndLN of CT26-Tyrp1 tumor-bearing mice treated for 5 days with TriNKET or control. FIG. 37B shows quantification (#cells) of NK cells, CD8a+ T cells and CD4+ T cells that express the indicated immune checkpoint markers in ndLN of CT26-Tyrp1 tumor-bearing mice treated for 5 days with TriNKET or control.

FIGS. 38A-38B show quantification of memory CD8+ CTL and CD4+ T cells in tumor draining and non-draining lymph nodes after TriNKET treatment. FIG. 38A shows absolute cell number of memory CD8+ CTLs and CD4+ T cells in the tumor-draining lymph nodes harvested from CT26-Tyrp1 tumor bearing mice after 5 days of treatment with TriNKET or control. FIG. 38B shows absolute cell number of memory CD8+ CTLs and CD4+ T cells in the non-draining lymph nodes harvested from CT26-Tyrp1 tumor bearing mice after 5 days of treatment with TriNKET or control.

FIG. 39 is schematic illustration of a clinical trial design, including phase 1 and phase 2 studies of A49-F3′-TriNKET-Trastuzumab.

### DETAILED DESCRIPTION

### Definitions

To facilitate an understanding of the present invention, a number of terms and phrases are defined below.

The terms “a” and “an” as used herein mean “one or more” and include the plural unless the context is inappropriate.

As used herein, the terms “Fab” and “scFv” refer to two different forms of protein fragments that each include an antigen-binding site. The term “antigen-binding site” refers to the part of the immunoglobulin molecule that participates in antigen binding. In human antibodies, the antigen-binding site is formed by amino acid residues of the N-terminal variable (“V”) regions of the heavy (“H”) and light (“L”) chains, which are also called “VH” and “VL,” respectively. Three highly divergent stretches within the V regions of the heavy and light chains are referred to as “hypervariable regions” which are interposed between more conserved flanking stretches known as “framework regions,” or “FR.” Thus the term “FR” refers to amino acid sequences which are naturally found between and adjacent to hypervariable regions in immunoglobulins. In a human antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementarity-determining regions,” or “CDRs.” In certain animals, such as camels and cartilaginous fish, the antigen-binding site is formed by a single antibody chain providing a “single domain antibody.” Antigen-binding sites can exist in an intact antibody, in an antigen-binding fragment of an antibody that retains the antigen-binding surface such as a Fab, or in a recombinant polypeptide such as an scFv, using a peptide linker to connect the heavy chain variable domain to the light chain variable domain in a single polypeptide. All the amino acid positions in heavy or light chain variable regions disclosed herein are numbered according to Kabat numbering, unless otherwise indicated.

The CDRs of an antigen-binding site can be determined by the methods described in Kabat et al., J. Biol. Chem. 252, 6609-6616 (1977) and Kabat et al., Sequences of protein of immunological interest. (1991), Chothia et al., J. Mol. Biol. 196:901-917 (1987), and MacCallum et al., J. Mol. Biol. 262:732-745 (1996). The CDRs determined under these definitions typically include overlapping or subsets of amino acid residues when compared against each other. In certain embodiments, the term “CDR” is a CDR as defined by MacCallum et al., J. Mol. Biol. 262:732-745 (1996) and Martin A., Protein Sequence and Structure Analysis of Antibody Variable Domains, in Antibody Engineering, Kontermann and Dubel, eds., Chapter 31, pp. 422-439, Springer-Verlag, Berlin (2001). In certain embodiments, the term “CDR” is a CDR as defined by Kabat et al., J. Biol. Chem. 252, 6609-6616 (1977) and Kabat et al., Sequences of protein of immunological interest. (1991). In certain embodiments, heavy chain CDRs and light chain CDRs of an antibody are defined using different conventions. For example, in certain embodiments, the heavy chain CDRs are defined according to MacCallum (supra), and the light CDRs are defined according to Kabat (supra). CDRH1, CDRH2 and CDRH3 denote the heavy chain CDRs, and CDRL1, CDRL2 and CDRL3 denote the light chain CDRs.

The term “tumor-associated antigen” or “TAA,” as used herein, means any antigen including but not limited to a protein, glycoprotein, ganglioside, carbohydrate, or lipid that is associated with cancer. In certain embodiments, a tumor-associated antigen is expressed on the surface of a cell. For example, a tumor-associated antigen can be expressed on malignant cells or in the tumor microenvironment, such as on tumor-associated blood vessels, extracellular matrix, mesenchymal stroma, or immune infiltrates.

The terms “HER2 high,” “HER2-high,” and “HER2-overexpressing,” as used interchangeably herein to characterize a cancer (e.g., breast cancer, gastric cancer, or esophageal cancer), means that the cancer is eligible for treatment with trastuzumab according to the ASCO/CAP or ASCP HER2 testing guideline (Wolff et al., (2007) J. Clin. Oncol. 25(1):118-45) and the 2018 update (Wolff et al., (2018) J. Clin. Oncol. 36(20):2105-22). A HER2-overexpressing cancer can have a HER2 expression level scored as 3+ by immunohistochemistry (IHC), or a HER2 expression level scored as 2+ by immunohistochemistry further supported by detection of ERBB2 gene amplification (e.g., determined by in situ hybridization (ISH), chromogenic in situ hybridization (CISH), quantitative PCR, or DNA sequencing).

The terms “HER2 low” and “HER2-low,” as used interchangeably herein to characterize a cancer (e.g., breast cancer, gastric cancer, or esophageal cancer), means that the cancer has detectable expression of HER2 assessed by IHC (e.g., Herceptest) in at least 1% of tumor cells, but has no ERBB2 gene amplification. It is understood that a HER2 low cancer can be “HER2 0” by Herceptest, given that a positive Herceptest result, with respect to certain cancer types (e.g., gastric cancer and breast cancer), may require more than 10% of cells stained positive for HER2.

The term “ERBB2 gene amplification,” as used herein to characterize a cancer, means the average copy number of ERBB2 gene in tumor cells is 4.0 or higher. ERBB2 gene amplification can be assessed by in situ hybridization (ISH) with an ERBB2 probe. An additional chromosome 17 enumeration probe (CEP17) may be used, in a dual-probe approach, to assist with the analysis. A detailed process for assessing ERBB2 gene amplification is provided in the 2018 ASCO/CAP guidelines.

As used herein, the term “pharmaceutical formulation” refers to the combination of an active agent with a carrier, inert or active, making the composition especially suitable for diagnostic or therapeutic use in vivo or ex vivo.

As used herein, the terms “subject” and “patient” refer to an organism to be treated by the methods and compositions described herein. Such organisms preferably include, but are not limited to, mammals (e.g., murines, simians, equines, bovines, porcines, primates, canines, felines, and the like), and more preferably include humans.

The terms “treat,” “treating,” or “treatment,” and other grammatical equivalents as used in this disclosure, include alleviating, abating, ameliorating, or preventing a disease, condition or symptoms, preventing additional symptoms, ameliorating or preventing the underlying metabolic causes of symptoms, inhibiting the disease or condition, e.g., arresting the development of the disease or condition, relieving the disease or condition, causing regression of the disease or condition, relieving a condition caused by the disease or condition, or stopping the symptoms of the disease or condition, and are intended to include prophylaxis. The terms further include achieving a therapeutic benefit and/or a prophylactic benefit. The term “therapeutic benefit” refers to eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient may still be afflicted with the underlying disorder.

As used herein, the term “effective amount” refers to the amount of a compound (e.g., a compound of the present invention) sufficient to effect beneficial or desired results. An effective amount can be administered in one or more administrations, applications or dosages and is not intended to be limited to a particular formulation or administration route.

As used herein, the term “monotherapy” means a single active agent is used to treat a disease or disorder in a subject as a line of therapy, in the absence of any other therapeutic agent administered to the subject to treat the same disease or disorder in the same line of therapy. It is understood that the subject may receive one or more other therapeutic agents to treat another disease, disorder, or condition. For example, when receiving a monotherapy, the subject may also receive one or more therapeutic agents to treat symptoms associated with the disease or disorder (e.g., inflammation, pain, weight loss, and/or general malaise associated with cancer) but not the underlying disease or disorder itself. In another example, when receiving a monotherapy, the subject may also receive one or more therapeutic agents to reduce adverse effects (e.g., infusion-related reactions) of the monotherapy.

As used herein, the term “PD-1 inhibitor” refers to a compound that inhibits PD-1 signaling, for example, by inhibiting the expression or activity of PD-1, PD-L1, and/or PD-L2, or by blocking the binding of PD-1 to PD-L1 or PD-L2. Exemplary PD-1 inhibitors include but are not limited to anti-PD-1 antibodies (e.g., nivolumab, pembrolizumab, or cemiplimab), anti-PD-L1 antibodies (e.g., atezolizumab, durvalumab, or avelumab), and anti-PD-L2 antibodies.

The term “about” refers to any minimal alteration in the concentration or amount of an agent that does not change the efficacy of the agent in preparation of a formulation and in treatment of a disease or disorder. In certain embodiments, the term “about” may include ±5%, ±10%, or ±15% of a specified numerical value or data point.

Ranges can be expressed in this disclosure as from “about” one particular value, and/or to “about” another particular value. When such a range is expressed, another aspect includes from the one particular value and/or to the other particular value. Similarly, when values are expressed as approximations, by use of the antecedent “about,” it is understood that the particular value forms another aspect. It is further understood that the endpoints of each of the ranges are significant both in relation to the other endpoint, and independently of the other endpoint. It is also understood that there are a number of values disclosed in this disclosure, and that each value is also disclosed as “about” that particular value in addition to the value itself. It is also understood that throughout the application, data are provided in a number of different formats and that this data represent endpoints and starting points and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units is also disclosed. For example, if 10 and 15 are disclosed, then 11, 12, 13, and 14 are also disclosed.

Throughout the description, where compositions are described as having, including, containing, incorporating, or comprising specific components, or where processes and methods are described as having, including, or comprising specific steps, it is intended that compositions and methods are inclusive or open-ended and do not exclude additional, unrecited components or steps. It is contemplated that, additionally, there are compositions of the present invention that consist essentially of, or consist of, the recited components, and that there are processes and methods according to the present invention that consist essentially of, or consist of, the recited steps.

As a general matter, compositions specifying a percentage are by weight unless otherwise specified. Further, if a variable is not accompanied by a definition, then the previous definition of the variable controls.

Multi-Specific Binding Proteins

The methods of treatment of the present disclosure use pharmaceutical compositions or pharmaceutical formulations that contain a multi-specific binding protein having an antigen-binding site that binds a tumor-associated antigen such as HER2, an antigen-binding site that binds NKG2D, and an antibody Fc domain or an antigen-binding site that binds CD16. Such multi-specific binding proteins are referred to as “TriNKET” herein. The multi-specific binding proteins and pharmaceutical formulations are useful in treating cancer, such as a locally advanced or metastatic solid tumor. The multi-specific binding proteins are capable of binding a target (e.g., HER2) on a cancer cell and NKG2D and CD16 on natural killer cells. Such binding brings the cancer cell into proximity with the natural killer cell, which facilitates direct and indirect destruction of the cancer cell by the natural killer cells.

The first component of the multi-specific binding proteins binds to NKG2D receptor-expressing cells, which can include but are not limited to NK cells, NKT cells, γδ T cells and CD8⁺ αβ T cells. Upon NKG2D binding, the multi-specific binding proteins may block natural ligands, such as ULBP6 and MICA, from binding to NKG2D and activating NK cells. The second component of the multi-specific binding proteins binds to HER2-expressing cells, which can include but are limited to breast, ovarian, esophageal, bladder and gastric cancer, salivary duct carcinoma, adenocarcinoma of the lung and aggressive forms of uterine cancer, such as uterine serous endometrial carcinoma. The third component of the multi-specific binding proteins is an antibody Fc domain, which binds to cells expressing CD16 such as NK cells, macrophages, neutrophils, eosinophils, mast cells, and follicular dendritic cells.

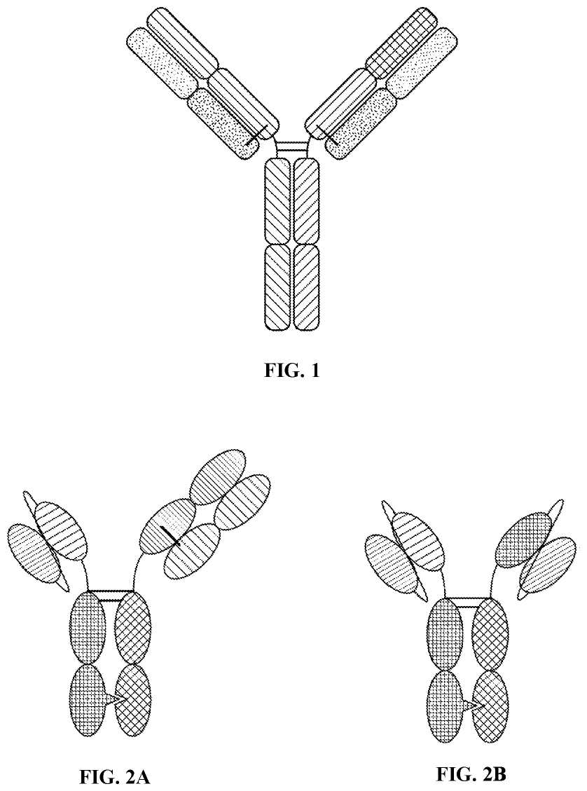

The multi-specific binding proteins described herein can take various formats. For example, one format involves a heterodimeric, multi-specific antibody including a first immunoglobulin heavy chain, a second immunoglobulin heavy chain and an immunoglobulin light chain (FIG. 1). The first immunoglobulin heavy chain includes a first Fc (hinge-CH2-CH3) domain fused via either a linker or an antibody hinge to an Fab fragment composed of a heavy chain portion including a heavy chain variable domain and a heavy chain CH1 domain, and a light chain portion including a light chain variable domain and a light chain constant domain (CL), and the heavy chain and light chain portions of the Fab fragment pair and bind NKG2D. The second immunoglobulin heavy chain includes a second Fc (hinge-CH2-CH3) domain fused via either a linker or an antibody hinge to a single-chain variable fragment (scFv) composed of a heavy chain variable domain and light chain variable domain which pair and bind the tumor-associate antigen.

In some embodiments, the single-chain variable fragment (scFv) described above is linked to the antibody constant domain via a hinge sequence. In some embodiments, the hinge has the amino acid sequence Ala-Ser. In some other embodiments, the hinge has the amino acid sequences Ala-Ser and Thr-Lys-Gly. The hinge sequence can provide flexibility of binding to the target antigen, and balance between flexibility and optimal geometry.

In some embodiments, the single-chain variable fragment (scFv) described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. For example, in some embodiments, the disulfide bridge is formed between a cysteine residue (naturally present or introduced by mutation) at position 44 (C44) of the VH of the scFv and a cysteine residue (naturally present or introduced by mutation) at position 100 (C100) of the VL of the scFv, numbered under the Kabat numbering scheme. In some embodiments, the heavy chain variable domain is linked to the light chain variable domain via a flexible linker. Any suitable linker can be used, for example, the (G4S)₄ linker (SEQ ID NO:143). In some embodiments of the scFv, the heavy chain variable domain is positioned at the N-terminus of the light chain variable domain. In some embodiments of the scFv, the heavy chain variable domain is positioned at the C terminus of the light chain variable domain.

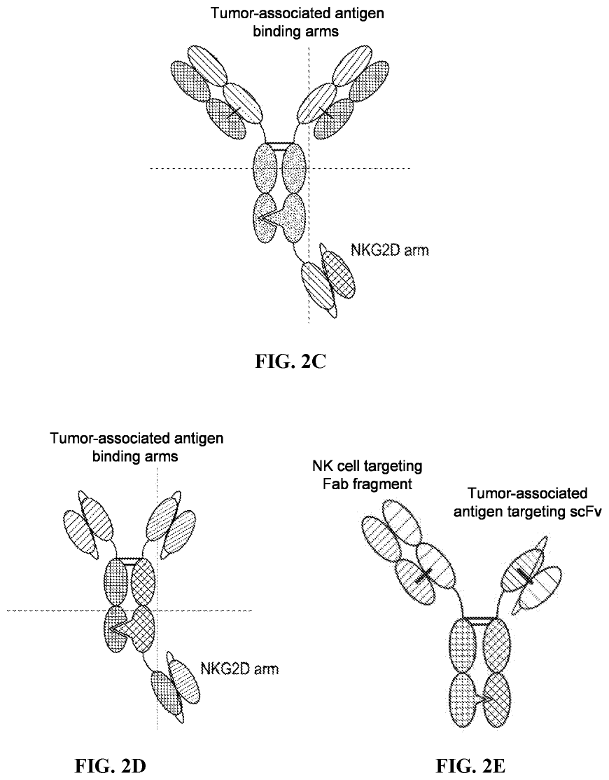

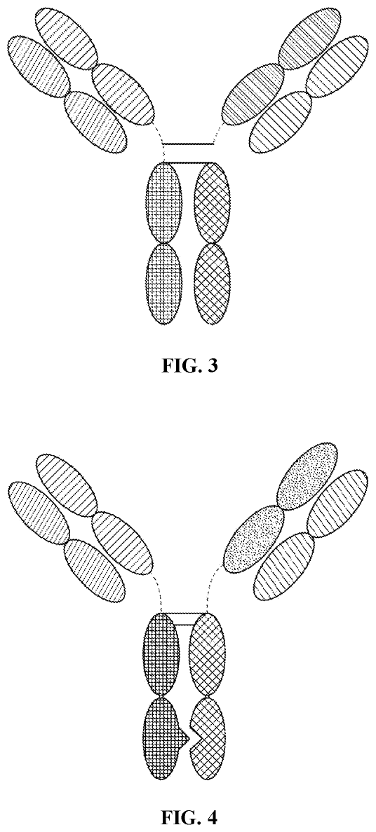

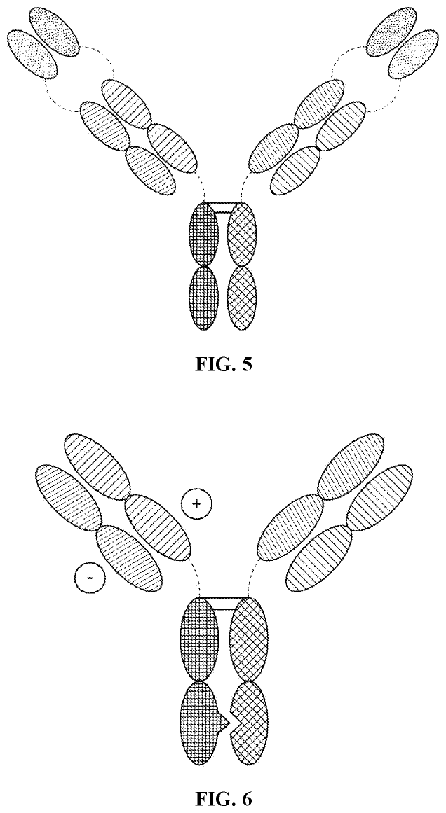

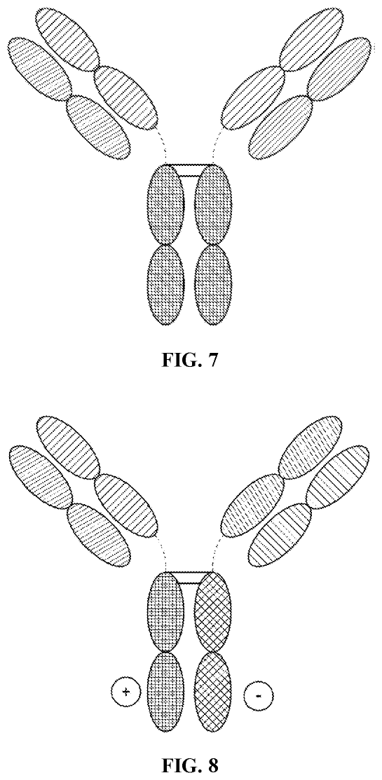

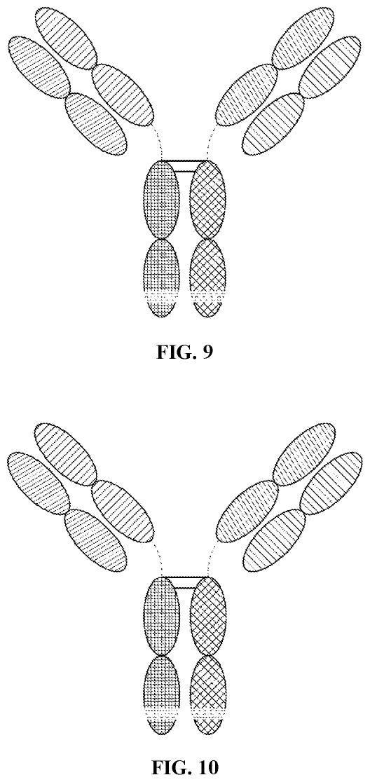

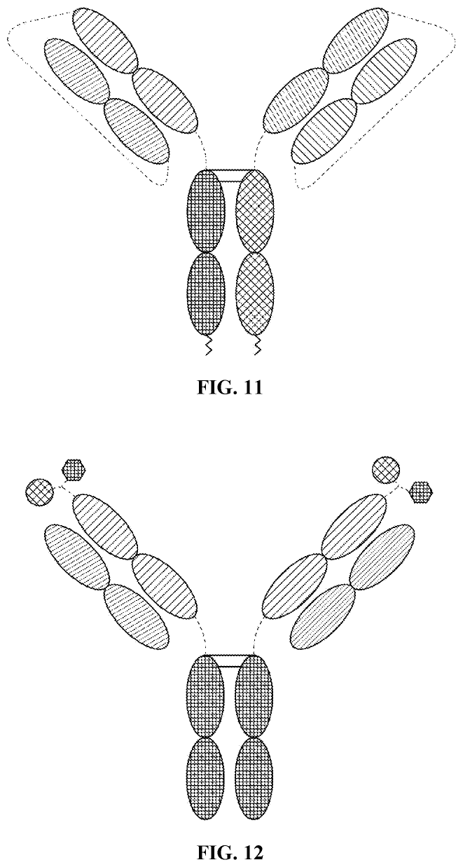

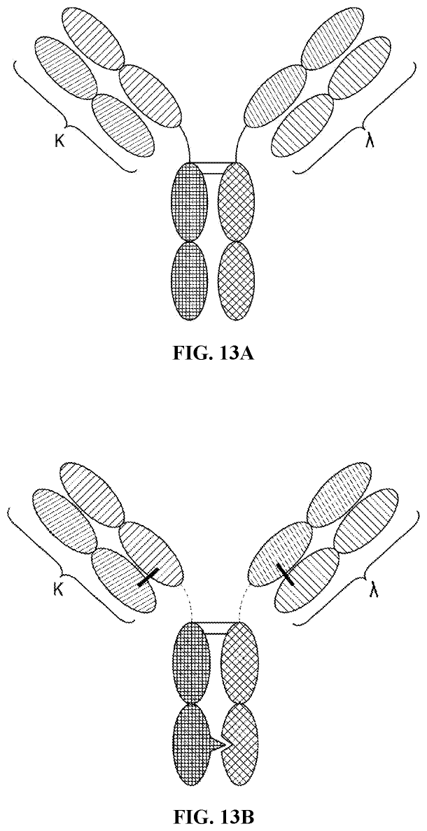

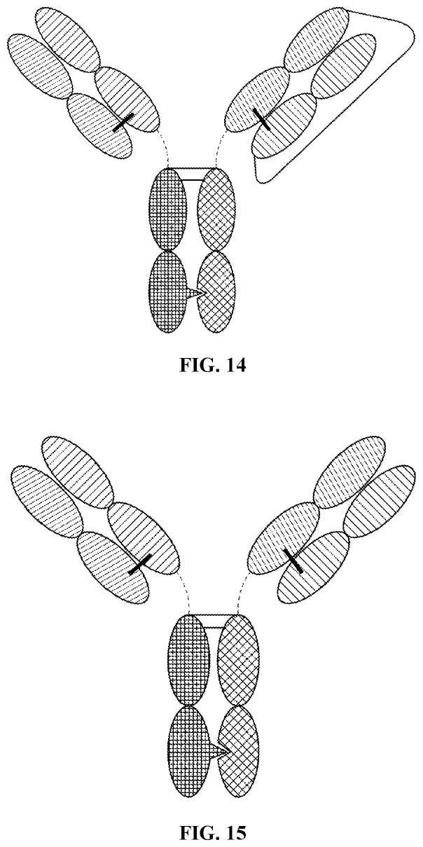

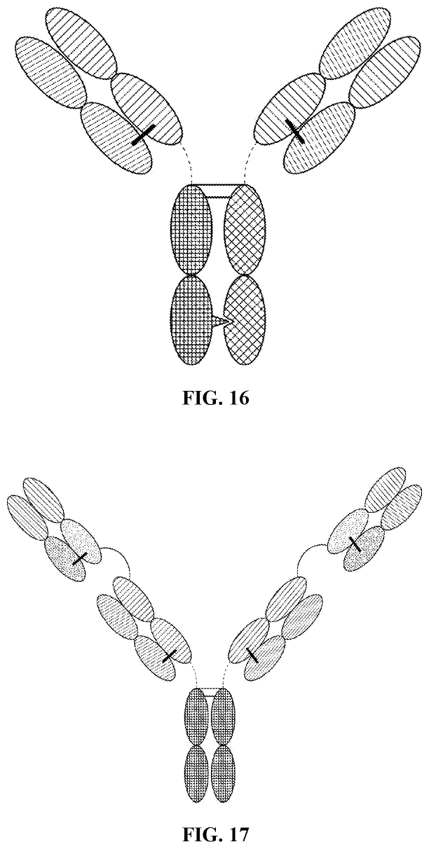

The multi-specific binding proteins described herein can further include one or more additional antigen-binding sites. The additional antigen-binding site(s) may be fused to the C-terminus of the constant region CH2 domain or to the C-terminus of the constant region CH3 domain, optionally via a linker sequence. In certain embodiments, the additional antigen-binding site(s) takes the form of a single-chain variable region (scFv) that is optionally disulfide-stabilized, resulting in a tetravalent or trivalent multi-specific binding protein. For example, a multi-specific binding protein includes an NKG2D-binding site, a TAA-binding site, a third antigen-binding site that binds a TAA, and an antibody constant region or a portion thereof sufficient to bind CD16, or a fourth antigen-binding site that binds CD16. Any one of these antigen-binding sites can either take the form of an Fab or an scFv, such as the scFv described above. In some embodiments, the third antigen-binding site binds a different TAA. In some embodiments, the third antigen-binding site binds to the same TAA, and the exemplary formats are shown in FIGS. 2C and 2D. Accordingly, the multi-specific binding proteins can provide bivalent engagement of the TAA. In certain embodiments, bivalent engagement of the TAA by the multi-specific proteins can stabilize the TAA on the cancer cell surface, and enhance cytotoxicity of NK cells towards the cancer cells. Bivalent engagement of the TAA by the multi-specific proteins can confer stronger binding of the multi-specific proteins to the cancer cells, thereby facilitating a stronger cytotoxic response of NK cells towards the cancer cells, especially towards cancer cells expressing a low level of the TAA.

Within the Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

In some embodiments, the antibody constant domain includes a CH2 domain and a CH3 domain of an IgG antibody, for example, a human IgG1 antibody. In some embodiments, mutations are introduced in the antibody constant domain to enable heterodimerization with another antibody constant domain. For example, if the antibody constant domain is derived from the constant domain of a human IgG1, the antibody constant domain can have an amino acid sequence at least 90% identical to amino acids 234-332 of a human IgG1 antibody that differs at one or more positions selected from Q347, Y349, L351, S354, E356, E357, K360, Q362, S364, T366, L368, K370, N390, K392, T394, D399, S400, D401, F405, Y407, K409, T411, and K439. All the amino acid positions in an Fc domain or hinge region disclosed herein are numbered according to EU numbering.

In some embodiments, the antibody constant domain can have an amino acid sequence at least 90% identical to amino acids 234-332 of a human IgG1 antibody that differs by one or more substitutions selected from Q347E, Q347R, Y349S, Y349K, Y349T, Y349D, Y349E, Y349C, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, D399R, D399K, D399V, S400K, S400R, D401K, F405A, F405T, Y407A, Y4071, Y407V, K409F, K409W, K409D, T411D, T411E, K439D, and K439E.

Individual components of the multi-specific binding proteins are described in more detail below.

NKG2D-Binding Site

Upon binding to the NKG2D receptor and CD16 receptor on natural killer cells and a TAA on cancer cells, the multi-specific binding proteins can engage more than one kind of NK-activating receptor, and may block the binding of natural ligands to NKG2D. In certain embodiments, the multi-specific binding proteins can agonize NK cells in humans. In some embodiments, the multi-specific binding proteins can agonize NK cells in humans and in other species such as rodents and cynomolgus monkeys.

Table 1 lists peptide sequences of heavy chain variable domains and light chain variable domains that, in combination, can bind to NKG2D. In some embodiments, the heavy chain variable domain and the light chain variable domain are arranged in Fab format. These NKG2D binding domains can vary in their binding affinity to NKG2D, nevertheless, they all activate human NK cells. Unless indicated otherwise, the CDR sequences provided in Table 1 are determined under Kabat.

TABLE 1
Exemplary NKG2D-Binding Sites

| | Heavy chain variable domain | Light chain variable domain |
|---|---|---|
| Clones | amino acid sequence | amino acid sequence |
| ADI-27705 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRESGSGSG |
| | KNQFSLKLSSVTAADTAVYYCARA | TEFTLTISSLQPDDFATYYCQQY |
| | RGPWSFDPWGQGTLVTVSS | NSYPITFGGGTKVEIK |
| | (SEQ ID NO: 1) | (SEQ ID NO: 2) |
| | CDR1 (SEQ ID NO: 3)- | |
| | GSFSGYYWS | |
| | CDR2 (SEQ ID NO: 4)- | |
| | EIDHSGSTNYNPSLKS | |
| | CDR3 (SEQ ID NO: 5)- | |
| | ARARGPWSFDP | |
| ADI-27724 | QVQLQQWGAGLLKPSETLSLTCAV | EIVLTQSPGTLSLSPGERATLSCR |
| | YGGSFSGYYWSWIRQPPGKGLEWI | ASQSVSSSYLAWYQQKPGQAPR |
| | GEIDHSGSTNYNPSLKSRVTISVDTS | LLIYGASSRATGIPDRESGSGSG |
| | KNQFSLKLSSVTAADTAVYYCARA | TDFTLTISRLEPEDFAVYYCQQY |
| | RGPWSFDPWGQGTLVTVSS | GSSPITFGGGTKVEIK |
| | (SEQ ID NO: 6) | (SEQ ID NO: 7) |
| ADI-27740 | QVQLQQWGAGLLKPSETLSLTCAV | DIQMTQSPSTLSASVGDRVTITC |
| (A40) | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YHSFYTFGGGTKVEIK |
| | (SEQ ID NO: 8) | (SEQ ID NO: 9) |
| ADI-27741 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | SNSYYTFGGGTKVEIK |
| | (SEQ ID NO: 10) | (SEQ ID NO: 11) |
| ADI-27743 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | NSYPTFGGGTKVEIK |
| | (SEQ ID NO: 12) | (SEQ ID NO: 13) |
| ADI-28153 | QVQLQQWGAGLLKPSETLSLTCAV | ELQMTQSPSSLSASVGDRVTITC |
| | YGGSFSGYYWSWIRQPPGKGLEWI | RTSQSISSYLNWYQQKPGQPPK |
| | GEIDHSGSTNYNPSLKSRVTISVDTS | LLIYWASTRESGVPDRESGSGSG |
| | KNQFSLKLSSVTAADTAVYYCARA | TDFTLTISSLQPEDSATYYCQQS |
| | RGPWGFDPWGQGTLVTVSS | YDIPYTFGQGTKLEIK |
| | (SEQ ID NO: 14) | (SEQ ID NO: 15) |
| ADI-28226 | QVQLQQWGAGLLKPSETLSLTCAV | DIQMTQSPSTLSASVGDRVTITC |
| (C26) | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | GSFPITFGGGTKVEIK |
| | (SEQ ID NO: 16) | (SEQ ID NO: 17) |
| ADI-28154 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TDFTLTISSLQPDDFATYYCQQS |
| | RGPWSFDPWGQGTLVTVSS | KEVPWTFGQGTKVEIK |
| | (SEQ ID NO: 18) | (SEQ ID NO: 19) |
| ADI-29399 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | NSFPTFGGGTKVEIK |
| | (SEQ ID NO: 20) | (SEQ ID NO: 21) |
| ADI-29401 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YDIYPTFGGGTKVEIK |
| | (SEQ ID NO: 22) | (SEQ ID NO: 23) |
| ADI-29403 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | DSYPTFGGGTKVEIK |
| | (SEQ ID NO: 24) | (SEQ ID NO: 25) |
| ADI-29405 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | GSFPTFGGGTKVEIK |
| | (SEQ ID NO: 26) | (SEQ ID NO: 27) |
| ADI-29407 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | QSFPTFGGGTKVEIK |
| | (SEQ ID NO: 28) | (SEQ ID NO: 29) |
| ADI-29419 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | SSFSTFGGGTKVEIK |
| | (SEQ ID NO: 30) | (SEQ ID NO: 31) |
| ADI-29421 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | ESYSTFGGGTKVEIK |
| | (SEQ ID NO: 32) | (SEQ ID NO: 33) |
| ADI-29424 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | DSFITFGGGTKVEIK |
| | (SEQ ID NO: 34) | (SEQ ID NO: 35) |
| ADI-29425 | QVQLQQWGAGLLKPSETLSLTCAV | DIQMTQSPSTLSASVGDRVTITC |
| | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | QSYPTFGGGTKVEIK |
| | (SEQ ID NO: 36) | (SEQ ID NO: 37) |
| ADI-29426 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YHSFPTFGGGTKVEIK |
| | (SEQ ID NO: 38) | (SEQ ID NO: 39) |
| ADI-29429 | QVQLQQWGAGLLKPSETLSLTCAV | DIQMTQSPSTLSASVGDRVTITC |
| | YGGSFSGYYWSWIRQPPGKGLEWI | RASQSIGSWLAWYQQKPGKAP |
| | GEIDHSGSTNYNPSLKSRVTISVDTS | KLLIYKASSLESGVPSRFSGSGS |
| | KNQFSLKLSSVTAADTAVYYCARA | GTEFTLTISSLQPDDFATYYCQQ |
| | RGPWSFDPWGQGTLVTVSS | YELYSYTFGGGTKVEIK |
| | (SEQ ID NO: 40) | (SEQ ID NO: 41) |
| ADI-29447 | QVQLQQWGAGLLKPSETLSLTCAV | DIQMTQSPSTLSASVGDRVTITC |
| (F47) | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCQQY |
| | RGPWSFDPWGQGTLVTVSS | DTFITFGGGTKVEIK |
| | (SEQ ID NO: 42) | (SEQ ID NO: 43) |
| ADI-27727 | QVQLVQSGAEVKKPGSSVKVSCKA | DIVMTQSPDSLAVSLGERATINC |
| | SGGTFSSYAISWVRQAPGQGLEWM | KSSQSVLYSSNNKNYLAWYQQ |
| | GGIIPIFGTANYAQKFQGRVTITADE | KPGQPPKLLIYWASTRESGVPD |
| | STSTAYMELSSLRSEDTAVYYCAR | RFSGSGSGTDFTLTISSLQAEDV |
| | GDSSIRHAYYYYGMDVWGQGTTV | AVYYCQQYYSTPITFGGGTKVE |
| | TVSS | IK |
| | (SEQ ID NO: 44) | (SEQ ID NO: 48) |
| | CDR1 (SEQ ID NO: 45)- | CDR1 (SEQ ID NO: 49)- |
| | GTFSSYAIS (non-Kabat) or SYAIS | KSSQSVLYSSNNKNYLA |
| | (SEQ ID NO: 158) | CDR2 (SEQ ID NO: 50)- |
| | CDR2 (SEQ ID NO: 46)- | WASTRES |
| | GIIPIFGTANYAQKFQG | CDR3 (SEQ ID NO: 51)- |
| | CDR3 (SEQ ID NO: 47)- | QQYYSTPIT |
| | ARGDSSIRHAYYYYGMDV (non- | |
| | Kabat) or GDSSIRHAYYYYGMDV | |
| | (SEQ ID NO: 159) | |
| ADI-29443 | QLQLQESGPGLVKPSETLSLTCTVS | EIVLTQSPATLSLSPGERATLSCR |
| (F43) | GGSISSSSYYWGWIRQPPGKGLEWI | ASQSVSRYLAWYQQKPGQAPR |
| | GSIYYSGSTYYNPSLKSRVTISVDTS | LLIYDASNRATGIPARFSGSGSG |
| | KNQFSLKLSSVTAADTAVYYCARG | TDFTLTISSLEPEDFAVYYCQQF |
| | SDRFHPYFDYWGQGTLVTVSS | DTWPPTFGGGTKVEIK |
| | (SEQ ID NO: 52) | (SEQ ID NO: 56) |
| | CDR1 (SEQ ID NO: 53)- | CDR1 (SEQ ID NO: 57)- |
| | GSISSSSYYWG (non-Kabat) or | RASQSVSRYLA |
| | SSSYYWG (SEQ ID NO: 160) CDR2 | CDR2 (SEQ ID NO: 58)- |
| | (SEQ ID NO: 54)- | DASNRAT |
| | SIYYSGSTYYNPSLKS | CDR3 (SEQ ID NO: 59)- |
| | CDR3 (SEQ ID NO: 55)- | QQFDTWPPT |
| | ARGSDRFHPYFDY (non-Kabat) or | |
| | GSDRFHPYFDY (SEQ ID NO: 161) | |
| ADI-29404 | QVQLQQWGAGLLKPSETLSLTCAV | DIQMTQSPSTLSASVGDRVTITC |
| (F04) | YGGSFSGYYWSWIRQPPGKGLEWI | RASQSISSWLAWYQQKPGKAPK |
| | GEIDHSGSTNYNPSLKSRVTISVDTS | LLIYKASSLESGVPSRFSGSGSG |
| | KNQFSLKLSSVTAADTAVYYCARA | TEFTLTISSLQPDDFATYYCEQY |
| | RGPWSFDPWGQGTLVTVSS | DSYPTFGGGTKVEIK |
| | (SEQ ID NO: 60) | (SEQ ID NO: 61) |
| ADI-28200 | QVQLVQSGAEVKKPGSSVKVSCKA | DIVMTQSPDSLAVSLGERATINC |
| | SGGTFSSYAISWVRQAPGQGLEWM | ESSQSLLNSGNQKNYLTWYQQ |
| | GGIIPIFGTANYAQKFQGRVTITADE | KPGQPPKPLIYWASTRESGVPD |
| | STSTAYMELSSLRSEDTAVYYCAR | RFSGSGSGTDFTLTISSLQAEDV |
| | RGRKASGSFYYYYGMDVWGQGTT | AVYYCQNDYSYPYTFGQGTKL |
| | VTVSS | EIK |
| | (SEQ ID NO: 62) | (SEQ ID NO: 66) |
| | CDR1 (SEQ ID NO: 63)- | CDR1 (SEQ ID NO: 67)- |
| | GTFSSYAIS (non-Kabat) or SYAIS | ESSQSLLNSGNQKNYLT |
| | (SEQ ID NO: 158) CDR2 (SEQ ID | CDR2 (SEQ ID NO: 68)- |
| | NO: 64)-GIIPIFGTANYAQKFQG | WASTRES |
| | CDR3 (SEQ ID NO: 65)- | CDR3 (SEQ ID NO: 69)- |
| | ARRGRKASGSFYYYYGMDV | QNDYSYPYT |
| ADI-29379 | QVQLVQSGAEVKKPGASVKVSCK | EIVMTQSPATLSVSPGERATLSC |
| (E79) | ASGYTFTSYYMHWVRQAPGQGLE | RASQSVSSNLAWYQQKPGQAP |
| | WMGIINPSGGSTSYAQKFQGRVTM | RLLIYGASTRATGIPARFSGSGS |
| | TRDTSTSTVYMELSSLRSEDTAVYY | GTEFTLTISSLQSEDFAVYYCQQ |
| | CARGAPNYGDTTHDYYYMDVWG | YDDWPFTFGGGTKVEIK |
| | KGTTVTVSS | (SEQ ID NO: 74) |
| | (SEQ ID NO: 70) | CDR1 (SEQ ID NO: 75)- |
| | CDR1 (SEQ ID NO: 71)- | RASQSVSSNLA |
| | YTFTSYYMH (non-Kabat) or SYYMH | CDR2 (SEQ ID NO: 76)- |
| | (SEQ ID NO: 162) | GASTRAT |
| | CDR2 (SEQ ID NO: 72)- | CDR3 (SEQ ID NO: 77)- |
| | IINPSGGSTSYAQKFQG | QQYDDWPFT |
| | CDR3 (SEQ ID NO: 73)- | |
| | ARGAPNYGDTTHDYYYMDV (non- | |
| | Kabat) or | |
| | GAPNYGDTTHDYYYMDV (SEQ ID | |
| | NO: 163) | |
| ADI-29463 | QVQLVQSGAEVKKPGASVKVSCK | EIVLTQSPGTLSLSPGERATLSCR |
| (F63) | ASGYTFTGYYMHWVRQAPGQGLE | ASQSVSSNLAWYQQKPGQAPR |
| | WMGWINPNSGGTNYAQKFQGRVT | LLIYGASTRATGIPARFSGSGSG |
| | MTRDTSISTAYMELSRLRSDDTAV | TEFTLTISSLQSEDFAVYYCQQD |
| | YYCARDTGEYYDTDDHGMDVWG | DYWPPTFGGGTKVEIK |
| | QGTTVTVSS | (SEQ ID NO: 82) |
| | (SEQ ID NO: 78) | CDR1 (SEQ ID NO: 75)- |
| | CDR1 (SEQ ID NO: 79)- | RASQSVSSNLA |
| | YTFTGYYMH (non-Kabat) or | CDR2 (SEQ ID NO: 76)- |
| | GYYMH (SEQ ID NO: 164) | GASTRAT |
| | CDR2 (SEQ ID NO: 80)- | CDR3 (SEQ ID NO: 85)- |
| | WINPNSGGTNYAQKFQG | QQDDYWPPT |
| | CDR3 (SEQ ID NO: 81)- | |
| | ARDTGEYYDTDDHGMDV (non- | |
| | Kabat) or DTGEYYDTDDHGMDV | |
| | (SEQ ID NO: 165) | |
| ADI-27744 | EVQLLESGGGLVQPGGSLRLSCAAS | DIQMTQSPSSVSASVGDRVTITC |
| (A44) | GFTFSSYAMSWVRQAPGKGLEWV | RASQGIDSWLAWYQQKPGKAP |
| | SAISGSGGSTYYADSVKGRFTISRD | KLLIYAASSLQSGVPSRFSGSGS |
| | NSKNTLYLQMNSLRAEDTAVYYC | GTDFTLTISSLQPEDFATYYCQQ |
| | AKDGGYYDSGAGDYWGQGTLVTV | GVSYPRTFGGGTKVEIK |
| | SS | (SEQ ID NO: 90) |
| | (SEQ ID NO: 86) | CDR1 (SEQ ID NO: 91)- |
| | CDR1 (SEQ ID NO: 87)- | RASQGIDSWLA |
| | FTFSSYAMS (non-Kabat) or SYAMS | CDR2 (SEQ ID NO: 92)- |
| | (SEQ ID NO: 166) | AASSLQS |
| | CDR2 (SEQ ID NO: 88)- | CDR3 (SEQ ID NO: 93)- |
| | AISGSGGSTYYADSVKG | QQGVSYPRT |
| | CDR3 (SEQ ID NO: 89)- | |
| | AKDGGYYDSGAGDY (non-Kabat) or | |
| | DGGYYDSGAGDY (SEQ ID NO: 167) | |
| ADI-27749 | EVQLVESGGGLVKPGGSLRLSCAA | DIQMTQSPSSVSASVGDRVTITC |
| (A49) | SGFTFSSYSMNWVRQAPGKGLEW | RASQGISSWLAWYQQKPGKAP |
| | VSSISSSSSYIYYADSVKGRFTISRD | KLLIYAASSLQSGVPSRESGSGS |
| | NAKNSLYLQMNSLRAEDTAVYYC | GTDFTLTISSLQPEDFATYYCQQ |
| | ARGAPMGAAAGWFDPWGQGTLVT | GVSFPRTFGGGTKVEIK |
| | VSS | (SEQ ID NO: 98) |
| | (SEQ ID NO: 94) | CDR1 (SEQ ID NO: 99)- |
| | CDR1 (SEQ ID NO: 95)- | RASQGISSWLA |
| | FTFSSYSMN (non-Kabat) or SYSMN | CDR2 (SEQ ID NO: 100)- |
| | (SEQ ID NO: 168) | AASSLQS |
| | CDR2 (SEQ ID NO: 96)- | CDR3 (SEQ ID NO: 101)- |
| | SISSSSSYIYYADSVKG | QQGVSFPRT |
| | CDR3 (SEQ ID NO: 97)- | |
| | ARGAPMGAAAGWFDP (non-Kabat) | |
| | or GAPMGAAAGWFDP (SEQ ID | |
| | NO: 169) | |
| ADI-29378 | QVQLVQSGAEVKKPGASVKVSCK | EIVLTQSPATLSLSPGERATLSCR |
| (E78) | ASGYTFTSYYMHWVRQAPGQGLE | ASQSVSSYLAWYQQKPGQAPR |
| | WMGIINPSGGSTSYAQKFQGRVTM | LLIYDASNRATGIPARFSGSGSG |
| | TRDTSTSTVYMELSSLRSEDTAVYY | TDFTLTISSLEPEDFAVYYCQQS |
| | CAREGAGFAYGMDYYYMDVWGK | DNWPFTFGGGTKVEIK |
| | GTTVTVSS | (SEQ ID NO: 106) |
| | (SEQ ID NO: 102) | CDR1 (SEQ ID NO: 107)- |
| | CDR1 (SEQ ID NO: 71)- | RASQSVSSYLA |
| | YTFTSYYMH (non-Kabat) or | CDR2 (SEQ ID NO: 108)- |
| | SYYMH (SEQ ID NO: 162) | DASNRAT |
| | CDR2 (SEQ ID NO: 72)- | CDR3 (SEQ ID NO: 109)- |
| | IINPSGGSTSYAQKFQG | QQSDNWPFT |
| | CDR3 (SEQ ID NO: 105)- | |
| | AREGAGFAYGMDYYYMDV (non- | |
| | Kabat) or EGAGFAYGMDYYYMDV | |
| | (SEQ ID NO: 170) | |
| A49MI | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPIGAAAGWFDPWGQGTLVT | GVSFPRTFGGGTKVEIK |
| | VSS | (SEQ ID NO: 98) |
| | (SEQ ID NO: 144) | CDR1 (SEQ ID NO: 99)- |
| | CDR1 (SEQ ID NO: 95)- | RASQGISSWLA |
| | FTFSSYSMN (non-Kabat) or SYSMN | CDR2 (SEQ ID NO: 100)- |
| | (SEQ ID NO: 168) | AASSLQS |
| | CDR2 (SEQ ID NO: 96)- | CDR3 (SEQ ID NO: 101)- |
| | SISSSSSYIYYADSVKG | QQGVSFPRT |
| | CDR3: (non-Kabat) | |
| | ARGAPIGAAAGWFDP (SEQ ID | |
| | NO: 172) or GAPIGAAAGWFDP (SEQ | |
| | ID NO: 173) | |
| A49MQ | EVOLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPQGAAAGWFDPWGQGTLVT | GVSFPRTFGGGTKVEIK |
| | VSS | (SEQ ID NO: 98) |
| | (SEQ ID NO: 174) | CDR1 (SEQ ID NO: 99)- |
| | CDR1 (SEQ ID NO: 95)- | RASQGISSWLA |
| | FTFSSYSMN (non-Kabat) or SYSMN | CDR2 (SEQ ID NO: 100)- |
| | (SEQ ID NO: 168) | AASSLQS |
| | CDR2 (SEQ ID NO: 96)- | CDR3 (SEQ ID NO: 101)- |
| | SISSSSSYIYYADSVKG | QQGVSFPRT |
| | CDR3 (non-Kabat) (SEQ ID | |
| | NO: 175)- | |
| | ARGAPQGAAAGWFDP or CDR3 | |
| | (SEQ ID NO: 176)- | |
| | GAPQGAAAGWFDP | |
| A49ML | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPLGAAAGWFDPWGQGTLVT | GVSFPRTFGGGTKVEIK |
| | VSS | (SEQ ID NO: 98) |
| | (SEQ ID NO: 177) | CDR1 (SEQ ID NO: 99)- |
| | CDR1 (SEQ ID NO: 95)- | RASQGISSWLA |
| | FTFSSYSMN (non-Kabat) or SYSMN | CDR2 (SEQ ID NO: 100)- |
| | (SEQ ID NO: 168) | AASSLQS |
| | CDR2 (SEQ ID NO: 96)- | CDR3 (SEQ ID NO: 101)- |
| | SISSSSSYIYYADSVKG | QQGVSFPRT |
| | CDR3 (non-Kabat) (SEQ ID | |
| | NO: 178)- | |
| | ARGAPLGAAAGWFDP or CDR3 | |
| | (SEQ ID NO: 179)- | |
| | GAPLGAAAGWFDP | |
| A49MF | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPFGAAAGWFDPWGQGTLVT | GVSFPRTFGGGTKVEIK |
| | VSS | (SEQ ID NO: 98) |
| | (SEQ ID NO: 180) | CDR1 (SEQ ID NO: 99)- |
| | CDR1 (SEQ ID NO: 95)- | RASQGISSWLA |
| | FTFSSYSMN (non-Kabat) or SYSMN | CDR2 (SEQ ID NO: 100)- |
| | (SEQ ID NO: 168) | AASSLQS |
| | CDR2 (SEQ ID NO: 96)- | CDR3 (SEQ ID NO: 101)- |
| | SISSSSSYIYYADSVKG | QQGVSFPRT |
| | CDR3 (non-Kabat) (SEQ ID | |
| | NO: 181)- | |
| | ARGAPFGAAAGWFDP or CDR3 | |
| | (SEQ ID NO: 182)- | |
| | GAPFGAAAGWFDP | |
| A49MV | EVQLVESGGGLVKPGGSLRLSCAA | DIQMTQSPSSVSASVGDRVTITC |
| | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPVGAAAGWFDPWGQGTLVT | GVSFPRTFGGGTKVEIK |
| | VSS | (SEQ ID NO: 98) |
| | (SEQ ID NO: 183) | CDR1 (SEQ ID NO: 99)- |
| | CDR1 (SEQ ID NO: 95)- | RASQGISSWLA |
| | FTFSSYSMN (non-Kabat) or SYSMN | CDR2 (SEQ ID NO: 100)- |
| | (SEQ ID NO: 168) | AASSLQS |
| | CDR2 (SEQ ID NO: 96)- | CDR3 (SEQ ID NO: 101)- |
| | SISSSSSYIYYADSVKG | QQGVSFPRT |
| | CDR3 (non-Kabat) (SEQ ID | |
| | NO: 184)- | |
| | ARGAPVGAAAGWFDP or CDR3 | |
| | (SEQ ID NO: 185)- | |
| | GAPVGAAAGWFDP | |
| A49- | EVQLVESGGGLVKPGGSLRLSCAA | DIQMTQSPSSVSASVGDRVTITC |
| consensus | SGFTFSSYSMNWVRQAPGKGLEW | RASQGISSWLAWYQQKPGKAP |
| | VSSISSSSSYIYYADSVKGRFTISRD | KLLIYAASSLQSGVPSRFSGSGS |
| | NAKNSLYLQMNSLRAEDTAVYYC | GTDFTLTISSLQPEDFATYYCQQ |
| | ARGAPXGAAAGWFDPWGQGTLVT | GVSFPRTFGGGTKVEIK |
| | VSS, wherein X is M, L, I, V, | (SEQ ID NO: 98) |
| | Q, or F (SEQ ID NO: 186) | CDR1 (SEQ ID NO: 99)- |
| | CDR1 (SEQ ID NO: 95)- | RASQGISSWLA |
| | FTFSSYSMN (non-Kabat) or SYSMN | CDR2 (SEQ ID NO: 100)- |
| | (SEQ ID NO: 168) | AASSLQS |
| | CDR2 (SEQ ID NO: 96)- | CDR3 (SEQ ID NO: 101)- |
| | SISSSSSYIYYADSVKG | QQGVSFPRT |
| | CDR3 (non-Kabat) (SEQ ID | |
| | NO: 187)- | |
| | ARGAPXGAAAGWFDP or CDR3 | |
| | (SEQ ID NO: 188)- | |
| | GAPXGAAAGWFDP, wherein X is M, | |
| | L, I, V, Q, or F | |
| NKG2D | QVQLVESGGGLVKPGGSLRLSCAA | QSALTQPASVSGSPGQSITISCSG |
| binder in | SGFTFSSYGMHWVRQAPGKGLEW | SSSNIGNNAVNWYQQLPGKAPK |
| US | VAFIRYDGSNKYYADSVKGRFTISR | LLIYYDDLLPSGVSDRFSGSKSG |
| 9,273,136 | DNSKNTLYLQMNSLRAEDTAVYY | TSAFLAISGLQSEDEADYYCAA |
| | CAKDRGLGDGTYFDYWGQGTTVT | WDDSLNGPVFGGGTKLTVL |
| | VSS (SEQ ID NO: 110) | (SEQ ID NO: 111) |
| NKG2D | QVHLQESGPGLVKPSETLSLTCTVS | EIVLTQSPGTLSLSPGERATLSCR |
| binder in | DDSISSYYWSWIRQPPGKGLEWIGH | ASQSVSSSYLAWYQQKPGQAPR |
| US | ISYSGSANYNPSLKSRVTISVDTSKN | LLIYGASSRATGIPDRFSGSGSG |
| 7,879,985 | QFSLKLSSVTAADTAVYYCANWD | TDFTLTISRLEPEDFAVYYCQQY |
| | DAFNIWGQGTMVTVSS (SEQ ID | GSSPWTFGQGTKVEIK (SEQ ID |
| | NO: 112) | NO: 113) |

In some embodiments, the Fab includes a heavy chain variable domain related to SEQ ID NO:94 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:94, and/or incorporate amino acid sequences identical to the CDR1 (SEQ ID NO:95 or 168), CDR2 (SEQ ID NO:96), and CDR3 (SEQ ID NO:97 or 169) sequences of SEQ ID NO:94. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab includes a heavy chain variable domain related to SEQ ID NO:144 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:144, and/or incorporate amino acid sequences identical to the CDR1 (SEQ ID NO:95 or 168), CDR2 (SEQ ID NO:96), and CDR3 (SEQ ID NO:172 or 173) sequences of SEQ ID NO:144. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab includes a heavy chain variable domain related to SEQ ID NO:174 and a light chain variable domain related to SEQ ID NO:98. For example, the 95%, 96%, 97%, 98%, 99%, or 100%) identical to SEQ ID NO:174, and/or incorporate amino acid sequences identical to the CDR1 (SEQ ID NO:95 or 168), CDR2 (SEQ ID NO:96), and CDR3 (SEQ ID NO:175 or 176) sequences of SEQ ID NO:174. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab includes a heavy chain variable domain related to SEQ ID NO:177 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:177, and/or incorporate amino acid sequences identical to the CDR1 (SEQ ID NO:95 or 168), CDR2 (SEQ ID NO:96), and CDR3 (SEQ ID NO:178 or 179) sequences of SEQ ID NO:177. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab includes a heavy chain variable domain related to SEQ ID NO:180 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:180, and/or incorporate amino acid sequences identical to the CDR1 (SEQ ID NO:95 or 168), CDR2 (SEQ ID NO:96), and CDR3 (SEQ ID NO:181 or 182) sequences of SEQ ID NO:180. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab includes a heavy chain variable domain related to SEQ ID NO:183 and a light chain variable domain related to SEQ ID NO:98. For example, the 95%, 96%, 97%, 98%, 99%, or 100%) identical to SEQ ID NO:183, and/or incorporate amino acid sequences identical to the CDR1 (SEQ ID NO:95 or 168), CDR2 (SEQ ID NO:96), and CDR3 (SEQ ID NO:184 or 185) sequences of SEQ ID NO:183. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab includes a heavy chain variable domain related to SEQ ID NO:186 and a light chain variable domain related to SEQ ID NO:98. For example, the heavy chain variable domain of the Fab can be at least 90% (e.g., 90%, 91%, 92%, 93%, 94%, 95%, 96%, 97%, 98%, 99%, or 100%) identical to SEQ ID NO:186, and/or incorporate amino acid sequences identical to the CDR1 (SEQ ID NO:95 or 168), CDR2 (SEQ ID NO:96), and CDR3 (SEQ ID NO:187 or 188) sequences of SEQ ID NO:186. Similarly, the light chain variable domain of the Fab can be at least 90% (e.g., 90%, 91%, 92%, 93%, 94%, 95%, 96%, 97%, 98%, 99%, or 100%) identical to SEQ ID NO:98, and/or incorporate amino acid sequences identical to the CDR1 (SEQ ID NO:99), CDR2 (SEQ ID NO:100), and CDR3 (SEQ ID NO:101) sequences of SEQ ID NO:98.

In some embodiments, the Fab includes a heavy chain variable domain related to SEQ ID NO:86 and a light chain variable domain related to SEQ ID NO:90. For example, the heavy chain variable domain of the Fab can be at least 90% (e.g., 90%, 91%, 92%, 93%, 94%, 95%, 96%, 97%, 98%, 99%, or 100%) identical to SEQ ID NO:86, and/or incorporate amino acid sequences identical to the CDR1 (SEQ ID NO:87 or 166), CDR2 (SEQ ID NO:88), and CDR3 (SEQ ID NO:89 or 167) sequences of SEQ ID NO:86. Similarly, the light chain variable domain of the Fab can be at least 90% (e.g., 90%, 91%, 92%, 93%, 94%, 95%, 96%, 97%, 98%, 99%, or 100%) identical to SEQ ID NO:90, and/or incorporate amino acid sequences identical to the CDR1 (SEQ ID NO:91), CDR2 (SEQ ID NO:92), and CDR3 (SEQ ID NO:93) sequences of SEQ ID NO:90.

In some embodiments, the Fab includes a heavy chain variable domain related to SEQ ID NO:102 and a light chain variable domain related to SEQ ID NO:106. For example, the 95%, 96%, 97%, 98%, 99%, or 100%) identical to SEQ ID NO:102, and/or incorporate amino acid sequences identical to the CDR1 (SEQ ID NO:71 or 162), CDR2 (SEQ ID NO:72), and CDR3 (SEQ ID NO:105 or 170) sequences of SEQ ID NO:102. Similarly, the light chain variable domain of the Fab can be at least 90% (e.g., 90%, 91%, 92%, 93%, 94%, 95%, 96%, 97%, 98%, 99%, or 100%) identical to SEQ ID NO:106, and/or incorporate amino acid sequences identical to the CDR1 (SEQ ID NO:107), CDR2 (SEQ ID NO:108), and CDR3 (SEQ ID NO:109) sequences of SEQ ID NO:106.

In some embodiments, the Fab includes a heavy chain variable domain related to SEQ ID NO:70 and a light chain variable domain related to SEQ ID NO:74. For example, the heavy chain variable domain of the Fab can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:71 or 162), CDR2 (SEQ ID NO:72), and CDR3 (SEQ ID NO:73 or 163) sequences of SEQ ID NO:70. Similarly, the light chain variable domain of the Fab can be at least 90% (e.g., 90%, 91%, 92%, 93%, 94%, 95%, 96%, 97%, 98%, 99%, or 100%) identical to SEQ ID NO:74, and/or incorporate amino acid sequences identical to the CDR1 (SEQ ID NO:75), CDR2 (SEQ ID NO:76), and CDR3 (SEQ ID NO:77) sequences of SEQ ID NO:74.

In some embodiments, the Fab includes a heavy chain variable domain related to SEQ ID NO:70 and a light chain variable domain related to SEQ ID NO:74. For example, the heavy chain variable domain of the Fab can be at least 90% (e.g., 90%, 91%, 92%, 93%, 94%, 95%, 96%, 97%, 98%, 99%, or 100%) identical to SEQ ID NO:70, and/or incorporate amino acid sequences identical to the CDR1 (SEQ ID NO:71 or 162), CDR2 (SEQ ID NO:72), and CDR3 (SEQ ID NO:73 or 163) sequences of SEQ ID NO:70. Similarly, the light chain variable domain of the Fab can be at least 90% (e.g., 90%, 91%, 92%, 93%, 94%, 95%, 96%, 97%, 98%, 99%, or 100%) identical to SEQ ID NO:74, and/or incorporate amino acid sequences identical to the CDR1 (SEQ ID NO:75), CDR2 (SEQ ID NO:76), and CDR3 (SEQ ID NO:77) sequences of SEQ ID NO:74.

In some embodiments, the Fab includes a heavy chain variable domain related to SEQ ID NO:78 and a light chain variable domain related to SEQ ID NO:82. For example, the 95%, 96%, 97%, 98%, 99%, or 100%) identical to SEQ ID NO:78, and/or incorporate amino acid sequences identical to the CDR1 (SEQ ID NO:79 or 164), CDR2 (SEQ ID NO:80), and CDR3 (SEQ ID NO:81 or 165) sequences of SEQ ID NO:78. Similarly, the light chain variable domain of the Fab can be at least 90% (e.g., 90%, 91%, 92%, 93%, 94%, 95%, 96%, 97%, 98%, 99%, or 100%) identical to SEQ ID NO:82, and/or incorporate amino acid sequences identical to the CDR1 (SEQ ID NO:75), CDR2 (SEQ ID NO:76), and CDR3 (SEQ ID NO:77) sequences of SEQ ID NO:82.

The multi-specific binding proteins can bind to NKG2D-expressing cells, which include but are not limited to NK cells, γδ T cells and CD8+αβ T cells. Upon NKG2D binding, the multi-specific binding proteins may block natural ligands, such as ULBP6 and MICA, from binding to NKG2D and activating NK cells.

In certain embodiments, the Fab or the multi-specific binding protein binds to NKG2D with an affinity of K_{D} of 2 nM to 120 nM, e.g., 2 nM to 110 nM, 2 nM to 100 nM, 2 nM to 90 nM, 2 nM to 80 nM, 2 nM to 70 nM, 2 nM to 60 nM, 2 nM to 50 nM, 2 nM to 40 nM, 2 nM to 30 nM, 2 nM to 20 nM, 2 nM to 10 nM, about 15 nM, about 14 nM, about 13 nM, about 12 nM, about 11 nM, about 10 nM, about 9 nM, about 8 nM, about 7 nM, about 6 nM, about 5 nM, about 4.5 nM, about 4 nM, about 3.5 nM, about 3 nM, about 2.5 nM, about 2 nM, about 1.5 nM, about 1 nM, between about 0.5 nM to about 1 nM, about 1 nM to about 2 nM, about 2 nM to 3 nM, about 3 nM to 4 nM, about 4 nM to about 5 nM, about 5 nM to about 6 nM, about 6 nM to about 7 nM, about 7 nM to about 8 nM, about 8 nM to about 9 nM, about 9 nM to about 10 nM, about 1 nM to about 10 nM, about 2 nM to about 10 nM, about 3 nM to about 10 nM, about 4 nM to about 10 nM, about 5 nM to about 10 nM, about 6 nM to about 10 nM, about 7 nM to about 10 nM, or about 8 nM to about 10 nM.

In certain embodiments, the Fab binds to NKG2D with a K_{D} of 2 nM to 120 nM, as measured by surface plasmon resonance. In certain embodiments, the multi-specific binding protein binds to NKG2D with a K_{D} of 2 nM to 120 nM, as measured by surface plasmon resonance. In certain embodiments, the Fab binds to NKG2D with a K_{D} of 10 nM to 62 nM, as measured by surface plasmon resonance. In certain embodiments, the multi-specific binding protein binds to NKG2D with a K_{D} of 10 nM to 62 nM, as measured by surface plasmon resonance.

In some embodiments, the Fab described above is linked to an antibody Fc polypeptide. In some embodiments, the heavy chain portion of the Fab is linked to the N-terminus of an antibody Fc polypeptide.

Table 2 lists amino acid sequences of heavy chain variable domains and light chain variable domains that, in combination, can bind to HER2. In certain embodiments, an antigen-binding site that binds HER2 takes the format of an scFv.

TABLE 2
Exemplary HER2-Binding Sites

| | Heavy chain variable domain | Light chain variable domain |
|---|---|---|
| Clones | amino acid sequence | amino acid sequence |
| Trastuzumab | EVQLVESGGGLVQPGGSLRLSCA | DIQMTQSPSSLSASVGDRVTITCR |
| | ASGFNIKDTYIHWVRQAPGKGLE | ASQDVNTAVAWYQQKPGKAPK |
| | WVARIYPTNGYTRYADSVKGRFT | LLIYSASFLYSGVPSRFSGSRSGT |
| | ISADTSKNTAYLQMNSLRAEDTA | DFTLTISSLQPEDFATYYCQQHY |
| | VYYCSRWGGDGFYAMDYWGQG | TTPPTFGQGTKVEIK |
| | TLVTVSS | (SEQ ID NO: 118) |
| | (SEQ ID NO: 114) | CDR1 (SEQ ID NO: 119)- |
| | CDR1 (SEQ ID NO: 115)-GFNIKDT | QDVNTAVA |
| | CDR2 (SEQ ID NO: 116)-YPTNGY | CDR2 (SEQ ID NO: 120)- |
| | CDR3 (SEQ ID NO: 117)- | SASFLYS |
| | WGGDGFYAMDY | CDR3 (SEQ ID NO: 121)- |
| | | QQHYTTPPT |
| Trastuzumab | EVQLVESGGGLVQPGGSLRLSCA | DIQMTQSPSSLSASVGDRVTITCR |
| (VH and VL | ASGFNIKDTYIHWVRQAPGKCLE | ASQDVNTAVAWYQQKPGKAPK |
| in scFv | WVARIYPTNGYTRYADSVKGRFT | LLIYSASFLYSGVPSRESGSRSGT |
| construct) | ISADTSKNTAYLQMNSLRAEDTA | DFTLTISSLQPEDFATYYCQQHY |
| | VYYCSRWGGDGFYAMDYWGQG | TTPPTFGCGTKVEIK |
| | TLVTVSS (SEQ ID NO: 195) | (SEQ ID NO: 196) |
| | CDR1 (SEQ ID NO: 115)-GFNIKDT | CDR1 (SEQ ID NO: 119)- |
| | CDR2 (SEQ ID NO: 116)-YPTNGY | QDVNTAVA |
| | CDR3 (SEQ ID NO: 117)- | CDR2 (SEQ ID NO: 120)- |
| | WGGDGFYAMDY | SASFLYS |
| | | CDR3 (SEQ ID NO: 121)- |
| | | QQHYTTPPT |
| Trastuzumab- | DIQMTQSPSSLSASVGDRVTITCRASQDVNTAVAWYQQKPGKAPKLLI | |
| scFv | YSASFLYSGVPSRFSGSRSGTDFTLTISSLQPEDFATYYCQQHYTTPPTF | |
| | GCGTKVEIKGGGGSGGGGSGGGGSGGGGSEVQLVESGGGLVQPGGSL | |
| | RLSCAASGFNIKDTYIHWVRQAPGKCLEWVARIYPTNGYTRYADSVK | |
| | GRFTISADTSKNTAYLQMNSLRAEDTAVYYCSRWGGDGFYAMDYWG | |
| | QGTLVTVSS (SEQ ID NO: 139) | |
| Pertuzumab | EVQLVESGGGLVQPGGSLRLSCA | DIQMTQSPSSLSASVGDRVTITCK |
| | ASGFTFTDYTMDWVRQAPGKGL | ASQDVSIGVAWYQQKPGKAPKL |
| | EWVADVNPNSGGSIYNQRFKGRF | LIYSASYRYTGVPSRFSGSGSGTD |
| | TLSVDRSKNTLYLQMNSLRAEDT | FTLTISSLQPEDFATYYCQQYYIY |
| | AVYYCARNLGPSFYFDYWGQGT | PYTFGQGTKVEIKR |
| | LVTVSSA | (SEQ ID NO: 126) |
| | (SEQ ID NO: 122) | CDR1 (SEQ ID NO: 127)- |
| | CDR1 (SEQ ID NO: 123)-GFTFTDY | QDVSIGVA |
| | CDR2 (SEQ ID NO: 124)-NPNSGG | CDR2 (SEQ ID NO: 128)- |
| | CDR3 (SEQ ID NO: 125)- | SASYRYT |
| | NLGPSFYFDY | CDR3 (SEQ ID NO: 129)- |
| | | QQYYIYPYT |
| Pertuzumab | EVQLVESGGGLVQPGGSLRLSCA | DIQMTQSPSSLSASVGDRVTITCK |
| (VH and VL | ASGFTFTDYTMDWVRQAPGKCL | ASQDVSIGVAWYQQKPGKAPKL |
| in scFv | EWVADVNPNSGGSIYNQRFKGRF | LIYSASYRYTGVPSRFSGSGSGTD |
| construct) | TLSVDRSKNTLYLQMNSLRAEDT | FTLTISSLQPEDFATYYCQQYYIY |
| | AVYYCARNLGPSFYFDYWGQGT | PYTFGCGTKVEIKR |
| | LVTVSSA (SEQ ID NO: 197) | (SEQ ID NO: 198) |
| | CDR1 (SEQ ID NO: 123)-GFTFTDY | CDR1 (SEQ ID NO: 127)- |
| | CDR2 (SEQ ID NO: 124)-NPNSGG | QDVSIGVA |
| | CDR3 (SEQ ID NO: 125)- | CDR2 (SEQ ID NO: 128)- |
| | NLGPSFYFDY | SASYRYT |
| | | CDR3 (SEQ ID NO: 129)- |
| | | QQYYIYPYT |
| Pertuzumab | DIQMTQSPSSLSASVGDRVTITCKASQDVSIGVAWYQQKPGKAPKLLI | |
| scFv | YSASYRYTGVPSRFSGSGSGTDFTLTISSLQPEDFATYYCQQYYIYPYTF | |
| | GCGTKVEIKRGGGGSGGGGSGGGGSGGGGSEVQLVESGGGLVQPGGS | |
| | LRLSCAASGFTFTDYTMDWVRQAPGKCLEWVADVNPNSGGSIYNQRF | |
| | KGRFTLSVDRSKNTLYLQMNSLRAEDTAVYYCARNLGPSFYFDYWG | |
| | QGTLVTVSSA | |
| | (SEQ ID NO: 189) | |
| MGAH22 | QVQLQQSGPELVKPGASLKLSCT | DIVMTQSHKFMSTSVGDRVSITC |
| (US | ASGFNIKDTYIHWVKQRPEQGLE | KASQDVNTAVAWYQQKPGHSP |
| 8,802,093) | WIGRIYPTNGYTRYDPKFQDKATI | KLLIYSASFRYTGVPDRFTGSRSG |
| | TADTSSNTAYLQVSRLTSEDTAV | TDFTFTISSVQAEDLAVYYCQQH |
| | YYCSRWGGDGFYAMDYWGQGA | YTTPPTFGGGTKVEIK |
| | SVTVSS | (SEQ ID NO: 134) |
| | (SEQ ID NO: 130) | CDR1 (SEQ ID NO: 135)- |
| | CDR1 (SEQ ID NO: 131)-GFNIKDT | QDVNTAVA |
| | CDR2 (SEQ ID NO: 132)-YPTNGY | CDR2 (SEQ ID NO: 136)- |
| | CDR3 (SEQ ID NO: 133)- | SASFRYT |
| | WGGDGFYAMDY | CDR3 (SEQ ID NO: 137)- |
| | | QQHYTTPPT |
| MGAH22 | QVQLQQSGPELVKPGASLKLSCT | DIVMTQSHKFMSTSVGDRVSITC |
| (VH and VL | ASGFNIKDTYIHWVKQRPEQCLE | KASQDVNTAVAWYQQKPGHSP |
| in scFv | WIGRIYPTNGYTRYDPKFQDKATI | KLLIYSASFRYTGVPDRFTGSRSG |
| construct) | TADTSSNTAYLQVSRLTSEDTAV | TDFTFTISSVQAEDLAVYYCQQH |
| | YYCSRWGGDGFYAMDYWGQGA | YTTPPTFGCGTKVEIKR (SEQ ID |
| | SVTVSSA (SEQ ID NO: 199) | NO: 200) |
| | CDR1 (SEQ ID NO: 131)-GFNIKDT | CDR1 (SEQ ID NO: 135)- |
| | CDR2 (SEQ ID NO: 132)-YPTNGY | QDVNTAVA |
| | CDR3 (SEQ ID NO: 133)- | CDR2 (SEQ ID NO: 136)- |
| | WGGDGFYAMDY | SASFRYT |
| | | CDR3 (SEQ ID NO: 137)- |
| | | QQHYTTPPT |

| | |
|---|---|
| MGAH22 | DIVMTQSHKFMSTSVGDRVSITCKASQDVNTAVAWYQQKPGHSPKLL |
| scFv | IYSASFRYTGVPDRFTGSRSGTDFTFTISSVQAEDLAVYYCQQHYTTPP |
| | TFGCGTKVEIKRGGGGSGGGGSGGGGSGGGGSQVQLQQSGPELVKPG |
| | ASLKLSCTASGFNIKDTYIHWVKQRPEQCLEWIGRIYPTNGYTRYDPKF |
| | QDKATITADTSSNTAYLQVSRLTSEDTAVYYCSRWGGDGFYAMDYW |
| | GQGASVTVSSA |
| | (SEQ ID NO: 171) |

Alternatively, novel antigen-binding sites that can bind to HER2 can be identified by screening for binding to the amino acid sequence defined by SEQ ID NO:138 or a mature extracellular fragment thereof.

(SEQ ID NO: 138)
MELAALCRWGLLLALLPPGAASTQVCTGTDMKLRLPASPETHLDMLRHL
YQGCQVVQGNLELTYLPTNASLSFLQDIQEVQGYVLIAHNQVRQVPLQR
LRIVRGTQLFEDNYALAVLDNGDPLNNTTPVTGASPGGLRELQLRSLTE
ILKGGVLIQRNPQLCYQDTILWKDIFHKNNQLALTLIDTNRSRACHPCS
PMCKGSRCWGESSEDCQSLTRTVCAGGCARCKGPLPTDCCHEQCAAGCT
GPKHSDCLACLHFNHSGICELHCPALVTYNTDTFESMPNPEGRYTFGAS
CVTACPYNYLSTDVGSCTLVCPLHNQEVTAEDGTQRCEKCSKPCARVCY
GLGMEHLREVRAVTSANIQEFAGCKKIFGSLAFLPESFDGDPASNTAPL
QPEQLQVFETLEEITGYLYISAWPDSLPDLSVFQNLQVIRGRILHNGAY
SLTLQGLGISWLGLRSLRELGSGLALIHHNTHLCFVHTVPWDQLFRNPH
QALLHTANRPEDECVGEGLACHQLCARGHCWGPGPTQCVNCSQFLRGQE
CVEECRVLQGLPREYVNARHCLPCHPECQPQNGSVTCFGPEADQCVACA
HYKDPPFCVARCPSGVKPDLSYMPIWKFPDEEGACQPCPINCTHSCVDL
DDKGCPAEQRASPLTSIISAVVGILLVVVLGVVFGILIKRRQQKIRKYT
MRRLLQETELVEPLTPSGAMPNQAQMRILKETELRKVKVLGSGAFGTVY
KGIWIPDGENVKIPVAIKVLRENTSPKANKEILDEAYVMAGVGSPYVSR
LLGICLTSTVQLVTQLMPYGCLLDHVRENRGRLGSQDLLNWCMQIAKGM
SYLEDVRLVHRDLAARNVLVKSPNHVKITDFGLARLLDIDETEYHADGG
KVPIKWMALESILRRRFTHQSDVWSYGVTVWELMTFGAKPYDGIPAREI
PDLLEKGERLPQPPICTIDVYMIMVKCWMIDSECRPRFRELVSEFSRMA
RDPQRFVVIQNEDLGPASPLDSTFYRSLLEDDDMGDLVDAEEYLVPQQG
FFCPDPAPGAGGMVHHRHRSSSTRSGGGDLTLGLEPSEEEAPRSPLAPS
EGAGSDVFDGDLGMGAAKGLQSLPTHDPSPLQRYSEDPTVPLPSETDGY
VAPLTCSPQPEYVNQPDVRPQPPSPREGPLPAARPAGATLERPKTLSPG
KNGVVKDVFAFGGAVENPEYLTPQGGAAPQPHPPPAFSPAFDNLYYWDQ
DPPERGAPPSTFKGTPTAENPEYLGLDVPV

In some embodiments, the scFv includes a heavy chain variable domain related to SEQ ID NO:195 and a light chain variable domain related to SEQ ID NO:196. For example, the heavy chain variable domain of the scFv can be at least 90% (e.g., 90%, 91%, 92%, 93%, 94%, 95%, 96%, 97%, 98%, 99%, or 100%) identical to SEQ ID NO:195, and/or incorporate amino acid sequences identical to the CDR1 (SEQ ID NO:115), CDR2 (SEQ ID NO:116), and CDR3 (SEQ ID NO:117) sequences of SEQ ID NO:195. Similarly, the light chain variable domain of the scFv can be at least 90% (e.g., 90%, 91%, 92%, 93%, 94%, 95%, 96%, 97%, 98%, 99%, or 100%) identical to SEQ ID NO:196, and/or incorporate amino acid sequences identical to the CDR1 (SEQ ID NO:119), CDR2 (SEQ ID NO:120), and CDR3 (SEQ ID NO:121) sequences of SEQ ID NO:196. In some embodiments, the scFv has the amino acid sequence of SEQ ID NO:139.

In some embodiments, the scFv includes a heavy chain variable domain related to SEQ ID NO:197 and a light chain variable domain related to SEQ ID NO:198. For example, the heavy chain variable domain of the scFv can be at least 90% (e.g., 90%, 91%, 92%, 93%, 94%, 95%, 96%, 97%, 98%, 99%, or 100%) identical to SEQ ID NO:197, and/or incorporate amino acid sequences identical to the CDR1 (SEQ ID NO:123), CDR2 (SEQ ID NO:124), and CDR3 (SEQ ID NO:125) sequences of SEQ ID NO:197. Similarly, the light chain variable domain of the scFv can be at least 90% (e.g., 90%, 91%, 92%, 93%, 94%, 95%, 96%, 97%, 98%, 99%, or 100%) identical to SEQ ID NO:198, and/or incorporate amino acid sequences identical to the CDR1 (SEQ ID NO:127), CDR2 (SEQ ID NO:128), and CDR3 (SEQ ID NO:129) sequences of SEQ ID NO:198. In some embodiments, the scFv has the amino acid sequence of SEQ ID NO:189.

In some embodiments, the scFv includes a heavy chain variable domain related to SEQ ID NO:199 and a light chain variable domain related to SEQ ID NO:200. For example, the heavy chain variable domain of the scFv can be at least 90% (e.g., 90%, 91%, 92%, 93%, 94%, 95%, 96%, 97%, 98%, 99%, or 100%) identical to SEQ ID NO:199, and/or incorporate amino acid sequences identical to the CDR1 (SEQ ID NO:131), CDR2 (SEQ ID NO:132), and CDR3 (SEQ ID NO:133) sequences of SEQ ID NO:199. Similarly, the light chain variable domain of the scFv can be at least 90% (e.g., 90%, 91%, 92%, 93%, 94%, 95%, 96%, 97%, 98%, 99%, or 100%) identical to SEQ ID NO:200, and/or incorporate amino acid sequences identical to the CDR1 (SEQ ID NO:135), CDR2 (SEQ ID NO:136), and CDR3 (SEQ ID NO:137) sequences of SEQ ID NO:200. In some embodiments, the scFv has the amino acid sequence of SEQ ID NO:171.

The scFv described above includes a heavy chain variable domain and a light chain variable domain. In some embodiments, the heavy chain variable domain forms a disulfide bridge with the light chain variable domain to enhance stability of the scFv. For example, a disulfide bridge can be formed between the C44 residue of the heavy chain variable domain and the C100 residue of the light chain variable domain, the amino acid positions numbered under Kabat. For example, in some embodiments, the disulfide bridge is formed between a cysteine residue (naturally present or introduced by mutation) at position 44 (C44) of the VH of the scFv and a cysteine residue (naturally present or introduced by mutation) at position 100 (C100) of the VL of the scFv, numbered under the Kabat numbering scheme.

The VH and VL of the scFv can be positioned in various orientations. In certain embodiments, the VL is positioned N-terminal to the VH. In certain embodiments, the VL is positioned C-terminal to the VH.

The VH and VL of the scFv can be connected via a linker, e.g., a peptide linker. In certain embodiments, the peptide linker is a flexible linker. Regarding the amino acid composition of the linker, peptides are selected with properties that confer flexibility, do not interfere with the structure and function of the other domains of the proteins of the present invention, and resist cleavage from proteases. For example, glycine and serine residues generally provide protease resistance. In certain embodiments, the VL is positioned N-terminal to the VH and is connected to the VH via a linker.

The length of the linker (e.g., flexible linker) can be “short,” e.g., 0, 1, 2, 3, 4, 5, 6, 7, 8, 9, 10, 11 or 12 amino acid residues, or “long,” e.g., at least 13 amino acid residues. In certain embodiments, the linker is 10-50, 10-40, 10-30, 10-25, 10-20, 15-50, 15-40, 15-30, 15-15-20, 20-50, 20-40, 20-30, or 20-25 amino acid residues in length.

In certain embodiments, the linker includes a (GS)ₙ (SEQ ID NO:204), (GGS)ₙ (SEQ ID NO:205), (GGGS)ₙ (SEQ ID NO:151), (GGSG)ₙ (SEQ ID NO:153), (GGSGG)ₙ (SEQ ID NO:156), and (GGGGS)ₙ (SEQ ID NO:157) sequence, wherein n is 1, 2, 3, 4, 5, 6, 7, 8, 9, 10, 11, 12, 13, 14, 15, 16, 17, 18, 19, or 20. In certain embodiments, the linker includes an amino acid sequence selected from SEQ ID NO:143, SEQ ID NO:201, SEQ ID NO:202, SEQ ID NO: 103, SEQ ID NO:104, SEQ ID NO:83, SEQ ID NO:84, SEQ ID NO:150, SEQ ID NO:152, and SEQ ID NO:154, as listed in Table 3.

TABLE 3
Exemplary Linkers

| SEQ ID | Amino Acid Sequence |
|---|---|
| SEQ ID | GSGSGSGSGSGSGSGSGSGS |
| NO: 201 | |
| SEQ ID | GGSGGSGGSGGSGGSGGSGGSGGSGGSGGS |
| NO: 202 | |
| SEQ ID | GGGSGGGSGGGSGGGSGGGSGGGSGGGSGGGSGGGS |
| NO: 103 | GGGS |
| SEQ ID | GGSGGGSGGGSGGGSGGGSGGGSGGGSGGGSGGGSG |
| NO: 104 | GGSG |
| SEQ ID | GGSGGGGSGGGGSGGGGSGGGGSGGGGSGGGGSGG |
| NO: 83 | GGSGGGGSGGGGSGG |
| SEQ ID | GGGGSGGGGSGGGGSGGGGSGGGGSGGGGSGGGGS |
| NO: 84 | GGGGSGGGGSGGGGS |
| SEQ ID | GGGGSGGGGSGGGGSGGGGS |
| NO: 143 | |
| SEQ ID | GGGGSGGGGSGGGGS |
| NO: 150 | |
| SEQ ID | GGGGSGGGGSGGGGSGGGGSGGGGSGGGGSGGGGS |
| NO: 152 | GGGGSGGGGSGGGGSGGGGSGGGGSGGGGSGGGGS |
| | GGGGSGGGGSGGGGSGGGGSGGGGSGGGGS |
| SEQ ID | GGSGGGGSGGGGSGGGGSGGGGSGGGGSGGGGSGG |
| NO: 154 | GGSGGGGSGGGGSGGGGSGGGGSGGGGSGGGGSGG |
| | GGSGGGGSGGGGSGGGGSGGGGSGGGGSGG |

In specific embodiments, the light chain variable domain is linked to the N-terminus of the heavy chain variable domain via a flexible linker, e.g., the (G₄S)₄ linker (SEQ ID NO:143).

In some embodiments, the scFv described above is linked to an antibody Fc polypeptide via a hinge sequence. In some embodiments, the hinge includes the amino acid sequence Ala-Ser. In some other embodiments, the hinge includes the amino acid sequences Ala-Ser and Thr-Lys-Gly. The hinge sequence can provide flexibility of binding to the target antigen and balance between flexibility and optimal geometry.

Fc Domain

The antibody Fc domain of the multi-specific binding protein includes a first antibody Fc polypeptide linked to the Fab and a second antibody Fc polypeptide linked to the scFv. The two antibody Fc polypeptides pair and form a dimer that binds CD16.

Within the antibody Fc domain, CD16 binding is mediated by the hinge region and the CH2 domain. For example, within human IgG1, the interaction with CD16 is primarily focused on amino acid residues Asp 265-Glu 269, Asn 297-Thr 299, Ala 327-Ile 332, Leu 234-Ser 239, and carbohydrate residue N-acetyl-D-glucosamine in the CH2 domain (see, Sondermann et al., Nature, 406 (6793):267-273). Based on the known domains, mutations can be selected to enhance or reduce the binding affinity to CD16, such as by using phage-displayed libraries or yeast surface-displayed cDNA libraries, or can be designed based on the known three-dimensional structure of the interaction.

The assembly of heterodimeric antibody heavy chains can be accomplished by expressing two different antibody heavy chain sequences in the same cell, which may lead to the assembly of homodimers of each antibody heavy chain as well as assembly of heterodimers. Promoting the preferential assembly of heterodimers can be accomplished by incorporating different mutations in the CH3 domain of each antibody heavy chain constant region as shown in U.S. Ser. No. 13/494,870, U.S. Ser. No. 16/028,850, U.S. Ser. No. 11/533,709, U.S. Ser. No. 12/875,015, U.S. Ser. No. 13/289,934, Ser. No. 14/773,418, U.S. Ser. No. 12/811,207, U.S. Ser. No. 13/866,756, U.S. Ser. No. 14/647,480, and U.S. Ser. No. 14/830,336. For example, mutations can be made in the CH3 domain based on human IgG1 through incorporating distinct pairs of amino acid substitutions within a first polypeptide and a second polypeptide that allow these two chains to selectively heterodimerize with each other. The positions of amino acid substitutions illustrated below are all numbered according to the EU index as in Kabat.

In one scenario, an amino acid substitution in the first polypeptide replaces the original amino acid with a larger amino acid, selected from arginine (R), phenylalanine (F), tyrosine (Y) or tryptophan (W), and at least one amino acid substitution in the second polypeptide replaces the original amino acid(s) with a smaller amino acid(s), chosen from alanine (A), serine (S), threonine (T), or valine (V), such that the larger amino acid substitution (a protuberance) fits into the surface of the smaller amino acid substitutions (a cavity). For example, one polypeptide can incorporate a T366W substitution, and the other can incorporate three substitutions including T366S, L368A, and Y407V.

An antibody heavy chain variable domain of the invention can optionally be coupled to an amino acid sequence at least 90% identical to an antibody constant region, such as an IgG constant region including hinge, CH2 and CH3 domains with or without a CH1 domain. In some embodiments, the amino acid sequence of the constant region is at least 90% identical to a human antibody constant region, such as a human IgG1 constant region, an IgG2 constant region, IgG3 constant region, or IgG4 constant region. In some other embodiments, the amino acid sequence of the constant region is at least 90% identical to an antibody constant region from another mammal, such as rabbit, dog, cat, mouse, or horse. One or more mutations can be incorporated into the constant region as compared to human IgG1 constant region, for example at Q347, Y349, L351, 5354, E356, E357, K360, Q362, 5364, T366, L368, K370, N390, K392, T394, D399, 5400, D401, F405, Y407, K409, T411 and/or K439. Exemplary substitutions include, for example, Q347E, Q347R, Y349S, Y349K, Y349T, Y349D, Y349E, Y349C, T350V, L351K, L351D, L351Y, S354C, E356K, E357Q, E357L, E357W, K360E, K360W, Q362E, S364K, S364E, S364H, S364D, T366V, T366I, T366L, T366M, T366K, T366W, T366S, L368E, L368A, L368D, K370S, N390D, N390E, K392L, K392M, K392V, K392F, K392D, K392E, T394F, T394W, D399R, D399K, D399V, S400K, S400R, D401K, F405A, F405T, Y407A, Y4071, Y407V, K409F, K409W, K409D, T411D, T411E, K439D, and K439E. All the amino acid positions in an Fc domain or hinge region disclosed herein are numbered according to EU numbering.

In certain embodiments, mutations that can be incorporated into the CH1 of a human IgG1 constant region may be at amino acid V125, F126, P127, T135, T139, A140, F170, P171, and/or V173. In certain embodiments, mutations that can be incorporated into the Cκ of a human IgG1 constant region may be at amino acid E123, F116, S176, V163, S174, and/or T164.

Amino acid substitutions could be selected from the following sets of substitutions shown in Table 4.

TABLE 4
Exemplary Fc Substitutions that Promote Heterodimerization

| | First Polypeptide | Second Polypeptide |
|---|---|---|
| Set 1 | S364E/F405A | Y349K/T394F |
| Set 2 | S364H/D401K | Y349T/T411E |
| Set 3 | S364H/T394F | Y349T/F405A |
| Set 4 | S364E/T394F | Y349K/F405A |
| Set 5 | S364E/T411E | Y349K/D401K |
| Set 6 | S364D/T394F | Y349K/F405A |
| Set 7 | S364H/F405A | Y349T/T394F |
| Set 8 | S364K/E357Q | L368D/K370S |
| Set 9 | L368D/K370S | S364K |
| Set 10 | L368E/K370S | S364K |
| Set 11 | K360E/Q362E | D401K |
| Set 12 | L368D/K370S | S364K/E357L |
| Set 13 | K370S | S364K/E357Q |
| Set 14 | F405L | K409R |
| Set 15 | K409R | F405L |

Alternatively, amino acid substitutions could be selected from the following sets of substitutions shown in Table 5.

TABLE 5
Exemplary Fc Substitutions that Promote Heterodimerization

| | First Polypeptide | Second Polypeptide |
|---|---|---|
| Set 1 | K409W | D399V/F405T |
| Set 2 | Y349S | E357W |
| Set 3 | K360E | Q347R |
| Set 4 | K360E/K409W | Q347R/D399V/F405T |
| Set 5 | Q347E/K360E/K409W | Q347R/D399V/F405T |
| Set 6 | Y349S/K409W | E357W/D399V/F405T |

Alternatively, amino acid substitutions could be selected from the following sets of substitutions shown in Table 6.

TABLE 6
Exemplary Fc Substitutions that Promote Heterodimerization

| | First Polypeptide | Second Polypeptide |
|---|---|---|
| Set 1 | T366K/L351K | L351D/L368E |
| Set 2 | T366K/L351K | L351D/Y349E |
| Set 3 | T366K/L351K | L351D/Y349D |
| Set 4 | T366K/L351K | L351D/Y349E/L368E |
| Set 5 | T366K/L351K | L351D/Y349D/L368E |
| Set 6 | E356K/D399K | K392D/K409D |

Alternatively, at least one amino acid substitution in each polypeptide chain could be selected from Table 7.

TABLE 7
Exemplary Fc Substitutions that Promote Heterodimerization

| First Polypeptide | Second Polypeptide |
|---|---|
| L351Y, D399R, D399K, | T366V, T366I, T366L, T366M, N390D, |
| S400K, S400R, Y407A, | N390E, K392L, K392M, K392V, K392F |
| Y407I, Y407V | K392D, K392E, K409F, K409W, T411D |
| | and T411E |

Alternatively, at least one amino acid substitution could be selected from the following sets of substitutions in Table 8, where the position(s) indicated in the First Polypeptide column is replaced by any known negatively-charged amino acid, and the position(s) indicated in the Second Polypeptide Column is replaced by any known positively-charged amino acid.

TABLE 8
Exemplary Fc Positions for Substitutions

| First Polypeptide | Second Polypeptide |
|---|---|
| K392, K370, K409, or K439 | D399, E356, or E357 |

Alternatively, at least one amino acid substitution could be selected from the following sets of substitutions in Table 9, where the position(s) indicated in the First Polypeptide column is replaced by any known positively-charged amino acid, and the position(s) indicated in the Second Polypeptide Column is replaced by any known negatively-charged amino acid.

TABLE 9
Exemplary Fc Positions for Substitutions

| First Polypeptide | Second Polypeptide |
|---|---|
| D399, E356, or E357 | K409, K439, K370, or K392 |

Alternatively, amino acid substitutions could be selected from the following sets in

Table 10.

TABLE 10
Exemplary Fc Substitutions that Promote Heterodimerization

| First Polypeptide | Second Polypeptide |
|---|---|
| T350V, L351Y, F405A, | T350V, T366L, K392L, |
| and Y407V | and T394W |

When selecting Fc substitutions, a skilled person would appreciate that the first polypeptide and the second polypeptide in Tables 4-10 may correspond to the first antibody Fc polypeptide and the second antibody Fc polypeptide, respectively. Alternatively, the first polypeptide and the second polypeptide in Tables 4-10 may correspond to the second antibody Fc polypeptide and the first antibody Fc polypeptide, respectively.

In some embodiments, the amino acid sequence of one polypeptide chain of the antibody constant region differs from the amino acid sequence of an IgG1 constant region at position T366, and the amino acid sequence of the other polypeptide chain of the antibody constant region differs from the amino acid sequence of an IgG1 constant region at one or more positions selected from T366, L368 and Y407.

In some embodiments, the amino acid sequence of one polypeptide chain of the antibody constant region differs from the amino acid sequence of an IgG1 constant region at one or more positions selected from T366, L368 and Y407, and the amino acid sequence of the other polypeptide chain of the antibody constant region differs from the amino acid sequence of an IgG1 constant region at position T366.

In some embodiments, the amino acid sequence of one polypeptide chain of the antibody constant region differs from the amino acid sequence of an IgG1 constant region at one or more positions selected from E357, K360, Q362, S364, L368, K370, T394, D401, F405, and T411, and the amino acid sequence of the other polypeptide chain of the antibody constant region differs from the amino acid sequence of an IgG1 constant region at one or more positions selected from Y349, E357, S364, L368, K370, T394, D401, F405 and T411.

In some embodiments, the amino acid sequence of one polypeptide chain of the antibody constant region differs from the amino acid sequence of an IgG1 constant region at one or more positions selected from Y349, E357, S364, L368, K370, T394, D401, F405 and T411, and the amino acid sequence of the other polypeptide chain of the antibody constant region differs from the amino acid sequence of an IgG1 constant region at one or more positions selected from E357, K360, Q362, S364, L368, K370, T394, D401, F405, and T411.

In some embodiments, the amino acid sequence of one polypeptide chain of the antibody constant region differs from the amino acid sequence of an IgG1 constant region at one or more positions selected from L351, D399, S400 and Y407, and the amino acid sequence of the other polypeptide chain of the antibody constant region differs from the amino acid sequence of an IgG1 constant region at one or more positions selected from T366, N390, K392, K409 and T411.

In some embodiments, the amino acid sequence of one polypeptide chain of the antibody constant region differs from the amino acid sequence of an IgG1 constant region at one or more positions selected from T366, N390, K392, K409 and T411, and the amino acid sequence of the other polypeptide chain of the antibody constant region differs from the amino acid sequence of an IgG1 constant region at one or more positions selected from L351, D399, S400 and Y407.

In some embodiments, the amino acid sequence of one polypeptide chain of the antibody constant region differs from the amino acid sequence of an IgG1 constant region at one or more positions selected from Q347, Y349, K360, and K409, and the amino acid sequence of the other polypeptide chain of the antibody constant region differs from the amino acid sequence of an IgG1 constant region at one or more positions selected from Q347, E357, D399 and F405.

In some embodiments, the amino acid sequence of one polypeptide chain of the antibody constant region differs from the amino acid sequence of an IgG1 constant region at one or more positions selected from Q347, E357, D399 and F405, and the amino acid sequence of the other polypeptide chain of the antibody constant region differs from the amino acid sequence of an IgG1 constant region at one or more positions selected from Y349, K360, Q347 and K409.

In some embodiments, the amino acid sequence of one polypeptide chain of the antibody constant region differs from the amino acid sequence of an IgG1 constant region at one or more positions selected from K370, K392, K409 and K439, and the amino acid sequence of the other polypeptide chain of the antibody constant region differs from the amino acid sequence of an IgG1 constant region at one or more positions selected from D356, E357 and D399.

In some embodiments, the amino acid sequence of one polypeptide chain of the antibody constant region differs from the amino acid sequence of an IgG1 constant region at one or more positions selected from D356, E357 and D399, and the amino acid sequence of the other polypeptide chain of the antibody constant region differs from the amino acid sequence of an IgG1 constant region at one or more positions selected from K370, K392, K409 and K439.

In some embodiments, the amino acid sequence of one polypeptide chain of the antibody constant region differs from the amino acid sequence of an IgG1 constant region at one or more positions selected from L351, E356, T366 and D399, and the amino acid sequence of the other polypeptide chain of the antibody constant region differs from the amino acid sequence of an IgG1 constant region at one or more positions selected from Y349, L351, L368, K392 and K409.

In some embodiments, the amino acid sequence of one polypeptide chain of the antibody constant region differs from the amino acid sequence of an IgG1 constant region at one or more positions selected from Y349, L351, L368, K392 and K409, and the amino acid sequence of the other polypeptide chain of the antibody constant region differs from the amino acid sequence of an IgG1 constant region at one or more positions selected from L351, E356, T366 and D399.

In some embodiments, the amino acid sequence of one polypeptide chain of the antibody constant region differs from the amino acid sequence of an IgG1 constant region by K360E and K409W substitutions, and the amino acid sequence of the other polypeptide chain of the antibody constant region differs from the amino acid sequence of an IgG1 constant region by Q347R, D399V and F405T substitutions.

In some embodiments, the amino acid sequence of one polypeptide chain of the antibody constant region differs from the amino acid sequence of an IgG1 constant region by Q347R, D399V and F405T substitutions, and the amino acid sequence of the other polypeptide chain of the antibody constant region differs from the amino acid sequence of an IgG1 constant region by K360E and K409W substitutions.

In some embodiments, the amino acid sequence of one polypeptide chain of the antibody constant region differs from the amino acid sequence of an IgG1 constant region by a T366W substitution, and the amino acid sequence of the other polypeptide chain of the antibody constant region differs from the amino acid sequence of an IgG1 constant region by T366S, T368A, and Y407V substitutions.

In some embodiments, the amino acid sequence of one polypeptide chain of the antibody constant region differs from the amino acid sequence of an IgG1 constant region by T366S, T368A, and Y407V substitutions, and the amino acid sequence of the other polypeptide chain of the antibody constant region differs from the amino acid sequence of an IgG1 constant region by a T366W substitution.

In some embodiments, the amino acid sequence of one polypeptide chain of the antibody constant region differs from the amino acid sequence of an IgG1 constant region by T350V, L351Y, F405A, and Y407V substitutions, and the amino acid sequence of the other polypeptide chain of the antibody constant region differs from the amino acid sequence of an IgG1 constant region by T350V, T366L, K392L, and T394W substitutions.

In some embodiments, the amino acid sequence of one polypeptide chain of the antibody constant region differs from the amino acid sequence of an IgG1 constant region by T350V, T366L, K392L, and T394W substitutions, and the amino acid sequence of the other polypeptide chain of the antibody constant region differs from the amino acid sequence of an IgG1 constant region by T350V, L351Y, F405A, and Y407V substitutions.

Alternatively, or additionally, the structural stability of a hetero-multimeric protein may be increased by introducing an S354C substitution on either of the first or second polypeptide chain, and a Y349C substitution on the opposing polypeptide chain, which forms an artificial disulfide bridge within the interface of the two polypeptides. In some embodiments, the amino acid sequence of one polypeptide chain of the antibody constant region differs from the amino acid sequence of an IgG1 constant region by an S354C substitution, and the amino acid sequence of the other polypeptide chain of the antibody constant region differs from the amino acid sequence of an IgG1 constant region by a Y349C substitution.

When selecting Fc substitutions, a skilled person would appreciate that the “one polypeptide chain” and “the other polypeptide chain” of an antibody constant region described above may correspond to the first antibody Fc polypeptide and the second antibody Fc polypeptide, respectively. Alternatively, the “one polypeptide chain” and “the other polypeptide chain” of an antibody constant region described above may correspond to the second antibody Fc polypeptide and the first antibody Fc polypeptide, respectively.

Exemplary Multi-Specific Binding Proteins

Listed below are examples of TriNKETs that include a HER2-binding scFv and an NKG2D-binding Fab each linked to an antibody constant region, and antibody constant regions incorporating mutations that enable heterodimerization of two Fc polypeptide chains. The scFv includes a heavy chain variable domain (VH) and a light chain variable domain (VL) derived from an anti-HER2 antibody (e.g., trastuzumab), and further includes substitution of Cys for the amino acid residues at position 100 of the VL and position 44 of the VH, thereby facilitating formation of a disulfide bridge between the VH and VL of the scFv. The VL is linked N-terminal to the VH via a (G₄S)₄ linker (SEQ ID NO:143), and the VH is linked N-terminal to an Fc via an Ala-Ser linker. The Ala-Ser linker is included at the elbow hinge region sequence to balance between flexibility and optimal geometry. In certain embodiments, an additional sequence, Thr-Lys-Gly, can be added N-terminal or C-terminal to the Ala-Ser sequence at the hinge. As used herein to describe these exemplary TriNKETs, the Fc domain includes an antibody hinge, CH2, and CH3.

Accordingly, each of the TriNKETs described below have the following three polypeptide chains:
Chain A, including from N-terminus to C-terminus: a VH of an NKG2D-binding Fab, a CH1, and an antibody Fc polypeptide;
Chain B, including from N-terminus to C-terminus: a VL of a HER2-binding scFv, a (G₄S)₄ linker (SEQ ID NO:143), a VH of the HER2-binding scFv, an Ala-Ser linker, and an antibody Fc polypeptide; and
Chain C, including from N-terminus to C-terminus: a VL of the NKG2D-binding Fab, and a light chain constant domain (CL).

The amino acid sequences of exemplary TriNKETs are summarized in Table 11.

In certain embodiments, a multi-specific binding protein of the present disclosure includes a first polypeptide chain, a second polypeptide chain, and a third polypeptide chain, having the amino acid sequences of Chain A, Chain B, and Chain C, respectively, of a TriNKET disclosed in Table 11.

In an exemplary embodiment, the antibody Fc polypeptide linked to the NKG2D-binding Fab fragment incorporates the mutations of Q347R, D399V, and F405T, and the antibody Fc polypeptide linked to the HER2 scFv incorporates matching mutations K360E and K409W for forming a heterodimer. In another exemplary embodiment, the antibody Fc polypeptide linked to the NKG2D-binding Fab fragment incorporates knob mutations T366S, L368A, and Y407V, and the antibody Fc polypeptide linked to the HER2-binding scFv incorporates a “hole” mutation T366W. In an exemplary embodiment, the antibody Fc polypeptide linked to the NKG2D-binding Fab fragment includes an S354C substitution in the CH3 domain, which forms a disulfide bond with a Y349C substitution on the antibody Fc polypeptide linked to the HER2-binding scFv.

TABLE 11
Exemplary Multi-Specific Binding Proteins

| | NKG2D | HER2 | | | | |
|---|---|---|---|---|---|---|
| TriNKET | Binding | Binding | Human | | | |
| Construct | Fab | scFv | IgG1 Fc | Chain A | Chain B | Chain C |
| A49-F3′- | A49 | Trastuzumab | EW-RVT | SEQ ID | SEQ ID | SEQ ID |
| TriNKET- | | | | NO: 141 | NO: 140 | NO: 142 |
| Trastuzumab | | | | | | |
| A49-F3′- | A49 | Trastuzumab | KiH | SEQ ID | SEQ ID | SEQ ID |
| KiH- | | | | NO: 147 | NO: 146 | NO: 142 |
| TriNKET- | | | | | | |
| Trastuzumab | | | | | | |
| A49-F3′- | A49 | Pertuzumab | EW-RVT | SEQ ID | SEQ ID | SEQ ID |
| TriNKET- | | | | NO: 141 | NO: 190 | NO: 142 |
| Pertuzumab | | | | | | |
| A49-F3′- | A49 | Pertuzumab | KiH | SEQ ID | SEQ ID | SEQ ID |
| KiH- | | | | NO: 147 | NO: 191 | NO: 142 |
| TriNKET- | | | | | | |
| Pertuzumab | | | | | | |
| A49-F3′- | A49 | MGAH22 | EW-RVT | SEQ ID | SEQ ID | SEQ ID |
| TriNKET- | | | | NO: 141 | NO: 192 | NO: 142 |
| MGAH22 | | | | | | |
| A49-F3′- | A49 | MGAH22 | KiH | SEQ ID | SEQ ID | SEQ ID |
| KiH- | | | | NO: 147 | NO: 193 | NO: 142 |
| TriNKET- | | | | | | |
| MGAH22 | | | | | | |
| A49MI-F3′- | A49MI | Trastuzumab | EW-RVT | SEQ ID | SEQ ID | SEQ ID |
| TriNKET- | | | | NO: 145 | NO: 140 | NO: 142 |
| Trastuzumab | | | | | | |
| A49MI-F3′- | A49MI | Trastuzumab | KiH | SEQ ID | SEQ ID | SEQ ID |
| KiH- | | | | NO: 194 | NO: 146 | NO: 142 |
| TriNKET- | | | | | | |
| Trastuzumab | | | | | | |
| A49MI-F3′- | A49MI | Pertuzumab | EW-RVT | SEQ ID | SEQ ID | SEQ ID |
| TriNKET- | | | | NO: 145 | NO: 190 | NO: 142 |
| Pertuzumab | | | | | | |
| A49MI-F3′- | A49MI | Pertuzumab | KiH | SEQ ID | SEQ ID | SEQ ID |
| KiH- | | | | NO: 194 | NO: 191 | NO: 142 |
| TriNKET- | | | | | | |
| Pertuzumab | | | | | | |
| A49MI-F3′- | A49MI | MGAH22 | EW-RVT | SEQ ID | SEQ ID | SEQ ID |
| TriNKET- | | | | NO: 145 | NO: 192 | NO: 142 |
| MGAH22 | | | | | | |
| A49MI-F3′- | A49MI | MGAH22 | KiH | SEQ ID | SEQ ID | SEQ ID |
| KiH- | | | | NO: 194 | NO: 193 | NO: 142 |
| TriNKET- | | | | | | |
| MGAH22 | | | | | | |
| A44-F3′- | A44 | Trastuzumab | EW-RVT | SEQ ID | SEQ ID | SEQ ID |
| TriNKET- | | | | NO: 155 | NO: 140 | NO: 149 |
| Trastuzumab | | | | | | |
| A44-F3′- | A44 | Trastuzumab | KiH | SEQ ID | SEQ ID | SEQ ID |
| KiH- | | | | NO: 148 | NO: 146 | NO: 149 |
| TriNKET- | | | | | | |
| Trastuzumab | | | | | | |
| A44-F3′- | A44 | Pertuzumab | EW-RVT | SEQ ID | SEQ ID | SEQ ID |
| TriNKET- | | | | NO: 155 | NO: 190 | NO: 149 |
| Pertuzumab | | | | | | |
| A44-F3′- | A44 | Pertuzumab | KiH | SEQ ID | SEQ ID | SEQ ID |
| KiH- | | | | NO: 148 | NO: 191 | NO: 149 |
| TriNKET- | | | | | | |
| Pertuzumab | | | | | | |
| A44-F3′- | A44 | MGAH22 | EW-RVT | SEQ ID | SEQ ID | SEQ ID |
| TriNKET- | | | | NO: 155 | NO: 192 | NO: 149 |
| MGAH22 | | | | | | |
| A44-F3′- | A44 | MGAH22 | KiH | SEQ ID | SEQ ID | SEQ ID |
| KiH- | | | | NO: 148 | NO: 193 | NO: 149 |
| TriNKET- | | | | | | |
| MGAH22 | | | | | | |

Specific TriNKETs and their polypeptide chains are described in more detail below. In the amino acid sequences, (G₄S)₄ (SEQ ID NO:143) and Ala-Ser linkers are bold-underlined; Cys residues in the scFv that form disulfide bridges are bold-italic-underlined; Fc heterodimerization mutations are bold-underlined; and CDR sequences under Kabat are underlined.

For example, a TriNKET of the present disclosure is A49-F3′-TriNKET-Trastuzumab. A49-F3′-TriNKET-Trastuzumab includes a single-chain variable fragment (scFv) (SEQ ID NO:139) derived from trastuzumab that binds HER2, linked via a hinge including Ala-Ser to a first antibody Fc polypeptide; and an NKG2D-binding Fab fragment derived from A49 linked to a second antibody Fc polypeptide. The Fab fragment includes a heavy chain portion having a heavy chain variable domain (SEQ ID NO:94) and a CH1 domain, and a light chain portion having a light chain variable domain (SEQ ID NO:98) and a light chain constant domain. The heavy chain variable domain is connected to the CH1 domain, and the CH1 domain is connected to the second antibody Fc polypeptide. A49-F3′-TriNKET-Trastuzumab includes three polypeptides having the sequences of SEQ ID NO:140, SEQ ID NO:141, and SEQ ID NO:142.

SEQ ID NO:140 represents the full sequence of the HER2-binding scFv linked to the first antibody Fc polypeptide via a hinge including Ala-Ser (scFv-Fc). The first antibody Fc polypeptide includes Q347R, D399V, and F405T substitutions for heterodimerization and an S354C substitution for forming a disulfide bond with a Y349C substitution in SEQ ID NO:141 as described below. The scFv (SEQ ID NO:139) includes a heavy chain variable domain of trastuzumab connected to the N-terminus of a light chain variable domain of trastuzumab via a (G₄S)₄ linker (SEQ ID NO:143), the scFv represented as VL-(G₄S)₄-VH (“(G₄S)₄” is represented by SEQ ID NO:143). The heavy and the light variable domains of the scFv are also connected through a disulfide bridge between C100 of VL and C44 of VH, as a result of Q100C and G44C substitutions in the VL and VH, respectively.

Trastuzumab scFv
(SEQ ID NO: 139)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG [symbol]
GTKVEIKGGGGSGGGGSGGGGSGGGGSEVQLVESGGGLVQPGGSLRLSCA
ASGFNIKDTYIHWVRQAPGK [symbol] LEWVARIYPTNGYTRYADSVKGRFTISAD
TSKNTAYLQMNSLRAEDTAVYYCSRWGGDGFYAMDYWGQGTLVTVSS
Trastuzumab scFv-Fc (RVT)
(SEQ ID NO: 140)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG [symbol]
GTKVEIKGGGGSGGGGSGGGGSGGGGSEVQLVESGGGLVQPGGSLRLSCA
ASGFNIKDTYIHWVRQAPGK [symbol] LEWVARIYPTNGYTRYADSVKGRFTISAD
TSKNTAYLQMNSLRAEDTAVYYCSRWGGDGFYAMDYWGQGTLVTVSSASD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPRVYTLPP [symbol] RDELTKNQVSLTCLVKG
FYPSDIAVEWESNGQPENNYKTTPPVLVSDGSFTLYSKLTVDKSRWQQGN
VFSCSVMHEALHNHYTQKSLSLSPG

SEQ ID NO:141 represents the heavy chain portion of the Fab fragment, which includes a heavy chain variable domain (SEQ ID NO:94) of an NKG2D-binding site and a CH1 domain, connected to the second antibody Fc polypeptide. The antibody Fc polypeptide in SEQ ID NO:141 includes a Y349C substitution in the CH3 domain, which forms a disulfide bond with an S354C substitution on the Fc polypeptide in SEQ ID NO:140. In SEQ ID NO:141, the antibody Fc polypeptide also includes K360E and K409W substitutions for heterodimerization with the Fc in SEQ ID NO:140.

A49 VH
(SEQ ID NO: 94)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSS
A49 VH-CH1-Fc (EW)
(SEQ ID NO: 141)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQV [symbol] TLPPSRDELTENQVSLTCLVKGFYPSDIAVEWESNGQPENNYKTTP
PVLDSDGSFFLYSWLTVDKSRWQQGNVFSCSVMHEALHNHYTQKSLSLSP
G

SEQ ID NO:142 represents the light chain portion of the Fab fragment including a light chain variable domain (SEQ ID NO:98) of an NKG2D-binding site and a light chain constant domain.

A49 VL
(SEQ ID NO: 98)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFGG
GTKVEIK
A49 VL-LC
(SEQ ID NO: 142)
DIQMTQSPSSVSASVGDRVTITCRASQGISSWLAWYQQKPGKAPKLLIYA
ASSLQSGVPSRFSGSGSGTDFTLTISSLQPEDFATYYCQQGVSFPRTFGG
GTKVEIKRTVAAPSPPSDEQLKSGTASVVCLLNNFYPREAKVQWKVDNAL
QSGNSQESVTEQDSKDSTYSLSSTLTLSKADYEKHKVYACEVTHQGLSSP
VTKSFNRGEC

Another TriNKET of the present disclosure is A49MI-F3′-TriNKET-Trastuzumab. A49MI-F3′-TriNKET-Trastuzumab includes the same HER2-binding scFv (SEQ ID NO:139) as in A49-F3′-TriNKET-Trastuzumab linked via a hinge including Ala-Ser to an antibody Fc polypeptide; and an NKG2D-binding Fab fragment derived from A49MI linked to a second antibody Fc polypeptide. The Fab fragment includes a heavy chain portion including a heavy chain variable domain (SEQ ID NO:144) and a CH1 domain, and a light chain portion including a light chain variable domain (SEQ ID NO:98) and a light chain constant domain. The heavy chain variable domain is connected to the CH1 domain, and the CH1 domain is connected to the second antibody Fc polypeptide. A49MI-F3′-TriNKET-Trastuzumab includes three polypeptides having the sequences of SEQ ID NO:140 (as in A49-F3′-TriNKET-Trastuzumab), SEQ ID NO:145, and SEQ ID NO:142 (as in A49-F3′-TriNKET-Trastuzumab).

SEQ ID NO:145 represents a heavy chain portion of the Fab fragment, which includes a heavy chain variable domain (SEQ ID NO:144) of an NKG2D-binding site and a CH1 domain, connected to the first antibody Fc polypeptide. In SEQ ID NO:144, a methionine in the CDR3 of SEQ ID NO:94 has been substituted by isoleucine (M→I substitution; shown within a third bracket [ ] in SEQ ID NO:144 and SEQ ID NO:145). The antibody Fc polypeptide in SEQ ID NO:145 includes a Y349C substitution in the CH3 domain, which forms a disulfide bond with an S354C substitution in the antibody Fc polypeptide in SEQ ID NO:140. In SEQ ID NO:145, the antibody Fc polypeptide also includes K360E and K409W substitutions.

A49MI VH
(SEQ ID NO: 144)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
P[I]GAAAGWFDPWGQGTLVTVSS
A49MI VH-CH1-Fc (EW)
(SEQ ID NO: 145)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
P[I]GAAAGWFDPWGQGTLVTVSSASTKGPSVFPLAPSSKSTSGGTAALG
CLVKDYFPEPVTVSWNSGALTSGVHTFPAVLQSSGLYSLSSVVTVPSSSL
GTQTYICNVNHKPSNTKVDKKVEPKSCDKTHTCPPCPAPELLGGPSVFLF
PPKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQP
REPQV [symbol] TLPPSRDELTENQVSLTCLVKGFYPSDIAVEWESNGQPENNYKT
TPPVLDSDGSFFLYSWLTVDKSRWQQGNVFSCSVMHEALHNHYTQKSLSL
SPG

Another TriNKET of the present disclosure is A49-F3′-KiH-TriNKET-Trastuzumab. KiH refers to the knobs-into-holes (KiH) Fc technology, which involves engineering of the CH3 domains to create either a “knob” or a “hole” in each heavy chain to promote heterodimerization. The concept behind the KiH Fc technology was to introduce a “knob” in one CH3 domain (CH3A) by substitution of a small residue with a bulky one (e.g., T366W_{CH3A} in EU numbering). To accommodate the “knob,” a complementary “hole” surface was created on the other CH3 domain (CH3B) by replacing the closest neighboring residues to the knob with smaller ones (e.g., T366S/L368A/Y407V_{CH3B}). The “hole” mutation was optimized by structure-guided phage library screening (Atwell S, Ridgway J B, Wells JA, Carter P., Stable heterodimers from remodeling the domain interface of a homodimer using a phage display library, J. Mol. Biol. (1997) 270(1):26-35). X-ray crystal structures of KiH Fc variants (Elliott J M, Ultsch M, Lee J, Tong R, Takeda K, Spiess C, et al., Antiparallel conformation of knob and hole aglycosylated half-antibody homodimers is mediated by a CH2-CH3 hydrophobic interaction. J. Mol. Biol. (2014) 426(9):1947-57; Mimoto F, Kadono S, Katada H, Igawa T, Kamikawa T, Hattori K. Crystal structure of a novel asymmetrically engineered Fc domain variant with improved affinity for FcγRs. Mol. Immunol. (2014) 58(1):132-8) demonstrated that heterodimerization is thermodynamically favored by hydrophobic interactions driven by steric complementarity at the inter-CH3 domain core interface, whereas the knob-knob and the hole-hole interfaces do not favor homodimerization owing to steric hindrance and disruption of the favorable interactions, respectively.

A49-F3′-KiH-TriNKET-Trastuzumab includes the same HER2-binding scFv (SEQ ID NO:139) as in A49-F3′-TriNKET-Trastuzumab linked via a hinge including Ala-Ser to a first antibody Fc polypeptide incorporating the “hole” substitutions of T366S, L368A, and Y407V; and the same NKG2D-binding Fab fragment as in A49-F3′-TriNKET-Trastuzumab, the CH1 domain of which is connected to a second antibody Fc polypeptide incorporating the “knob” substitution of T366W. A49-F3′-KiH-TriNKET-Trastuzumab includes three polypeptides having the sequences of SEQ ID NO:146, SEQ ID NO:147, and SEQ ID NO:142 (as in A49-F3′-TriNKET-Trastuzumab).

SEQ ID NO:146 represents the full sequence of the HER2-binding scFv (SEQ ID NO:139) linked to the first antibody Fc polypeptide via a hinge including Ala-Ser (scFv-Fc). The first antibody Fc polypeptide includes T366S, L368A, and Y407V substitutions for heterodimerization and an S354C substitution for forming a disulfide bond with a Y349C substitution in SEQ ID NO:147 as described below.

Trastuzumab scFv-Fc (KiH)
(SEQ ID NO: 146)
DIQMTQSPSSLSASVGDRVTITCRASQDVNTAVAWYQQKPGKAPKLLIYS
ASFLYSGVPSRFSGSRSGTDFTLTISSLQPEDFATYYCQQHYTTPPTFG [symbol]
GTKVEIKGGGGSGGGGSGGGGSGGGGSEVQLVESGGGLVQPGGSLRLSCA
ASGFNIKDTYIHWVRQAPGK [symbol] LEWVARIYPTNGYTRYADSVKGRFTISAD
TSKNTAYLQMNSLRAEDTAVYYCSRWGGDGFYAMDYWGQGTLVTVSSASD
KTHTCPPCPAPELLGGPSVFLFPPKPKDTLMISRTPEVTCVVVDVSHEDP
EVKFNWYVDGVEVHNAKTKPREEQYNSTYRVVSVLTVLHQDWLNGKEYKC
KVSNKALPAPIEKTISKAKGQPREPQV [symbol] TLPPSRDELTKNQVSLSCAVKG
FYPSDIAVEWESNGQPENNYKTTPPVLDSDGSFFLVSKLTVDKSRWQQGN
VFSCSVMHEALHNHYTQKSLSLSPG

SEQ ID NO:147 represents the heavy chain portion of a Fab fragment having a heavy chain variable domain (SEQ ID NO:94) of an NKG2D-binding site derived from A49 and a CH1 domain, connected to the second antibody Fc polypeptide. The antibody Fc polypeptide in SEQ ID NO:147 includes an S354C substitution, which forms a disulfide bond with a Y349C substitution in the CH3 domain of the first antibody Fc polypeptide. In SEQ ID NO:147, the antibody Fc polypeptide also includes a T366W substitution.

A49 VH-CH1-Fc (KiH)
(SEQ ID NO: 147)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PMGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPP [symbol] RDELTKNQVSLWCLVKGFYPSDIAVEWESNGQPENNYKTTP
PVLDSDGSFFLYSKLTVDKSRWQQGNVFSCSVMHEALHNHYTQKSLSLSP
G

Another TriNKET of the present disclosure is A49MI-F3′-KiH-TriNKET-Trastuzumab. A49MI-F3′-KiH-TriNKET-Trastuzumab includes the same HER2-binding scFv (SEQ ID NO:139) as in A49-F3′-TriNKET-Trastuzumab linked via a hinge including Ala-Ser to a first antibody Fc polypeptide incorporating the “hole” substitutions of T366S, L368A, and Y407V; and the same NKG2D-binding Fab fragment as in A49MI-F3′-TriNKET-Trastuzumab, the CH1 domain of which is connected to a second antibody Fc polypeptide incorporating the “knob” substitution of T366W. A49MI-F3′-KiH-TriNKET-Trastuzumab includes three polypeptides having the sequences of SEQ ID NO:146 (as in A49-F3′-KiH-TriNKET-Trastuzumab), SEQ ID NO:194, and SEQ ID NO:142 (as in A49-F3′-TriNKET-Trastuzumab).

SEQ ID NO:194 represents the heavy chain portion of a Fab fragment having a heavy chain variable domain (SEQ ID NO:144) of an NKG2D-binding site derived from A49MI and a CH1 domain, connected to the second antibody Fc polypeptide. The antibody Fc polypeptide in SEQ ID NO:194 includes an S354C substitution, which forms a disulfide bond with a Y349C substitution in the CH3 domain of the first antibody Fc polypeptide. In SEQ ID NO:194, the antibody Fc polypeptide also includes a T366W substitution.

A49MI VH-CH1-Fc (KiH)
(SEQ ID NO: 194)
EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCARGA
PIGAAAGWFDPWGQGTLVTVSSASTKGPSVFPLAPSSKSTSGGTAALGCL
VKDYFPEPVTVSWNSGALTSGVHTFPAVLQSSGLYSLSSVVTVPSSSLGT
QTYICNVNHKPSNTKVDKKVEPKSCDKTHTCPPCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPP [symbol] RDELTKNQVSLWCLVKGFYPSDIAVEWESNGQPENNYKTTP
PVLDSDGSFFLYSKLTVDKSRWQQGNVFSCSVMHEALHNHYTQKSLSLSP
G

Another exemplary TriNKET of the present disclosure is A44-F3′-TriNKET-Trastuzumab. A44-F3′-TriNKET-Trastuzumab includes the same HER2-binding scFv (SEQ ID NO:139) as in A49-F3′-TriNKET-Trastuzumab linked via a hinge including Ala-Ser to a first antibody Fc polypeptide; and an NKG2D-binding Fab fragment derived from A44 linked to a second antibody Fc polypeptide. The Fab fragment includes a heavy chain portion having a heavy chain variable domain (SEQ ID NO:86) and a CH1 domain, and a light chain portion having a light chain variable domain (SEQ ID NO:90) and a light chain constant domain. The heavy chain variable domain is connected to the CH1 domain, and the CH1 domain is connected to the second antibody Fc polypeptide. A44-F3′-TriNKET-Trastuzumab includes three polypeptides having the sequences of SEQ ID NO:140 (as in A49-F3′-TriNKET-Trastuzumab), SEQ ID NO:155, and SEQ ID NO:149.

SEQ ID NO:155 represents a heavy chain variable domain (SEQ ID NO:86) of an NKG2D-binding site derived from A44, connected to the second antibody Fc polypeptide. The antibody Fc polypeptide in SEQ ID NO:155 includes a Y349C substitution in the CH3 domain, which forms a disulfide bond with an S354C substitution on the first antibody Fc polypeptide. In SEQ ID NO:155, the antibody Fc polypeptide also includes K360E and K409W substitutions.

A44 VH
(SEQ ID NO: 86)
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDG
GYYDSGAGDYWGQGTLVTVSS
A44 VH-CH1-Fc (EW)
(SEQ ID NO: 155)
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDG
GYYDSGAGDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QV [symbol] TLPPSRDELTENQVSLTCLVKGFYPSDIAVEWESNGQPENNYKTTPP
VLDSDGSFFLYSWLTVDKSRWQQGNVFSCSVMHEALHNHYTQKSLSLSPG

SEQ ID NO:149 represents the light chain portion of the Fab fragment including a light chain variable domain (SEQ ID NO:90) of an NKG2D-binding site and a light chain constant domain.

A44 VL
(SEQ ID NO: 90)
DIQMTQSPSSVSASVGDRVTITCRASQGIDSWLAWYQQKPGKAPKLLIYA
ASSLQSGVPSRFSGSGSGTDFTLTISSLQPEDFATYYCQQGVSYPRTFGG
GTKVEIK
A44 VL-CL
(SEQ ID NO: 149)
DIQMTQSPSSVSASVGDRVTITCRASQGIDSWLAWYQQKPGKAPKLLIYA
ASSLQSGVPSRFSGSGSGTDFTLTISSLQPEDFATYYCQQGVSYPRTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

Another exemplary TriNKET of the present disclosure is A44-F3′-KiH-TriNKET-Trastuzumab. A44-F3′-KiH-TriNKET-Trastuzumab includes the same HER2-binding scFv (SEQ ID NO:139) as in A49-F3′-TriNKET-Trastuzumab linked via a hinge including Ala-Ser to a first antibody Fc polypeptide incorporating the “hole” substitutions of T366S, L368A, and Y407V; and the same NKG2D-binding Fab fragment as in A44-F3′-TriNKET-Trastuzumab, the CH1 domain of which is connected to a second antibody Fc polypeptide incorporating the “knob” substitution of T366W. A44-F3′-KiH-TriNKET-Trastuzumab includes three polypeptides having the sequences of SEQ ID NO:146 (as in A49-F3′-KiH-TriNKET-Trastuzumab), SEQ ID NO:148, and SEQ ID NO:149 (as in A44-F3′-TriNKET-Trastuzumab).

SEQ ID NO:148 represents a heavy chain variable domain (SEQ ID NO:86) of an NKG2D-binding site derived from A44, connected to the second antibody Fc polypeptide. The antibody Fc polypeptide in SEQ ID NO:148 includes a Y349C substitution in the CH3 domain, which forms a disulfide bond with an S354C substitution on the first antibody Fc polypeptide. In SEQ ID NO:148, the antibody Fc polypeptide also includes a T366W substitution.

A44 VH-CH1-Fc (KiH)
(SEQ ID NO: 148)
EVQLLESGGGLVQPGGSLRLSCAASGFTFSSYAMSWVRQAPGKGLEWVSA
ISGSGGSTYYADSVKGRFTISRDNSKNTLYLQMNSLRAEDTAVYYCAKDG
GYYDSGAGDYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPP [symbol] RDELTKNQVSLWCLVKGFYPSDIAVEWESNGQPENNYKTTPP
VLDSDGSFFLYSKLTVDKSRWQQGNVFSCSVMHEALHNHYTQKSLSLSPG

Another TriNKET of the present disclosure is A49-F3′-TriNKET-Pertuzumab. A49-F3′-TriNKET-Pertuzumab includes an scFv (SEQ ID NO:189) derived from pertuzumab that binds HER2, linked via a hinge including Ala-Ser to a first antibody Fc polypeptide; and the same NKG2D-binding Fab fragment as in A49-F3′-TriNKET-Trastuzumab, the CH1 domain of which is connected to a second antibody Fc polypeptide. The first antibody Fc polypeptide incorporates Q347R, D399V, and F405T substitutions, and the second antibody Fc polypeptide incorporates K360E and K409W substitutions. A49-F3′-TriNKET-Pertuzumab includes three polypeptides, having the sequences of SEQ ID NO:190, SEQ ID NO:141 (as in A49-F3′-TriNKET-Trastuzumab), and SEQ ID NO:142 (as in A49-F3′-TriNKET-Trastuzumab).

SEQ ID NO:190 represents the full sequence of the HER2-binding scFv linked to the first antibody Fc polypeptide via a hinge including Ala-Ser (scFv-Fc). The first antibody Fc polypeptide incorporates Q347R, D399V, and F405T substitutions for heterodimerization and an S354C substitution for forming a disulfide bond with a Y349C substitution in SEQ ID NO:141 as described above. The scFv (SEQ ID NO:189) includes a heavy chain variable domain of pertuzumab connected to the N-terminus of a light chain variable domain of pertuzumab via a (G₄S)₄ linker (SEQ ID NO:143), the scFv represented as VL-(G₄S)₄-VH (“(G₄S)₄” is represented by SEQ ID NO:143). The heavy and the light variable domains of the scFv are also connected through a disulfide bridge between C100 of VL and C44 of VH, as a result of Q100C and G44C substitutions in the VL and VH, respectively.

Pertuzumab scFv
(SEQ ID NO: 189)
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFG [symbol]
GTKVEIKRGGGGSGGGGSGGGGSGGGGSEVQLVESGGGLVQPGGSLRLSC
AASGFTFTDYTMDWVRQAPGK [symbol] LEWVADVNPNSGGSIYNQRFKGRFTLSV
DRSKNTLYLQMNSLRAEDTAVYYCARNLGPSFYFDYWGQGTLVTVSSA
Pertuzumab scFv-Fc
(SEQ ID NO: 190)
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFG [symbol]
GTKVEIKRGGGGSGGGGSGGGGSGGGGSEVQLVESGGGLVQPGGSLRLSC
AASGFTFTDYTMDWVRQAPGK [symbol] LEWVADVNPNSGGSIYNQRFKGRFTLSV
DRSKNTLYLQMNSLRAEDTAVYYCARNLGPSFYFDYWGQGTLVTVSSAAS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPRVYTLPP [symbol] RDELTKNQVSLTCLVK
GFYPSDIAVEWESNGQPENNYKTTPPVLVSDGSFTLYSKLTVDKSRWQQG
NVFSCSVMHEALHNHYTQKSLSLSPG

Another exemplary TriNKET of the present disclosure is A49MI-F3′-TriNKET-Pertuzumab. A49MI-F3′-TriNKET-Pertuzumab includes the same HER2-binding scFv (SEQ ID NO:189) as in A49-F3′-TriNKET-Pertuzumab linked via a hinge including Ala-Ser to a first antibody Fc polypeptide; and the same NKG2D-binding Fab fragment as in A49MI-F3′-TriNKET-Trastuzumab, the CH1 domain of which is connected to a second antibody Fc polypeptide. The first antibody Fc polypeptide incorporates Q347R, D399V, and F405T substitutions, and the second antibody Fc polypeptide incorporates K360E and K409W substitutions. A49MI-F3′-TriNKET-Pertuzumab includes three polypeptides having the sequences of SEQ ID NO:190 (as in A49-F3′-KiH-TriNKET-Pertuzumab), SEQ ID NO:145 (as in A49MI-F3′-TriNKET-Trastuzumab), and SEQ ID NO:142 (as in A49-F3′-TriNKET-Trastuzumab).

Another exemplary TriNKET of the present disclosure is A49-F3′-KiH-TriNKET-Pertuzumab. A49-F3′-KiH-TriNKET-Pertuzumab includes the same HER2-binding scFv (SEQ ID NO:189) as in A49-F3′-TriNKET-Pertuzumab linked via a hinge including Ala-Ser to a first antibody Fc polypeptide; and the same NKG2D-binding Fab fragment as in A49-F3′-TriNKET-Trastuzumab, the CH1 domain of which is connected to a second antibody Fc polypeptide. The first antibody Fc polypeptide incorporates the “hole” substitutions of T366S, L368A, and Y407V, and the second antibody Fc polypeptide incorporates the “knob” substitution of T366W. A49-F3′-KiH-TriNKET-Pertuzumab includes three polypeptides, having the sequences of SEQ ID NO:191, SEQ ID NO:147 (as in A49-F3′-KiH-TriNKET-Trastuzumab), and SEQ ID NO:142 (as in A49-F3′-TriNKET-Trastuzumab).

SEQ ID NO:191 represents the full sequence of the HER2-binding scFv (SEQ ID NO:189) linked to the first antibody Fc polypeptide via a hinge including Ala-Ser (scFv-Fc). The first antibody Fc polypeptide incorporates T366S, L368A, and Y407V substitutions for heterodimerization and an S354C substitution for forming a disulfide bond with a Y349C substitution in SEQ ID NO:191 as described above.

Pertuzumab scFv-Fc (KiH)
(SEQ ID NO: 191)
DIQMTQSPSSLSASVGDRVTITCKASQDVSIGVAWYQQKPGKAPKLLIYS
ASYRYTGVPSRFSGSGSGTDFTLTISSLQPEDFATYYCQQYYIYPYTFG [symbol]
GTKVEIKRGGGGSGGGGSGGGGSGGGGSEVQLVESGGGLVQPGGSLRLSC
AASGFTFTDYTMDWVRQAPGK [symbol] LEWVADVNPNSGGSIYNQRFKGRFTLSV
DRSKNTLYLQMNSLRAEDTAVYYCARNLGPSFYFDYWGQGTLVTVSSAAS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQV [symbol] TLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPG

Another exemplary TriNKET of the present disclosure is A49MI-F3′-KiH-TriNKET-Pertuzumab. A49MI-F3′-KiH-TriNKET-Pertuzumab includes the same HER2-binding scFv (SEQ ID NO:189) as in A49-F3′-TriNKET-Pertuzumab linked via a hinge including Ala-Ser to a first antibody Fc polypeptide; and the same NKG2D-binding Fab fragment as in A49MI-F3′-TriNKET-Trastuzumab, the CH1 domain of which is connected to a second antibody Fc polypeptide. The first antibody Fc polypeptide incorporates the “hole” substitutions of T366S, L368A, and Y407V, and the second antibody Fc polypeptide incorporates the “knob” substitution of T366W. A49MI-F3′-KiH-TriNKET-Pertuzumab includes three polypeptides having the sequences of SEQ ID NO:191 (as in A49-F3′-KiH-TriNKET-Pertuzumab), SEQ ID NO:194 (as in A49MI-F3′-KiH-TriNKET-Trastuzumab), and SEQ ID NO:142 (as in A49-F3′-TriNKET-Trastuzumab).

Another exemplary TriNKET of the present disclosure is A44-F3′-TriNKET-Pertuzumab. A44-F3′-TriNKET-Pertuzumab includes the same HER2-binding scFv (SEQ ID NO:189) as in A49-F3′-TriNKET-Pertuzumab linked via a hinge including Ala-Ser to a first antibody Fc polypeptide; and the same NKG2D-binding Fab fragment as in A44-F3′-TriNKET-Trastuzumab, the CH1 domain of which is connected to a second antibody Fc polypeptide. The first antibody Fc polypeptide incorporates Q347R, D399V, and F405T substitutions, and the second antibody Fc polypeptide incorporates K360E and K409W substitutions. A44-F3′-TriNKET-Pertuzumab includes three polypeptides having the sequences of SEQ ID NO:190 (as in A49-F3′-KiH-TriNKET-Pertuzumab), SEQ ID NO:155 (as in A44-F3′-TriNKET-Trastuzumab), and SEQ ID NO:149 (as in A44-F3′-TriNKET-Trastuzumab).

Another exemplary TriNKET of the present disclosure is A44-F3′-KiH-TriNKET-Pertuzumab. A44-F3′-KiH-TriNKET-Pertuzumab includes the same HER2-binding scFv (SEQ ID NO:189) as in A49-F3′-TriNKET-Pertuzumab linked via a hinge including Ala-Ser to a first antibody Fc polypeptide; and the same NKG2D-binding Fab fragment as in A44-F3′-TriNKET-Trastuzumab, the CH1 domain of which is connected to a second antibody Fc polypeptide. The first antibody Fc polypeptide incorporates the “hole” substitutions of T366S, L368A, and Y407V, and the second antibody Fc polypeptide incorporates the “knob” substitution of T366W. A44-F3′-KiH-TriNKET-Pertuzumab includes three polypeptides having the sequences of SEQ ID NO:191 (as in A49-F3′-KiH-TriNKET-Pertuzumab), SEQ ID NO:148 (as in A44-F3′-KiH-TriNKET-Trastuzumab), and SEQ ID NO:149 (as in A44-F3′-TriNKET-Trastuzumab).

Another TriNKET of the present disclosure is A49-F3′-TriNKET-MGAH22. A49-F3′-TriNKET-MGAH22 includes an scFv (SEQ ID NO:171) derived from MGAH22 that binds HER2, linked via a hinge including Ala-Ser to a first antibody Fc polypeptide; and the same NKG2D-binding Fab fragment as in A49-F3′-TriNKET-Trastuzumab, the CH1 domain of which is connected to a second antibody Fc polypeptide. The first antibody Fc polypeptide incorporates Q347R, D399V, and F405T substitutions, and the second antibody Fc polypeptide linked to the Fab fragment incorporates K360E and K409W substitutions. A49-F3′-TriNKET-MGAH22 includes three polypeptides having the sequences of SEQ ID NO:192, SEQ ID NO:141 (as in A49-F3′-TriNKET-Trastuzumab), and SEQ ID NO:142 (as in A49-F3′-TriNKET-Trastuzumab).

SEQ ID NO:192 represents the full sequence of the HER2-binding scFv linked to the first antibody Fc polypeptide via a hinge including Ala-Ser (scFv-Fc). The first antibody Fc domain incorporates Q347R, D399V, and F405T substitutions for heterodimerization and an S354C substitution for forming a disulfide bond with a Y349C substitution in SEQ ID NO:141 as described above. The scFv (SEQ ID NO:171) includes a heavy chain variable domain of pertuzumab connected to the N-terminus of a light chain variable domain of pertuzumab via a (G₄S)₄ linker (SEQ ID NO:143), the scFv represented as VL-(G₄S)₄-VH (“(G₄S)₄” is represented by SEQ ID NO:143). The heavy and the light variable domains of the scFv are also connected through a disulfide bridge between C100 of VL and C44 of VH, as a result of G100C and G44C substitutions in the VL and VH, respectively.

MGAH22 scFv
(SEQ ID NO: 171)
DIVMTQSHKFMSTSVGDRVSITCKASQDVNTAVAWYQQKPGHSPKLLIYS
ASFRYTGVPDRFTGSRSGTDFTFTISSVQAEDLAVYYCQQHYTTPPTFG [symbol]
GTKVEIKRGGGGSGGGGSGGGGSGGGGSQVQLQQSGPELVKPGASLKLSC
TASGFNIKDTYIHWVKQRPEQ [symbol] LEWIGRIYPTNGYTRYDPKFQDKATITA
DTSSNTAYLQVSRLTSEDTAVYYCSRWGGDGFYAMDYWGQGASVTVSSA
MGAH22 scFv-Fc
(SEQ ID NO: 192)
DIVMTQSHKFMSTSVGDRVSITCKASQDVNTAVAWYQQKPGHSPKLLIYS
ASFRYTGVPDRFTGSRSGTDFTFTISSVQAEDLAVYYCQQHYTTPPTFG [symbol]
GTKVEIKRGGGGSGGGGSGGGGSGGGGSQVQLQQSGPELVKPGASLKLSC
TASGFNIKDTYIHWVKQRPEQ [symbol] LEWIGRIYPTNGYTRYDPKFQDKATITA
DTSSNTAYLQVSRLTSEDTAVYYCSRWGGDGFYAMDYWGQGASVTVSSAA
SDKTHTCPPCPAPELLGGPSVFLFPPKPKDTLMISRTPEVTCVVVDVSHE
DPEVKFNWYVDGVEVHNAKTKPREEQYNSTYRVVSVLTVLHQDWLNGKEY
KCKVSNKALPAPIEKTISKAKGQPREPRVYTLPP [symbol] RDELTKNQVSLTCLV
KGFYPSDIAVEWESNGQPENNYKTTPPVLVSDGSFTLYSKLTVDKSRWQQ
GNVFSCSVMHEALHNHYTQKSLSLSPG

Another TriNKET of the present disclosure is A49MI-F3′-TriNKET-MGAH22. A49MI-F3′-TriNKET-MGAH22 includes the same HER2-binding scFv (SEQ ID NO:171) as in A49-F3′-TriNKET-MGAH22 linked via a hinge including Ala-Ser to a first antibody Fc polypeptide; and the same NKG2D-binding Fab fragment as in A49MI-F3′-TriNKET-Trastuzumab, the CH1 domain of which is connected to a second antibody Fc polypeptide. The first antibody Fc polypeptide incorporates Q347R, D399V, and F405T substitutions, and the second antibody Fc polypeptide incorporates K360E and K409W substitutions. A49MI-F3′-KiH-TriNKET-MGAH22 includes three polypeptides, having the sequences of SEQ ID NO:192 (as in A49-F3′-TriNKET-MGAH22), SEQ ID NO:145 (as in A49MI-F3′-TriNKET-Trastuzumab), and SEQ ID NO:142 (as in A49-F3′-TriNKET-Trastuzumab).

Another TriNKET of the present disclosure is A49-F3′-KiH-TriNKET-MGAH22. A49-F3′-KiH-TriNKET-MGAH22 includes the same HER2-binding scFv (SEQ ID NO:171) as in A49-F3′-TriNKET-MGAH22 linked via a hinge including Ala-Ser to a first antibody Fc polypeptide; and the same NKG2D-binding Fab fragment as in A49-F3′-TriNKET-Trastuzumab, the CH1 domain of which is connected to a second antibody Fc polypeptide. The first antibody Fc polypeptide incorporates the “hole” substitutions of T366S, L368A, and Y407V, and the second antibody Fc polypeptide linked to the Fab fragment incorporates the “knob” substitution of T366W. A49-F3′-KiH-TriNKET-MGAH22 includes three polypeptides having the sequences of SEQ ID NO:193, SEQ ID NO:147 (as in A49-F3′-KiH-TriNKET-Trastuzumab), and SEQ ID NO:142 (as in A49-F3′-TriNKET-Trastuzumab).

SEQ ID NO:193 represents the full sequence of the HER2-binding scFv (SEQ ID NO:171) linked to the first antibody Fc polypeptide via a hinge including Ala-Ser (scFv-Fc). The first antibody Fc polypeptide incorporates T366S, L368A, and Y407V substitutions for heterodimerization and an S354C substitution for forming a disulfide bond with a Y349C substitution in SEQ ID NO:147 as described above.

MGAH22 scFv-Fc (KiH)
(SEQ ID NO: 193)
DIVMTQSHKFMSTSVGDRVSITCKASQDVNTAVAWYQQKPGHSPKLLIYS
ASFRYTGVPDRFTGSRSGTDFTFTISSVQAEDLAVYYCQQHYTTPPTFG [symbol]
GTKVEIKRGGGGSGGGGSGGGGGGGGSQVQLQQSGPELVKPGASLKLSCT
ASGFNIKDTYIHWVKQRPEQ [symbol] LEWIGRIYPTNGYTRYDPKFQDKATITAD
TSSNTAYLQVSRLTSEDTAVYYCSRWGGDGFYAMDYWGQGASVTVSSAAS
DKTHTCPPCPAPELLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQV [symbol] TLPPSRDELTKNQVSLSCAVK
GFYPSDIAVEWESNGQPENNYKTTPPVLDSDGSFFLVSKLTVDKSRWQQG
NVFSCSVMHEALHNHYTQKSLSLSPG

Another exemplary TriNKET of the present disclosure is A49MI-F3′-KiH-TriNKET-MGAH22. A49MI-F3′-KiH-TriNKET-MGAH22 includes the same HER2-binding scFv (SEQ ID NO:171) as in A49-F3′-TriNKET-MGAH22 linked via a hinge including Ala-Ser to a first antibody Fc polypeptide; and the same NKG2D-binding Fab fragment as in A49MI-F3′-TriNKET-Trastuzumab, the CH1 domain of which is connected to a second antibody Fc polypeptide. The first antibody Fc polypeptide incorporates the “hole” substitutions of T366S, L368A, and Y407V, and the second antibody Fc polypeptide incorporates the “knob” substitution of T366W. A49MI-F3′-KiH-TriNKET-MGAH22 includes three polypeptides having the sequences of SEQ ID NO:193 (as in A49-F3′-KiH-TriNKET-MGAH22), SEQ ID NO:194 (as in A49MI-F3′-KiH-TriNKET-Trastuzumab), and SEQ ID NO:142 (as in A49-F3′-TriNKET-Trastuzumab).

Another exemplary TriNKET of the present disclosure is A44-F3′-TriNKET-MGAH22. A44-F3′-TriNKET-MGAH22 includes the same HER2-binding scFv (SEQ ID NO:171) as in A49-F3′-TriNKET-MGAH22 linked via a hinge including Ala-Ser to a first antibody Fc polypeptide; and the same NKG2D-binding Fab fragment as in A44-F3′-TriNKET-Trastuzumab, the CH1 domain of which is connected to a second antibody Fc polypeptide. The first antibody Fc polypeptide incorporates Q347R, D399V, and F405T substitutions, and the second antibody Fc polypeptide incorporates K360E and K409W substitutions. A44-F3′-TriNKET-MGAH22 includes three polypeptides having the sequences of SEQ ID NO:192 (as in A49-F3′-TriNKET-MGAH22), SEQ ID NO:155 (as in A44-F3′-TriNKET-Trastuzumab), and SEQ ID NO:149 (as in A44-F3′-TriNKET-Trastuzumab).

Another exemplary TriNKET of the present disclosure is A44-F3′-KiH-TriNKET-MGAH22. A44-F3′-KiH-TriNKET-MGAH22 includes the same HER2-binding scFv (SEQ ID NO:171) as in A49-F3′-TriNKET-MGAH22 linked via a hinge including Ala-Ser to a first antibody Fc polypeptide; and the same NKG2D-binding Fab fragment as in A44-F3′-TriNKET-Trastuzumab, the CH1 domain of which is connected to a second antibody Fc polypeptide. The first antibody Fc polypeptide incorporates the “hole” substitutions of T366S, L368A, and Y407V, and the second antibody Fc polypeptide incorporates the “knob” substitution of T366W. A44-F3′-KiH-TriNKET-MGAH22 includes three polypeptides having the sequences of SEQ ID NO:193 (as in A49-F3′-KiH-TriNKET-MGAH22), SEQ ID NO:148 (as in A44-F3′-KiH-TriNKET-Trastuzumab), and SEQ ID NO:149 (as in A44-F3′-TriNKET-Trastuzumab).

In certain embodiments, a TriNKET of the present disclosure is identical to one of the exemplary TriNKETs described above that includes the EW-RVT Fc mutations, except that the antibody Fc polypeptide linked to the NKG2D-binding Fab fragment incorporates the substitutions of Q347R, D399V, and F405T, and the antibody Fc polypeptide linked to the HER2-binding scFv incorporates matching substitutions K360E and K409W for forming a heterodimer. In certain embodiments, a TriNKET of the present disclosure is identical to one of the exemplary TriNKETs described above that includes the KiH Fc mutations, except that the antibody Fc polypeptide linked to the NKG2D-binding Fab fragment incorporates the “hole” substitutions of T366S, L368A, and Y407V, and the antibody Fc polypeptide linked to the HER2-binding scFv incorporates the “knob” substitution of T366W for forming a heterodimer.

In certain embodiments, a TriNKET of the present disclosure is identical to one of the exemplary TriNKETs described above, except that the antibody Fc polypeptide linked to the NKG2D-binding Fab fragment includes an S354C substitution in the CH3 domain, and the antibody Fc polypeptide linked to the HER2-binding scFv includes a matching Y349C substitution in the CH3 domain for forming a disulfide bond.

As described in International Application No. PCT/US2019/045561, the multi-specific binding proteins disclosed herein are effective in reducing tumor growth and killing cancer cells in in vitro assays and animal models. For example, A49-F3′-TriNKET-Trastuzumab is superior to trastuzumab in inducing NK cell-mediated cytotoxicity against various human cancer cell lines, such as 786-0 cells that express low levels of HER2 (HER2+), H661 cells that express moderate levels of HER2 (HER2++), and SkBr3 cells that express high levels of HER2 (HER2+++). Furthermore, the multi-specific binding proteins do not significantly induce NK-mediated killing of healthy non-cancerous human cells (e.g., human cardiomyocytes).

Production of Multi-Specific Binding Proteins

The multi-specific binding proteins described above can be made using recombinant DNA technology well known to a skilled person in the art. For example, a first nucleic acid sequence encoding the first immunoglobulin heavy chain can be cloned into a first expression vector; a second nucleic acid sequence encoding the second immunoglobulin heavy chain can be cloned into a second expression vector; a third nucleic acid sequence encoding the immunoglobulin light chain can be cloned into a third expression vector; and the first, second, and third expression vectors can be stably transfected together into host cells to produce the multimeric proteins.

A skilled person in the art would appreciate that during production and/or storage of proteins, N-terminal glutamate (E) or glutamine (Q) can be cyclized to form a lactam (e.g., spontaneously or catalyzed by an enzyme present during production and/or storage). Accordingly, in some embodiments where the N-terminal residue of an amino acid sequence of a polypeptide is E or Q, a corresponding amino acid sequence with the E or Q replaced with pyroglutamate is also contemplated herein.

A skilled person in the art would also appreciate that during protein production and/or storage, the C-terminal lysine (K) of a protein can be removed (e.g., spontaneously or catalyzed by an enzyme present during production and/or storage). Such removal of K is often observed with proteins that include an Fc domain at their C-termini. Accordingly, in some embodiments where the C-terminal residue of an amino acid sequence of a polypeptide (e.g., an antibody Fc polypeptide) is K, a corresponding amino acid sequence with the K removed is also contemplated herein.

To achieve the highest yield of the multi-specific binding protein, different ratios of the first, second, and third expression vector can be explored to determine the optimal ratio for transfection into the host cells. After transfection, single clones can be isolated for cell bank generation using methods known in the art, such as limited dilution, ELISA, FACS, microscopy, or Clonepix.

Clones can be cultured under conditions suitable for bio-reactor scale-up and maintained expression of the multi-specific binding protein. The multi-specific binding proteins can be isolated and purified using methods known in the art including centrifugation, depth filtration, cell lysis, homogenization, freeze-thawing, affinity purification, gel filtration, ion exchange chromatography, hydrophobic interaction exchange chromatography, and mixed-mode chromatography.

Pharmaceutical Formulations

The methods of treatment of the present disclosure can use pharmaceutical compositions and pharmaceutical formulations that contain a multi-specific binding protein disclosed herein (e.g., A49-F3′-TriNKET-Trastuzumab) at a concentration of 10-250 mg/mL (e.g., 10-50 mg/mL, 10-25 mg/mL, about 15 mg/mL, or 50-250 mg/mL). The pharmaceutical formulation contains one or more excipients and is maintained at a certain pH. The term “excipient,” as used herein, means any non-therapeutic agent added to the formulation to provide a desired physical or chemical property, for example, pH, osmolarity, viscosity, clarity, color, isotonicity, odor, sterility, stability, rate of dissolution or release, adsorption, or penetration.

Excipients and pH

The one or more excipients in the pharmaceutical composition or pharmaceutical formulation of the present invention contains a buffering agent. The term “buffering agent,” as used herein, refers to one or more components that when added to an aqueous solution is able to protect the solution against variations in pH when adding acid or alkali, or upon dilution with a solvent. In addition to phosphate buffers, glycinate, carbonate, citrate, histidine buffers and the like can be used, in which case, sodium, potassium or ammonium ions can serve as counterion.

In certain embodiments, the buffer or buffer system includes at least one buffer that has a buffering range that overlaps fully or in part with the range of pH 5.5-7.4. In certain embodiments, the buffer has a pKa of about 6.0±0.5. In certain embodiments, the buffer contains a histidine buffer. In certain embodiments, the histidine is present at a concentration of 5 to 100 mM, 10 to 100 mM, 15 to 100 mM, 20 to 100 mM, 5 to 50 mM, 10 to 50 mM, 15 to 100 mM, 20 to 100 mM, 5 to 25 mM, 10 to 25 mM, 15 to 25 mM, 20 to 25 mM, 5 to 20 mM, 10 to 20 mM, or 15 to 20 mM. In certain embodiments, the histidine is present at a concentration of 5 mM, 10 mM, 15 mM, 20 mM, 25 mM, or 50 mM. In certain embodiments, the histidine is present at a concentration of 20 mM.

The pharmaceutical composition or pharmaceutical formulation disclosed herein may have a pH of 5.5 to 6.5. For example, in certain embodiments, the pharmaceutical composition or pharmaceutical formulation has a pH of 5.5 to 6.5 (i.e., 6.0±0.5), 5.6 to 6.4 (i.e., 6.0±0.4), 5.7 to 6.3 (i.e., 6.0±0.3), 5.8 to 6.2 (i.e., 6.0±0.2), 5.9 to 6.1 (i.e., 6.0±0.1), or 5.95 to 6.05 (i.e., 6.0±0.05). In certain embodiments, the pharmaceutical composition or pharmaceutical formulation has a pH of 5.5, 5.6, 5.7, 5.8, 5.9, 6.0, 6.1, 6.2, 6.3, 6.4, or 6.5. In certain embodiments, the pharmaceutical composition or pharmaceutical formulation has a pH of 6.0. Under the rules of scientific rounding, a pH greater than or equal to 5.95 and smaller than or equal to 6.05 is rounded as 6.0.

In certain embodiments, the buffer system of the pharmaceutical composition or pharmaceutical formulation contains histidine at 10 to 25 mM, at a pH of 6.0±0.2. In certain embodiments, the buffer system of the pharmaceutical composition or pharmaceutical formulation contains histidine at 20 mM, at a pH of 6.0±0.2. In certain embodiments, the buffer system of the pharmaceutical composition or pharmaceutical formulation contains histidine at 10 to 25 mM, at a pH of 6.0±0.05. In certain embodiments, the buffer system of the pharmaceutical composition or pharmaceutical formulation contains histidine at 20 mM, at a pH of 6.0±0.05.

The one or more excipients in the pharmaceutical composition or pharmaceutical formulation disclosed herein may further contains a sugar or sugar alcohol. Sugars and sugar alcohols are useful in pharmaceutical composition or pharmaceutical formulations as a thermal stabilizer. In certain embodiments, the pharmaceutical composition or pharmaceutical formulation contains a sugar, for example, a monosaccharide (glucose, xylose, or erythritol), a disaccharide (e.g., sucrose, trehalose, maltose, or galactose), or an oligosaccharide (e.g., stachyose). In specific embodiments, the pharmaceutical composition or pharmaceutical formulation contains sucrose. In certain embodiments, the pharmaceutical composition contains a sugar alcohol, for example, a sugar alcohol derived from a monosaccharide (e.g., mannitol, sorbitol, or xylitol), a sugar alcohol derived from a disaccharide (e.g., lactitol or maltitol), or a sugar alcohol derived from an oligosaccharide. In specific embodiments, the pharmaceutical composition or pharmaceutical formulation contains sorbitol.

The amount of the sugar or sugar alcohol contained within the formulation can vary depending on the specific circumstances and intended purposes for which the formulation is used. In certain embodiments, the pharmaceutical composition or pharmaceutical formulation contains 50 to 300 mM, 50 to 250 mM, 100 to 300 mM, 100 to 250 mM, 150 to 300 mM, 150 to 250 mM, 200 to 300 mM, 200 to 250 mM, or 250 to 300 mM of the sugar or sugar alcohol. In certain embodiments, the pharmaceutical composition or pharmaceutical formulation contains 50 mM, 75 mM, 100 mM, 125 mM, 150 mM, 200 mM, 250 mM, or 300 mM of the sugar or sugar alcohol. In specific embodiments, the pharmaceutical composition or pharmaceutical formulation contains 250 mM of the sugar or sugar alcohol (e.g., sucrose or sorbitol).

The one or more excipients in the pharmaceutical composition or pharmaceutical formulation disclosed herein further contains a surfactant. The term “surfactant,” as used herein, refers to a surface active molecule containing both a hydrophobic portion (e.g., alkyl chain) and a hydrophilic portion (e.g., carboxyl and carboxylate groups). Surfactants are useful in pharmaceutical composition or pharmaceutical formulations for reducing aggregation of a therapeutic protein. Surfactants suitable for use in the pharmaceutical composition or pharmaceutical formulations are generally non-ionic surfactants and include, but are not limited to, polysorbates (e.g. polysorbates 20 or 80); poloxamers (e.g. poloxamer 188); sorbitan esters and derivatives; Triton; sodium laurel sulfate; sodium octyl glycoside; lauryl-, myristyl-, linoleyl-, or stearyl-sulfobetadine; lauryl-, myristyl-, linoleyl- or stearyl-sarcosine; linoleyl-, myristyl-, or cetyl-betaine; lauramidopropyl-cocamidopropyl-, linoleamidopropyl-, myristamidopropyl-, palmidopropyl-, or isostearamidopropylbetaine (e.g., lauroamidopropyl); myristamidopropyl-, palmidopropyl-, or isostearamidopropyl-dimethylamine; sodium methyl cocoyl-, or disodium methyl oleyl-taurate; and the MONAQUAT™ series (Mona Industries, Inc., Paterson, N.J.), polyethylene glycol, polypropyl glycol, and copolymers of ethylene and propylene glycol (e.g., Pluronics, PF68 etc.). In certain embodiments, the surfactant is a polysorbate. In certain embodiments, the surfactant is polysorbate 80.

The amount of a non-ionic surfactant contained within the pharmaceutical composition or pharmaceutical formulation of the present invention may vary depending on the specific properties desired of the formulation, as well as the particular circumstances and purposes for which the formulations are intended to be used. In certain embodiments, the pharmaceutical composition or pharmaceutical formulation contains 0.005% to 0.5%, 0.005% to 0.005% to 0.1%, 0.005% to 0.05%, 0.005% to 0.02%, 0.005% to 0.01%, 0.01% to 0.5%, to 0.2%, 0.01% to 0.1%, 0.01% to 0.05%, or 0.01% to 0.02% of the non-ionic surfactant (e.g., polysorbate 80). In certain embodiments, the pharmaceutical composition or pharmaceutical formulation contains 0.005%, 0.01%, 0.02%, 0.03%, 0.04%, 0.05%, 0.06%, 0.08%, 0.09%, 0.1%, 0.15%, 0.2%, 0.25%, 0.3%, 0.35%, 0.4%, 0.45%, or 0.5% of the non-ionic surfactant (e.g., polysorbate 80). The concentrations of non-ionic surfactant are provided as % (w/v) values.

In certain embodiments, the pharmaceutical composition or pharmaceutical formulation is isotonic. An “isotonic” formulation is one which has essentially the same osmotic pressure as human blood. Isotonic formulations generally have an osmotic pressure from about 250 to 350 mOsmol/kgH2O. Isotonicity can be measured using a vapor pressure or ice-freezing type osmometer. In certain embodiments, the osmolarity of the pharmaceutical composition or pharmaceutical formulation is 250 to 350 mOsmol/kgH2O. In certain embodiments, the osmolarity of the pharmaceutical composition or pharmaceutical formulation is 300 to 350 mOsmol/kgH2O.

Substances such as a sugar, a sugar alcohol, and NaCl can be included in the pharmaceutical composition or pharmaceutical formulation for desired osmolarity. In certain embodiments, the concentration of NaCl in the pharmaceutical composition or pharmaceutical formulation, if any, is equal to or lower than 10 mM, 9 mM, 8 mM, 7 mM, 6 mM, 5 mM, 4 mM, 3 mM, 2 mM, 1 mM, 0.5 mM, 0.1 mM, 50 μM, 10 μM, 5 μM, or 1 μM. In certain embodiments, the concentration of NaCl in the pharmaceutical composition or pharmaceutical formulation is below the detection limit. In certain embodiments, no NaCl is added when preparing the pharmaceutical composition or pharmaceutical formulation.

The pharmaceutical composition or pharmaceutical formulation disclosed herein may further include one or more other substances, such as a bulking agent or a preservative. A “bulking agent” is a compound which adds mass to a lyophilized mixture and contributes to the physical structure of the lyophilized cake (e.g., facilitates the production of an essentially uniform lyophilized cake which maintains an open pore structure). Illustrative bulking agents include mannitol, glycine, polyethylene glycol and sorbitol. The lyophilized formulations of the present invention may contain such bulking agents. A preservative reduces bacterial action and may, for example, facilitate the production of a multi-use (multiple-dose) formulation.

The multi-specific binding protein can be formulated in the pharmaceutical composition or pharmaceutical formulation at high concentrations, for example, greater than 50 mg/mL. In certain embodiments, the pharmaceutical composition or pharmaceutical formulation contains greater than or equal to 60 mg/mL, greater than or equal to 70 mg/mL, greater than or equal to 80 mg/mL, greater than or equal to 90 mg/mL, greater than or equal to 100 mg/mL, greater than or equal to 125 mg/mL, greater than or equal to 150 mg/mL, greater than or equal to 175 mg/mL, or greater than or equal to 200 mg/mL of the multi-specific binding protein. In certain embodiments, the pharmaceutical composition or pharmaceutical formulation contains mg/mL, 60-225 mg/mL, 60-200 mg/mL, 60-175 mg/mL, 50-150 mg/mL, 60-150 mg/mL, mg/mL, 60-100 mg/mL, 60-90 mg/mL, 60-80 mg/mL, 60-70 mg/mL, 70-250 mg/mL, 70-225 mg/mL, 70-200 mg/mL, 70-175 mg/mL, 70-150 mg/mL, 70-150 mg/mL, 70-125 mg/mL, mg/mL, 70-90 mg/mL, 70-80 mg/mL, 80-250 mg/mL, 80-225 mg/mL, 80-200 mg/mL, mg/mL, 80-150 mg/mL, 80-150 mg/mL, 80-125 mg/mL, 80-100 mg/mL, 80-90 mg/mL, mg/mL, 90-225 mg/mL, 90-200 mg/mL, 90-175 mg/mL, 90-150 mg/mL, 90-150 mg/mL, mg/mL, 90-100 mg/mL, 100-250 mg/mL, 100-225 mg/mL, 100-200 mg/mL, 100-175 mg/mL, 100-150 mg/mL, 100-125 mg/mL, 125-250 mg/mL, 125-225 mg/mL, 125-200 mg/mL, 125-175 mg/mL, 125-150 mg/mL, 150-250 mg/mL, 150-225 mg/mL, 150-200 mg/mL, 150-175 mg/mL, 175-250 mg/mL, 175-225 mg/mL, 175-200 mg/mL, 200-250 mg/mL, or 200-225 mg/mL of the multi-specific binding protein. In certain embodiments, the pharmaceutical composition or pharmaceutical formulation contains 60 mg/mL, 70 mg/mL, 80 mg/mL, 90 mg/mL, 100 mg/mL, 125 mg/mL, 150 mg/mL, 175 mg/mL, 200 mg/mL, or 220 mg/mL of the multi-specific binding protein. In certain embodiments, the pharmaceutical composition or pharmaceutical formulation contains at least 10 mg/mL, 10 mg/mL to equal to or greater than 50 mg/mL of the multi-specific binding protein.

Exemplary Formulations

In certain embodiments, the pharmaceutical composition or pharmaceutical formulation of the present invention contains the multi-specific binding protein, histidine, a sugar or sugar alcohol (e.g., sucrose or sorbitol), and a polysorbate (e.g., polysorbate 80), at pH 5.5 to 6.5.

In certain embodiments, the pharmaceutical composition or pharmaceutical formulation contains 10 to 250 mg/mL of the multi-specific binding protein, 10 to 25 mM of histidine, 200 to 300 mM of a sugar or sugar alcohol (e.g., sucrose or sorbitol), and 0.005% to of a polysorbate (e.g., polysorbate 80), at pH 5.5 to 6.5. In certain embodiments, the pharmaceutical composition or pharmaceutical formulation contains 10 to 250 mg/mL of the multi-specific binding protein, 20 mM of histidine, 250 mM of a sugar or sugar alcohol (e.g., sucrose or sorbitol), and 0.01% of a polysorbate (e.g., polysorbate 80), at pH 5.5 to 6.5. In certain embodiments, the pharmaceutical composition or pharmaceutical formulation contains 10 to 250 mg/mL of the multi-specific binding protein, 20 mM of histidine, 250 mM of a sugar or sugar alcohol (e.g., sucrose or sorbitol), and 0.01% of a polysorbate (e.g., polysorbate 80), at pH to 6.2. In certain embodiments, the pharmaceutical composition or pharmaceutical formulation contains 10 to 250 mg/mL of the multi-specific binding protein, 20 mM of histidine, 250 mM of a sugar or sugar alcohol (e.g., sucrose or sorbitol), and 0.01% of a polysorbate (e.g., polysorbate 80), at pH 5.95 to 6.05.

In certain embodiments, the pharmaceutical composition or pharmaceutical formulation contains 10 to 250 mg/mL of the multi-specific binding protein, 10 to 25 mM of histidine, 200 to 300 mM of sucrose, and 0.005% to 0.05% of polysorbate 80, at pH 5.5 to 6.5. In certain embodiments, the pharmaceutical composition or pharmaceutical formulation contains to 250 mg/mL of the multi-specific binding protein, 20 mM of histidine, 250 mM of sucrose, and 0.01% of polysorbate 80, at pH 5.5 to 6.5. In certain embodiments, the pharmaceutical composition or pharmaceutical formulation contains 10 to 250 mg/mL of the multi-specific binding protein, 20 mM of histidine, 250 mM of sucrose, and 0.01% of polysorbate 80, at pH 5.8 to 6.2. In certain embodiments, the pharmaceutical composition or pharmaceutical formulation contains 10 to 250 mg/mL of the multi-specific binding protein, 20 mM of histidine, 250 mM of sucrose, and 0.01% of polysorbate 80, at pH 5.95 to 6.05.

In certain embodiments, the pharmaceutical composition or pharmaceutical formulation contains 10 to 250 mg/mL of the multi-specific binding protein, 10 to 25 mM of histidine, 200 to 300 mM of sorbitol, and 0.005% to 0.05% of polysorbate 80, at pH 5.5 to 6.5. In certain embodiments, the pharmaceutical composition or pharmaceutical formulation contains to 250 mg/mL of the multi-specific binding protein, 20 mM of histidine, 250 mM of sorbitol, and 0.01% of polysorbate 80, at pH 5.5 to 6.5. In certain embodiments, the pharmaceutical composition or pharmaceutical formulation contains 10 to 250 mg/mL of the multi-specific binding protein, 20 mM of histidine, 250 mM of sorbitol, and 0.01% of polysorbate 80, at pH 5.8 to 6.2. In certain embodiments, the pharmaceutical composition or pharmaceutical formulation contains 10 to 250 mg/mL of the multi-specific binding protein, 20 mM of histidine, 250 mM of sorbitol, and 0.01% of polysorbate 80, at pH 5.95 to 6.05.

In certain embodiments, the pharmaceutical composition or pharmaceutical formulation contains 10 to 50 mg/mL of the multi-specific binding protein, 5 mM to 50 mM of histidine, 50 mM tp 300 mM sucrose, and about 0.005% to 0.05% (w/v) of polysorbate 80, at pH to 6.5. In certain embodiments, the pharmaceutical composition or pharmaceutical formulation contains 10 to 25 mg/mL of the multi-specific binding protein, 10 mM to 25 mM of histidine, 150 mM tp 300 mM sucrose, and about 0.005% to 0.02% (w/v) of polysorbate 80, at pH 5.8 to 6.2. In certain embodiments, the pharmaceutical composition or pharmaceutical formulation contains about 15 mg/mL of the multi-specific binding protein, about 20 mM of histidine, about 250 mM sucrose, and about 0.01% (w/v) of polysorbate 80, at about pH 6.0.

Stability of the Multi-Specific Binding Protein

The pharmaceutical compositions or pharmaceutical formulations disclosed herein exhibit high levels of stability. A pharmaceutical formulation is stable when the multi-specific binding protein within the formulation retains an acceptable degree of physical property, chemical structure, and/or biological function after storage under defined conditions.

Stability can be measured by determining the percentage of the multi-specific binding protein in the formulation that remains in a native conformation after storage for a defined amount of time at a defined temperature. The percentage of a protein in a native conformation can be determined by, for example, size exclusion chromatography (e.g., size exclusion high performance liquid chromatography), where a protein in the native conformation is not aggregated (eluted in a high molecular weight fraction) or degraded (eluted in a low molecular weight fraction). In certain embodiments, more than 95%, 96%, 97%, 98%, or 99% of the multi-specific binding protein has native conformation, as determined by size-exclusion chromatography, after incubation at 4° C. for 3 weeks. In certain embodiments, more than 90%, 91%, 92%, 93%, 94%, 95%, 96%, 97%, 98%, or 99% of the multi-specific binding protein has native conformation, as determined by size-exclusion chromatography, after incubation at 50° C. for 3 weeks. In certain embodiments, less than 0.5%, 0.6%, 0.7%, 0.8%, 0.9%, or 1% of the multi-specific binding protein forms a high molecular weight complex (i.e., having a higher molecular weight than the native protein), as determined by size-exclusion chromatography, after incubation at 4° C. for 3 weeks. In certain embodiments, less than 1%, 2%, 3%, 4%, or 5% of the multi-specific binding protein form a high molecular weight complex (i.e., having a higher molecular weight than the native protein), as determined by size-exclusion chromatography, after incubation at 50° C. for 3 weeks. In certain embodiments, less than 0.5%, 0.6%, 0.7%, 0.8%, or 1% of the multi-specific binding protein is degraded (i.e., having a lower molecular weight than the native protein), as determined by size-exclusion chromatography, after incubation at 4° C. for 3 weeks. In certain embodiments, less than 1%, 1.5%, 2%, 2.5%, or 3% of the multi-specific binding protein is degraded (i.e., having a lower molecular weight than the native protein), as determined by size-exclusion chromatography, after incubation at 50° C. for 3 weeks.

Stability can also be measured by determining the percentage of multi-specific binding protein present in a more acidic fraction (“acidic form”) relative to the main fraction of protein (“main charge form”). While not wishing to be bound by theory, deamidation of a protein may cause it to become more negatively charged and thus more acidic relative to the non-deamidated protein (see, e.g., Robinson, Protein Deamidation, (2002) PNAS 99(8):5283-88). The percentage of the acidic form of a protein can be determined by ion exchange chromatography (e.g., cation exchange high performance liquid chromatography) or imaged capillary isoelectric focusing (icIEF). In certain embodiments, at least 50%, 60%, 70%, 80%, or 90% of the multi-specific binding protein in the pharmaceutical composition or pharmaceutical formulation is in the main charge form after incubation at 4° C. for 3 weeks. In certain embodiments, at least 15%, 20%, 25%, 30%, 40%, 50%, 60%, 70%, 80%, or 90% of the multi-specific binding protein in the pharmaceutical composition or pharmaceutical formulation is in the main charge form after incubation at 50° C. for 3 weeks. In certain embodiments, no more than 10%, 20%, 30%, 40%, or 50% of the multi-specific binding protein in the pharmaceutical composition or pharmaceutical formulation is in an acidic form after incubation at 4° C. for 3 weeks. In certain embodiments, no more than 10%, 20%, 30%, 40%, 50%, 60%, 70%, 75%, 80%, or 85% of the multi-specific binding protein in the pharmaceutical composition or pharmaceutical formulation is in an acidic form after incubation at 50° C. for 3 weeks.

Stability can also be measured by determining the purity of the multi-specific binding protein by electrophoresis after denaturing the protein with sodium dodecyl sulfate (SDS). The protein sample can be denatured in the presence or absence of an agent that reduces protein disulfide bonds (e.g., β-mercaptoethanol). In certain embodiments, the purity of the multi-specific binding protein in the pharmaceutical composition or pharmaceutical formulation, as measured by capillary electrophoresis after denaturing the protein sample under reducing conditions (e.g., in the presence of β-mercaptoethanol), is at least 95%, 96%, 97%, 98%, or 99% after incubation at 4° C. for 3 weeks. In certain embodiments, the purity of the multi-specific binding protein in the pharmaceutical composition or pharmaceutical formulation, as measured by capillary electrophoresis after denaturing the protein sample under reducing conditions (e.g., in the presence of (3-mercaptoethanol), is at least 90%, 91%, 92%, 93%, 94%, 95%, 96%, 97%, 98%, or 99% after incubation at 50° C. for 3 weeks. In certain embodiments, the purity of the multi-specific binding protein in the pharmaceutical composition or pharmaceutical formulation, as measured by capillary electrophoresis after denaturing the protein sample under non-reducing conditions, is at least 95%, 96%, 97%, 98%, or 99% after incubation at 4° C. for 3 weeks. In certain embodiments, the purity of the multi-specific binding protein in the pharmaceutical composition or pharmaceutical formulation, as measured by capillary electrophoresis after denaturing the protein sample under non-reducing conditions, is at least 85%, 86%, 87%, 88%, 89%, 90%, 91%, 92%, 93%, 94%, 95%, 96%, 97%, 98%, or 99% after incubation at 50° C. for 3 weeks.

Stability can also be measured by determining the parameters of a protein solution by dynamic light scattering. The Z-average and polydispersity index (PDI) values indicate the average diameter of particles in a solution and these measures increase when aggregates are present in the solution. The monomer % Pd value indicates the spread of different monomers detected, where lower values indicate a monodispere solution, which is preferred. The monomer size detected by DLS is useful in confirming that the main population is monomer and to characterize any higher order aggregates that may be present. In certain embodiments, the Z-average value of the pharmaceutical composition or pharmaceutical formulation does not increase by more than 5%, 10%, or 15% after incubation at 4° C. for 3 weeks. In certain embodiments, the Z-average value of the pharmaceutical composition or pharmaceutical formulation does not increase by more than 5%, 10%, 15%, 20%, or 25% after incubation at 50° C. for 3 weeks. In certain embodiments, the PDI value of the pharmaceutical composition or pharmaceutical formulation does not increase by more than 10%, 20%, 30%, 40%, or 50% after incubation at 4° C. for 3 weeks. In certain embodiments, the PDI value of the pharmaceutical composition or pharmaceutical formulation does not increase by more than 2-fold, 3-fold, 4-fold, or 5-fold after incubation at 50° C. for 3 weeks.

Exemplary methods to determine stability of the multi-specific binding protein in the pharmaceutical composition or pharmaceutical formulation are described in Example 1 of the present disclosure. Additionally, stability of the protein can be assessed by measuring the binding affinity of the multi-specific binding protein to its targets or the biological activity of the multi-specific binding protein in certain in vitro assays, such as the NK cell activation assays and cytotoxicity assays described in WO 2018/152518.

Dosage Forms

The pharmaceutical composition or pharmaceutical formulation can be prepared and stored as a liquid formulation or a lyophilized form. In certain embodiments, the pharmaceutical composition or pharmaceutical formulation is a liquid formulation for storage at 2-8° C. (e.g., 4° C.) or a frozen formulation for storage at −20° C. or lower. The sugar or sugar alcohol in the formulation is used as a lyoprotectant.

Prior to pharmaceutical use, the pharmaceutical composition or pharmaceutical formulation can be diluted or reconstituted in an aqueous carrier suitable for the route of administration. Other exemplary carriers include sterile water for injection (SWFI), bacteriostatic water for injection (BWFI), a pH buffered solution (e.g., phosphate-buffered saline), sterile saline solution, Ringer's solution, or dextrose solution. For example, when prepared for intravenous administration, the pharmaceutical composition or pharmaceutical formulation can be diluted in a 0.9% sodium chloride (NaCl) solution, or a 0.9% NaCl solution and 0.01% polysorbate 80. In certain embodiments, the diluted pharmaceutical composition or pharmaceutical formulation is isotonic and suitable for administration by intravenous infusion.

The pharmaceutical composition or pharmaceutical formulation contains the multi-specific binding protein at a concentration suitable for storage. In certain embodiments, the pharmaceutical composition or pharmaceutical formulation contains the multi-specific binding protein at a concentration of 10-50 mg/mL, 10-40 mg/mL, 10-30 mg/mL, 10-25 mg/mL, 10-20 mg/mL, 10-15 mg/mL, 15-50 mg/mL, 15-40 mg/mL, 15-30 mg/mL, 15-25 mg/mL, 15-20 mg/mL, 20-50 mg/mL, 20-40 mg/mL, 20-30 mg/mL, 20-25 mg/mL, 30-50 mg/mL, 30-40 mg/mL, or 40-50 mg/mL. In certain embodiments, the pharmaceutical composition or pharmaceutical formulation contains the multi-specific binding protein at a concentration of 10 mg/mL, 11 mg/mL, 12 mg/mL, 13 mg/mL, 14 mg/mL, 15 mg/mL, 16 mg/mL, 17 mg/mL, 18 mg/mL, 19 mg/mL, 20 mg/mL, 25 mg/mL, 30 mg/mL, 35 mg/mL, 40 mg/mL, 45 mg/mL, or 50 mg/mL.

In certain embodiments, the pharmaceutical composition or pharmaceutical formulation is packaged in a container (e.g., a vial, bag, pen, or syringe). In certain embodiments, the formulation may be a lyophilized formulation or a liquid formulation. In certain embodiments, the amount of multi-specific binding protein in the container is suitable for administration as a single dose. In certain embodiments, the amount of multi-specific binding protein in the container is suitable for administration in multiple doses. In certain embodiments, the pharmaceutical composition or pharmaceutical formulation contains 0.1 to 2000 mg of the multi-specific binding protein. In certain embodiments, the pharmaceutical composition or pharmaceutical formulation contains 1 to 2000 mg, 10 to 2000 mg, 20 to 2000 mg, 50 to 2000 mg, 100 to 2000 mg, 200 to 2000 mg, 500 to 2000 mg, 1000 to 2000 mg, 0.1 to 1000 mg, 1 to 1000 mg, 10 to 1000 mg, 20 to 1000 mg, 50 to 1000 mg, 100 to 1000 mg, 200 to 1000 mg, 500 to 1000 mg, 0.1 to 500 mg, 1 to 500 mg, 10 to 500 mg, 20 to 500 mg, 50 to 500 mg, 100 to 500 mg, 200 to 500 mg, 0.1 to 200 mg, 1 to 200 mg, 10 to 200 mg, 20 to 200 mg, 50 to 200 mg, 100 to 200 mg, 0.1 to 100 mg, 1 to 100 mg, 10 to 100 mg, 20 to 100 mg, 50 to 100 mg, 0.1 to 50 mg, 1 to 50 mg, 10 to 50 mg, 20 to 50 mg, 0.1 to 20 mg, 1 to 20 mg, 10 to 20 mg, 0.1 to 10 mg, 1 to mg, or 0.1 to 1 mg of the multi-specific binding protein. In certain embodiments, the pharmaceutical composition or pharmaceutical formulation contains 0.1 mg, 1 mg, 2 mg, 5 mg, mg, 20 mg, 30 mg, 40 mg, 50 mg, 60 mg, 70 mg, 80 mg, 90 mg, 100 mg, 150 mg, 200 mg, 250 mg, 300 mg, 400 mg, 500 mg, 600 mg, 700 mg, 800 mg, 900 mg, 1000 mg, 1500 mg, or 2000 mg of the multi-specific binding protein.

Dosage Regimens

In certain embodiments, the method includes administering to a subject in need thereof a multi-specific binding protein, pharmaceutical composition, or pharmaceutical formulation disclosed herein (e.g., A49-F3′-TriNKET-Trastuzumab) in an initial four-week treatment cycle on day 1, day 8, and day 15. In certain embodiments, the multi-specific binding protein, pharmaceutical composition, or pharmaceutical formulation is administered to the subject only on these three days in the initial four-week treatment cycle. In specific embodiments, the multi-specific binding protein, pharmaceutical composition, or pharmaceutical formulation is not administered to the subject on day 22. This regimen is a dose intensification schedule, which is designed to reach maximal saturation of the target as early as possible during the course of the treatment while minimizing the infusion burden for the patient.

In certain embodiments, the method further includes administering to the subject the multi-specific binding protein, pharmaceutical composition, or pharmaceutical formulation on Day 1 and Day 15 in each of one or more subsequent four-week treatment cycles after the initial treatment cycle. In certain embodiments, the multi-specific binding protein, pharmaceutical composition, or pharmaceutical formulation is administered to the subject only on these two days in each subsequent four-week treatment cycle. In specific embodiments, the multi-specific binding protein, pharmaceutical composition, or pharmaceutical formulation is not administered to the subject on day 8 or day 22. The subsequent treatment cycles, in which the subject receives administration of the multi-specific binding protein, pharmaceutical composition, or pharmaceutical formulation once every two weeks, are designed to maintain a certain level of the multi-specific binding protein, pharmaceutical composition, or pharmaceutical formulation in the subject. In certain embodiments, the subject receives at least 1, 2, 3, 4, 5, 6, 7, 8, 9, 10, 11, 12, 13, 14, or 15 subsequent treatment cycles. In certain embodiments, the subject receives subsequent treatment cycles until regression of the cancer.

In certain embodiments, the method includes administering to a subject in need thereof a multi-specific binding protein, pharmaceutical composition, or pharmaceutical formulation disclosed herein (e.g., A49-F3′-TriNKET-Trastuzumab) as a monotherapy. In certain embodiments, the method includes administering the multi-specific binding protein, pharmaceutical composition, or pharmaceutical formulation disclosed herein (e.g., A49-F3′-TriNKET-Trastuzumab) intravenously as a one hour infusion in four-week treatment cycles.

In certain embodiments, the method includes administering to a subject in need thereof a multi-specific binding protein, pharmaceutical composition, or pharmaceutical formulation disclosed herein (e.g., A49-F3′-TriNKET-Trastuzumab) over a 45 to 75 min., 50 to min., 55 to 75 min., 60 to 75 min., 65 to 75 min., 70 to 75 min., 45 to 70 min., 50 to 70 min., to 70 min., 60 to 70 min., 65 to 70 min., 45 to 65 min., 50 to 65 min., 55 to 65 min., 60 to 65 min., 45 to 60 min., 50 to 60 min., 55 to 60 min., 45 to 135 min., 60 to 135 min., 75 to 135 min., to 135 min., 105 to 135 min., 120 to 135 min., 45 to 120 min., 60 to 120 min., 75 to 120 min., to 120 min., 105 to 120 min., 45 to 105 min., 60 to 105 min., 75 to 105 min., 90 to 105 min., to 90 min., 60 to 90 min., 75 to 90 min., 45 to 75 min., 60 to 75 min., 45 to 60 min., about 45 min., about 50 min., about 55 min., about 60 min., about 65 min., about 70 min., about 75 min., about 90 min., about 105 min., or about 120 min. period.

In certain embodiments, one or more doses of the multi-specific binding protein, pharmaceutical composition, or pharmaceutical formulation in the initial and subsequent treatment cycles contain 0.1-20 mg/kg, 0.1-10 mg/kg, 0.1-5 mg/kg, 0.1-2 mg/kg, 0.1-1 mg/kg, mg/kg, 0.1-0.2 mg/kg, 0.2-20 mg/kg, 0.2-10 mg/kg, 0.2-5 mg/kg, 0.2-2 mg/kg, 0.2-1 mg/kg, 0.2-0.5 mg/kg, 0.5-20 mg/kg, 0.5-10 mg/kg, 0.5-5 mg/kg, 0.5-2 mg/kg, 0.5-1 mg/kg, 1-20 mg/kg, 1-10 mg/kg, 1-5 mg/kg, or 1-2 mg/kg of the multi-specific binding protein relative to the body weight of the subject. In certain embodiments, one or more doses of the multi-specific binding protein, pharmaceutical composition, or pharmaceutical formulation in the initial and subsequent treatment cycles contain 0.1 mg/kg, 0.15 mg/kg, 0.2 mg/kg, 0.3 mg/kg, 0.4 mg/kg, mg/kg, 0.6 mg/kg, 0.7 mg/kg, 0.8 mg/kg, 0.9 mg/kg, 1 mg/kg, 1.5 mg/kg, 2 mg/kg, 2.5 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg of the multi-specific binding protein relative to the body weight of the subject.

In certain embodiments, each of the doses of the multi-specific binding protein, pharmaceutical composition, or pharmaceutical formulation in the initial and subsequent treatment cycles contain 0.1-20 mg/kg, 0.1-10 mg/kg, 0.1-5 mg/kg, 0.1-2 mg/kg, 0.1-1 mg/kg, mg/kg, 0.1-0.2 mg/kg, 0.2-20 mg/kg, 0.2-10 mg/kg, 0.2-5 mg/kg, 0.2-2 mg/kg, 0.2-1 mg/kg, 0.2-0.5 mg/kg, 0.5-20 mg/kg, 0.5-10 mg/kg, 0.5-5 mg/kg, 0.5-2 mg/kg, 0.5-1 mg/kg, 1-20 mg/kg, 1-10 mg/kg, 1-5 mg/kg, or 1-2 mg/kg of the multi-specific binding protein relative to the body weight of the subject. In certain embodiments, each of the doses of the multi-specific binding protein, pharmaceutical composition, or pharmaceutical formulation in the initial and subsequent treatment cycles contain a same amount in the range of 0.1-20 mg/kg, 0.1-10 mg/kg, mg/kg, 0.1-2 mg/kg, 0.1-1 mg/kg, 0.1-0.5 mg/kg, 0.1-0.2 mg/kg, 0.2-20 mg/kg, 0.2-10 mg/kg, 0.2-5 mg/kg, 0.2-2 mg/kg, 0.2-1 mg/kg, 0.2-0.5 mg/kg, 0.5-20 mg/kg, 0.5-10 mg/kg, 0.5-mg/kg, 0.5-2 mg/kg, 0.5-1 mg/kg, 1-20 mg/kg, 1-10 mg/kg, 1-5 mg/kg, or 1-2 mg/kg of the multi-specific binding protein relative to the body weight of the subject.

In certain embodiments, each of the doses in the initial and subsequent treatment cycles contain 0.1 mg/kg, 0.15 mg/kg, 0.2 mg/kg, 0.3 mg/kg, 0.4 mg/kg, 0.5 mg/kg, 0.6 mg/kg, mg/kg, 0.8 mg/kg, 0.9 mg/kg, 1 mg/kg, 1.5 mg/kg, 2 mg/kg, 2.5 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg of the multi-specific binding protein. In certain embodiments, each of the doses in the initial and subsequent treatment cycles contains 0.1 mg/kg, 0.15 mg/kg, 0.2 mg/kg, 0.3 mg/kg, 0.4 mg/kg, 0.5 mg/kg, mg/kg, 0.7 mg/kg, 0.8 mg/kg, 0.9 mg/kg, 1 mg/kg, 1.5 mg/kg, 2 mg/kg, 2.5 mg/kg, 3 mg/kg, 4 mg/kg, 5 mg/kg, 6 mg/kg, 7 mg/kg, 8 mg/kg, 9 mg/kg, 10 mg/kg, 11 mg/kg, 12 mg/kg, 13 mg/kg, 14 mg/kg, 15 mg/kg, 16 mg/kg, 17 mg/kg, 18 mg/kg, 19 mg/kg, or 20 mg/kg of the multi-specific binding protein.

In certain embodiments, each of the doses in the initial and subsequent treatment cycles contains 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1.6 mg/kg, 5.2 mg/kg, mg/kg, or 20 mg/kg of the multi-specific binding protein. In certain embodiments, each of the doses in the initial and subsequent treatment cycles contains 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1 mg/kg, 1.6 mg/kg, 5.2 mg/kg, 10 mg/kg, 20 mg/kg, or 50 mg/kg of the multi-specific binding protein. In certain embodiments, each of the doses in the initial and subsequent treatment cycles contains about 5 mg/kg, about 10 mg/kg, about 15 mg/kg, or about mg/kg of the multi-specific binding protein. In certain embodiments, each of the doses in the initial and subsequent treatment cycles contains 5 mg/kg, 10 mg/kg, 15 mg/kg, or 20 mg/kg of the multi-specific binding protein.

In a specific embodiment, each of the doses in the initial and subsequent treatment cycles contains 15 mg/kg of the multi-specific binding protein. In another specific embodiment, each of the doses in the initial and subsequent treatment cycles contains 20 mg/kg of the multi-specific binding protein.

It is contemplated that a priming dose can be used to reduce adverse effects (e.g., infusion-related reactions) of the multi-specific binding protein. It has been observed in a clinical study that the majority of infusion-related reactions occurred at the first dose. Subsequent doses were much better tolerated. It has also been observed that 5 mg/kg of the multi-specific binding protein was pharmacodynamically active in many tumors. Accordingly, in certain embodiments, where a subject is scheduled to receive greater than 5 mg/kg of the multi-specific binding protein in initial and subsequent treatment cycles (e.g., 10 mg/kg, 15 mg/kg, or 20 mg/kg), the first dose can be reduced to 5 mg/kg. The initially-scheduled higher dose can be given in subsequent doses (e.g., starting from the second dose on Day 8 of the initial treatment cycle).

In certain embodiments, the multi-specific binding protein, pharmaceutical composition, or pharmaceutical formulation is administered intravenously. For example, in certain embodiments, the multi-specific binding protein, pharmaceutical composition, or pharmaceutical formulation is administered by intravenous infusion, e.g., with a prefilled bag, a prefilled pen, or a prefilled syringe. In certain embodiments, the multi-specific binding protein, in a pharmaceutical composition or pharmaceutical formulation disclosed herein, is diluted prior to administration. For example, in certain embodiments, the pharmaceutical composition or pharmaceutical formulation is diluted with sodium chloride and is administered intravenously from a 250 ml saline bag. The intravenous infusion may be for about one hour (e.g., 50 to 80 minutes), 1-4 hours, 1-3 hours, 1-2 hours, 2-4 hours, 2-3 hours, or 3-4 hours. In certain embodiments, the multi-specific binding protein is administered by intravenous infusion over a period of 1-2 hours. In certain embodiments, the bag is connected to a channel including a tube and/or a needle.

It is contemplated that slower intravenous infusion may reduce adverse effects (e.g., infusion-related reactions) of the multi-specific binding protein. Accordingly, in certain embodiments, administration of early doses (e.g., the doses of an initial four-week treatment cycle) can be slower than administration of subsequent doses. This safety measure may be particularly helpful to subjects who receive high-dose therapies. In certain embodiments, where a subject is scheduled to receive 5 mg/kg or higher dose of the multi-specific binding protein in initial and subsequent treatment cycles (e.g., 5 mg/kg, 10 mg/kg, 15 mg/kg, or 20 mg/kg), the multi-specific binding protein is administered to the subject by intravenous infusion over a period of 3-4 hours in one or more initial doses (e.g., in the first dose, in the first two doses, or all the doses in the initial four-week treatment cycle). The period of intravenous infusion can be reduced to 1-2 hours in subsequent doses.

In certain embodiments, the multi-specific binding protein, pharmaceutical composition, or pharmaceutical formulation is administered subcutaneously. For example, in certain embodiments, the multi-specific binding protein, pharmaceutical composition, or pharmaceutical formulation is administered by subcutaneous injection using a syringe or an auto-injector. In certain embodiments, the multi-specific binding protein, pharmaceutical composition, or pharmaceutical formulation is administered by subcutaneous injection at the abdomen, arm, or thigh. In specific embodiments, the pharmaceutical composition or pharmaceutical formulation suitable for subcutaneous administration contains the multi-specific binding protein at a concentration greater than 50 mg/mL as described in the Pharmaceutical Formulations subsection above. In certain embodiments, the pharmaceutical composition or pharmaceutical formulation contains greater than or equal to 60 mg/mL, greater than or equal to 70 mg/mL, greater than or equal to 80 mg/mL, greater than or equal to 90 mg/mL, greater than or equal to 100 mg/mL, greater than or equal to 125 mg/mL, greater than or equal to 150 mg/mL, greater than or equal to 175 mg/mL, or greater than or equal to 200 mg/mL of the multi-specific binding protein. In certain embodiments, the pharmaceutical composition or pharmaceutical formulation contains 60-250 mg/mL, 60-225 mg/mL, 60-200 mg/mL, 60-175 mg/mL, 50-150 mg/mL, 60-150 mg/mL, 60-125 mg/mL, 60-100 mg/mL, 60-90 mg/mL, 60-80 mg/mL, 60-70 mg/mL, 70-250 mg/mL, 70-225 mg/mL, 70-200 mg/mL, 70-175 mg/mL, 70-150 mg/mL, 70-150 mg/mL, 70-125 mg/mL, 70-100 mg/mL, 70-90 mg/mL, 70-80 mg/mL, 80-250 mg/mL, 80-225 mg/mL, 80-200 mg/mL, 80-175 mg/mL, 80-150 mg/mL, 80-150 mg/mL, 80-125 mg/mL, 80-100 mg/mL, 80-90 mg/mL, 90-250 mg/mL, 90-225 mg/mL, 90-200 mg/mL, 90-175 mg/mL, 90-150 mg/mL, 90-150 mg/mL, 90-125 mg/mL, 90-100 mg/mL, 100-250 mg/mL, 100-225 mg/mL, 100-200 mg/mL, 100-175 mg/mL, 100-150 mg/mL, 100-125 mg/mL, 125-250 mg/mL, 125-225 mg/mL, 125-200 mg/mL, 125-175 mg/mL, 125-150 mg/mL, 150-250 mg/mL, 150-225 mg/mL, 150-200 mg/mL, 150-175 mg/mL, 175-250 mg/mL, 175-225 mg/mL, 175-200 mg/mL, 200-250 mg/mL, or 200-225 mg/mL of the multi-specific binding protein. In certain embodiments, the pharmaceutical composition or pharmaceutical formulation contains 60 mg/mL, 70 mg/mL, 80 mg/mL, 90 mg/mL, 100 mg/mL, 125 mg/mL, 150 mg/mL, 175 mg/mL, 200 mg/mL, or 220 mg/mL of the multi-specific binding protein.

HER2 Positive Cancers Suitable for Treatment

In specific embodiments, the types of cancer that can be treated with the multi-specific binding protein, pharmaceutical composition, or pharmaceutical formulation disclosed herein, where the second antigen-binding site of the multi-specific binding protein binds HER2, include but are not limited to breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, NSCLC, glioblastoma, esophageal cancer, squamous carcinoma of the skin, carcinoma of the salivary gland, biliary tract cancer, lung squamous, mesothelioma, liver cancer, sarcoma, bladder cancer, and gallbladder cancer. In certain embodiments, the cancer is a solid tumor. In certain embodiments, the cancer is a locally advanced or metastatic solid tumor. In certain embodiments, the cancer is gastric cancer (e.g., HER2-overexpressing or HER2 3+ gastric cancer). In certain embodiments, the cancer is esophageal cancer (e.g., HER2-overexpressing or HER2 3+ esophageal cancer). In certain embodiments, the cancer is urothelial bladder cancer (e.g., urothelial bladder cancer expressing HER2). In certain embodiments, the cancer is metastatic breast cancer (e.g., metastatic triple negative breast cancer). In certain embodiments, the cancer is a solid tumor (e.g., HER2-expressing or HER2 3+ solid tumor).

Methods of determining HER2 expression in a cancer include but are not limited to immunohistochemistry (IHC). Anti-HER2 antibodies (e.g., Ventana 4B5 antibody and Bond Oracle CB11 antibody) have been approved by the FDA for detecting HER2, and immunohistochemistry kits (e.g., HercepTest™) are commercially available. The level of HER2 expression in a tumor sample, as detected by immunohistochemistry, can be quantified and scored as 1+, 2+, or 3+ according to the ASCO/CAP guidelines (e.g., Wolff et al., (2007) J. Clin. Oncol. 25(1):118-45) and applicable update (e.g., the 2018 update according to Wolff et al., (2018) J. Clin. Oncol. 36(20):2105-22).

With respect to breast cancer, under the 2018 ASCO/CAP guideline, a cancer or tumor is scored as HER2 3+ if in a sample, circumferential membrane staining of HER2 is complete, intense and in >10% of tumor cells, which is readily appreciated using a low power objective and observed within a homogeneous and contiguous invasive cell population. A cancer or tumor is scored as HER2 2+ if weak to moderate complete membrane staining of HER2 is observed in >10% of tumor cells in a sample. A cancer or tumor is scored as HER2 1+ if incomplete membrane staining of HER2 is faint or barely perceptible and in >10% of tumor cells in a sample. A cancer or tumor is scored as HER2 0 if no HER2 staining is observed or membrane staining is incomplete and is faint or barely perceptible and in <10% of tumor cells. HER2 3+ IHC cancer or tumor is classified as HER2 high (“positive” according to the ASCO/CAP guideline). HER2 1+ or 0 IHC cancer or tumor is classified as HER2 low (“negative” according to the ASCO/CAP guideline). Where a cancer or tumor is scored as HER2 2+ in the initial IHC assessment, a reflex test (same specimen using ISH) or a new test (new specimen if available, using IHC or ISH) must be ordered. Based on the result of the reflex test or the new test, the cancer or tumor may be classified as HER2 high (“positive” according to the ASCO/CAP guideline) or HER2 low (“negative” according to the ASCO/CAP guidelines).

With respect to gastric cancer (e.g., gastroesophageal adenocarcinoma), under the 2016 CAP-ASCP-ASCO guideline (see Bartley et al., (2016) Arch. Pathol. Lab. Med. 140:1345-63), where surgical specimen is available, a cancer or tumor is scored as HER2 3+ if in the surgical specimen, strong, complete basolateral or lateral membranous reactivity is detected in ≥10% of tumor cells. A cancer or tumor is scored as HER2 2+ if weak to moderate, complete basolateral or lateral membranous reactivity is detected in ≥10% of tumor cells in the specimen. A cancer or tumor is scored as HER2 1+ if faint or barely perceptible membranous reactivity is detected in ≥10% of tumor cells in the specimen, including cells reactive only in part of their membrane. A cancer or tumor is scored as HER2 0 if no reactivity is detected, or membranous reactivity is detected in <10% of tumor cells. Slightly different standards are provided by the guidelines where the available sample is a biopsy specimen. HER2 3+ IHC cancer or tumor is classified as HER2 high (“positive” according to the CAP-ASCP-ASCO guideline). HER2 1+ or IHC cancer or tumor is classified as HER2 low (“negative” according to the CAP-ASCP-ASCO guideline). Where a cancer or tumor is scored as HER2 2+ in the initial IHC assessment, an ISH test must be ordered. Based on the result of the ISH test, the cancer or tumor may be classified as HER2 high (“positive” according to the CAP-ASCP-ASCO guideline) or HER2 low (“negative” according to the CAP-ASCP-ASCO guideline).

Medical guidelines may not be readily available to classify HER2 levels as “high” or “positive” vs. “low” or “negative” in certain other types of cancer (e.g., urothelial bladder cancer or non-small-cell lung cancer). With respect to these cancers, HER2 levels required by certain patient populations are described by IHC results on HER2 protein expression and/or ISH results on ERBB2 gene amplification. In certain embodiments, the determination of IHC 3+, 2+, 1+, and 0 generally follows the CAP-ASCP-ASCO guideline for gastric cancer. For example, HER2 1+ or higher requires that at least 10% of tumor cells stain positive for HER2, wherein the positive staining does not require complete membrane staining.

In certain embodiments, the subject treated by the method disclosed herein has a HER2 high cancer or tumor (e.g., gastric cancer, esophageal cancer, or breast cancer). In certain embodiments, the HER2 high cancer or tumor is eligible for treatment with trastuzumab. In certain embodiments, the subject treated by the method disclosed herein has a HER2 low cancer or tumor (e.g., gastric cancer, esophageal cancer, or breast cancer). In certain embodiments, the HER2 low cancer or tumor is ineligible for treatment with trastuzumab.

It is understood that a tumor with a HER2 level scored as 0 by IHC can nevertheless express HER2. A fraction of Herceptest 0 patients do express HER2 and may have up to 10% of the tumor cells with a faint staining for HER2. Accordingly, in certain embodiments, the present disclosure provides a method of treating cancer (e.g., solid tumor, e.g., breast cancer, gastric cancer, or esophageal cancer) using a multi-specific binding protein disclosed herein, wherein the HER2 status of the cancer is IHC 0, optionally wherein HER2 expression is still detectable by IHC (e.g., in at least 1%, 2%, 3%, 4%, 5%, 6%, 7%, 8%, or 9% of tumor cells). In certain embodiments, the cancer does not have ERBB2 gene amplification.

The contemplated ability of the multi-specific binding protein to treat non-HER2-high cancers may also allow treatment of cancer without first assessing HER2 status of the cancer. Such method is suitable for treating tumors that are, for example, difficult to take biopsy (e.g., preoperative biopsy or surgical specimen) of. Accordingly, in certain embodiments, the present disclosure provides a method of treating cancer (e.g., solid tumor, e.g., breast cancer, gastric cancer, or esophageal cancer) using a multi-specific binding protein disclosed herein, without first assessing HER2 expression level and/or ERBB2 gene amplification status of the cancer. In certain embodiments, the method does not comprise obtaining a biopsy (e.g., preoperative biopsy or surgical specimen) of the cancer prior to the administration.

It is understood that ERBB2 gene amplification is generally correlated with HER2 overexpression. Determining whether ERBB2 gene is amplified in a cancer tissue sample may help reduce false-positive results from immunohistochemistry of the same sample (see, e.g., Sarode et al., (2015) Arch. Pathol. Lab. Med. 139:922-28). Accordingly, in certain embodiments, the cancer or tumor in the subject has been assessed by ERBB2 gene amplification. In certain embodiments, the cancer or tumor harbors ERBB2 gene amplification, for example, an average ERBB2 gene copy number greater than or equal to 4.0 signals per cell (e.g., greater than or equal to 6.0 signals per cell). In certain embodiments, the cancer or tumor (e.g., a HER2 1+ cancer or tumor) has an average ERBB2 gene copy number less than 4.0 signals per cell. In certain embodiments, the cancer or tumor (e.g., a HER2 1+ cancer or tumor) does not have ERBB2 gene amplification. ERBB2 gene amplification can be assessed by in situ hybridization (ISH) with an ERBB2 probe. An additional chromosome 17 enumeration probe (CEP17) may be used, in a dual-probe approach, to assist with the analysis. A detailed process for assessing ERBB2 gene amplification is provided in the 2018 ASCO/CAP guidelines. Other methods of detecting gene amplification include but are not limited to fluorescent in situ hybridization (FISH), chromogenic in situ hybridization (CISH), quantitative PCR, and DNA sequencing. In certain embodiments, ERBB2 gene amplification is determined by FISH. In certain embodiments, ERBB2 gene amplification is determined by DNA sequencing (e.g., deep sequencing).

New technologies can be employed to assess HER2 levels in patient samples. For example, the automated quantitative analysis technology can quantitatively assess HER2 expression by measuring the intensity of antibody-conjugated fluorophores. The HERmark technology measures HER2 expression through a proximity-based release of antibody-bound fluorescent tags. The quantitative IHC technology converts antibody/antigen complexes into red dots, subsequently counted to quantify HER2 expression. The time-resolved fluorescence energy transfer technology enables assessment of HER2 expression through the detection of fluorescence emitted by two fluorophores in close proximity. The quantitative real-time polymerase chain reaction technology enables quantitative measurement of the amount of HER2 mRNA in a sample. The flow cytometry technology enables measurement of the number of HER2 proteins on the surface of a cell. These assays can complement the results of the IHC or FISH assays, thereby obtaining more accurate assessment of the HER2 level in the cancer or tumor.

For example, in certain embodiments, the cancer or tumor can be assessed by flow cytometry. As described in Example 3 below, Molecules of Equivalent Soluble Fluorochrome (MESF) beads with manufacturer predetermined fluorophore amounts can be used to generate a calibration curve. This calibration curve can be used to correlate the geometric mean fluorescence intensity (MFI) of a given cell population to standardized molecules numbers. The reagent detecting the HER2 proteins can be a protein that binds HER2 (e.g., a HER2 TriNKET disclosed herein) coupled with a fluorophore. Where the sample is a cell line, the geometric mean of the number of HER2 proteins on the cells can be determined. Cell lines with determined levels of HER2 can then be used as references, e.g., in an IHC assay, to determine the level of HER2 in tumor samples. In fact, SKBR3, MDA-MB-175 and MDA-MB-231 cell lines are used as references to set level 3, level 1 and level 0 staining intensity for the HercepTest. In certain embodiments, a cell line having a geometric mean of 500,000 or more (e.g., 600,000 or more, 700,000 or more, 800,000 or more, or 900,000 or more) HER2 proteins on each cell corresponds to the cells that have complete, intense circumferential membrane staining of HER2 in a HER2 3+ sample; a cell line having a geometric mean of 75,000 to 499,999 (e.g., 80,000 to 499,999, 90,000 to 499,999, 100,000 to 499,999, 110,000 to 499,999, 120,000 to 499,999, 85,000 to 399,999, 90,000 to 399,999, 100,000 to 399,999, 110,000 to 399,999, 115,000 to 399,999, 120,000 to 399,999, 85,000 to 299,999, 90,000 to 299,999, 95,000 to 299,999, 100,000 to 299,999, 105,000 to 299,999, 110,000 to 299,999, 85,000 to 199,999, to 199,999, 95,000 to 199,999, 100,000 to 199,999, 105,000 to 199,999, or 110,000 to 199,999,) HER2 proteins on each cell corresponds to the cells that have weak to moderate complete membrane staining of HER2 in a HER2 2+ sample; a cell line having a geometric mean of 10,000 to 74,999 (e.g., 11,000 to 74,999, 11,000 to 69,999, 11,000 to 64,999, 11,000 to 59,999, or 11,000 to 54,999) HER2 proteins on each cell corresponds to the cells that have faint or barely perceptible, incomplete membrane staining of HER2 in a HER2 1+ sample. A cell line having a geometric mean of less than 10,000 (e.g., 9000 or less, 8000 or less, 7000 or less, 6000 or less, 5000 or less, 4000 or less) HER2 proteins on each cell corresponds to negative membrane staining in a HER2 0 sample. According to the 2018 ASCO/CAP guideline, more than 10% of tumor cells in a sample must meet the required HER2 level threshold.

In certain embodiments, the subject treated by the method disclosed herein has a cancer or tumor (e.g., any one of the types of cancer disclosed in the preceding three paragraphs) with HER2 level scored as 1+, 2+, or 3+. In certain embodiments, the subject treated by the method disclosed herein has a cancer or tumor (e.g., any one of the types of cancer disclosed in the preceding three paragraphs) with HER2 level scored as 1+ or 2+. In certain embodiments, the subject treated by the method disclosed herein has a cancer or tumor (e.g., any one of the types of cancer disclosed in the preceding three paragraphs) with HER2 level scored as 2+ or 3+. In certain embodiments, the subject treated by the method disclosed herein has a cancer or tumor (e.g., any one of the types of cancer disclosed in the preceding three paragraphs) with HER2 level scored as 1+. In certain embodiments, the subject treated by the method disclosed herein has a cancer or tumor (e.g., any one of the types of cancer disclosed in the preceding three paragraphs) with HER2 level scored as 2+. In certain embodiments, the subject treated by the method disclosed herein has a cancer or tumor (e.g., any one of the types of cancer disclosed in the preceding three paragraphs) with HER2 level scored as 3+. In certain embodiments, the HER2 level is determined by immunohistochemistry (e.g., HercepTest™). In certain embodiments, the subject treated by the method disclosed herein has a tumor that shows HER2 expression at least as a faint/barely perceptible membrane staining detected in at least or more than 10% of the tumor cells. In certain embodiments, the subject treated by the method disclosed herein has a tumor that shows HER2 expression at least as a weak to moderate complete membrane staining detected in at least or more than 10% of the tumor cells. In certain embodiments, the subject treated by the method disclosed herein has a tumor that shows HER2 expression at least as a strong complete membrane staining detected in at least or more than 10% of the tumor cells.

In certain embodiments, the subject treated by the method disclosed herein has a cancer or tumor with a HER2 level scored as 1+, and the cancer or tumor is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, uterine cancer, a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the subject treated by the method disclosed herein has a cancer or tumor with a HER2 level scored as 1+, and the cancer or tumor is a hematologic malignancy such as leukemia, acute myeloid leukemia (AML), acute lymphoblastic leukemia (ALL), myelodysplasia, myelodysplastic syndromes, acute T-lymphoblastic leukemia, acute promyelocytic leukemia, chronic myelomonocytic leukemia, or myeloid blast crisis of chronic myeloid leukemia. In certain embodiments, the subject treated by the method disclosed herein has a cancer or tumor with a HER2 level scored as 1+, and the cancer or tumor is breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, NSCLC, glioblastoma, esophageal cancer, squamous carcinoma of the skin, carcinoma of the salivary gland, biliary tract cancer, lung squamous, mesothelioma, liver cancer, sarcoma, bladder cancer, gallbladder cancer, urothelial bladder cancer, or metastatic breast cancer. In certain embodiments, the subject treated by the method disclosed herein has a cancer or tumor with a HER2 level scored as 1+, and the cancer or tumor is a solid tumor, such as a locally advanced or metastatic solid tumor. In certain embodiments, the cancer or tumor is a triple negative breast cancer with a HER2 level scored as 1+. In certain embodiments, the cancer or tumor is positive for a hormone receptor (i.e., positive for estrogen receptor, progesterone receptor, or both) and negative for HER2 (e.g., as assessed by IHC). According to the 2018 ASCO/CAP HER2 testing guideline update, breast cancer is considered HER2 negative in cases of IHC 0 and 1+ results, or IHC 2+ with a negative ISH assay. Thus, in certain embodiments, the cancer or tumor is a triple negative breast cancer negative for estrogen receptor and progesterone receptor (e.g., as assessed by IHC) with a HER2 level scored as IHC 1+. In certain embodiments, the cancer or tumor is a hormone receptor positive breast cancer (e.g., as assessed by IHC) with a HER2 level scored as IHC 1+. In certain embodiments, the HER2 1+ cancer or tumor has an average ERBB2 gene copy number less than 4.0 signals per cell. In certain embodiments, the HER2 1+ cancer or tumor does not have ERBB2 gene amplification.

In certain embodiments, the subject treated by the method disclosed herein has a cancer or tumor with a HER2 level scored as 2+, and the cancer or tumor is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, uterine cancer, a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the subject treated by the method disclosed herein has a cancer or tumor with a HER2 level scored as 2+, and the cancer or tumor is a hematologic malignancy such as leukemia, acute myeloid leukemia (AML), acute lymphoblastic leukemia (ALL), myelodysplasia, myelodysplastic syndromes, acute T-lymphoblastic leukemia, acute promyelocytic leukemia, chronic myelomonocytic leukemia, or myeloid blast crisis of chronic myeloid leukemia. In certain embodiments, the subject treated by the method disclosed herein has a cancer or tumor with a HER2 level scored as 2+, and the cancer or tumor is breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, NSCLC, glioblastoma, esophageal cancer, squamous carcinoma of the skin, carcinoma of the salivary gland, biliary tract cancer, lung squamous, mesothelioma, liver cancer, sarcoma, bladder cancer, gallbladder cancer, urothelial bladder cancer, or metastatic breast cancer. In certain embodiments, the subject treated by the method disclosed herein has a cancer or tumor with a HER2 level scored as 2+, and the cancer or tumor is a solid tumor, such as a locally advanced or metastatic solid tumor. In certain embodiments, the cancer or tumor is a triple negative breast cancer with a HER2 level scored as IHC 2+ with a negative ISH assay. In certain embodiments, the cancer or tumor is a hormone receptor positive breast cancer (e.g., as assessed by IHC) with a HER2 level scored as IHC 2+ with a negative ISH assay. In certain embodiments, the HER2 2+ cancer or tumor has an average ERBB2 gene copy number less than 4.0 signals per cell. In certain embodiments, the HER2 2+ cancer or tumor does not have ERBB2 gene amplification.

In certain embodiments, the subject treated by the method disclosed herein has a cancer or tumor with a HER2 level scored as 1+ or 2+, and the cancer or tumor is brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, uterine cancer, a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the subject treated by the method disclosed herein has a cancer or tumor with a HER2 level scored as 1+ or 2+, and the cancer or tumor is a hematologic malignancy such as leukemia, acute myeloid leukemia (AML), acute lymphoblastic leukemia (ALL), myelodysplasia, myelodysplastic syndromes, acute T-lymphoblastic leukemia, acute promyelocytic leukemia, chronic myelomonocytic leukemia, or myeloid blast crisis of chronic myeloid leukemia. In certain embodiments, the subject treated by the method disclosed herein has a cancer or tumor with a HER2 level scored that is not high (e.g., scored as 1+ or 2+), and the cancer or tumor is breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, NSCLC, glioblastoma, esophageal cancer, squamous carcinoma of the skin, carcinoma of the salivary gland, biliary tract cancer, lung squamous, mesothelioma, liver cancer, sarcoma, bladder cancer, gallbladder cancer, urothelial bladder cancer, and metastatic breast cancer. In certain embodiments, the subject treated by the method disclosed herein has a cancer or tumor with a HER2 level scored as 1+ or 2+, and the cancer or tumor is a solid tumor, such as a locally advanced or metastatic solid tumor. In certain embodiments, the cancer or tumor is a triple negative breast cancer with a HER2 level scored as IHC 1+, or IHC 2+ with a negative ISH assay. In certain embodiments, the cancer or tumor is a hormone receptor positive breast cancer (e.g., as assessed by IHC) with a HER2 level scored as IHC 1+, or IHC 2+ with a negative ISH assay. In certain embodiments, the cancer or tumor with a HER2 level scored as 1+ or 2+ has an average ERBB2 gene copy number less than 4.0 signals per cell. In certain embodiments, the cancer or tumor with a HER2 level scored as 1+ or 2+ does not have ERBB2 gene amplification.

In certain embodiments, the subject treated in accordance with the methods disclosed herein has not received prior therapy for treating the cancer. In certain embodiments, the subject treated in accordance with the methods disclosed herein has not received prior chemotherapy or immunotherapy for treating the cancer. In certain embodiments, the subject treated in accordance with the methods disclosed herein has received a prior therapy (e.g., a chemotherapy or immunotherapy) but continues to experience cancer progression despite the prior therapy. In certain embodiments, the subject treated in accordance with the methods disclosed herein has experienced cancer regression after receiving a prior therapy (e.g., a chemotherapy or immunotherapy), but later experienced cancer relapse. In certain embodiments, the subject treated in accordance with the methods disclosed herein is intolerant to a prior therapy (e.g., a chemotherapy or immunotherapy).

Monotherapies and Combinational Use with Other Cancer Therapies

The multi-specific binding protein disclosed herein can be used as a monotherapy or in combination with one or more therapies. In certain embodiments, the multi-specific binding protein is used as a monotherapy in accordance with the dosage regimen disclosed herein. In other embodiments, the multi-specific binding protein is used in combination with one or more therapies administered in accordance with a dosage regimen known to be suitable for treating the particular subject with the particular cancer, and the multi-specific binding protein is administered in accordance with the dosage regimen disclosed herein. In certain embodiments, the method of treatment disclosed herein is used as an adjunct to surgical removal of the primary lesion.

Combination Therapy with PD-1 Inhibitor

In certain embodiments, the method of the present invention further includes administering to the subject a PD-1 inhibitor (e.g., an anti-PD-1 antibody). Many anti-PD-1 antibodies have been developed for therapeutic purposes and are described in, for example, Gong et al., (2018) J. ImmunoTher Cancer 6:8.

In certain embodiments, the anti-PD-1 antibody is nivolumab. In certain embodiments, 0.1 to 10 mg/kg, 0.1 to 3 mg/kg, 0.1 to 1 mg/kg, 0.1 to 0.3 mg/kg, 0.3 to 10 mg/kg, 0.3 to 3 mg/kg, 0.3 to 1 mg/kg, 1 to 10 mg/kg, 1 to 3 mg/kg, or 3 to 10 mg/kg of nivolumab is administered to the subject. In certain embodiments, 120 to 600 mg, 120 to 480 mg, 120 to 360 mg, 120 to 240 mg, 240 to 600 mg, 240 to 480 mg, 240 to 360 mg, 360 to 600 mg, 360 to 480 mg, or 480 to 600 mg of nivolumab is administered to the subject. In certain embodiments, 120 mg, 240 mg, 360 mg, 480 mg, or 600 mg of nivolumab is administered to the subject. In certain embodiments, 480 mg of nivolumab is administered on day 1 of the initial treatment cycle. In certain embodiments, if the subject receives one or more subsequent treatment cycles, 480 mg of nivolumab is administered once every four weeks in the subsequent treatment cycles, starting from day 1 of each subsequent treatment cycle. In certain embodiments, nivolumab is administered as a 30 minute, 35 minute, 45 minute, 50 minute, 60 minute, 65 minute, 70 minute, 75 minute, 80 minute, 85 minute, or 90 minute intravenous infusion in four-week treatment cycles.

In certain embodiments, a multi-specific binding protein, pharmaceutical composition, or pharmaceutical formulation disclosed herein is administered as a 30 min., 45 min., 60 min., 75 min. 90 min., 1-2 hour, or 3-4 hour intravenous infusion, and nivolumab is administered as a 30 min., 45 min., 60 min., 75 min., or 90 min. intravenous infusion in four-week treatment cycles. In certain embodiments, the multi-specific binding protein, pharmaceutical composition, or pharmaceutical formulation disclosed herein is administered as a 60 min. intravenous infusion, and nivolumab is administered as a 30 min. intravenous infusion in four-week treatment cycles. In certain embodiments, the multi-specific binding protein, pharmaceutical composition, or pharmaceutical formulation disclosed herein is administered as a 3-4 hour intravenous infusion, and nivolumab is administered as a 30 min. intravenous infusion in an initial four-week treatment cycle. In certain embodiments, the multi-specific binding protein, pharmaceutical composition, or pharmaceutical formulation disclosed herein is administered as a 1-2 hour intravenous infusion, and nivolumab is administered as a 30 min. intravenous infusion in subsequent four-week treatment cycles. Where nivolumab is administered on the same day as the multi-specific binding protein (e.g., on day 1 of each four-week treatment cycle), in certain embodiments, nivolumab is administered prior to the multi-specific binding protein, for example, within 30 min., 1 hour, 2 hours, 3 hours, 4 hours before administration of the multi-specific binding protein, or during the same visit as administration of the multi-specific binding protein.

In certain embodiments, a multi-specific binding protein, pharmaceutical composition, or pharmaceutical formulation disclosed herein is administered in a first treatment cycle in combination with nivolumab, where the multi-specific binding protein, pharmaceutical composition, or pharmaceutical formulation and nivolumab are both administered on day 1, and the multi-specific binding protein, pharmaceutical composition, or pharmaceutical formulation is administered alone at day 8. In certain embodiments, the multi-specific binding protein, pharmaceutical composition, or pharmaceutical formulation is administered in combination with nivolumab in subsequent-week treatment cycles after an initial cycle, where the multi-specific binding protein, pharmaceutical composition, or pharmaceutical formulation and nivolumab are both administered on day 1.

Treatment of Cold Tumors with a Combination Therapy

The present disclosure provides a method of treating a cold solid tumor expressing a tumor-associated antigen (TAA) in a subject in need thereof by administering effective amounts of a multi-specific binding protein that targets the TAA and an immune checkpoing inhibitor. The multi-specific binding protein has a first antigen-binding site that binds NKG2D, a second antigen-binding site that binds the TAA, and an antibody Fc domain or a portion thereof sufficient to bind CD16, or a third antigen-binding site that binds CD16.

Cold solid tumors generally do not trigger strong immune responses in the absence of therapeutic intervention. Such tumors are unlikely to respond adequately to immune checkpoint inhibitors, given the lack or paucity of infiltrating lymphocytes (e.g., T cells) in the tumor. It has been observed in a cold tumor animal model, as described in Example 4 below, that a multi-specific binding protein disclosed herein can induce immune cell infiltration. In addition to an increase of absolute number of immune cells, the infiltration leads to a reprogramming of the tumor microenvironment by recruiting higher fractions of immune cells that have anti-tumor effects, such as NK cells, CD8+ T cells, and tumor-associated macrophages. The infiltrated lymphocytes (e.g., NK cells, CD8+ T cells, and CD4+ T cells) express immune checkpoint proteins, such as PD-1, LAG-3, TIGIT, and TIM-3. Moreover, the average amount of PD-1 expressed on each infiltrating lymphocyte (e.g., CD8+ T cell or CD4+ T cell) is increased by the multi-specific binding protein. Based on these observations and without wishing to be bound by theory, it has been contemplated that a multi-specific binding protein disclosed herein can change the microenvironment of cold tumors by attracting immune cells that will contribute to the control of the tumor, which will engage the IFNγ pathway, resulting in an expression of PD-L1 by both the tumor cells and the cells present in the micro-environment. Accordingly, a combination therapy, including a multi-specific binding protein and an immune checkpoint inhibitor, can be used to treat cold tumors with a synergistic effect.

Cold tumors can be identified clinically by various means. For example, a solid tumor can be identified as a cold tumor if it has progressed during or after treatment with a previously administered immune checkpoint inhibitor (e.g., PD-1 inhibitor, e.g., an anti-PD-1 antibody). A solid tumor can also be identified as a cold tumor if a biopsy (e.g., preoperative biopsy or surgical specimen) shows that the tumor has a reduced amount of lymphocyte infiltration, a reduced amount of immune checkpoint (e.g., PD-1) expression, or a reduced amount of immune checkpoint ligand (e.g., PD-L1) expression, e.g., reduced by at least 20%, at least 30%, at least 40%, at least 50%, at least 60%, at least 70%, at least 80%, or at least 90 relative to a hot tumor (e.g., a tumor responsive to an immune checkpoint inhibitor) of the same type.

It is contemplated that the ability of multi-specific binding proteins to reprogram tumor microenvironments relates to co-engagement of a TAA on a tumor cell and NKG2D and CD16 on an NK cell. Thus, the mechanism is applicable to various TAAs, including but not limited to ANO1, BCMA, EpCAM, CAIX, CEA, CCR4, CD2, CD123, CD133, CD19, CD20, CD22, CD25, CD30, CD33, CD37, CD38, CD40, CD52, CD70, CLAUDIN-18.2, DLL3, EGFR/ERBB1, GD2, IGF1R, HER2, HER3/ERBB3, HER4/ERBB4, cMET, SLAMF7, PSMA, mesothelin, MICA, MICB, TRAILR1, TRAILR2, TROP2, MAGE-A3, B7.1, B7.2, CTLA4, PD1, 5T4, GPNMB, FR-alpha, PAPP-A, FLT3, GPC3, CXCR4, ROR1, ROR2, HLA-E, PD-L1, VLA4, CD44, CD13, CD15, CD47, CLL1, CD81, CD23, CD79a, CD79b, CD80, CRLF2, SLAMF7, CD138, CA125, NaPi2b, Nectin4, ADAMS, ADAMS, SLC44A4, CA19-9, LILRB1, LILRB2, LILRB3, LILRB4, LILRB5, LILRA1, LILRA2, LILRA3, LILRA4, LILRA5, LILRA6, CCR8, CD7, CTLA4, CX3CR1, ENTPDI, HAVCR2. IL-1R2., PDCD1LG2, TIGIT. TNFRSF4, TNFRSF8, TN FRSF9, GEM, NT5E, TNFRST1 8, P-cadherin, Plexin-A1, TNFRSF10B, STEAP1, CDCP1, PTK7, Axl, erbB-3, EDNRB, Tyrp1, CD14, CD163, CSF3R, Siglec-9, ITGAM, VISTA, B7-H4 (VTCN1), CCR1, LRRC25, PTAFR, SIRPB1, TLR2, TLR4, CD300LB, ATP1A3, CCR5, MUC1 (or MUC1-C), Plexin-A1, TNFRSF10B, STEAP1, CDCP1, PTK7, AXL, EDNRB, OLR1, and TYRP1. The features of multi-specific binding proteins disclosed herein that bind HER2 are also applicable to similar multi-specific binding proteins that bind another TAA.

Specific HER2-expressing solid tumors have been contemplated for the combination therapy disclosed herein. A phase 2 clinical study has been designed to treat (i) gastric cancer, e.g., a HER2-low gastric cancer; (ii) esophageal cancer, e.g., a HER2-low esophageal cancer, e.g., adenocarcinoma of the esophagus; (iii) urothelial bladder cancer, e.g., with HER2 level scored 1+ or higher as assessed by immunohistochemistry; and (iv) non-small-cell lung cancer (NSCLC), e.g., having a HER2 level scored 1+ or higher as assessed by immunohistochemistry and having no ERBB2 gene amplification.

Other solid tumors are also contemplated, including but are not limited to brain cancer, bladder cancer, breast cancer, cervical cancer, colon cancer, colorectal cancer, endometrial cancer, esophageal cancer, leukemia, lung cancer, liver cancer, melanoma, ovarian cancer, pancreatic cancer, prostate cancer, rectal cancer, renal cancer, stomach cancer, testicular cancer, uterine cancer, a vascularized tumor, squamous cell carcinoma, adenocarcinoma, small cell carcinoma, melanoma, glioma, neuroblastoma, sarcoma (e.g., an angiosarcoma or chondrosarcoma), larynx cancer, parotid cancer, biliary tract cancer, thyroid cancer, acral lentiginous melanoma, actinic keratoses, acute lymphocytic leukemia, acute myeloid leukemia, adenoid cystic carcinoma, adenomas, adenosarcoma, adenosquamous carcinoma, anal canal cancer, anal cancer, anorectum cancer, astrocytic tumor, Bartholin gland carcinoma, basal cell carcinoma, biliary cancer, bone cancer, bone marrow cancer, bronchial cancer, bronchial gland carcinoma, carcinoid, cholangiocarcinoma, chondrosarcoma, choroid plexus papilloma/carcinoma, chronic lymphocytic leukemia, chronic myeloid leukemia, clear cell carcinoma, connective tissue cancer, cystadenoma, digestive system cancer, duodenum cancer, endocrine system cancer, endodermal sinus tumor, endometrial hyperplasia, endometrial stromal sarcoma, endometrioid adenocarcinoma, endothelial cell cancer, ependymal cancer, epithelial cell cancer, Ewing's sarcoma, eye and orbit cancer, female genital cancer, focal nodular hyperplasia, gallbladder cancer, gastric antrum cancer, gastric fundus cancer, gastrinoma, glioblastoma, glucagonoma, heart cancer, hemangioblastomas, hemangioendothelioma, hemangiomas, hepatic adenoma, hepatic adenomatosis, hepatobiliary cancer, hepatocellular carcinoma, Hodgkin's disease, ileum cancer, insulinoma, intraepithelial neoplasia, intraepithelial squamous cell neoplasia, intrahepatic bile duct cancer, invasive squamous cell carcinoma, jejunum cancer, joint cancer, Kaposi's sarcoma, pelvic cancer, large cell carcinoma, large intestine cancer, leiomyosarcoma, lentigo maligna melanomas, lymphoma, male genital cancer, malignant melanoma, malignant mesothelial tumors, medulloblastoma, medulloepithelioma, meningeal cancer, mesothelial cancer, metastatic carcinoma, mouth cancer, mucoepidermoid carcinoma, multiple myeloma, muscle cancer, nasal tract cancer, nervous system cancer, neuroepithelial adenocarcinoma nodular melanoma, non-epithelial skin cancer, non-Hodgkin's lymphoma, oat cell carcinoma, oligodendroglial cancer, oral cavity cancer, osteosarcoma, papillary serous adenocarcinoma, penile cancer, pharynx cancer, pituitary tumors, plasmacytoma, pseudosarcoma, pulmonary blastoma, rectal cancer, renal cell carcinoma, respiratory system cancer, retinoblastoma, rhabdomyosarcoma, sarcoma, serous carcinoma, sinus cancer, skin cancer, small cell carcinoma, small intestine cancer, smooth muscle cancer, soft tissue cancer, somatostatin-secreting tumor, spine cancer, squamous cell carcinoma, striated muscle cancer, submesothelial cancer, superficial spreading melanoma, T cell leukemia, tongue cancer, undifferentiated carcinoma, ureter cancer, urethra cancer, urinary bladder cancer, urinary system cancer, uterine cervix cancer, uterine corpus cancer, uveal melanoma, vaginal cancer, verrucous carcinoma, VIPoma, vulva cancer, well differentiated carcinoma, or Wilms tumor. In certain embodiments, the solid tumor is breast cancer, thyroid cancer, gastric cancer, renal cell carcinoma, adenocarcinoma of the lung, prostate cancer, cholangiocarcinoma, uterine cancer, pancreatic cancer, colorectal cancer, ovarian cancer, cervical cancer, head and neck cancer, NSCLC, glioblastoma, esophageal cancer, squamous carcinoma of the skin, carcinoma of the salivary gland, biliary tract cancer, lung squamous, mesothelioma, liver cancer, sarcoma, bladder cancer, gallbladder cancer, urothelial bladder cancer, or metastatic breast cancer.

Inhibitors of various immune checkpoint proteins are useful in the combination therapy. In certain embodiments, the immune checkpoint inhibitor includes a PD-1 inhibitor, e.g., an anti-PD-1 antibody (e.g., nivolumab, pembrolizumab, or cemiplimab), an anti-PD-L1 antibody (e.g., atezolizumab, durvalumab, or avelumab), or an anti-PD-L2 antibody. In certain embodiments, the immune checkpoint inhibitor includes a LAG-3 inhibitor, e.g., an anti-LAG-3 antibody (e.g., relatlimab). In certain embodiments, the immune checkpoint inhibitor includes a TIGIT inhibitor, e.g., an anti-TIGIT antibody. In certain embodiments, the immune checkpoint inhibitor includes a TIM-3 inhibitor, e.g., an anti-TIM-3 antibody. Variants of these antibodies, such as antigen-binding fragments thereof, optionally fused with one or more other proteins or fragments thereof, are also contemplated for use in a combination therapy disclosed herein.

In addition, the present disclosure provides a method of treating cancer (e.g., a solid tumor) expressing a TAA (e.g., HER2) in a subject in need thereof by administering effective amounts of a multi-specific binding protein that targets the TAA and a TIGIT inhibitor (e.g., an anti-TIGIT antibody).

Combination Therapy with Chemotherapy

In certain embodiments, the method of the present invention further includes administering to the subject a chemotherapeutic agent (e.g., a cytoskeletal-disrupting chemotherapeutic agent). Many such agents have been developed for therapeutic purposes and are described in, for example, Ong et al., (2020) Cancers 12(1): 238.

In certain embodiments, the chemotherapeutic agent includes a cytoskeletal-disrupting chemotherapeutic agent (e.g., nab-paclitaxel). In certain embodiments, the cytoskeletal-disrupting chemotherapeutic agent is nab-paclitaxel. In certain embodiments, 50 to 300 mg/m², 50 to 200 mg/m², 50 to 150 mg/m², 50 to 100 mg/m², 100 to 300 mg/m², 100 to 200 mg/m², 100 to 150 mg/m², 150 to 300 mg/m², 150 to 200 mg/m 2, or 200 to 300 mg/m 2 of nab-paclitaxel is administered to the subject. In certain embodiments, 80 mg/m², 100 mg/m², 125 mg/m², 200 mg/m 2, or 260 mg/m 2 of nab-paclitaxel is administered to the subject.

In some embodiments, the cytoskeletal-disrupting chemotherapeutic agent (e.g., nab-paclitaxel) is administered three times every four weeks. In some embodiments, the cytoskeletal-disrupting chemotherapeutic agent (e.g., nab-paclitaxel) is administered on day 1, day 8, and day 15 of each four-week treatment cycle. In some embodiments, the cytoskeletal-disrupting chemotherapeutic agent (e.g., nab-paclitaxel) is not administered on day 22 of any of the treatment cycles. In certain embodiments, 100 mg/m 2 of nab-paclitaxel is administered on day 1, day 8, and day 15 of the initial treatment cycle. In certain embodiments, if the subject receives one or more subsequent treatment cycles, 100 mg/m 2 of nab-paclitaxel is administered three times every four weeks in the subsequent treatment cycles, on day 1, day 8, and day 15 of each subsequent treatment cycle. In some embodiments, the cytoskeletal-disrupting chemotherapeutic agent (e.g., nab-paclitaxel) is administered on the same day as the multi-specific binding protein and prior to the administration of the multi-specific binding protein.

In certain embodiments, nab-paclitaxel is administered as a 30 minute, 35 minute, minute, 50 minute, 60 minute, 65 minute, 70 minute, 75 minute, 80 minute, 85 minute, or 90 minute intravenous infusion in four-week treatment cycles.

In certain embodiments, a multi-specific binding protein, pharmaceutical composition, or pharmaceutical formulation disclosed herein is administered as a 30 min., 45 min., 60 min., 75 min. 90 min., 1-2 hour, or 3-4 hour intravenous infusion, and nab-paclitaxel is administered as a 30 min., 45 min., 60 min., 75 min., or 90 min. intravenous infusion in four-week treatment cycles. In certain embodiments, the multi-specific binding protein, pharmaceutical composition, or pharmaceutical formulation disclosed herein is administered as a min. intravenous infusion, and nab-paclitaxel is administered as a 30 min. intravenous infusion in four-week treatment cycles. In certain embodiments, the multi-specific binding protein, pharmaceutical composition, or pharmaceutical formulation disclosed herein is administered as a 3-4 hour intravenous infusion, and nab-paclitaxel is administered as a 30 min. intravenous infusion in an initial four-week treatment cycle. In certain embodiments, the multi-specific binding protein, pharmaceutical composition, or pharmaceutical formulation disclosed herein is administered as a 1-2 hour intravenous infusion, and nab-paclitaxel is administered as a min. intravenous infusion in subsequent four-week treatment cycles. Where nab-paclitaxel is administered on the same day as the multi-specific binding protein (e.g., on day 1, day 8, and day of the initial four-week treatment cycle and day 1 and day 15 of each subsequent four-week treatment cycle), in certain embodiments, nab-paclitaxel is administered prior to the multi-specific binding protein, for example, within 30 min., 1 hour, 2 hours, 3 hours, 4 hours before administration of the multi-specific binding protein, or during the same visit as administration of the multi-specific binding protein.

Exemplary Therapeutic Uses

The present disclosure provides exemplary methods of treating specific cancer patients, such as HER2 high gastric cancer, HER2 low gastric cancer, HER2 high esophageal cancer, HER2 low esophageal cancer, HER2 high breast cancer, HER2 low breast cancer, triple negative breast cancer (TNBC), urothelial bladder cancer with HER2 level scored 1+ or higher, non-small-cell lung cancer (NSCLC) with HER2 level scored 2+ or higher and ERBB2 gene amplification, or NSCLC with HER2 level scored 1+ or higher and no ERBB2 gene amplification. The methods can include administering a multi-specific binding protein including a first antigen-binding site that binds NKG2D, a second antigen-binding site that binds HER2, and an antibody Fc domain or a portion thereof sufficient to bind CD16, or a third antigen-binding site that binds CD16, e.g., as described below.

The multi-specific binding protein can be used as a first-line therapy, a second-line therapy, a third-line therapy, etc. It is understood that a line of therapy refers to at least one complete cycle of a single agent, a regimen of a combination therapy, or a planned sequential therapy of various regimens (see Rajkumar et al., Blood (2015) 126 (7): 921-22). Where the multi-specific binding protein is used after a previous therapy, as described herein, it is understood that the previous therapy need not be a complete line of therapy unless indicated otherwise. The present disclosure provides various methods of treatment using the multi-specific binding protein after one or more previous lines of therapy. In such instances, the same methods are contemplated except that the one or more previous therapies are not completed lines of therapy. In other words, where it is disclosed that the multi-specific binding protein can be used as a second or higher line of therapy, use of the multi-specific binding protein as an adjuvant therapy (before completion of the previous therapeutic regimen) is also contemplated.

In some embodiments, the first antigen-binding site that binds NKG2D includes a heavy chain variable domain (VH) with complementarity-determining region 1 (CDR1), complementarity-determining region 2 (CDR2), and complementarity-determining region 3 (CDR3) having the amino acid sequences of SEQ ID NOs: 168, 96, and 188, respectively; and a light chain variable domain (VL) with CDR1, CDR2, and CDR3 having the amino acid sequences of SEQ ID NOs: 99, 100, and 101, respectively. In some embodiments, the first antigen-binding site that binds NKG2D includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:94, and a VL with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:98. In some embodiments, the first antigen-binding site that binds NKG2D is a Fab.

In some embodiments, the second antigen-binding site that binds HER2 includes a VH with CDR1, CDR2, and CDR3 having the amino acid sequences of SEQ ID NOs: 115, 116, and 117, respectively; and a VL with CDR1, CDR2, and CDR3 having the amino acid sequences of SEQ ID NOs: 119, 120, and 121, respectively. In some embodiments, the second antigen-binding site that binds HER2 includes a VH with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:195, and a VL with an amino acid sequence at least 90% (e.g., at least 91%, at least 92%, at least 93%, at least 94%, at least 95%, at least 96%, at least 97%, at least 98%, at least 99%, or 100%) identical to the amino acid sequence of SEQ ID NO:196. In some embodiments, the second antigen-binding site that binds HER2 is a single chain variable fragment (scFv). In some embodiments, the VL of the scFv is linked to the VH of the scFv via a flexible linker. In some embodiments, the flexible linker has the amino acid sequence of SEQ ID NO:143. In some embodiments, the VL of the scFv is positioned to the N-terminus of the VH of the scFv. In some embodiments, the VH of the scFv forms a disulfide bridge with the VL of the scFv (e.g., between residues C44 of the VH of the scFv and C100 of the VL of the scFv). In some embodiments, the scFv has the amino acid sequence of SEQ ID NO:139.

In some embodiments, the antibody Fc domain includes a first antibody Fc sequence linked to the Fab that binds NKG2D and a second antibody Fc sequence linked to the scFv that binds HER2. In some embodiments, the first antibody Fc sequence is linked to the heavy chain portion of the Fab. In some embodiments, the scFv is linked to the second antibody Fc sequence via a hinge comprising Ala-Ser. In some embodiments, the first and second antibody Fc sequences each comprise a hinge and a CH2 domain of a human IgG1 antibody. In some embodiments, the first and second antibody Fc sequences each comprise an amino acid sequence at least 90% identical to amino acids 234-332 of a wild-type human IgG1 antibody. In some embodiments, the first and second antibody Fc sequences comprise different mutations that promote heterodimerization. In some embodiments, the first antibody Fc sequence is a human IgG1 Fc sequence comprising K360E and K409W substitutions. In some embodiments, the second antibody Fc sequence is a human IgG1 Fc sequence comprising Q347R, D399V, and F405T substitutions.

In some embodiments, the multi-specific binding protein includes a first polypeptide, second polypeptide, and third polypeptide having amino acid sequences of SEQ ID NO:141, SEQ ID NO:140, and SEQ ID NO:142, respectively.

It is contemplated that the multi-specific binding protein used in the methods disclosed herein can be formulated as a pharmaceutical composition as described below.

Some pharmaceutical compositions of the present disclosure contain: (i) 10 mg/mL to 50 mg/mL of the multi-specific binding protein including a first antigen-binding site that binds NKG2D, a second antigen-binding site that binds HER2, and an antibody Fc domain; (ii) 5 to 50 mM histidine; (iii) 50 to 300 mM sucrose; and (iv) 0.005% to 0.05% (w/v) polysorbate-80, at pH to 6.5.

Some pharmaceutical compositions of the present disclosure contain: (i) 10 mg/mL to 25 mg/mL of the multi-specific binding protein including a first antigen-binding site that binds NKG2D, a second antigen-binding site that binds HER2, and an antibody Fc domain; (ii) 10 to mM histidine; (iii) 150 to 300 mM sucrose; and (iv) 0.005% to 0.02% (w/v) polysorbate-80, at pH 5.5 to 6.5.

Some pharmaceutical compositions of the present disclosure contain: (i) about 15 mg/mL of the multi-specific binding protein including a first antigen-binding site that binds NKG2D, a second antigen-binding site that binds HER2, and an antibody Fc domain; (ii) about mM histidine; (iii) about 250 mM sucrose; and (iv) about 0.01% (w/v) polysorbate-80, at pH 6.0.

Without wishing to be bound by theory, it is contemplated that these pharmaceutical compositions may improve stability of the multi-specific binding protein during long-term and short-term storage. It is further understood that prior to administration (e.g., intravenous infusion) to a patient, the pharmaceutical composition can be diluted, for example, with 0.9% sodium chloride.

Phase 1 Clinical Trial Cohorts

In certain embodiments, the subject treated in accordance with the methods disclosed herein meets one or more of the inclusion criteria of a phase 1 clinical trial cohort (e.g., the accelerated titration cohort, a “3+3” dose escalation cohort, or a safety/PK/PD expansion cohort described in Example 2). In certain embodiments, the subject treated in accordance with the methods disclosed herein meets all the inclusion criteria of the clinical trial cohort.

In certain embodiments, the subject has one or more (e.g., all) of the following characteristics:
histologically or cytologically proven locally advanced or metastatic solid tumors, for which no standard therapy exists, or standard therapy has failed;
primary tumors having documented HER2 expression detected by immunohistochemistry;
detectable erbb2 amplification and/or erbb2 activating mutations in a tumor biopsy;
has HER2-expressing tumors (at least 1+at the time of screening) as determined by immunohistochemistry using a CLIA accredited (or equivalent) method;
has disease measurable with at least one unidimensional measurable lesion by RECIST 1.1;
ECOG performance status of 0 or 1 at study entry and an estimated life expectancy of at least 3 months;
baseline left ventricular ejection fraction (LVEF)≥55% as measured by echocardiography or multigated acquisition (MUGA) scan;
adequate hematological function defined by white blood cell (WBC) count ≥3×10⁹/L with absolute neutrophil count (ANC)≥1.5×10⁹/L, lymphocyte count ≥0.5×10⁹/L, platelet count ≥75×10⁹/L, and hemoglobin ≥9 g/dL (may have been transfused);
adequate hepatic function defined by a total bilirubin level ≤1.5×the upper limit of normal (ULN), an aspartate aminotransferase (AST) level ≤2.5×ULN, and an alanine aminotransferase (ALT) level ≤2.5×ULN or, for patients with documented metastatic disease to the liver, AST and ALT levels ≤5×ULN; and/or
adequate renal function defined by an estimated creatinine clearance >50 mL/min according to the Cockcroft-Gault formula.

In certain embodiments, the subject has one or more (e.g., all) of the following characteristics:
is eligible to receive nivolumab per its label for a malignancy of epithelial origin; or has a tumor that does not have a standard therapy or standard therapy has failed; and
has received anti-PD-1 or anti-PD-L1 as a previous line of therapy; or has not received anti-PD-1 or anti-PD-L1 as a previous line of therapy and has experienced a grade 3 or 4 drug-related toxicity or a grade 2 drug related toxicity impacting the lungs or the neurological system, related to the prior anti-PD-1 or anti-PD-L1 therapy.

In certain embodiments, the subject has one or more (e.g., all) of the following characteristics:
is eligible to receive nab-paclitaxel per its label after failure of combination therapy for metastatic disease or relapse within 6 months of adjuvant chemotherapy, and has not been exposed to taxanes within the last six months;
has a tumor that has no standard therapy or standard therapy has failed; or
has an advanced (unresectable/recurrent/metastatic) triple negative breast cancer (TNBC).

In certain embodiments, the subject has a malignancy of epithelial origin and is eligible for treatment with nivolumab in accordance with its label. In certain embodiments, the subject has a metastatic breast cancer, failed to respond to a combination chemotherapy for metastatic disease or relapsed within 6 months of the adjuvant chemotherapy, did not have exposure to taxanes in the last 6 months, and is eligible for treatment with nab-paclitaxel in accordance with its label.

In certain embodiments, the subject treated in accordance with the methods disclosed herein does not meet one or more of the exclusion criteria described herein (e.g., in Example 2). In certain embodiments, the subject treated in accordance with the methods disclosed herein does not meet any of the exclusion criteria described herein (e.g., in Example 2).

In certain embodiments, the subject:
is not currently being treated with an immunotherapy, an immunosuppressive drug such as a chemotherapy or systemic corticosteroid (except for a short term treatment of allergic reactions or for the treatment of immunotherapy-related adverse event (irAE) with a systemic steroid, or treatment with a topical or inhalatory steroid with no or minimal systemic effect), or other experimental pharmaceutical product;
is not currently being treated with a tyrosine kinase inhibitor targeting HER2, or any recombinant molecule targeting HER2 or NKG2D;
is not currently being treated with a growth factor such as granulocyte colony stimulating factor or granulocyte macrophage colony stimulating factor (except for erythropoietin and erythropoietin analogs);
is not currently being treated with bisphosphonate or denosumab unless treatment was initiated more than 14 days prior to receiving the first administration of multi-specific binding protein;
has not previously been treated with drugs that specifically target the HER2 pathway (except subjects treated with a monoclonal antibody and provided a four week washout period, or subjects treated with a tyrosine kinase inhibitor providing a two week washout period);
is not currently being treated with cytoreductive therapy, radiotherapy (except palliative bone directed radiotherapy), cytokine therapy (except for erythropoietin), major surgery (except diagnostic biopsy), or use of any investigational drug within 28 days prior to treatment with multi-specific binding protein;
has not previously had a malignant disease within the last three years other than the target malignancy to treated by the multi-specific binding protein (except basal or squamous cell carcinoma of the skin or cervical carcinoma in situ);
does not have rapidly progressive disease;
does not have active or a history of central nervous system (CNS) metastases;
has not previously received an organ transplant (including autologous or allogenic stem cell transplantation);
does not have significant acute or chronic infections (including a positive test for human immunodeficiency virus (HIV)), or active or latent hepatitis B or active hepatitis C;
does not have a pre-existing autoimmune disease (except subjects with vitiligo), needing treatment with systemic immunosuppressive agents for more than 28 days within the last three years or clinically relevant immunodeficiencies (e.g., dys-gammaglobulinemia or congenital immunodeficiencies), or fever within seven days;
does not have known severe hypersensitivity reactions to mAbs (≥Grade 3 NCI-CTCAE v5.0), any history of anaphylaxis, or uncontrolled asthma (e.g., three or more features of partly controlled asthma);
does not have persisting toxicity related to prior therapy >Grade 1 NCI-CTCAE v5.0 (except alopecia and sensory neuropathy ≤Grade 2);
is not a pregnant or lactating female;
does not have known alcohol or drug abuse; and/or
does not have serious cardiac illness or medical conditions including, but not limited to:
   a history of New York Heart Association class III or IV heart failure or systolic dysfunction (LVEF <55%);
   high-risk uncontrolled arrhythmias, i.e. tachycardia with a heart rate >100/min at rest;
   significant ventricular arrhythmia (ventricular tachycardia) or higher-grade AV-block (second degree AV-block Type 2 (Mobitz 2) or third-degree AV-block);
   angina pectoris requiring anti-anginal medication;
   clinically significant valvular heart disease;
   evidence of transmural infarction on electrocardiogram (ECG); or
poorly controlled hypertension (defined by: systolic >180 mm Hg or diastolic >100 mm Hg).

It is contemplated that the method disclosed herein of treating a patient having a cancer specified above can be performed with or without combination with a corticosteroid for reducing one or more infusion-related reactions.

Gastric Cancer

A multi-specific binding protein targeting HER2 of the present disclosure or a pharmaceutical formulation thereof is useful in a method of treating gastric cancer, such as HER2-high (e.g., HER2-positive according to the ASCP guidelines) gastric cancer, gastric cancer having a HER2 expression level scored as 3+(e.g., as determined by immunohistochemistry), or HER2-low gastric cancer, in a subject in need thereof, the method including administering (e.g. via intravenous infusion) an effective amount of the multi-specific binding protein or the pharmaceutical formulation.

It is contemplated that the multi-specific binding protein can be used as a monotherapy. Alternatively, it can be used in a combination therapy. In some embodiments, the method further includes administering an effective amount of a PD-1 inhibitor (e.g., an anti-PD-1 antibody, e.g., nivolumab). In some embodiments, the nivolumab is administered (e.g., via intravenous infusion) at a dose of 120 to 600 mg (e.g., 480 mg). In some embodiments, nivolumab is administered once every four weeks. In some embodiments, nivolumab is administered on the same day as the multi-specific binding protein and prior to the administration of the multi-specific binding protein. In certain embodiments, the method further includes administering an anti-PD-1 antibody (e.g., nivolumab) and a cytoskeletal-disrupting chemotherapeutic agent (e.g., nab-paclitaxel).

In some embodiments, the multi-specific binding protein or a pharmaceutical formulation thereof is administered (e.g. via intravenous infusion) to the subject according to a dosage regimen disclosed herein. In some embodiments, the multi-specific binding protein or the pharmaceutical formulation is administered in an initial four-week treatment cycle on day 1, day 8, and day 15. In some embodiments, the multi-specific binding protein or the pharmaceutical formulation is not administered on day 22 of the initial treatment cycle. In some embodiments, after completion of the initial four-week treatment cycle, the multi-specific binding protein or the pharmaceutical formulation is administered (e.g. via intravenous infusion) on day 1 and day 15 of each subsequent four-week treatment cycle. In some embodiment, each of the doses in the initial and subsequent treatment cycles contains 5 mg/kg, 10 mg/kg, 15 mg/kg, or 20 mg/kg of the multi-specific binding protein.

In some embodiments, the gastric cancer (e.g., advanced gastric cancer or cancer of the gastro-esophageal junction) is HER2-high (e.g., HER2-positive according to the ASCP guidelines). In some embodiments, the gastric cancer is an advanced (e.g., unresectable, recurrent, or metastatic) gastric cancer or a cancer of the gastro-esophageal junction, e.g., per the 7th AJCC classification. In some embodiments, the gastric cancer is measurable with at least 1 unidimensional measurable lesion by RECIST 1.1. In some embodiments, the gastric cancer is not known to be microsatellite instability-high. In some embodiments, the subject has previously received a first line of therapy (e.g., a platinum salt and a fluoropyridine in combination with trastuzumab or a biosimilar to trastuzumab). In some embodiments, the gastric cancer has progressed after the first line of therapy. In some embodiments, the subject has received only one line of therapy for metastatic disease. In some embodiments, the multi-specific binding protein is administered as a monotherapy. In other embodiments, the multi-specific binding protein is administered in combination with a PD-1 inhibitor (e.g., an anti-PD-1 antibody, e.g., nivolumab).

In some embodiments, the gastric cancer (e.g., advanced gastric cancer or a cancer of the gastro-esophageal junction) is HER2-low, e.g., according to the ASCP guidelines based on: (a) in situ hybridization non-amplified (ratio of ERBB2 to CEP17<2.0 or single probe average ERBB2 gene copy number <4 signals/cell), or (b) immunohistochemistry score of 0, 1+ or 2+(if the immunohistochemistry score is 0, HER2 should be detected in at least 1% of the tumor cells). In some embodiments, the gastric cancer is an advanced (e.g., unresectable, recurrent, or metastatic) gastric cancer or a cancer of the gastro-esophageal junction, e.g., per the 7th AJCC classification. In some embodiments, the gastric cancer is measurable with at least 1 unidimensional measurable lesion by RECIST 1.1. In some embodiments, the gastric cancer is not known to be microsatellite instability-high. In some embodiments, the subject has previously received a first line of therapy. In some embodiments, the first line of therapy includes a platinum salt and a fluoropyridine in combination with a PD-1 inhibitor. In some embodiments, the gastric cancer has progressed after treatment with the first line of therapy. In some embodiments, the progression has occurred within 6 months prior to the treatment with the multi-specific binding protein. In some embodiments, the subject has received only one line of therapy for metastatic disease. In some embodiments, the multi-specific binding protein is administered as a monotherapy. In some embodiments, the multi-specific binding protein is administered in combination with a PD-1 inhibitor (e.g., an anti-PD-1 antibody, e.g., nivolumab).

Esophageal Cancer

A multi-specific binding protein targeting HER2 of the present disclosure or a pharmaceutical formulation thereof is useful in a method of treating esophageal cancer, such as HER2-high (e.g., HER2-positive according to the ASCP guidelines) esophageal cancer, esophageal cancer having a HER2 expression level scored as 3+(e.g., as determined by immunohistochemistry), or HER2-low esophageal cancer, in a subject in need thereof, the method including administering (e.g., via intravenous infusion) an effective amount of the multi-specific binding protein or the pharmaceutical formulation.

It is contemplated that the multi-specific binding protein can be used as a monotherapy. Alternatively, it can be used in a combination therapy. In some embodiments, the method further includes administering (an effective amount of a PD-1 inhibitor (e.g., an anti-PD-1 antibody, e.g., nivolumab). In some embodiments, the nivolumab is administered (e.g., via intravenous infusion) at a dose of 120 to 600 mg (e.g., 480 mg). In some embodiments, nivolumab is administered once every four weeks. In some embodiments, nivolumab is administered on the same day as the multi-specific binding protein and prior to the administration of the multi-specific binding protein. In certain embodiments, the method further includes administering an anti-PD-1 antibody (e.g., nivolumab) and a cytoskeletal-disrupting chemotherapeutic agent (e.g., nab-paclitaxel).

In some embodiments, the multi-specific binding protein or a pharmaceutical formulation thereof, is administered (e.g., via intravenous infusion) to the subject according to a dosage regimen disclosed herein. In some embodiments, the multi-specific binding protein or the pharmaceutical formulation is administered in an initial four-week treatment cycle on day 1, day 8, and day 15. In some embodiments, the multi-specific binding protein or the pharmaceutical formulation is not administered on day 22 of the initial treatment cycle. In some embodiments, after completion of the initial four-week treatment cycle, the multi-specific binding protein or the pharmaceutical formulation is administered (e.g., via intravenous infusion) on day 1 and day 15 of each subsequent four-week treatment cycle. In some embodiment, each of the doses in the initial and subsequent treatment cycles contains 5 mg/kg, 10 mg/kg, 15 mg/kg, or 20 mg/kg of the multi-specific binding protein.

In some embodiments, provided herein is a method for treatment of an advanced esophageal adenocarcinoma. In some embodiments, the method provided herein is for treatment of an advanced esophageal adenocarcinoma in a subject who has previously received a first line of therapy. In some embodiments, the first line of therapy includes a platinum salt and a fluoropyridine in combination with trastuzumab or a biosimilar to trastuzumab. In some embodiments, the first line therapy did not include an anti-PD-1 therapeutic.

Disclosed herein, in various embodiments, is a method of treating esophageal cancer having a HER2 expression level scored as 3+(e.g., as determined by immunohistochemistry) in a subject in need thereof, that includes administering (e.g., via intravenous infusion) to the subject the multi-specific binding protein at an amount selected from 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1.0 mg/kg, 1.6 mg/kg, 5.2 mg/kg, 10 mg/kg, 15 mg/kg, 20 mg/kg, or 50 mg/kg.

In some embodiments, the esophageal cancer (e.g., advanced esophageal cancer or adenocarcinoma of the esophagus) is HER2-high (e.g., HER2-positive according to the ASCP guidelines). In some embodiments, the esophageal cancer is an advanced (e.g., unresectable, recurrent, or metastatic) esophageal cancer or adenocarcinoma of the esophagus, e.g., per the 7th AJCC classification. In some embodiments, the esophageal cancer is measurable with at least 1 unidimensional measurable lesion by RECIST 1.1. In some embodiments, the esophageal cancer is not known to be microsatellite instability-high. In some embodiments, the subject has previously received a first line of therapy (e.g., a platinum salt and a fluoropyridine in combination with trastuzumab or a biosimilar to trastuzumab). In some embodiments, the first line of therapy did not include a PD-1 inhibitor. In some embodiments, the esophageal cancer has progressed after the first line of therapy. In some embodiments, the subject has received only one line of therapy for metastatic disease. In some embodiments, the multi-specific binding protein is administered as a monotherapy. In other embodiments, the multi-specific binding protein is administered in combination with a PD-1 inhibitor (e.g., an anti-PD-1 antibody, e.g., nivolumab).

In some embodiments, the esophageal cancer (e.g., advanced esophageal cancer or adenocarcinoma of the esophagus) is HER2-low, e.g., according to the ASCP guidelines based on: (a) in situ hybridization non-amplified (ratio of ERBB2 to CEP17<2.0 or single probe average ERBB2 gene copy number <4 signals/cell), or (b) immunohistochemistry score of 0, 1+, or 2+(if the immunohistochemistry score is 0, HER2 should be detected in at least 1% of the tumor cells). In some embodiments, the esophageal cancer is an advanced (e.g., unresectable, recurrent, or metastatic) esophageal cancer or adenocarcinoma of the esophagus, e.g., per the 7th AJCC classification. In some embodiments, the esophageal cancer is measurable with at least 1 unidimensional measurable lesion by RECIST 1.1. In some embodiments, the esophageal cancer is not known to be microsatellite instability-high. In some embodiments, the subject has previously received a first line of therapy. In some embodiments, the first line of therapy includes a platinum salt and a fluoropyridine in combination with a PD-1 inhibitor. In some embodiments, the esophageal cancer has progressed after treatment with the first line of therapy. In some embodiments, the progression has occurred while receiving a PD-1 inhibitor within 6 months prior to the treatment with the multi-specific binding protein. In some embodiments, the subject has received only one line of therapy for metastatic disease. In some embodiments, the multi-specific binding protein is administered as a monotherapy. In some embodiments, the multi-specific binding protein is administered in combination with a PD-1 inhibitor (e.g., an anti-PD-1 antibody, e.g., nivolumab).

Breast Cancer

A multi-specific binding protein targeting HER2 of the present disclosure or a pharmaceutical formulation thereof is useful in a method of treating breast cancer, such as HER2-high (e.g., HER2-positive according to the ASCP guidelines) breast cancer, breast cancer having a HER2 expression level scored as 3+(e.g., as determined by immunohistochemistry), or HER2-low breast cancer, in a subject in need thereof, the method including administering (e.g. via intravenous infusion) an effective amount of the multi-specific binding protein or the pharmaceutical formulation.

In some embodiments, the breast cancer is HER2-high, e.g., based on (a) ERBB2 amplification measured by ISH, per the 2018 ASCO/CAP guidelines or equivalent or (b) immunohistochemistry score of 3+ or 2+(if the immunohistochemistry score is 2+, ISH should demonstrate ERBB2 amplification). In some embodiments, the breast cancer is a locally advanced or metastatic breast cancer. In some embodiments, the breast cancer is measurable with at least 1 unidimensional measurable lesion by RECIST 1.1. In some embodiments, the subject has previously received trastuzumab, pertuzumab, and a HER2-targeting ADC or other anti-HER2 lines of therapy. In some embodiments, the breast cancer has progressed after one line of systemic chemotherapy. In some embodiments, the subject has received radiation therapy or endocrine therapy for metastatic disease. In some embodiments, the multi-specific binding protein is administered as a monotherapy. In other embodiments, the multi-specific binding protein is administered in combination with a PD-1 inhibitor (e.g., an anti-PD-1 antibody, e.g., nivolumab).

In some embodiments, the breast cancer is HER2-low based on: (a) absence of ERBB2 amplification by ISH, per the 2018 ASCO/CAP guidelines or equivalent or (b) immunohistochemistry score of 0, 1+, or 2+(if the immunohistochemistry score is 0, HER2 should be detected by immunohistochemistry on at least 1% of the tumor cells). In some embodiments, the breast cancer is a locally advanced or metastatic breast cancer. In some embodiments, the breast cancer is measurable with at least 1 unidimensional measurable lesion by RECIST 1.1. In some embodiments, the breast cancer is hormone receptor-positive and the subject has previously received hormone therapy and has progressed while receiving hormone therapy. In some embodiments, the breast cancer has progressed after one line of systemic chemotherapy or a trastuzumab-containing ADC. In some embodiments, the subject has received radiation therapy or endocrine therapy for metastatic disease. In some embodiments, the multi-specific binding protein is administered as a monotherapy. In some embodiments, the multi-specific binding protein is administered in combination with a PD-1 inhibitor (e.g., an anti-PD-1 antibody, e.g., nivolumab).

Triple Negative Breast Cancer

A multi-specific binding protein targeting HER2 of the present disclosure or a pharmaceutical formulation thereof is useful in a method of treating a triple negative breast cancer (TNBC) in a subject in need thereof, the method including administering (e.g., via intravenous infusion) an effective amount of the multi-specific binding protein or the pharmaceutical formulation.

It is contemplated that the multi-specific binding protein can be used as a monotherapy. Alternatively, it can be used in a combination therapy. In some embodiments, the method includes administering (e.g., via intravenous infusion) an effective amount of a multi-specific binding protein or pharmaceutical formulation thereof in combination with nab-paclitaxel. In some embodiments, the method further includes administering (e.g., via intravenous infusion) an effective amount of a PD-1 inhibitor (e.g., an anti-PD-1 antibody, e.g., nivolumab). In some embodiments, the nivolumab is administered (e.g., via intravenous infusion) at a dose of 120 to 600 mg (e.g., 480 mg). In some embodiments, nivolumab is administered once every four weeks. In some embodiments, nivolumab is administered on the same day as the multi-specific binding protein and prior to the administration of the multi-specific binding protein. In certain embodiments, the method further includes administering nab-paclitaxel and nivolumab.

In some embodiments, the multi-specific binding protein or a pharmaceutical formulation thereof, is administered (e.g., via intravenous infusion) to the subject according to a dosage regimen disclosed herein. In some embodiments, the multi-specific binding protein or the pharmaceutical formulation is administered in an initial four-week treatment cycle on day 1, day 8, and day 15. In some embodiments, the multi-specific binding protein or a pharmaceutical formulation is not administered on day 22 of the initial treatment cycle.

In some embodiments, after completion of the initial four-week treatment cycle, the multi-specific binding protein or the pharmaceutical formulation is administered (e.g., via intravenous infusion) on day 1 and day 15 of each subsequent four-week treatment cycle. In some embodiment, each of the doses in the initial and subsequent treatment cycles contains 5 mg/kg, 10 mg/kg, 15 mg/kg, or 20 mg/kg of the multi-specific binding protein.

In some embodiments, the TNBC has a HER2 status making the subject ineligible for trastuzumab as defined per the American College of Physicians (ACP) guidelines or equivalent, negative ER expression, and negative PR expression, e.g., based on (a) lack of HER2 amplification as determined by in situ hybridization (ratio of HER2 to CEP17<2.0 or single probe average HER2 gene copy number <4 signals/cell), or HER2 expression level 0, 1+, or 2+ as measured by immunohistochemistry and (b) ER and PR negativity are defined as <1% of cells expressing hormonal receptors via immunohistochemistry analysis. In some embodiments, the breast cancer has documented HER2 expression by immunohistochemistry using a CLIA accredited or equivalent method. In some embodiments, the breast cancer has a HER2 score of at least 1+ as measured by immunohistochemistry or has a HER2 score of 0 and HER2 is detected by immunohistochemistry on at least 1% of the tumor cells. In some embodiments, the breast cancer has PD-L1 score (CPS) less than 10 as measured by immunohistochemistry, (e.g., using Agilent/Dako's PD-L1 immunohistochemistry 22C3 pharmDx assay). In some embodiments, the breast cancer is a locally advanced or metastatic breast cancer. In some embodiments, the breast cancer is a locally advanced or metastatic solid tumor for which no standard therapy exists or standard therapy has failed. In some embodiments, the breast cancer is measurable with at least 1 unidimensional measurable lesion by RECIST 1.1. In some embodiments, the subject has received no prior chemotherapy or targeted systemic therapy for inoperable locally advanced or metastatic TNBC. In some embodiments, the subject has previously received radiation therapy or endocrine therapy for metastatic disease. In some embodiments, the patient has received prior chemotherapy in the neoadjuvant or adjuvant setting at least 12 months prior to administration of the multi-specific binding protein. In some embodiments, the multi-specific binding protein is administered as a monotherapy.

In some embodiments, the multi-specific binding protein is administered in combination with a PD-1 inhibitor (e.g., an anti-PD-1 antibody, e.g., nivolumab). In some embodiments, the breast cancer has no standard therapy or standard therapy has failed. In some embodiments, the subject has received a PD-1 inhibitor and has not experienced either (a) a Grade 3 or 4 drug related toxicity during the treatment with the PD-1 inhibitor, or (b) a Grade 2 drug related toxicity related to prior checkpoint therapy that impacted either the lungs or the neurological system.

In some embodiments, the multi-specific binding protein is administered in combination with a cytoskeletal-disrupting chemotherapeutic agent (e.g., nab-paclitaxel). In some embodiments the patient (a) is eligible for treatment with nab-paclitaxel per its label after failure of combination chemotherapy for metastatic disease or relapse within 6 months of adjuvant chemotherapy and has had no exposure to taxanes for at least 6 months prior to administration of the multi-specific binding protein, (b) has a tumor for which no standard therapy exists or standard therapy has failed and has had no exposure to taxanes for at least 6 months prior to administration of the multi-specific binding protein, or (c) has had first line advanced (e.g., unresectable, recurrent, or metastatic) TNBC.

In some embodiments, provided herein is a method for treatment of a metastatic or locally advanced TNBC. In some embodiments, provided herein is a method for treatment of a patient who is ineligible for treatment with an anti-PD-L1 combination therapy. In some embodiments, the patient has a PD-L1 score (CPS) less than 10 (e.g., as determined by immunohistochemistry).

In some embodiments, provided herein is a method for treatment of an advanced, optionally unresectable, recurrent, or metastatic TNBC. In some embodiments, the method provided herein is for treatment of an advanced, optionally unresectable, recurrent, or metastatic TNBC in a subject for whom treatment with a combination chemotherapy for metastatic disease has previously failed, or where disease has relapsed within 6 months of adjuvant chemotherapy. In some embodiments, the subject has not previously received a chemotherapy or targeted systemic therapy.

Disclosed herein, in various embodiments, is a method of treating TNBC in a subject in need thereof, that includes administering (e.g., via intravenous infusion) to the subject the multi-specific binding protein at an amount selected from 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×⁻¹ mg/kg, 0.52 mg/kg, 1.0 mg/kg, 1.6 mg/kg, 5.2 mg/kg, 10 mg/kg, 15 mg/kg, 20 mg/kg, or 50 mg/kg.

Urothelial Bladder Cancer

A multi-specific binding protein targeting HER2 of the present disclosure or a pharmaceutical formulation thereof is useful in a method of treating urothelial bladder cancer in a subject in need thereof, the method including administering (e.g. via intravenous infusion) an effective amount of the multi-specific binding protein or the pharmaceutical formulation.

In some embodiments, the urothelial bladder cancer has a HER2 expression level scored at least 1+ as measured by immunohistochemistry (e.g., using a CLIA-accredited or equivalent method). In some embodiments, the urothelial bladder cancer is an advanced or metastatic transitional cell carcinoma of the urotheliam (e.g., the renal pelvis, ureters, urinary urothelial, or urethra). In some embodiments, the urothelial bladder cancer is measurable with at least 1 unidimensional measurable lesion by RECIST 1.1. In some embodiments, the subject has radiographic disease progression after their last line of therapy. In some embodiments, the subject has previously received a platinum containing chemotherapy and a PD-1 inhibitor (e.g., as a neo-adjuvant, adjuvant treatment, or as a treatment for advanced (e.g., recurrent, unresectable, or metastatic) disease. In some embodiments, the patient has experienced radiographic disease progression during treatment with a PD-1 inhibitor. In some embodiments, the multi-specific binding protein is administered as a monotherapy. In other embodiments, the multi-specific binding protein is administered in combination with a PD-1 inhibitor (e.g., an anti-PD-1 antibody, e.g., nivolumab). In some embodiments, subjects who are administered the multi-specific binding protein in combination with a PD-1 inhibitor have experienced progression treatment with a PD-1 inhibitor within 6 months of administration of the multi-specific binding protein.

Non-Small-Cell Lung Cancer (NSCLC)

A multi-specific binding protein targeting HER2 of the present disclosure or a pharmaceutical formulation thereof is useful in a method of treating non-small cell lung cancer (NSCLC) in a subject in need thereof, the method including administering (e.g. via intravenous infusion) an effective amount of the multi-specific binding protein.

In some embodiments, the NSCLC has ERBB2 amplification (e.g., using a CLIA approved genomic test or equivalent). In some embodiments, the NSCLC has a HER2 expression level scored at least 2+ as measured by immunohistochemistry. In some embodiments, the NSCLC meets stage criteria for stage IIIB, stage IV, or recurrent disease. In some embodiments, the NSCLC is measurable with at least 1 unidimensional measurable lesion by RECIST 1.1. In some embodiments, the NSCLC is Stage IIIB and the subject is ineligible for local therapies with curative intent (e.g., radiotherapy or surgery). In some embodiments, the subject has received a PD-1 inhibitor and progressed on or after treatment with the PD-1 inhibitor. In some embodiments, the status of actionable mutations (e.g., in EGFR, ALK, ROS1, or RET) in the tumor is known. In some embodiments, a subject having actionable mutations has received and progressed on, has been intolerant to, or is not candidate for standard tyrosine kinase inhibitors. In some embodiments, the multi-specific binding protein is administered as a monotherapy. In other embodiments, the multi-specific binding protein is administered in combination with a PD-1 inhibitor (e.g., an anti-PD-1 antibody, e.g., nivolumab).

In some embodiments, the NSCLC has a HER2 expression level scored at least 1+as measured by immunohistochemistry (e.g., using a CLIA approved genomic test or equivalent) and does not have an ERBB2 amplification. In some embodiments, the NSCLC meets stage criteria for stage IIIB, stage IV, or recurrent disease and has been confirmed to have HER2 expression. In some embodiments, the NSCLC is measurable with at least 1 unidimensional measurable lesion by RECIST 1.1. In some embodiments, the NSCLC is Stage IIIB and the subject is ineligible for local therapies with curative intent (e.g., radiotherapy or surgery). In some embodiments, the subject has received a PD-1 inhibitor and progressed on or after treatment with the PD-1 inhibitor within 6 months prior to the administration of the multi-specific binding protein. In some embodiments, the NSCLC is recurrent or progressive during or after platinum doublet-based chemotherapy or within 6 months after completing platinum-based chemotherapy for local disease. In some embodiments, the status of actionable mutations (e.g., in EGFR, ALK, ROS1, or RET) in the tumor is known. In some embodiments, subjects having actionable mutations have received and progressed on, have been intolerant to, or not be a candidate for standard tyrosine kinase inhibitors. In some embodiments, the multi-specific binding protein is administered as a monotherapy. In other embodiments, the multi-specific binding protein is administered in combination with a PD-1 inhibitor (e.g., an anti-PD-1 antibody, e.g., nivolumab).

Additional Phase 2 Clinical Trial Cohorts

In certain embodiments, the subject treated in accordance with the methods disclosed herein meets one or more of the inclusion criteria of a phase 2 clinical trial cohort (e.g., the metastatic breast cancer (MBC) cohort or the solid tumors with high HER2 expression (HER2 3+) cohort). In certain embodiments, the subject treated in accordance with the methods disclosed herein meets all the inclusion criteria of the clinical trial cohort.

In certain embodiments, the subject has metastatic breast cancer (MBC) and one or more (e.g., all) of the following characteristics:
histologically confirmed MBC;
no more than 3 prior lines of cytotoxic therapy for metastatic disease;
previous received treatment with taxane and an anthracycline, unless anthracycline is contraindicated;
a tumor scoring 1+ or 2+ by IHC, and if scoring is 2+, and the existence of tumor amplification of ERBB2 must be ruled by an FDA approved method; and/or
radiographic progression after the last line of systemic therapy.

In certain embodiments, the subject has a HER2 3+ tumor and one or more (e.g., all) of the following characteristics:
any solid tumor except breast cancer or gastric cancer and a history of erbb2 amplification within the tumor and one of the following 1) HER2 3+ scoring by immunohistochemistry documented in the subject's most recent biopsy within 6 months, post radiographic progression on the last line of therapy or 2) HER2 3+ scoring by immunohistochemistry during the screening window; and
treatment with at least one line of an approved or established therapy.

In certain embodiments, the subject has a solid tumor with ERBB2 gene amplification. In some embodiments, the solid tumor has documented ERBB2 gene ammplification confirmed by a CLIA-accredited or equivalent method. In some embodiments, the subject has non-breast, non-gastric, non-esophageal cancer that have progressed after treatment with a HER2 targeting antibody-drug-conjugate. In some embodiments, the subject has a carcinoma of the salivary glands that has progressed after one line of systemic therapy. In some embodiments, the solid tumor is measurable with at least 1 unidimensional measurable lesion by RECIST 1.1. In some embodiments, the subject has received at lease one line of an approved or established therapy. In some embodiments, the multi-specific binding protein is administered as a monotherapy. In other embodiments, the multi-specific binding protein is administered in combination with a PD-1 inhibitor (e.g., an anti-PD-1 antibody, e.g., nivolumab).

Efficacy

The methods of treatment disclosed herein are expected to effectively treat the subjects with cancer. Efficacy can be assessed by overall response rate. Overall response rate can be determined by a medical expert according to RECIST 1.1. The overall response rate can be evaluated over a whole clinical trial period.

Other indications of efficacy include but are not limited to best overall response (BOR); duration of response (e.g., according to RECIST 1.1), which can be calculated for each patient with a confirmed response and analyzed using the Kaplan-Meier method; progression free survival (PFS) time, which can be calculated using the Kaplan-Meier method; and overall survival (OS) time, which can be calculated using the Kaplan-Meier method.

In certain embodiments, one or more of the efficacy indications above of a method of treatment disclosed herein is better than no treatment, a placebo treatment, or a standard of care therapy for the same type of cancer.

Prophylactic Premedication

Prophylactic premedications are contemplated for reducing one or more adverse effects of (e.g., infusion-related reactions to) the multi-specific binding protein administered according to the methods disclosed herein. It is understood that the prophylactic premedications can be used when the multi-specific binding protein is provided as either a monotherapy or in combination with another cancer treatment.

Corticosteroids

In one aspect, the present disclosure provides a method of treating cancer, the method including administering to a subject in need thereof a therapeutically effective amount of a multi-specific binding protein, pharmaceutical composition, or pharmaceutical formulation disclosed herein (administered as a monotherapy or in a combination therapy) and a therapeutically effective amount of a corticosteroid to reduce one or more infusion-related reactions to the multi-specific binding protein, pharmaceutical composition, or pharmaceutical formulation. The present disclosure also provides a multi-specific binding protein, pharmaceutical composition, or pharmaceutical formulation disclosed herein (administered as a monotherapy or in a combination therapy) for use in a method of treating cancer in combination with a therapeutically effective amount of a corticosteroid to reduce one or more infusion-related reactions to the multi-specific binding protein, pharmaceutical composition, or pharmaceutical formulation. In a combination therapy, the multi-specific binding protein, pharmaceutical composition, or pharmaceutical formulation disclosed herein can be combined with an immunotherapy, such as an anti-PD-1 antibody (e.g., nivolumab, pembrolizumab), and/or a chemotherapy, such as a cytoskeletal-disrupting chemotherapeutic agent (e.g., nab-paclitaxel, paclitaxel, docetaxel).

The corticosteroids that are useful in the present invention generally include steroids produced by the adrenocortex, such as glucocorticoids and mineralocorticoids, and synthetic analogs and derivatives of naturally occurring corticosteroids having anti-inflammatory activity. In certain embodiments, the corticosteroid is a glucocorticoid. Glucocorticoids bind the glucocorticoid receptor and reduce inflammation by inhibiting the immune response. In certain embodiments, the corticosteroid is a mineralocorticoid. Mineral corticoids bind the mineralocorticoid receptor and act to regulate Na⁺/K⁺ concentrations in the serum. Some corticosteroids can have both glucocorticoid and mineralocorticoid functions. Examples of corticosteroids are disclosed in U.S. Pat. No. 10,799,599. In certain embodiments, the corticosteroid used in the method disclosed herein is selected from methylprednisolone, dexamethasone, hydrocortisone, prednisone, prednisolone, fluticasone, flumethasone, fluocinolone, budesonide, beclomethasone, ciclesonide, cortisone, triamcinolone, betamethasone, deflazacort, difluprednate, loteprednol, paramethasone, tixocortol, aldosterone, cloprednol, cortivazol, deoxycortone, desonide, desoximetasone, difluorocortolone, fluclorolone, fludrocortisone, fluocinonide, fluocortin butyl, fluorocortisone. fluorocortolone, fluorometholone, flurandrenolone, halcinonide, icomethasone, meprednisone, mometasone, rofteponide, RPR 106541, and their respective pharmaceutically acceptable derivatives, such as beclomethasone dipropionate (anhydrous or monohydrate), beclomethasone monopropionate, dexamethasone 21-isonicotinate, fluticasone propionate, icomethasone enbutate, tixocortol 21-pivalate, and triamcinolone acetonide, and pharmaceutically acceptable salts and/or derivatives thereof.

In certain embodiments, the glucocorticoid is methylprednisolone. Exemplary effective amounts of methylprednisolone can be in the range of 8 to 200 mg, 20 to 200 mg, 25 to 200 mg, 50 to 200 mg, 75 to 200 mg, 100 to 200 mg, 125 to 200 mg, 150 to 200 mg, 175 to 200 mg, 25 to 175 mg, 50 to 175 mg, 75 to 175 mg, 100 to 175 mg, 125 to 175 mg, 150 to 175 mg, to 150 mg, 25 to 150 mg, 50 to 150 mg, 75 to 150 mg, 100 to 150 mg, 125 to 150 mg, 25 to 125 mg, 50 to 125 mg, 75 to 125 mg, 100 to 125 mg, 25 to 100 mg, 50 to 100 mg, 75 to 100 mg, to 75 mg, 50 to 75 mg, 25 to 50 mg, about 25 mg, about 50 mg, about 75 mg, about 100 mg, about 125 mg, about 150 mg, about 175 mg, or about 200 mg. In certain embodiments, the effective amount of methylprednisolone is about 125 mg. In certain embodiments, the effective amount of methylprednisolone by oral administration is 8 mg, 16 mg 32 mg, 48 mg, 64 mg, 80 mg, 96 mg, or 120 mg.

In certain embodiments, the glucocorticoid is dexamethasone. Exemplary effective amounts of dexamethasone can be in the range of 8-200 mg, 20-200 mg, 50-200 mg, 100-200 mg, 20-150 mg, 50-150 mg, 50-100 mg, or 100-150 mg. In certain embodiments, the effective amount of dexamethasone by intravenous administration is 20 mg, 30 mg, 40 mg, 50 mg, 60 mg, mg, 80 mg, 90 mg, 100 mg, 125 mg, or 150 mg. In certain embodiments, the effective amount of dexamethasone by oral administration is 8 mg, 16 mg, 32 mg, 48 mg, 64 mg, 80 mg, 96 mg, or 120 mg.

In certain embodiments, the corticosteroid is administered parenterally. In certain embodiments, the corticosteroid is administered intravenously. In certain embodiments, the corticosteroid is administered orally.

The corticosteroid can be administered prior to, simultaneously with, or subsequent to the administration of the multi-specific binding protein. In certain embodiments, the corticosteroid is administered within 6 hours, within 5 hours, within 4 hours, within 3 hours, within 2 hours, within 1 hour, within 30 minutes, within 15 minutes, or immediately prior to the administration of the multi-specific binding protein (e.g., prior to the beginning of the administration of the multi-specific binding protein). In certain embodiments, the corticosteroid is administered within 1 hour prior to the administration of the multi-specific binding protein (e.g., prior to the beginning of the administration of the multi-specific binding protein). In certain embodiments, the corticosteroid is administered simultaneously with the administration of the multi-specific binding protein. In certain embodiments, the corticosteroid and the multi-specific binding protein are diluted into a single pharmaceutical composition administered to the subject. In certain embodiments, the duration of administration of the corticosteroid and the duration of administration of the multi-specific binding protein completely or partially overlap. In certain embodiments, the corticosteroid is administered within 2 hours, 1 hour, or 30 minutes subsequent to the administration of the multi-specific binding protein (e.g., subsequent to the beginning of the administration of the multi-specific binding protein).

In certain embodiments, the corticosteroid is administered on day 1 of the first cycle (i.e., in combination with the first dose of the multi-specific binding protein). In certain embodiments, the corticosteroid is administered only on day 1 of the first cycle (i.e., in combination with the first dose of the multi-specific binding protein). In certain embodiments, the corticosteroid is further administered if an infusion-related reaction persists or recurs. In certain embodiments, infusion-related reactions include a persistent rash, diarrhea, colitis, autoimmune hepatitis, arthritis, glomerulonephritis, cardiomyopathy, or uveitis or another inflammatory eye conditions.

In certain embodiments, the corticosteroid (e.g., methylprednisolone) is administered 30 to 90 min., 40 to 90 min., 50 to 90 min., 60 to 90 min., 70 to 90 min., 80 to 90 min., 30 to 80 min., 40 to 80 min., 50 to 80 min., 60 to 80 min., 70 to 80 min., 30 to 70 min., 40 to 70 min., 50 to 70 min., 60 to 70 min., 30 to 60 min., 40 to 60 min., 50 to 60 min., 30 to 50 min., 40 to 50 min., 30 to 40 min., about 30 min., about 40 min., about 50 min., about 60 min., about 70 min., about 80 min., or about 90 min., prior to administration of the multi-specific binding protein, pharmaceutical composition, or pharmaceutical formulation disclosed herein.

In certain embodiments, subjects receive premedication treatment including about 125 mg of methylprednisolone administered intravenously within 60 minutes of administration of the multi-specific binding protein, pharmaceutical composition, or pharmaceutical formulation disclosed herein. In certain embodiments, premedication treatment further includes intravenous or oral administration of 40 to 50 mg diphenhydramine and 800 to 1000 mg of acetaminophen 30 to 60 minutes prior to administration of the multi-specific binding protein, pharmaceutical composition, or pharmaceutical formulation disclosed herein.

Exemplary infusion-related reactions to a multi-specific binding protein disclosed herein include cytokine release syndrome, anaphylaxis, chills, fever/pyrexia, hypotension, hypertension, rigors, headache, dizziness, itching, sore throat, laryngeal edema, angioedema, redness/flushing, rash/urticaria, bronchospasm, tachycardia, bradycardia, auricular fibrillation, hypoxia, respiratory distress/dyspnea/shortness of breath/breathless sensation, chest tightness, nausea, vomiting, pain (e.g., chest pain, back pain), shivering, tremors, myalgia, tiredness, insomnia, asthenia, hypersensitivity, and diarrhea. Clinical presentations of cytokine release syndrome are described in Shimabukuro-Vornhagen et al., include but are not limited to fever (e.g., high fever), fatigue, headache, rash, arthralgia, myalgia, hypotension, vasopressor-requiring circulatory shock, vascular leakage, disseminated intravascular coagulation, and multi-organ system failure. In certain embodiments, the co-administration of the corticosteroid reduces one or more of the infusion-related reactions in the subject.

Antihistamines

An antihistamine can be used to avoid or mitigate an allergic response (e.g., anaphylaxis) to the multi-specific binding protein. Accordingly, in certain embodiments, the method further includes administering to the subject a therapeutically effective amount of an antihistamine. Exemplary antihistamines are disclosed in U.S. Pat. No. 10,898,693. In certain embodiments, the antihistamine used in the method disclosed herein is selected from crivastine, azelastine, bilastine, brompheniramine, buclizine, bromodiphenhydramine, carbinoxamine, cetirizine, cyclizine, chlorpheniramine, chlorodiphenhydramine, clemastine, cromolyn, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocetirizine, loratadine, nedocromil, olopatadine, phenindamine, pheniramine, phenyltoloxamine, promethazine, pyrilamine, rupatadine, tripelennamine, triprolidine, and combinations thereof. In certain embodiments, the antihistamine is diphenhydramine. In certain embodiments, the therapeutically effective amount of diphenhydramine is 10 to 100 mg, 20 to 100 mg, 30 to 100 mg, 40 to 100 mg, 50 to 100 mg, 60 to 100 mg, 70 to 100 mg, 80 to 100 mg, to 100 mg, 10 to 90 mg, 20 to 90 mg, 30 to 90 mg, 40 to 90 mg, 50 to 90 mg, 60 to 90 mg, 70 to 90 mg, 80 to 90 mg, 10 to 80 mg, 20 to 80 mg, 30 to 80 mg, 40 to 80 mg, 50 to 80 mg, 60 to mg, 70 to 80 mg, 10 to 70 mg, 20 to 70 mg, 30 to 70 mg, 40 to 70 mg, 50 to 70 mg, 60 to 70 mg, 10 to 60 mg, 20 to 60 mg, 30 to 60 mg, 40 to 60 mg, 50 to 60 mg, 10 to 50 mg, 20 to 50 mg, to 50 mg, 40 to 50 mg, 20 to 40 mg, 30 to 40 mg, 20 to 30 mg, about 20 mg, about 30 mg, about 40 mg, about 50 mg, about 60 mg, about 70 mg, about 80 mg, about 90 mg, or about 100 mg. In certain embodiments, the therapeutically effective amount of diphenhydramine is 40 to mg.

In certain embodiments, the antihistamine is administered parenterally. In certain embodiments, the antihistamine is administered intravenously. In certain embodiments, the antihistamine is administered orally.

The antihistamine can be administered prior to, simultaneously with, or subsequent to the administration of the multi-specific binding protein, pharmaceutical composition, or pharmaceutical formulation disclosed herein. In certain embodiments, the antihistamine is administered within 2 hours, within 1.5 hours, within 1 hour (60 minutes), within 45 minutes, within 30 minutes, within 15 minutes, or immediately prior to the administration of the multi-specific binding protein (e.g., prior to the beginning of the administration of the multi-specific binding protein), pharmaceutical composition, or pharmaceutical formulation disclosed herein. In certain embodiments, the antihistamine is administered with every dose of the multi-specific binding protein.

In certain embodiments, the antihistamine (e.g., diphenhydramine) is administered to 90 min., 40 to 90 min., 50 to 90 min., 60 to 90 min., 70 to 90 min., 80 to 90 min., 30 to 80 min., 40 to 80 min., 50 to 80 min., 60 to 80 min., 70 to 80 min., 30 to 70 min., 40 to 70 min., 50 to 70 min., 60 to 70 min., 30 to 60 min., 40 to 60 min., 50 to 60 min., 30 to 50 min., 40 to 50 min., 30 to 40 min., about 30 min., about 40 min., about 50 min., about 60 min., about 70 min., about 80 min., or about 90 min., prior to administration of the multi-specific binding protein, pharmaceutical composition, or pharmaceutical formulation disclosed herein.

In certain embodiments, a subject receives premedication treatment including 40 to mg of diphenhydramine administered intravenously or orally 30 to 60 minutes prior to administration of the multi-specific binding protein, pharmaceutical composition, or pharmaceutical formulation disclosed herein. In certain embodiments, premedication treatment further includes intravenous or oral administration of 800 to 100 mg acetaminophen prior to administration of the multi-specific binding protein, pharmaceutical composition, or pharmaceutical formulation disclosed herein.

Where the method of treatment disclosed herein includes multiple doses (e.g., five or more doses) of the multi-specific binding protein, in certain embodiments, the antihistamine is administered with the first dose, the first two doses, the first three doses, the first four doses, or the first five doses of the multi-specific binding protein.

Analgesics and Antipyretics

An analgesic can be used to relieve pain as a result of the administration of the multi-specific binding protein, pharmaceutical composition, or pharmaceutical formulation disclosed herein, whether administered as a monotherapy or as a combination therapy. Accordingly, in certain embodiments, the method further includes administering to the subject a therapeutically effective amount of an analgesic. Exemplary analgesics are disclosed in U.S. Patent Application Publication No. 2015/0342989 and U.S. Pat. No. 10,899,834. In certain embodiments, the analgesic used in the method disclosed herein is selected from acetaminophen, salicylamide, salicyl salicytate, methyl salicylate, magnesium salicylate, faisiamine, ethenzarnide, diflunisal, choline magnesium salicylate, benorylateibenotilatem and amoxiprin, acetylsalicylate, ceelofenac, acemetacin, alclofenac, bromfenac, diclofenac, etodolac, indomethacin, nabumetone, oxametacin, proghtmetacin, sulindac, toknetin, iminoprofen, benoxaprofen, carprofen, dexihuprofen, dexketoprofen, fenbufen, fenoprofen, fhmoxaprofen, flurbiprofen, ibuprofen, ihuproxam, indoprofen, ketoprofen, ketorolac, ioxoprofen, naproxen, oxaprozin, pirprofen, suprofen, tiaprofenic acid, mefenamic acid, flufenamic acid, meclofenamie acid, toiferiamic acid, droxicarn, iornoxicarn, meloxicam, piroxicam, and tenoxicam, mpyrone, azapropazone, cleffezone, kebnzone, metamizolle, mofebutazone, oxyphenbutazone, phenazone, phenylbutazone, sulfinpyrazone, decoxib, rofecoxib, parecoxib, etoricoxib, codeine, dihydrocodeine, morphine or a morphine derivative or pharmaceutically acceptable salt thereof, diacetylmorphine, hydrocodone, hydromorphone, levorphanol, oxymorphone, alfentanil, buprenorphine, butorphanol, fentanyl, sufentanil, meperidine, methadone, nalbuphine, propoxyphene, and pentazocine, and pharmaceutically acceptable salts thereof. In certain embodiments, the analgesic is acetaminophen. In certain embodiments, the therapeutically effective amount of acetaminophen is in the range of 325-1000 mg, 400-1000 mg, 500-1000 mg, 600-1000 mg, 700-1000 mg, 800-1000 mg, 900-1000 mg, 325-800 mg, 400-800 mg, 500-800 mg, 600-800 mg, 700-800 mg, 325-600 mg, 400-600 mg, or 500-600 mg. In certain embodiments, the effective amount of acetaminophen is 325 mg, 500 mg, 650 mg, 700 mg, 800 mg, 900 mg, or 1000 mg.

In certain embodiments, the analgesic is administered parenterally. In certain embodiments, the analgesic is administered intravenously. In certain embodiments, the analgesic is administered orally.

The analgesic can be administered prior to, simultaneously with, or subsequent to the administration of the multi-specific binding protein, pharmaceutical composition, or pharmaceutical formulation disclosed herein. In certain embodiments, the analgesic is administered within 2 hours, within 1.5 hours, within 1 hour (60 minutes), within 45 minutes, within 30 minutes, within 15 minutes, or immediately prior to the administration of the multi-specific binding protein (e.g., prior to the beginning of the administration of the multi-specific binding protein). In certain embodiments, the analgesic is administered simultaneously with the administration of the multi-specific binding protein. In certain embodiments, the analgesic and the multi-specific binding protein are diluted into a single pharmaceutical composition administered to the subject. In certain embodiments, the analgesic (e.g., acetaminophen) is administered with every dose of the multi-specific binding protein.

In certain embodiments, the analgesic (e.g., acetaminophen) is administered 30 to min., 40 to 90 min., 50 to 90 min., 60 to 90 min., 70 to 90 min., 80 to 90 min., 30 to 80 min., to 80 min., 50 to 80 min., 60 to 80 min., 70 to 80 min., 30 to 70 min., 40 to 70 min., 50 to 70 min., 60 to 70 min., 30 to 60 min., 40 to 60 min., 50 to 60 min., 30 to 50 min., 40 to 50 min., 30 to 40 min., about 30 min., about 40 min., about 50 min., about 60 min., about 70 min., about 80 min., or about 90 min., prior to administration of the multi-specific binding protein, pharmaceutical composition, or pharmaceutical formulation disclosed herein.

In certain embodiments, the duration of administration of the analgesic and the duration of administration of the multi-specific binding protein completely or partially overlap. In certain embodiments, the analgesic is administered within 2 hours, 1 hour, or 30 minutes subsequent to the administration of the multi-specific binding protein (e.g., subsequent to the beginning of the administration of the multi-specific binding protein).

An antipyretic can be used to prevent or reduce fever as a result of the administration of the multi-specific binding protein, pharmaceutical composition, or pharmaceutical formulation disclosed herein, whether administered as a monotherapy or as a combination therapy. Accordingly, in certain embodiments, the method further includes administering to the subject a therapeutically effective amount of an antipyretic. Exemplary antipyretics are disclosed in U.S. Patent Application Publication No. 2015/0342989. In certain embodiments, the antipyretic used in the method disclosed herein is selected from acetaminophen, salicylamide, salicyl salicylate, methyl salicylate, magnesium salicylate, faislamine, ethenzamide, diflunisal, choline magnesium salicylate, benorylate/benorilatem and amoxiprin, acetylsalicylate, ceclofenac, acemetacin, alclofenac, bromfenac, diclofenac, etodolac, indomethacin, nabumetone, oxametacin, proglumetacin, sulindac, tolmetin, iminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, naproxen, oxaprozin, pirprofen, suprofen, tiaprofenic acid, mefenamic acid, flufenamic acid, meclofenamic acid, tolfenamic acid, droxicam, lornoxicam, meloxicam, piroxicam, and tenoxicam, mpyrone, azapropazone, clofezone, kebuzone, metamizole, mofebutazone, oxyphenbutazone, phenazone, phenylbutazone, sulfinpyrazone, decoxib, rofecoxib, parecoxib, and etoricoxib. In certain embodiments, the antipyretic is acetaminophen. In certain embodiments, the therapeutically effective amount of acetaminophen is in the range of 325-1000 mg, 400-1000 mg, 500-1000 mg, 600-1000 mg, 700-1000 mg, 800-1000 mg, 900-1000 mg, 325-800 mg, 400-800 mg, 500-800 mg, 600-800 mg, 700-800 mg, 325-600 mg, 400-600 mg, or 500-600 mg. In certain embodiments, the effective amount of acetaminophen is 325 mg, 500 mg, 650 mg, 700 mg, 800 mg, 900 mg, or 1000 mg.

In certain embodiments, the antipyretic is administered parenterally. In certain embodiments, the antipyretic is administered intravenously. In certain embodiments, the antipyretic is administered orally.

The antipyretic can be administered prior to, simultaneously with, or subsequent to the administration of the multi-specific binding protein. In certain embodiments, the antipyretic is administered within 2 hours, 1.5 hours, 1 hour (60 minutes), 45 minutes, 30 minutes, or 15 minutes prior to the administration of the multi-specific binding protein (e.g., prior to the beginning of the administration of the multi-specific binding protein). In certain embodiments, the antipyretic is administered simultaneously with the administration of the multi-specific binding protein. In certain embodiments, the antipyretic and the multi-specific binding protein are diluted into a single pharmaceutical composition administered to the subject. In certain embodiments, the duration of administration of the antipyretic and the duration of administration of the multi-specific binding protein completely or partially overlap. In certain embodiments, the antipyretic is administered within 2 hours, 1 hour, or 30 minutes subsequent to the administration of the multi-specific binding protein (e.g., subsequent to the beginning of the administration of the multi-specific binding protein). In certain embodiments, the antipyretic (e.g., acetaminophen) is administered with every dose of the multi-specific binding protein.

The antipyretic can be administered prior to, simultaneously with, or subsequent to the administration of the multi-specific binding protein, pharmaceutical composition, or pharmaceutical formulation disclosed herein. In certain embodiments, the analgesic (e.g., acetaminophen) is administered 30 to 90 min., 40 to 90 min., 50 to 90 min., 60 to 90 min., 70 to min., 80 to 90 min., 30 to 80 min., 40 to 80 min., 50 to 80 min., 60 to 80 min., 70 to 80 min., to 70 min., 40 to 70 min., 50 to 70 min., 60 to 70 min., 30 to 60 min., 40 to 60 min., 50 to 60 min., 30 to 50 min., 40 to 50 min., 30 to 40 min., about 30 min., about 40 min., about 50 min., about 60 min., about 70 min., about 80 min., or about 90 min., prior to administration of the multi-specific binding protein, pharmaceutical composition, or pharmaceutical formulation disclosed herein.

In certain embodiments, subjects receive premedication treatment including 800 to 1000 mg of acetaminophen administered intravenously or orally 30 to 60 minutes prior to administration of the multi-specific binding protein, pharmaceutical composition, or pharmaceutical formulation disclosed. In certain embodiments, premedication treatment further includes intravenous or oral administration of 40 to 50 mg diphenhydramine prior to administration of the multi-specific binding protein, pharmaceutical composition, or pharmaceutical formulation disclosed.

### EXAMPLES

The disclosure now being generally described, will be more readily understood by reference to the following examples, which are included merely for purposes of illustration of certain aspects and embodiments of the present disclosure, and are not intended to limit the scope of the disclosure in any way.

### Example 1: High-Concentration Formulations of A49-F3′-TriNKET-Trastuzumab

The feasibility of a high-concentration A49-F3′-TriNKET-Trastuzumab formulation was assessed in two phases: an assessment of the solubility limit and short-term stability of the formulation.

To assess solubility, A49-F3′-TriNKET-Trastuzumab drug substance from the ultrafiltration/diafiltration (UF/DF) load was exchanged into a pH 6 buffer of 20 mM histidine and 250 mM sucrose, identical to a pharmaceutical formulation selected in Example 1 and used in the clinical study described herein (e.g., in Example 2), but without polysorbate-80. The A49-F3′-TriNKET-Trastuzumab was then concentrated to approximately 233 mg/mL and held at both 5° C. and 25° C. with intermittent checks on the A49-F3′-TriNKET-Trastuzumab protein concentration. Table 12 shows the average concentrations of A49-F3′-TriNKET-Trastuzumab in each storage condition at all timepoints.

TABLE 12
Concentrations of A49-F3′-TriNKET-Trastuzumab
in high-concentration formulations

| | | Average |
|---|---|---|
| | | concentration |
| Sample name | Storage | (mg/mL) |
| GMP UF/DF load material | NA | 4.4 |
| Buffer exchanged material | NA | 5.4 |
| Concentrated material | T0 | 234 |
| (measured neat) | T = 24 h, | 233 |
| | 2-8° C. | |
| | T = 48 h, | 223 |
| | 2-8° C. | |
| | T = 5 days, | 223 |
| | 2-8° C. | |
| | T = 24 h, | 232 |
| | 25° C. | |
| | T = 48 h, | 223 |
| | 25° C. | |
| | T = 5 days, | 223 |
| | 25° C. | |

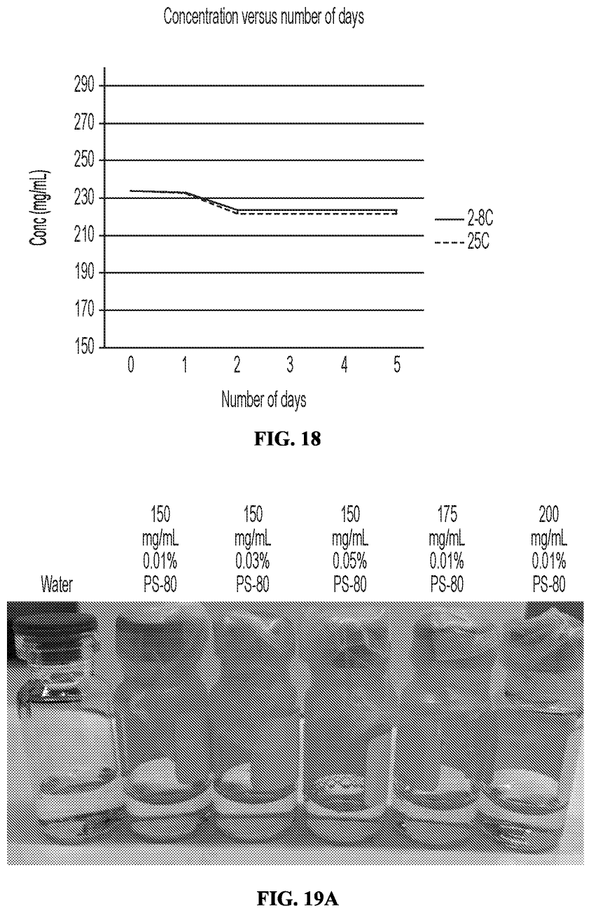

The A49-F3′-TriNKET-Trastuzumab concentration settled to approximately 222-223 mg/mL after 48 hours at each condition and remained at 222-223 mg/mL after 120 hours (FIG. 18). This suggests that the solubility limit of A49-F3′-TriNKET-Trastuzumab in this formulation is approximately 222 mg/mL.

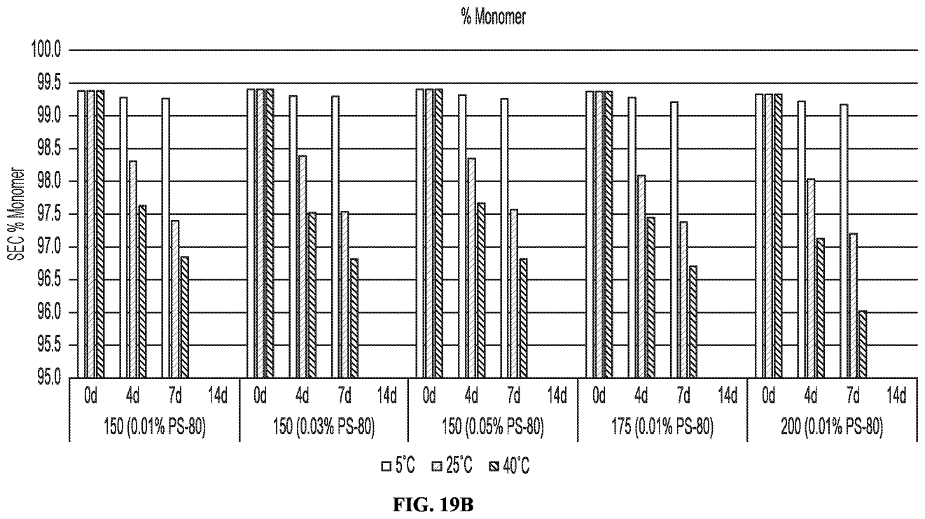

The short-term stability was assessed by preparing samples as described above to protein concentrations of 150 mg/mL, 175 mg/mL, and 200 mg/mL. The solution at each concentration was spiked with 0.01% (w/v) polysorbate-80, identical to a pharmaceutical formulation selected in Example 1 and used in the clinical study described herein (e.g., in Example 2). Two additional solutions at the 150 mg/mL concentration were prepared with 0.3% (w/v) polysorbate-80 and 0.05% (w/v) polysorbate-80, respectively, to assess the impact of polysorbate-80 concentration on the stability of high concentration A49-F3′-TriNKET-Trastuzumab. Each condition was stored at 5° C., 25° C., and 40° C. and sampled weekly for limited product quality testing, consisting of appearance (FIG. 19A), A49-F3′-TriNKET-Trastuzumab concentration, and purity by size-exclusion high-performance liquid chromatography (SE-HPLC) (FIG. 19B). The results demonstrate that A49-F3′-TriNKET-Trastuzumab remains stable in these formulations at both 5° C. and 25° C. for two weeks.

### Example 2: Treatment of Locally Advanced or Metastatic Solid Tumor with A49-F3′-TriNKET-Trastuzumab

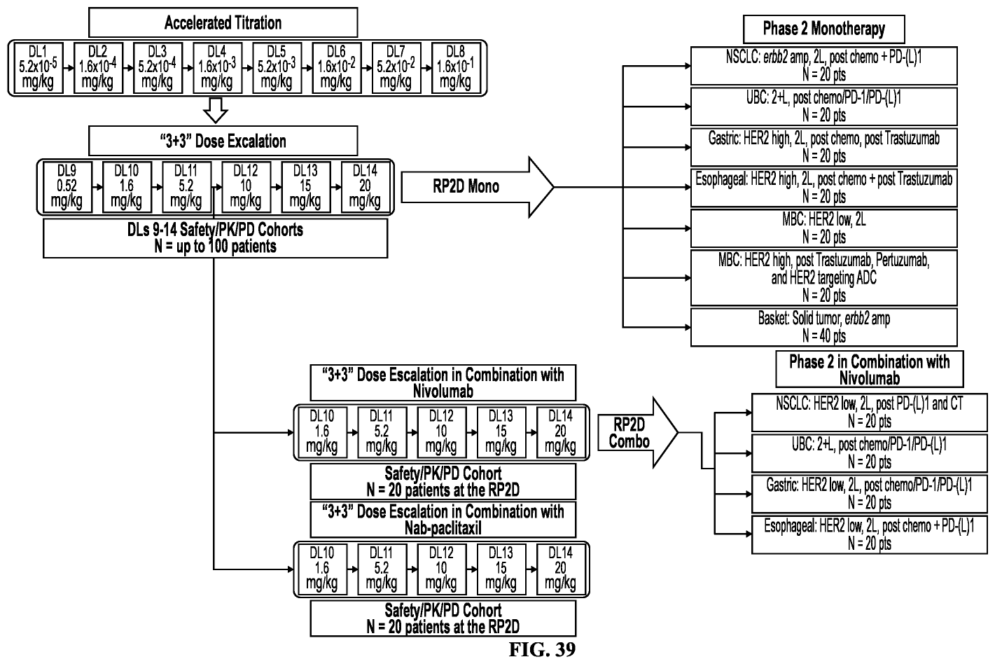

This example describes a phase 1 clinical study to assess the safety and tolerability of A49-F3′-TriNKET-Trastuzumab. Design of this clinical trial, and a related phase 2 clinical trial, is illustrated in FIG. 39. The study according to the protocol described in this example has been ongoing, and certain results are also described at the end of this example.

### Objectives

The primary objective of the phase 1 clinical study is to assess the safety and tolerability of A49-F3′-TriNKET-Trastuzumab, and to determine the maximum tolerated dose of A49-F3′-TriNKET-Trastuzumab in patients with advanced (unresectable, recurrent or metastatic) solid tumors for whom no effective standard therapy exists or have recurrent or are intolerant of standard therapy(ies), to assess the safety and tolerability of the A49-F3′-TriNKET-Trastuzumab and nivolumab combination therapy, and to assess the safety and tolerability of the A49-F3′-TriNKET-Trastuzumab and nab-paclitaxel combination therapy.

The secondary objectives of this clinical study are:
to characterize the pharmacokinetics of A49-F3′-TriNKET-Trastuzumab;
to evaluate immunogenicity of A49-F3′-TriNKET-Trastuzumab and to correlate its immunogenicity to its exposure and clinical activity;
to assess duration of response (DOR) for confirmed responses of A49-F3′-TriNKET-Trastuzumab (efficacy expansion cohorts);
to assess confirmed best overall response (BOR) (efficacy expansion cohorts);
to assess progression free survival (PFS) for A49-F3′-TriNKET-Trastuzumab (efficacy expansion cohorts);
to assess confirmed ORR, DOR for confirmed responses, confirmed BOR and PFS for patients enrolled in the dose escalation phase and in the efficacy expansion phase; and
to assess overall survival (OS) time.

Study Design

This study is a Phase I, open-label, dose escalation study with a consecutive parallel-group efficacy expansion study, designed to determine the safety, tolerability, pharmacokinetic(s) (PK), pharmacodynamic(s) (PD), and preliminary anti-tumor activity of A49-F3′-TriNKET-Trastuzumab alone and in combination with pembrolizumab, nivolumab, or nab-paclitaxel. This dose-escalation study is divided into the following three phases:
(A) accelerated titration;
(B) “3+3” dose escalation with A49-F3′-TriNKET-Trastuzumab administered as a monotherapy, or as a combination therapy (e.g., with nivolumab, or nab-paclitaxel); and
(C) safety/pharmacokinetic(s) (PK)/pharmacodynamic(s) (PD) expansion cohorts with A49-F3′-TriNKET-Trastuzumab administered as a monotherapy, or as a combination therapy (e.g., with nivolumab, or nab-paclitaxel).

Inclusion Criteria

Patients in the dose escalation phase (“accelerated titration” and “3+3” dose escalation parts) have the following inclusion criteria.
Histologically or cytologically proven locally advanced or metastatic solid tumors, for which no standard therapy exists, or standard therapy has failed. HER2 expression by immunohistochemistry and/or erbb2 amplification and/or erbb2 activating mutations must be documented on either archival tissue or fresh tumor biopsy.

Patients in the nivolumab “3+3” cohort have the following inclusion criteria.
Patients are eligible to receive nivolumab per its label for a malignancy of epithelial origin or have a tumor that does not have a standard therapy or standard therapy has failed.
Patients have received an anti PD-1 or an anti PD-L1 as a previous line of therapy; or has not received anti PD-1 or anti-PD-L1 as a previous line of therapy and has experienced a Grade 3 or 4 drug related toxicity, or a Grade 2 drug related toxicity related to prior checkpoint therapy impacting the lungs or the neurological system related to the prior anti-PD-1 or anti PD-L1 therapy.

Patients in the nab-paclitaxel “3+3” cohort have the following inclusion criteria.
Patients are eligible for treatment with nab-paclitaxel per its label, which includes metastatic breast cancer, after failure of combination chemotherapy for metastatic disease or relapse within 6 months of adjuvant chemotherapy. In this case, additional inclusion criteria also includes no exposure to taxanes in the last 6 months.

### OR

Patients have a tumor that does not have a standard therapy or standard therapy has failed. In this case, patients should also not have been treated with a taxane over the last 6 months.

### OR

Patients have a first line advanced (unresectable/recurrent/metastatic) TNBC.

Patients in the safety/PK/PD expansion part (dose escalation phase) have the following inclusion criteria.
Histologically or cytologically proven locally advanced or metastatic solid tumors, for which no standard therapy exists, or standard therapy has failed. A patient tumor must have documented HER2 expression by immunohistochemistry using a CLIA accredited (or equivalent) method.
Disease must be measurable with at least 1 unidimensional measurable lesion by RECIST 1.1.
Availability of a screening biopsy obtained from relapsed metastatic or locally advanced disease (if applicable), obtained within the screening window. Tumor tissue from bone metastases is not evaluable for PD-L1 expression and is not acceptable.
HER2 levels measured by immunohistochemistry of at least 1+at screening (archival tissue is acceptable to determine HER2 status). Patients with breast cancer are eligible even with a Herceptest score of 0, as long as the expression of HER2 can be detected by IHC in at least 1% of the tumor cells.

Patients to receive a nivolumab combination therapy in the safety/PK/PD expansion cohort additionally have the following inclusion criteria.
Patients are eligible to receive nivolumab per its label for a malignancy of epithelial origin.

### OR

There is no standard therapy for the patient, or standard therapy has failed.
A grade 3 or 4 drug related toxicity during the treatment with the anti-PD-1 or anti-PD-L1.
A grade 2 drug related toxicity related to prior checkpoint therapy that impacted either the lungs or the neurological system.

Patients to receive a nab-paclitaxel combination therapy in the safety/PK/PD expansion cohort have the following inclusion criteria.
Patients are eligible for treatment with nab-paclitaxel per its label, which includes metastatic breast cancer, after failure of combination chemotherapy for metastatic disease or relapse within 6 months of adjuvant chemotherapy. In this case, additional inclusion criteria also includes no exposure to taxanes in the last 6 months.

### OR

Patients have a tumor that does not have a standard therapy or standard therapy has failed. In this case, patients should also not have been treated with a taxane over the last 6 months.

### OR

Patients have a first line advanced (unresectable/recurrent/metastatic) TNBC.

Exclusion Criteria

The exclusion criteria for patients enrolled in the clinical study of this example include:
Concurrent treatment with a non-permitted drug as in Non-Permitted Medicines and Therapies section. Previous treatment with drugs that specifically target the HER2 pathway (mAb or Tyrosine Kinase Inhibitor [TKI]) is acceptable providing washout period (4 weeks or 5 half-lives for mAbs or protein therapeutics and 2 weeks or 5 half-lives for a TKI).
Concurrent anticancer treatment (e.g., cytoreductive therapy, radiotherapy [with the exception of palliative bone directed radiotherapy], immune therapy, or cytokine therapy except for erythropoietin), major surgery (excluding prior diagnostic biopsy), concurrent systemic therapy with steroids or other immunosuppressive agents, or use of any investigational drug within 28 days or 5 half-lives (whichever is shorter) prior to the start of study treatment. Short-term administration of systemic steroids (i.e., for allergic reactions or the management of irAEs) is allowed. Note: Patients receiving bisphosphonates or denosumab are eligible provided treatment was initiated at least 14 days before the first dose of F3′-TriNKET-Trastuzumab.
Previous malignant disease other than the target malignancy to be investigated in this study within the last 3 years, with the exception of basal or squamous cell carcinoma of the skin, low-grade prostate cancer (Gleason score 6 or less), or cervical carcinoma in situ.
Life expectancy of less than 3 months.
Patients with brain metastases are excluded unless all of the following criteria are met:
   a. CNS lesions are asymptomatic and previously treated
   b. Patient does not require ongoing steroid treatment daily for replacement for adrenal insufficiency (except oral steroids at a dose less than ≤10 mg prednisone [or equivalent])
   c. Imaging demonstrates stability of disease 28 days from last treatment for CNS metastases
Receipt of any organ transplantation including autologous or allogeneic stem-cell transplantation.
Significant acute or chronic infections (including historic positive test for human immunodeficiency virus [HIV], or active or latent hepatitis B or active hepatitis C tested during the screening window). If HBsAg is negative and the anti-hepatitis B core antibody is positive, then hepatitis B viral DNA load must be undetectable.
Preexisting autoimmune disease (except for patients with vitiligo) needing treatment with systemic immunosuppressive agents for more than 28 days within the last 3 years or clinically relevant immunodeficiencies (e.g., dys-gammaglobulinemia or congenital immunodeficiencies), or fever Grade 2 or higher within 7 days of day 1. Patients with a history of autoimmune-related hypothyroidism on a stable dose of thyroid replacement hormone may be eligible for this study. Patients with controlled Type 1 diabetes mellitus on a stable insulin regimen may be eligible for this study.
Known severe hypersensitivity reactions to mAbs (≥Grade 3 NCI-CTCAE v5.0), any history of anaphylaxis, or uncontrolled asthma (i.e., 3 or more features of partly controlled asthma).
Persisting toxicity related to prior therapy ≤Grade 1 NCI-CTCAE v5.0, however alopecia, endocrinopathies ≤Grade 2, and sensory neuropathy ≤Grade 2 is acceptable.
Pregnancy or lactation in females during the study.
Known alcohol or drug abuse.
Serious cardiac illness or medical conditions including but not limited to:
   a. History of New York Heart Association class III or IV heart failure or systolic dysfunction (LVEF <55%).
   b. High-risk uncontrolled arrhythmias i.e., tachycardia with a heart rate >100/min at rest.
   c. Significant ventricular arrhythmia (ventricular tachycardia) or higher-grade AV-block (second degree AV-block Type 2 [Mobitz 2] or third-degree AV-block).
   d. Angina pectoris requiring anti-anginal medication.
   e. Clinically significant valvular heart disease.
   f. Evidence of transmural infarction on ECG.
   g. Poorly controlled hypertension (defined by: systolic >180 mm Hg or diastolic >100 mm Hg).
   h. Clinically relevant uncontrolled cardiac risk factors, clinically relevant pulmonary disease or any clinically relevant medical condition that may limit participation in this study.
Severe dyspnea at rest due to complications of advanced malignancy or requiring supplementary oxygen therapy.
All other significant diseases (e.g., inflammatory bowel disease), which might impair the patient's ability to participate.
Any psychiatric condition that would prohibit the understanding or rendering of informed consent.
Legal incapacity or limited legal capacity.
Incapability of giving signed informed consent, which includes compliance with the requirements and restrictions listed in the informed consent form (ICF) and in this protocol.
Patients assigned to receive A49-F3′-TriNKET-Trastuzumab in combination with nivolumab must not have experienced:
   a Grade 3 or 4 drug related toxicity during the previous treatment with the anti PD-1 or anti PD-L1 therapy, or neither
   a Grade 2 drug related toxicity related to prior anti PD-1 or anti PD-Lltherapy that impacted either the lungs or the neurological system.

Dose Limiting Toxicities

At each cohort, safety and tolerability is assessed. Dose-limiting toxicity (DLT) is evaluated in the first 21 days for the patients enrolled in the dose escalation part and in the pembrolizumab combination cohort. A DLT is a ≥grade 3 adverse drug reaction according to the National Cancer Institute-Common Terminology Criteria for Adverse Events (NCI-CTCAE) v5.0, occurring in the DLT evaluation period of the dose escalation cohorts. Adverse drug reactions may be adverse events suspected to be related to A49-F3′-TriNKET-Trastuzumab by the investigator and/or sponsor. DLT is defined as any of the following occurring within the first 21 days of treatment for the patients enrolled in the dose escalation part and in the pembrolizumab combination cohort:
Any grade 3 to 4 non-hematological toxicity except:
   i. grade 3 nausea, vomiting, and diarrhea lasting <72 hours in the absence of maximal medical therapy;
   ii. grade 4 vomiting and diarrhea lasting <72 hours in the absence of maximal medical therapy;
   iii. grade 3 fatigue <5 days;
   iv. grade 3 hypertension in the absence of maximal medical therapy.
Any of the following hematological toxicities:
   i. grade 4 neutropenia >5 days;
   ii. grade 3 thrombocytopenia with hemorrhage;
   iii. grade 4 thrombocytopenia.
Exception for single laboratory values out of normal range that are unlikely related to study treatment according to the Investigator, do not have any clinical correlate, and resolve to ≤Grade 1 within 7 days with adequate medical management.

The observation period for DLTs may include the first 3 weeks of A49-F3′-TriNKET-Trastuzumab treatment in the dose escalation part for all dose cohorts for all patients with data used for implementing the dose-escalation algorithm for determination of the maximum tolerated dose (MTD). Additional patients may be enrolled in the dose escalation phase and may have adverse events collected; optionally, these patients may not have a specific DLT observation period. Safety monitoring committee may adopt a conservative approach in ascribing the relevance of the treatment related-toxicity to drug. A treatment-related serious adverse event is ascribed as related to drug except where a clear relationship to the underlying disease or recognized co-morbidities is evident.

Safety is assessed through the recording, reporting, and analysis of baseline medical conditions, adverse events (AEs), physical examination findings, including vital signs and determination of left ventricular ejection fraction, electrocardiogram, and laboratory tests.

Dosage and Administration

A49-F3′-TriNKET-Trastuzumab

Prophylactic medication on Cycle 1, Day 1 (C1D1) consists of acetaminophen, diphenhydramine, and a corticosteroid (methylprednisolone). From Cycle 1, Day 8 (C1D8) onwards, prophylactic premedication consists of acetaminophen and diphenhydramine only unless corticosteroid continuation has been discussed with and approved by sponsor.

For patients at DL10 and below, patients receive A49-F3′-TriNKET-Trastuzumab as an IV infusion over a period of at least 1 hours and up to 2 hours at all infusions. Patients receive the full dosage of A49-F3′-TriNKET-Trastuzumab according to their assigned dose level (DL) at all infusions.

For patients at DL11, patients receive A49-F3′-TriNKET-Trastuzumab as an IV infusion over a period of at least 3 hours and up to 4 hours at all infusions. Patients receive the full dosage of A49-F3′-TriNKET-Trastuzumab according to their assigned DL at all infusions.

For patients at DL12 and above, at Cycle 1 Day 1, patients receive a priming dose of A49-F3′-TriNKET-Trastuzumab at 5 mg/kg. Beginning at C1D8 and for the remainder of the time on study treatment, the dosage of A49-F3′-TriNKET-Trastuzumab is given at the respective DL dosage for the assigned cohort (e.g., a patient enrolling at DL12 receive 5 mg/kg of A49-F3′-TriNKET-Trastuzumab on C1D1, and 10 mg/kg on C1D8 and for all infusions thereafter). Patients receive A49-F3′-TriNKET-Trastuzumab as an IV infusion over a period of at least 3 hours and up to 4 hours on Days 1, 8, and 15 of C1 and up to 2 hours but no less than 1 hour for all subsequent infusions. Patients receive A49-F3′-TriNKET-Trastuzumab as an IV infusion over a period of at least 3 hours and up to 4 hours on Days 1, 8, and 15 of C1 and up to 2 hours but no less than 1 hour for all subsequent infusions. Such mode of administration is applicable whenever A49-F3′-TriNKET-Trastuzumab is administered at DL12 or above, as a monotherapy or in combination with nivolumab or nab-paclitaxel.

At each administration, it is permissible to extend the infusion longer if necessary for the management of patient comfort or tolerability.

Dose Escalation

In the accelerated titration phase, patients are administered with an initial eight doses of A49-F3′-TriNKET-Trastuzumab following an accelerated dose escalation with escalation steps no greater than 3.3-fold. Table 13 outlines the starting dose according to body weight (mg/kg) and dose levels (DL) of the escalation scheme.

TABLE 13
Exemplary DLs (in mg/kg body weight) in “accelerated titration” escalation phase.
“accelerated titration”

| DL1 | DL2 | DL3 | DL4 | DL5 | DL6 | DL7 | DL8 |
|---|---|---|---|---|---|---|---|
| 5.2 × 10⁻⁵ | 1.6 × 10⁻⁴ | 5.2 × 10⁻⁴ | 1.6 × 10⁻³ | 5.2 × 10⁻³ | 1.6 × 10⁻² | 5.2 × 10⁻² | 1.6 × 10⁻¹ |

In the “3+3” dose escalation part, the next six dose levels are described in Table 14.

TABLE 14
Exemplary DLs (in mg/kg body weight)
in “3 + 3” dose escalation phase.

| | “3 + 3” dose escalation | | | | |
|---|---|---|---|---|---|
| DL9 | DL10 | DL11 | DL12 | DL13 | DL14 |
| 0.52 | 1.6 | 5.2 | 10 | 15 | 20 |

Nivolumab

Nivolumab is used at the approved dose of 480 mg (as per its label) in the Combination Therapy with Nivolumab Cohort of the Dose Escalation Part (Phase I) of the study.

Nivolumab is administered on day 1 of each 28 day cycle, as described in its label.

In the combination therapy with nivolumab cohorts, patients receive nivolumab on Day 1 of each four-week cycle at a dose of 480 mg, administered as a 30-minute infusion according to its package insert. Nivolumab is administered prior to the administration of A49-F3′-TriNKET-Trastuzumab.

Nab-Paclitaxel

In the nab-paclitaxel combination therapy cohorts, patients receive nab-paclitaxel (Abraxane®) on Day 1, Day 8 and Day 15 of each four-week cycle at a dose of 100 mg/m 2, as a infusion according to its package insert. Nab-paclitaxel is administered prior to the administration of A49-F3′-TriNKET-Trastuzumab.

Endpoints

The study is designed to evaluate primary and secondary endpoints to assess clinical benefits of A49-F3′-TriNKET-Trastuzumab, optionally in combination with nivolumab or nab-paclitaxel b as treatment for patients with locally advanced or metastatic solid tumors.

A primary endpoint of the dose escalation study is occurrence of dose-limiting toxicities (DLTs) during the first three weeks of treatment is measured as a primary endpoint in the dose escalation part.

Secondary endpoints for the study include the following:
number, severity, and duration of treatment-emergent adverse events for all dose groups/indications according to the NCI-CTCAE v5.0;
number, severity, and duration of treatment related adverse events according to NCI-CTCAE v5.0;
duration of response for confirmed responses according to RECIST 1.1;
pharmacokinetics profile;
confirmed best overall response according to RECIST 1.1;
progression free survival according to RECIST 1.1;
overall survival time;
progressive disease profile;
serum titers of anti-A49-F3′-TriNKET-Trastuzumab antibodies;
expression of HER2 on tumor tissue;
ERBB2 status (amplified/non-amplified, mutated/non-mutated);
unconfirmed response at Week 13 according to RECIST 1.1 (for Safety/PK/PD expansion cohorts); and
confirmed best overall response according to RECIST 1.1, per IERC, DOR for confirmed responses according to RECIST 1.1, per IERC, and progression free survival time, according to RECIST 1.1; duration of response according to RECIST 1.1 (for the efficacy expansion cohorts).

Safety endpoints for the study include the following:
Laboratory parameters
Electrocardiograms (ECGs)
Vital signs
Eastern Cooperative Oncology Group (ECOG) performance status
Left ventricular ejection fraction (LVEF)

Pharmacokinetic Profile

Serum concentrations of A49-F3′-TriNKET-Trastuzumab is determined by a validated method. The following PK parameters are estimated and reported:
AUC0→t: Area under the concentration-time curve from the time of dosing to the time of the last observation (calculated by linear trapezoidal summation);
AUC0→∞: Area under the curve from the time of dosing extrapolated to infinity (calculated by the linear trapezoidal summation and extrapolated to infinity using Clast/λz);
λz: Terminal elimination rate constant. The value of λz is determined from the slope of the regression line of log (concentration) vs. time with the following constraints: (i) there must be at least 3 consecutive measurable concentrations, (ii) all concentrations must be declining with time, and (iii) the correlation coefficient (r) of regression must be ≥0.95;
Cmax: Maximum serum concentration observed post-dose;
tmax: Time at which the Cmax occurs; and
t ½: Elimination half-life, determined as 0.693/λz.

The PK parameters are summarized using descriptive statistics. Individual as well as mean concentration-time plots are depicted. Unresolved missing data may be imputed when the analysis integrity is affected. The conservative principle is used for data imputation.

Serum titers of anti-drug antibodies: The safety immunogenicity testing strategy is implemented and conducted in line with:
Immunogenicity Assessment of Biotechnology-Derived Therapeutic Proteins (see Guideline on Immunogenicity Assessment of Therapeutic Proteins. 18 May 2017 EMEA/CHMP/BMWP/14327/2006 Rev 1 Committee for Medicinal Products for Human Use (CHMP); European Medicines Agency);
Immunogenicity assessment of mAbs intended for in vivo clinical use (see Guideline on Immunogenicity Assessment of Monoclonal Antibodies Intended for In Vivo Clinical Use. 24 May 2012 EMA/CHMP/BMWP/86289/2010 Committee for Medicinal Products for Human Use (CHMP); European Medicines Agency);
FDA (2009, draft) Guidance for Industry: Assay Development for Immunogenicity Testing of Therapeutic Proteins.

A qualified method that uses an acid dissociation step to detect anti-drug (i.e., anti-A49-F3′-TriNKET-Trastuzumab) antibodies in the presence of excess drug in human serum is applied. Removal of drug after acid treatment is not required. ADA titers of positive samples is determined.

Biomarkers

Summary statistics for biomarkers are provided for all preplanned timepoints, separately for each DL or cohort. Changes to baseline levels are presented as applicable. Profiles over time are displayed on a per patient basis.

Safety Analyses

The extent of exposure to A49-F3′-TriNKET-Trastuzumab is characterized by duration (weeks), number of administrations, cumulative dose (mg/kg), dose intensity (mg/kg/week), relative dose intensity (actual dose given/planned dose), number of dose reductions, and number of dose delays. Safety analyses are performed on the safety population. The safety endpoints are tabulated by DL and cohort, using descriptive statistics. Safety assessments are based on review of the incidence of adverse events including adverse events of special interest, adverse drug reactions, and changes in vital signs, electrocardiograms, body weight, and laboratory values (hematology and serum chemistry). The on-treatment period is defined as the time from the first dose of study treatment to the last dose of study treatment+30 days, or the earliest date of new anticancer therapy −1 day, whichever occurs first.

Adverse Events (AEs)

Adverse events are coded according to Medical Dictionary for Regulatory Activities (MedDRA). Severity of AEs is graded using the NCI-CTCAE v5.0 toxicity grading scale. Treatment-emergent adverse events (TEAEs) are those AEs with onset dates during the on-treatment period, or if the worsening of an event is during the on-treatment period. The incidence of TEAEs regardless of attribution and AEs defined as possibly related to A49-F3′-TriNKET-Trastuzumab are summarized by preferred term and system organ class and described in terms of intensity and relationship to A49-F3′-TriNKET-Trastuzumab. All premature/permanent discontinuations are summarized by primary reason for study withdrawal. Duration TEAEs is defined as the time between onset and resolution to baseline. Duration of Grade 3 and 4 is defined by the time period during which a particular TEAE reaches a Grade 3 or 4 severity during its course. Descriptive statistics are examined for indications of dose-related ADRs.

Laboratory Variables

Laboratory results are classified by Grade according to NCI-CTCAE. The worst on-trial Grades after the first trial treatment are summarized. Shifts in toxicity grading from first treatment to highest grade are displayed. Results for variables that are not part of NCI-CTCAE are presented as below, within, or above normal limits. Only patients with post-baseline laboratory values are included in these analyses.

Physical Examination

Physical examination data, including vital signs (body temperature, respiratory rate, heart rate, and blood pressure) and cardiac safety assessments (12-lead electrocardiograms (ECG), and transthoracic echocardiography (TT-ECHO) or multigated acquisition scan (MUGA)) are recorded.

Safety Data

Safety data through DL13 (15 mg/kg) shows that the safety profile of A49-F3′-TriNKET-Trastuzumab is manageable. The data described below is based on a data cut-off of Feb. 1, 2023. Adverse events (AE) observed are mostly low grade, non-serious, and infrequently lead to treatment discontinuation. Infusion-related reactions (IRRs) are the most frequently reported AEs. IRRs are low grade (Grade 1 or 2), reversible, mostly occur with first dose, and recur infrequently. Five events of cytokine release syndrome (CRS) were reported. All events of CRS were low grade (Grade 1 or 2), reversible, and occurred after first dose. Five events of left ventricular ejection fraction (LVEF) were observed. These event were all asymptomatic, Grade 1, and diagnosed on routine cardiac imaging.

TABLE 15
Safety Data with A49-F3′-TriNKET-Trastuzumab Through DL13 as of Feb. 1, 2023

| | | | | | 3 + 3 | |
|---|---|---|---|---|---|---|
| | | | | | DE and | |
| | | | | | SPKPD | |
| | | | | | Exp & | |
| | | | | | Eff Exp | |
| Acc Titr | 3 + 3 DE and SPKPD Exp Cohorts | | | | Cohorts | |
| Phase | DL9 | DL10 | DL11 | DL12 | DL13 | |
| DL1-8 | 0.5 mg/kg | 1.6 mg/kg | 5.2 mg/kg | 10 mg/kg | 15 mg/kg | Total |
| (n = 10) | (n = 16) | (n = 13) | (n = 24) | (n = 20) | (n = 32) | (n = 115) |

Number of patients with at least one event (percent)

| | | | | | | | |
|---|---|---|---|---|---|---|---|
| DLTs | 0 | 0 | 0 | 0 | 0 | 0 | 0 |
| AEs | 9 (90.0%) | 16 (100.0%) | 13 (100.0%) | 24 (100.0%) | 20 (100.0%) | 29 (90.6%) | 111 (96.5%) |
| Drug-related AE | 7 (70.0%) | 12 (75.0%) | 11 (84.6%) | 18 (75.0%) | 17 (85.0%) | 23 (71.9%) | 88 (76.5%) |
| Grade 3-5 AEs | 3 (30.0%) | 6 (37.5%) | 8 (61.5%) | 14 (58.3%) | 9 (45.0%) | 8 (25.0%) | 48 (41.7%) |
| Drug-related Grade 3-5 | 0 | 0 | 1 (7.7%) | 5 (20.8%) | 3 (15.0%) | 1 (3.1%) | 10 (8.7%) |
| AEs* | | | | | | | |
| SAEs | 3 (30.0%) | 9 (56.3%) | 5 (38.5%) | 9 (37.5%) | 7 (35.0%) | 7 (21.9%) | 40 (34.8%) |
| Drug-related SAEs | 1 (10.0%) | 3 (18.8%) | 1 (7.7%) | 4 (16.7%) | 4 (20.0%) | 1 (3.1%) | 14 (12.2%) |
| AEs leading to | 2 (20.0%) | 3 (18.8%) | 1 (7.7%) | 1 (4.2%) | 3 (15.0%) | 2 (6.3%) | 12 (10.4%) |
| discontinuation | | | | | | | |
| Related AEs leading to | 0 | 1 (6.3%) | 1 (7.7%) | 1 (4.2%) | 2 (10.0%) | 2 (6.3%) | 7 (6.1%) |
| discontinuation | | | | | | | |
| AEs leading to Death | 0 | 2 (12.5%) | 1 (7.7%) | 1 (4.2%) | 0 | 0 | 4 (3.5%) |
| Related AEs leading to | 0 | 0 | 0 | 0 | 0 | 0 | 0 |
| Death | | | | | | | |
| Adverse Events of | 5 (50.0%) | 9 (56.3%) | 2 (15.4%) | 10 (41.7%) | 8 (40.0%) | 8 (25.0%) | 42 (36.5%) |
| Special Interest* | | | | | | | |

*AESI: RR, CRS and LVEF Decreased

In order to mitigate infusion-related reactions and cytokine release syndrome, premedication guidelines were implemented for both the monotherapy and combination cohorts.

Efficacy Data

Thirty paired biopsies were collected and analyzed during the “3+3” Dose-escalation and Safety/PK/PD expansion cohorts. Changes in the tumor microenvironment consistent with the mechanism of action of A49-F3′-TriNKET-Trastuzumab observed in mice were observed in 20 out of 30 patients who received A49-F3′-TriNKET-Trastuzumab at doses ranging from 0.5 to 15 mg/kg, and who entered in the study with levels of expression of HER2 ranging from 0 to 3, when measured using immunohistochemistry (IHC).

The following information were of particular relevance for the further evaluation of the A49-F3′-TriNKET-Trastuzumab:
Increases in CD8+ T cells, CD4+ T cells and NK cells were observed using multiplex IF and IHC methods.
Increases in the expression of PD-1 and PD-L1 were reported in most biopsies.
Activation of IFN-gamma responsive genes using NanoString.

Emerging signs of clinical activity in Phase 1 included:
A patient with lung adenocarcinoma with an amplification of ERBB2 entered in the study after disease progression on pemetrexed and carboplatin. The patient experienced a partial response (PR) at the first CT scan which was confirmed at the following CT scan (after 8 weeks on study). Patient experienced a PR for 15 months before experiencing disease progression in the brain.
A patient with HER2+ metastatic breast cancer entered the study after having received trastuzumab, pertuzumab with trastuzumab, and chemotherapy, and failing to respond to T-DM1 (best response on T-DM1 was progressive disease (PD), with the patient discontinuing treatment after 42 days). This patient remained with stable disease (SD) for more than 6 months while receiving A49-F3′-TriNKET-Trastuzumab at a dose of 0.5 mg/kg.
A patient with HR+ HER2 low (1+), ERBB2 non-amplified metastatic breast cancer who had progressed after multiple lines of endocrine therapy, CDK4/6 inhibitor, and chemotherapy experienced a PR at Cycle 1, confirmed by a subsequent CT scan performed at least 4 weeks after the CT scan that led to the first observation of a response.
Two additional patients with HR+ HER2 low metastatic breast cancer at diagnosis, who had progressed after multiple lines of endocrine therapy, CDK4/6 inhibitor and chemotherapy, experienced PRs at Cycle 1 while receiving treatment with A49-F3′-TriNKET-Trastuzumab. These two PRs have not been confirmed as of Feb. 6, 2023, with one patient experiencing progressive disease after a long treatment delay due to adverse event, while the other patient only recently completed the original scan and continues on treatment.

### Example 3: Cytotoxicity of A49-F3′-TriNKET-Trastuzumab

Human cancer cell lines expressing HER2 were used to assess tumor antigen binding of a TriNKET (A49-F3′-TriNKET-Trastuzumab and monoclonal antibodies in the presence of NK cells. The cell lines used are listed in Table 16.

TABLE 16
Cell Lines used in Cytolysis Assays

| Cell Line Name | Description | ATCC Cat# |
|---|---|---|
| SKBR-3 | HER2+ Breast adenocarcinoma | HTB-30 |
| H661 | Large cell lung cancer | HTB-183 |
| 786-0 | Renal cell adenocarcinoma | CRL-1932 |
| ZR-75-1 | Luminal A invasive ductal carcinoma | CRL-1500 |
| MCF-7 | Luminal A invasive ductal carcinoma | HTB-22 |
| BT20 | TNBC Basal A | HTB-19 |
| Hs578T | TNBC Basal B | HTB-126 |
| DU4475 | TNBC Basal A | HTB-123 |

The absolute numbers of HER2 proteins expressed on the surface of each cell of these cell lines were measured by flow cytometry. A49-F3′-TriNKET-Trastuzumab was labeled using an Alexa Fluor 647 Antibody Labeling Kit (Thermo Fisher, A20186). Saturating concentrations of the labeled TriNKET were determined by dissociating target cells with TrypLE in prewarmed culture media. Cells were resuspended in 1×PBS, adjusted to 1×10⁶/ml and 50 μl (5×10⁴) cells were plated in each well in duplicate. Live/Dead staining mix was prepared by diluting stock Zombie NIR (423105) or Zombie Aqua Fixable Viability dye (423102) 1:1000 in 1×PBS. Cells were pelleted, and resuspended in 50 μl Live/Dead staining mix, and incubated for 15 min at RT. Cells were washed 2× with FACS buffer, and resuspended in 50 μl serially diluted (3-fold; starting at 500 nM) labeled TriNKETs in FACS buffer. Cells were incubated for 1 hour in the dark on ice. Cells were washed 2× with FACS buffer, and resuspended in 50 μl fixation buffer (BioLegend) Cells were fixed for 10 min at RT. Cells were washed 2× with FACS buffer and analyzed on a BD FACSCelesta2 HTS machine.

For surface receptor quantitation, target-expressing cells were dissociated, plated in duplicate, and stained with Live/Dead dye as described above. Cells were then washed 2× with FACS buffer, and incubated with saturating concentrations of labeled TriNKET for 1 hour in the dark on ice. Cells were then washed, fixed and prepared for analysis on a BD FACSCelesta2 HTS machine as described above. Quantum Alexa Fluor 647 MESF beads (Bangs Laboratories, 647) were run on the same analysis setting to establish a calibration curve. Data was analyzed in FlowJo v10.7.1 (BD). Cells were gated for singlets (FSC-H vs FSC-A), and live cells (FSC-H vs Live/Dead). Manufacturer pre-defined MESF values and geometric mean fluorescence intensity (gMFI) values of Alexa Fluor 647 beads were log-transformed in Prism 9.0 using a non-linear fit to establish a calibration curve. Log-transformed gMFI values from TriNKET-stained cells were interpolated from calibration curve in Prism 9.0. Interpolated values were converted to non-log values, background was subtracted (as determined by the unstained cell population), and normalized for the degree of labeling. Rounded average receptor number of duplicate wells are reported in Table 17.

TABLE 17
HER2 expression on Cell Lines used in Cytolysis Assays

| Cell Line | Average Receptor Number | HER2 Expression |
|---|---|---|
| SKBR-3 | 678,876 | High |
| H661 | 44,066 | Medium |
| 786-0 | 10,881 | Low |
| ZR-75-1 | 57,692 | Medium |
| MCF-7 | 9,078 | Low |
| BT20 | 9,197 | Low |
| Hs578T | 4,146 | Low |
| DU4475 | 647 | Low |

To prepare NK cells, PBMCs were isolated from human peripheral blood buffy coats using density gradient centrifugation and were washed. NK cells were isolated from the PBMCs using a MACS negative selection technique with magnetic beads. NK cell (CD3⁻CD56⁺) purity was routinely confirmed to be greater than 90% of the harvested cells. The isolated NK cells were rested overnight and used the following day in cytotoxicity assays.

To conduct the DELFIA cytotoxicity assay, the HER2-expressing human cancer cell lines described above were harvested from culture. The cells were washed with HBS, and resuspended in growth media at 10⁶ cells/mL for labeling with BATDA reagent (Perkin Elmer AD0116). Manufacturer instructions were followed for labeling of the target cells. After labeling, the cells were washed three times with HBS, and were resuspended at 0.5×10⁵/mL in primary NK cell culture media.

Rested human NK cells or KHYG-1-CD16V-expressing cells were removed from culture and pelleted, the cells were resuspended in NK cell culture media at 0.1-2×10⁶ cells/mL depending upon the desired effector to target ratio (E:T). Assays using NK cells were performed using a 5:1 E:T ratio and a 10:1 E:T ratio with KHYG-1-CD16V cells. 4×A49-F3′-TriNKET-Trastuzumab and trastuzumab were prepared in primary NK cell culture media. In a round bottom TC 96-well plate, 100 ul of labeled target cells, 50 μl of 4×TriNKET/mAb, and 50 μl of effector cells were added. Control wells for background were prepared by pelleting labeled target cells, and 100 μl of the supernatant was added to background wells, containing 100 μl of primary NK cell culture media. Spontaneous release wells were prepared by adding 100 μl of labeled target cells to wells containing 100 μl of primary NK cell culture media. Maximum release wells were prepared by adding 100 μl of labeled target cells to wells containing 80 μl of primary NK cell culture media and 20 μl of 10% TritonX-100 solution. The assay plate was incubated at 37° C. with 5% CO₂ for 2-3 hours.

After culturing for 2-3 hours, the plate was removed from the incubator and the cells were pelleted by centrifugation at 300 g for 3 minutes. 20 μl of culture supernatant were transferred to a clean microplate provided from the manufacturer, and 200 μl of room temperature europium solution were added to each well. The plate was protected from light and incubated on a plate shaker at 250 rpm for 15 minutes. Fluorescence levels were read using either Victor 3 or SpectraMax i3X instruments.

The percentage of specific lysis was calculated as: % Specific lysis=(Experimental release−Spontaneous release)/(Maximum release−Spontaneous release)*100%

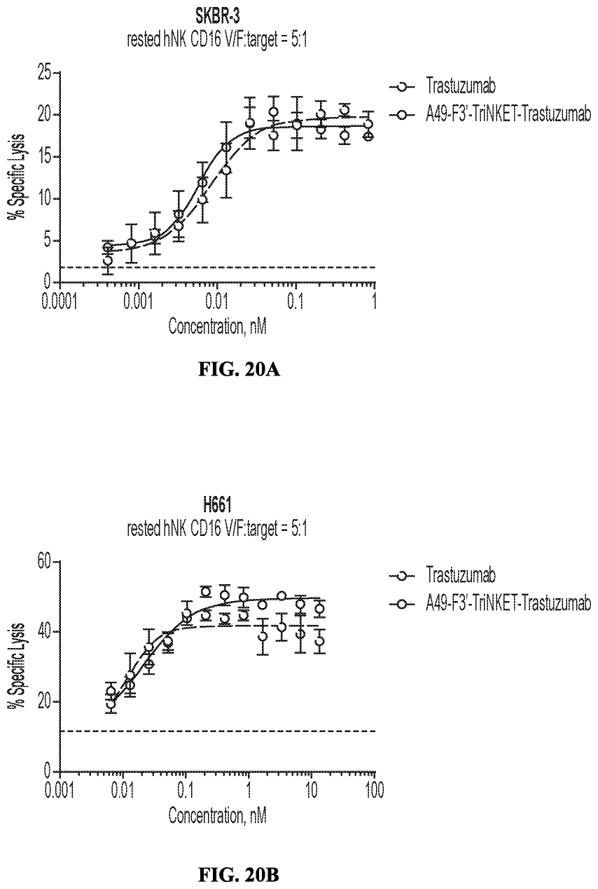

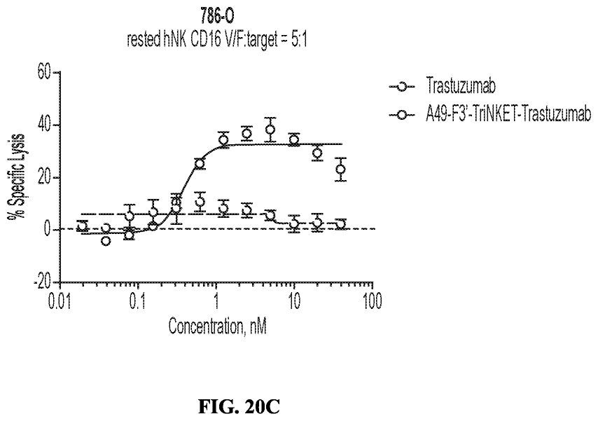

As shown in FIG. 20A-FIG. 20C, A49-F3′-TriNKET-Trastuzumab was more potent at promoting NK cell-mediated cytolysis of SKBR-3 (FIG. 20A) and 786-0 (FIG. 20C) cells than trastuzumab alone and comparable cytolysis against H661 cells (FIG. 20B). Against SKBR-3 cells (high level of HER2), A49-F3′-TriNKET-Trastuzumab demonstrated an EC50 of 0.008±0.003 nM across 3 healthy donors in enhancing NK cell-mediated cytolysis, and exhibited a 2.5-fold higher potency than trastuzumab in triggering NK cell-mediated killing. Maximum lysis for A49-F3′-TriNKET-Trastuzumab and trastuzumab varied among experiments and individual donors. Against H661 cells (medium level of HER2), A49-F3′-TriNKET-Trastuzumab and trastuzumab exhibited similar potency (EC50 of 0.054±0.032 nM) in triggering NK cell-mediated killing, with maximum lysis in the presence of A49-F3′-TriNKET-Trastuzumab being higher. Against the 786-0 renal cell carcinoma cells (low level of HER2), only A49-F3′-TriNKET-Trastuzumab, but not trastuzumab, was effective in inducing target cell lysis (EC50 0.638±0.461 nM).

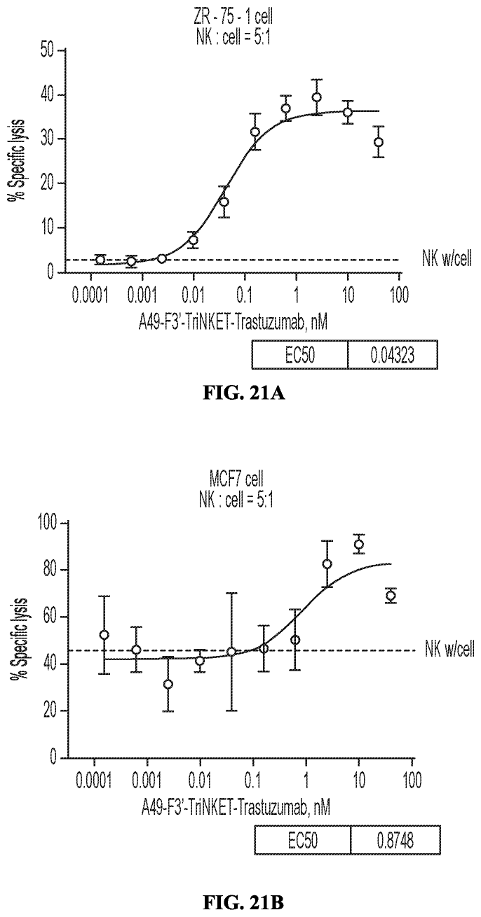

The ability of A49-F3′-TriNKET-Trastuzumab to enhance NK cell-mediated killing was assessed using two hormone receptor (HR)+ HER2− luminal A breast cancer cell lines. The ZR75-1 cells are estrogen receptor (ER) positive, progesterone receptor (PR)+/− and HER2 medium. MCF-7 cells are ER+, PR+ and HER2 low. As shown in FIG. 21A, A49-F3′-TriNKET-Trastuzumab demonstrated high potency in lysis of ZR75-1 cells with an EC50 of 0.043 nM. A49-F3′-TriNKET-Trastuzumab also enhanced NK cell-mediated killing against MCF-7 cells compared to NK cells alone with an EC50 of 0.878 nM (FIG. 21B).

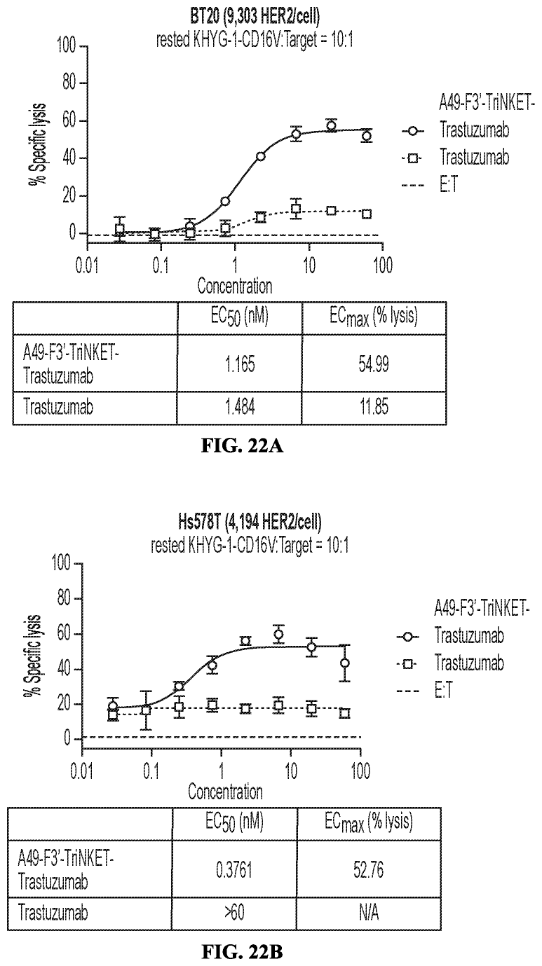

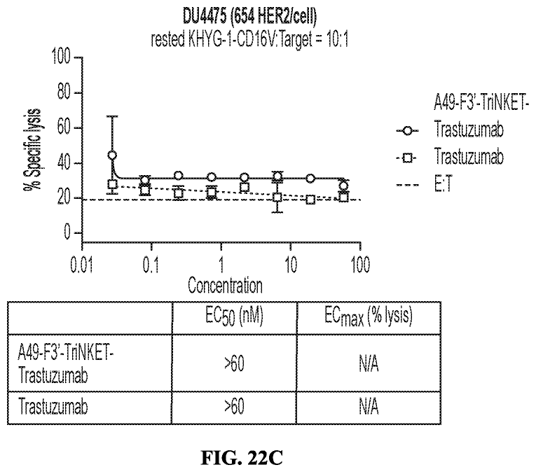

As shown in FIG. 22A-22B, A49-F3′-TriNKET-Trastuzumab was more potent at promoting cell-mediated cytolysis by KHYG-1-CD16V cells of BT-20 and Hs578T TNBC cells than trastuzumab alone. A49-F3′-TriNKET-Trastuzumab demonstrated an EC50 of 1.16 nM and a max lysis of 54.99% compared to trastuzumab (1.48 nM and 11.85% max lysis) against the BT-20 cell line (FIG. 22A). While trastuzumab had no impact on KHYG-1-CD16V cell-mediated killing against Hs578T cells, A49-F3′-TriNKET-Trastuzumab enhanced KHYG-1-CD16V cell killing with an EC50 of 0.376 nM and 52.76% max lysis (FIG. 22B). Neither A49-F3′-TriNKET-Trastuzumab nor trastuzumab enhanced KHYG-1-CD16V cell-mediated killing of DU4475 cells (FIG. 22C). All three cell lines assessed had low levels of HER2 expression.

### Example 4: Enhancement of Tumor Lymphocyte Infiltration with TriNKET Therapy

This example is designed to study the impact of a TriNKET treatment on the immune cell composition within the tumor and lymph nodes of tumor-bearing mice. B16F10 subcutaneous tumor is a model of relatively “cold” tumor and has been found to be unresponsive to many immunotherapies such as checkpoint blockers. CT26-Tyrp-1 is a relatively “hot” or inflamed tumor. The TriNKET used in this example is called mcFAE-C26.99, a heterodimer of C26 (an antibody that binds NKG2D) and TA99 (an antibody that binds Tyrp-1) with mouse IgG2c as the Fc. The Fc region includes mutations to promote heterodimerization (“GmA” and “GmB”). Amino acid sequences of mcFAE-C26.99 are provided below. Where clear from the context, this TriNKET is also referred to as “Tyrp1-TriNKET” or “TriNKET” in Example 4.

mC26_hvL_mCL
(SEQ ID NO: 206)
DIQMTQSPSTLSASVGDRVTITCRASQSISSWLAWYQQKPGKAPKLLIYK
ASSLESGVPSRFSGSGSGTEFTLTISSLQPDDFATYYCQQYGSFPITFGG
GTKVEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC
mC26_hvH_IgG2CGmB
(SEQ ID NO: 207)
QVQLQQWGAGLLKPSETLSLTCAVYGGSFSGYYWSWIRQPPGKGLEWIGE
IDHSGSTNYNPSLKSRVTISVDTSKNQFSLKLSSVTAADTAVYYCARARG
PWSFDPWGQGTLVTVSSAKTTAPSVYPLAPVCGGTTGSSVTLGCLVKGYF
PEPVTLTWNSGSLSSGVHTFPALLQSGLYTLSSSVTVTSNTWPSQTITCN
VAHPASSTKVDKKIEPRVPITQNPCPPLKECPPCAAPDLLGGPSVFIFPP
KIKDVLMISLSPMVTCVVVDVSEDDPDVQISWFVNNVEVHTAQTQTHRED
YNSTLRVVSALPIQHQDWMSGKEFKCKVNNRALPSPIEKTISKPRGPVRA
PQVYVLPPPAEEMTKKEFSLTCMI [symbol] GFLPAEIAVDWTSNGRTEQNYKNTA
TVLDSDGSYFMYS [symbol] LRVQKSTWERGSLFACSVVHEGLHNHLTTKTISRSL
GK
TA99_mvL_mCL
(SEQ ID NO: 208)
DIQMSQSPASLSASVGETVTITCRASGNIYNYLAWYQQKQGKSPHLLVYD
AKTLADGVPSRFSGSGSGTQYSLKISSLQTEDSGNYYCQHFWSLPFTFGS
GTKLEIKRADAAPTVSIFPPSSEQLTSGGASVVCFLNNFYPKDINVKWKI
DGSERQNGVLNSWTDQDSKDSTYSMSSTLTLTKDEYERHNSYTCEATHKT
STSPIVKSFNRNEC
TA99_mvH_IgG2CGmA
(SEQ ID NO: 209)
EVQLQQSGAELVRPGALVKLSCKTSGFNIKDYFLHWVRQRPDQGLEWIGW
INPDNGNTVYDPKFQGTASLTADTSSNTVYLQLSGLTSEDTAVYFCTRRD
YTYEKAALDYWGQGASVIVSSAKTTAPSVYPLAPVCGGTTGSSVTLGCLV
KGYFPEPVTLTWNSGSLSSGVHTFPALLQSGLYTLSSSVTVTSNTWPSQT
ITCNVAHPASSTKVDKKIEPRVPITQNPCPPLKECPPCAAPDLLGGPSVF
IFPPKIKDVLMISLSPMVTCVVVDVSEDDPDVQISWFVNNVEVHTAQTQT
HREDYNSTLRVVSALPIQHQDWMSGKEFKCKVNNRALPSPIEKTISKPRG
PVRAPQVYVLPPPAEEMTKKEFSLTCMITGFLPAEIAVDWTSNGRTEQNY
KNTATVLDSDGSY [symbol] MYSKL [symbol] VQKSTWERGSLFACSVVHEGLHNHLTTKTI
SRSLGK

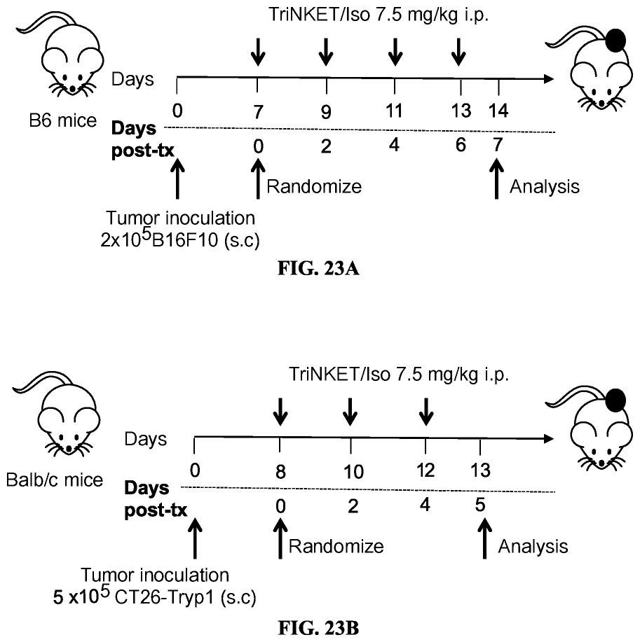

B16F10 tumor bearing mice were randomized on day 7 post-tumor inoculation into two treatment groups and treated intraperitoneally (IP) as follows: (1) mouse IgG2a isotype control (7.5 mg/kg every other day; n=8) and (2) Tyrp1-TriNKET (7.5 mg/kg every other day; n=8) (FIG. 23A). Simultaneously, CT26-Tyrp1 tumor bearing mice were randomized on day 7 post-tumor inoculation into 2 treatment groups and received treatment IP as follows:) mouse IgG2a isotype control (7.5 mg/kg every other day; n=10) and (2) Tyrp1-TriNKET (7.5 mg/kg every other day; n=10) (FIG. 23B). In both models, tumor size was measured on the day of randomization and subsequently 3 times a week using a digital caliper. On the day of analysis, mice were sacrificed, and tumors, draining lymph nodes and non-draining lymph nodes were harvested to isolate single cell suspensions for flow cytometric analysis.

The B16F10 tumor bearing mice were sacrificed on Day 14 (Day 7 post-initiation of treatment) for analysis. Tumor tissues were harvested and weighed for endpoint measurement.

Tumor draining and non-draining lymph nodes were also harvested and processed with the tumors to generate single-cell suspensions. Briefly, B16F10 tumor tissue was placed in a 10-cm plate, diced into small pieces using a razor blade, and filtered through a 75-μm cell strainer placed on top of a 50 mL conical tube. Similarly, lymph node tissue was placed on a pre-wet 75-μm cell strainer placed on top of a 50 mL conical tube and plunged through with 1 ml syringe plunger. Single cell suspensions were washed, resuspended in staining buffer (0.5% BSA in PBS), and cell counts were determined using an automated ViCell counter.

CT26-Tyrp1 tumor bearing mice were sacrificed on Day 13 (Day 5 post-initiation of treatment) for analysis. Tumor tissues were harvested and weighed as described above. Similarly, tumor-draining and non-draining lymph nodes were also harvested and processed to make single cell suspensions. For the CT26 tumors, an enzymatic dissociation protocol was utilized to generate single cell suspensions. Briefly, tumors were mechanically dissociated with razor blades followed by enzymatic digestion for 30 mins at 37° C. by collagenase A, 1.0 mg/mL, and DNAse I, 100 U/mL, dissolved in HBSS. Collagenase A enzyme activity was quenched by adding EDTA (final [1 mM]) to the digestion mixture followed by filtration through 0.7 μm nylon strainers. Single cell suspensions were washed, resuspended in staining buffer (0.5% BSA in PBS), and cell counts were determined using an automated ViCell counter.

For both models, single cell suspensions generated from tumor tissue and lymph nodes (both tumor draining and non-draining) were stained with fluorochrome-conjugated antibodies for flow cytometry analysis. Briefly, 100 μL of cells in staining buffer (1-2.5 million cells/test) were added to a 96-well round bottomed plate and incubated with Fc blocking antibody (0.5 μg/test) and live/dead stain for 10 mins at 4° C. Subsequently, cells were washed and resuspended in 100 μL of an antibody cocktail and incubated for 30 mins at 4° C. in the dark. Cells were washed and fixed in stabilizing fixative for future analysis with the BD LSRFortessa cell analyzer.

All flow cytometry analysis was performed using the FlowJo program (Treestar). Data were analyzed using GraphPad Prism V7.0 software and compared using Mann-Whitney test, One-way ANOVA and Student's t-test where appropriate. P-values <0.05 were considered significant.

B16F10 Tumor Model

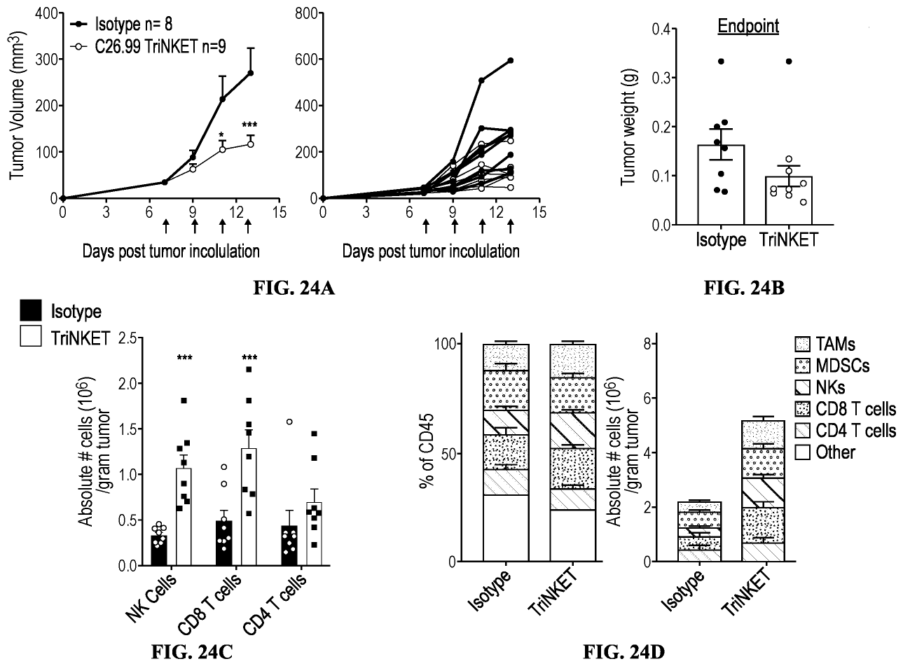

In the B16F10 tumor model group, treatment of mice with TriNKET delayed tumor progression as indicated by reduced average tumor volume and weights at endpoint (Day 14 post-tumor inoculation) compared to control treated mice (FIGS. 24A-24B). Heterogeneous responses to TriNKET treatment can be appreciated by the differing individual tumor volumes and weights in the TriNKET treatment group. To determine changes in the number of tumor infiltrating immune cells, immune cell subsets were quantified by flow cytometry analysis. TriNKET treated tumor tissue had a higher number of tumor-infiltrating NK cells (3.3-fold), CD8+ T cells (2.7-fold), CD4+ T cells (1.6-fold) and myeloid cells including tumor associated macrophages (2.6-fold; TAMs), and myeloid derived suppressor cells (2.2-fold; MDSCs) as compared to isotype treated tumors (FIGS. 24C-24D). However, the frequencies of NK cells, CD8+ T cells and TAMs within the CD45+ population were higher in the TriNKET treated group compared to the isotype treated group, while CD4+ T cell and MDSC frequencies were reduced (FIG. 24D). These data indicate that TriNKET treatment reprograms the tumor microenvironment of non-immunogenic B16F10 tumors that are minimally infiltrated by immune cells into “hot” tumors with approximately 2 times more total immune cells per gram of tumor tissue (FIG. 24C).

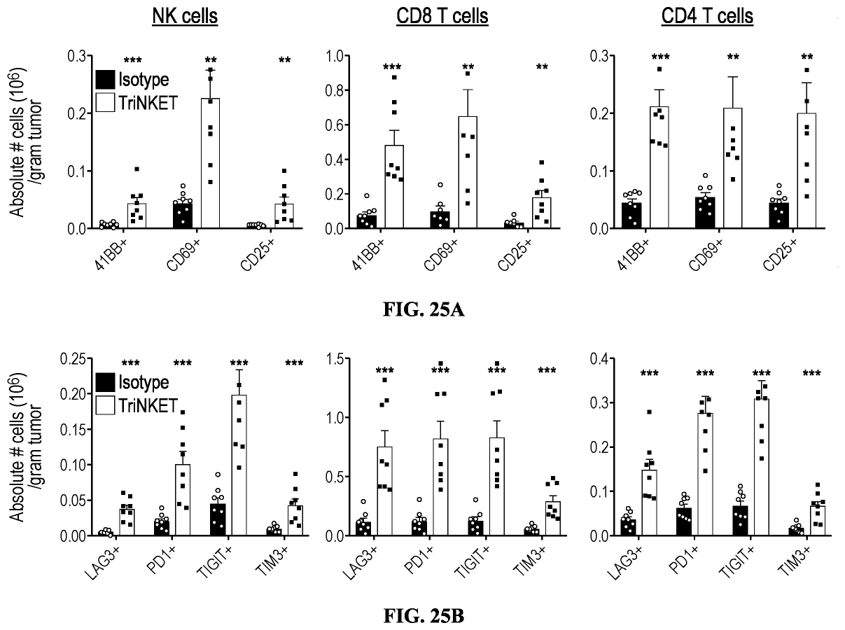

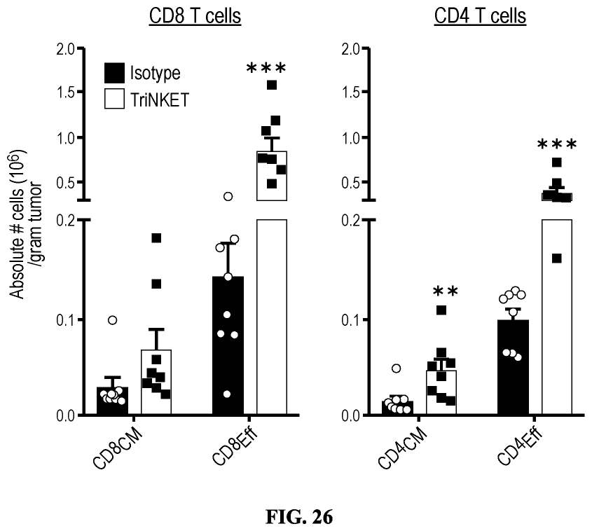

Further analyses of the composition of tumor immune infiltrates indicated that there are significantly higher numbers of NK cells, CD8+ T cells and CD4+ T cells expressing activation markers including 41BB, CD69, and CD25 (FIG. 25A). Additionally, TriNKET therapy significantly expanded NK cells, CD8+ T cells, and CD4+ T cells that express immune checkpoint markers including LAG3, PD1, TIGIT, and TIM3 (FIG. 25B). In addition to increasing activated NK and T cell numbers, TriNKET therapy also increased CD8+ CTL effector memory (CD44^{hi}CD62L^{neg}) and CD4+ central (CD44 hi CD62L hi) and effector memory cell counts (FIG. 26). Overall, this data suggests that TriNKET therapy reprograms the tumor microenvironment of B16F10 melanoma to tip the balance towards cytotoxic anti-tumor immune cells. Additionally, the higher number of immune-checkpoint expressing infiltrates suggests that TriNKETs may synergize with anti-PD1 blockade or other checkpoint therapies, to which B16F10 tumors are normally resistant.

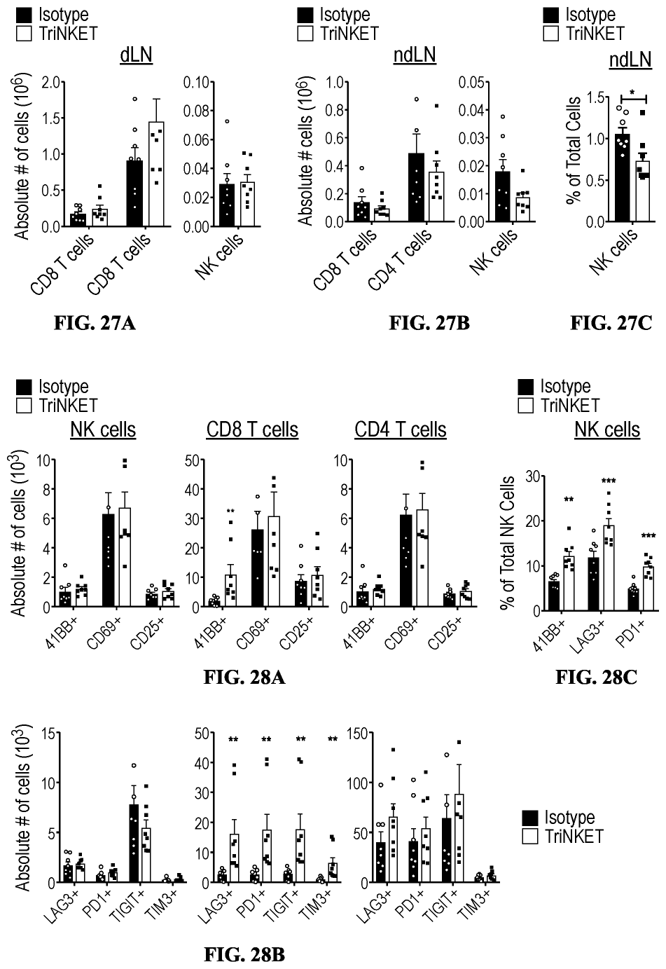

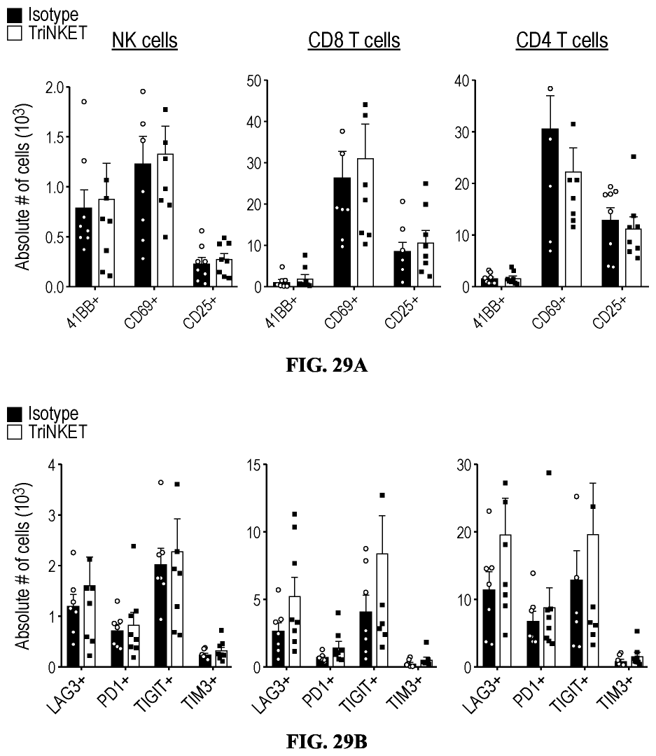

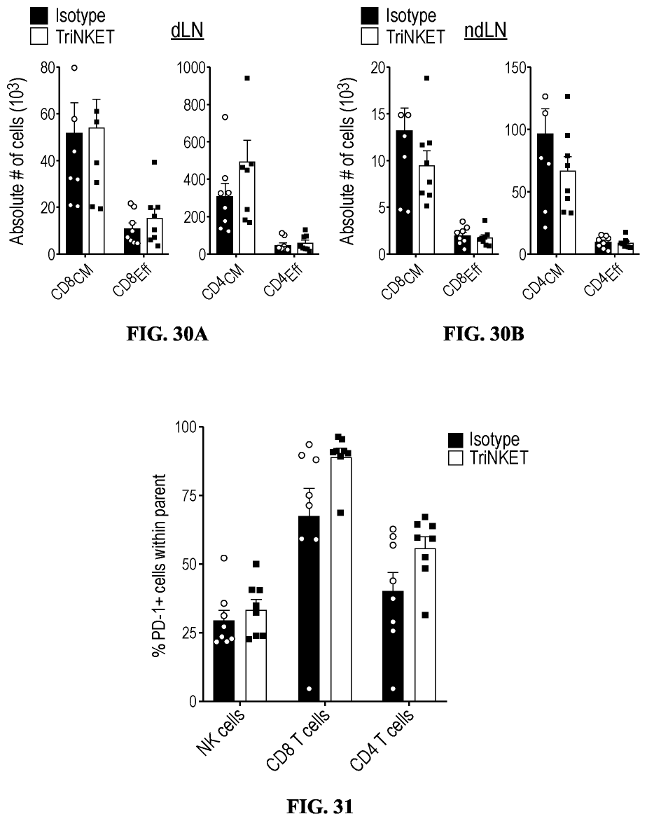

We next examined immune cell changes in the B16F10 tumor draining lymph nodes (dLN) and non-draining LN (ndLN). TriNKET treatment did not significantly change the number of T cells or NK cells in the dLN; however, a trend towards higher CD8+ T cells and CD4+ T cells was observed (FIG. 27A). Additionally, TriNKETs reduced the absolute number of NK cells (FIG. 27B) and their frequency within the ndLN (FIG. 27C), possibly due to their migration to the tumor tissue. Interestingly, TriNKETs induced a significant expansion of activated 41BB expressing CD8+ T cells in the dLN (FIG. 28A) as well as elevated cells that have a checkpoint-engaged phenotype (FIG. 28C). Although the absolute numbers of NK cells in the dLN remained unchanged with TriNKET treatment, the frequencies of NK cells expressing 41BB, LAG3 or PD1 were significantly higher within the parent NK cells in the tumor dLN (FIG. 28B). In ndLN, no significant changes in cell numbers were observed of T cells or NK cells that express activation markers (41BB, CD69 or CD25; FIG. 29A), however there was a trend of higher counts of CD8+ T cells and CD4+ cells expressing immune-checkpoint markers in the ndLN (FIG. 29B). No significant changes in the number of central memory or effector memory CD8+ T cells or CD4+ T cells were observed in the B16F10 tumor dLN or ndLN. (FIGS. 30A-30B).

To determine whether TriNKET treatment changes the level of immune-checkpoint markers, the percentages of PD-1 positive cells were analyzed within NK cells and T cell populations in the B16F10 tumors. It was observed that TriNKET treatment resulted in a significant increase of PD-1+ cells within the CD8 T cell population and elevated numbers of PD-1+ cells within the CD4 T cell and NK cell populations (FIG. 31).

CT26-Tyrp1 Tumor Model

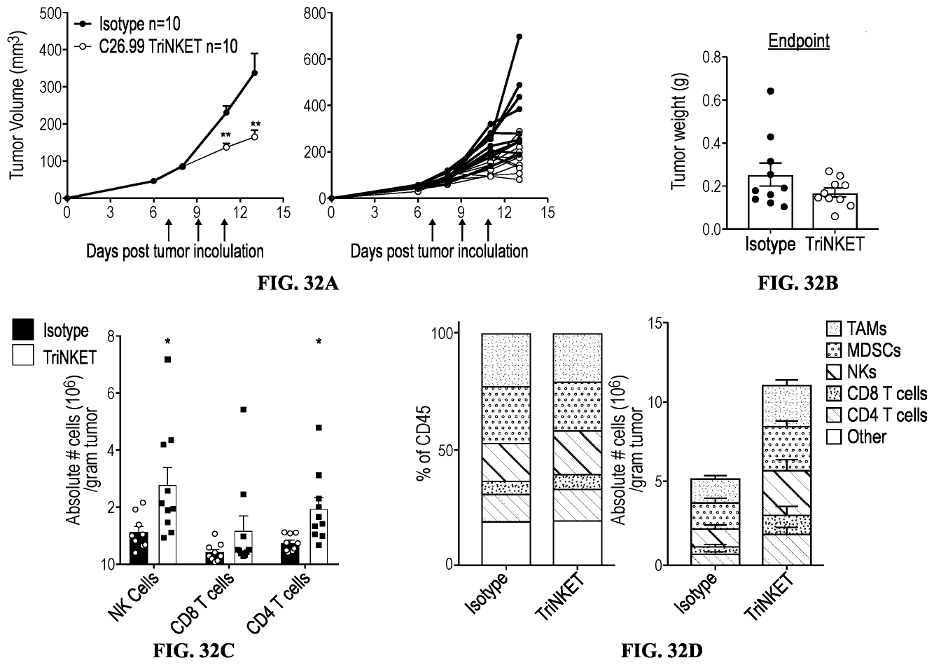

Similar to the B16F10 model, treatment with TriNKET delayed CT26-Tyrp1 tumor progression as indicated by reduced average tumor volume and weights at endpoint (Day 13 post-tumor inoculation) compared to isotype control treatment (FIGS. 32A-32B). Differential response to TriNKET treatment is indicated by the differing individual tumor volumes and weights in the TriNKET treatment group.

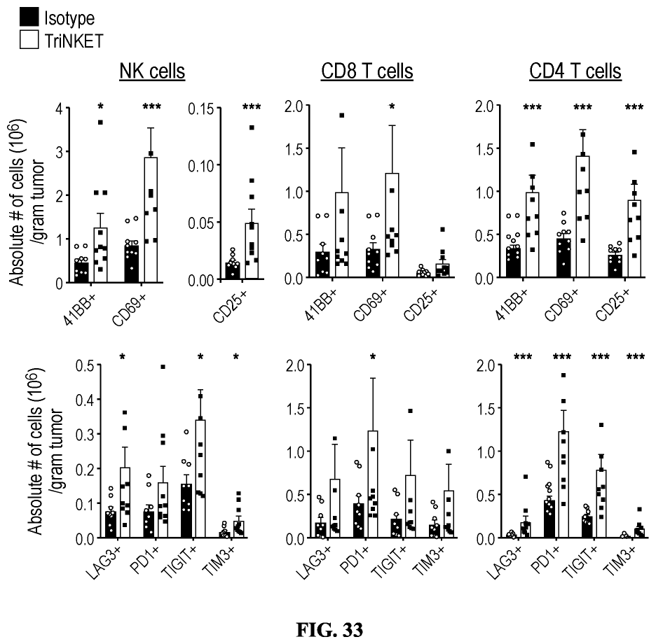

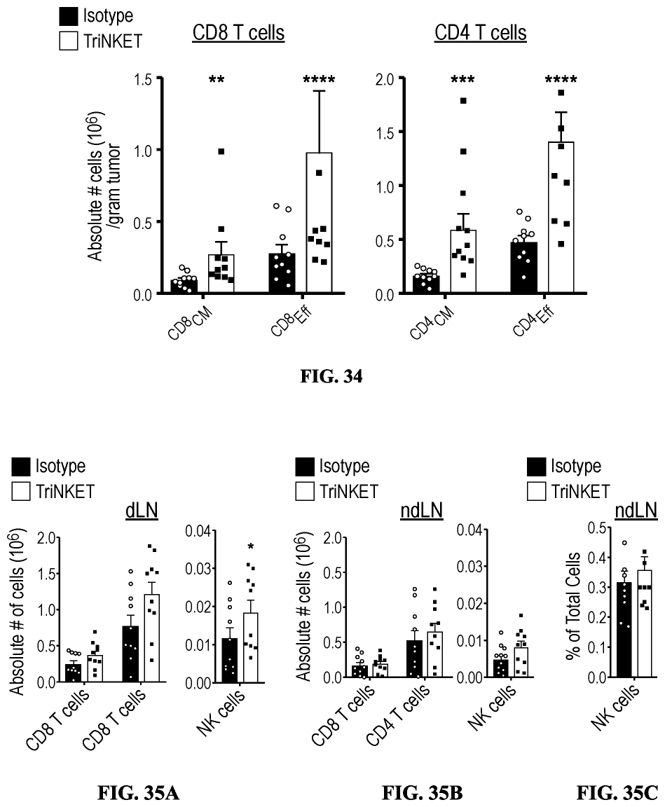

Similar to the B16F10 model, TriNKET treatment of mice bearing CT26-Tyrp1 tumors resulted in increased numbers of total NK cells (2.5-fold), CD8+ T cells (2.8-fold), CD4+ T cells (2.6-fold), TAMs (2.5-fold), and MDSCs (1.4-fold) FIGS. 32C-32D) as compared to isotype treated control. Notably, TriNKETs induced a more robust expansion of CD4+ T cells in CT26-Tyrp1, as compared to the B16F10 model, resulting in a higher ratio of CD4+ T cells to CD8+ T cells in the tumor. Moreover, TriNKETs increased the frequencies of NK cells and CD4 T cells within the CD45+ population and reduced TAMs and MDSCs frequencies (FIG. 32D). In addition to increasing tumor infiltrates, TriNKETs also increased activated NK cells, CD8+ T cells and CD4 T cells with an immune-checkpoint engaged phenotype immune-checkpoint (FIG. 33). Increases in CD44 hi CD62L hi (TCM) or CD44^{hi}CD62L^{lo} (T_{eff}) in CD8+ T cells and CD4+ T cells were also observed, suggesting that activated T cells may have a “memory” phenotype similar to the B16F10 model (FIG. 34).

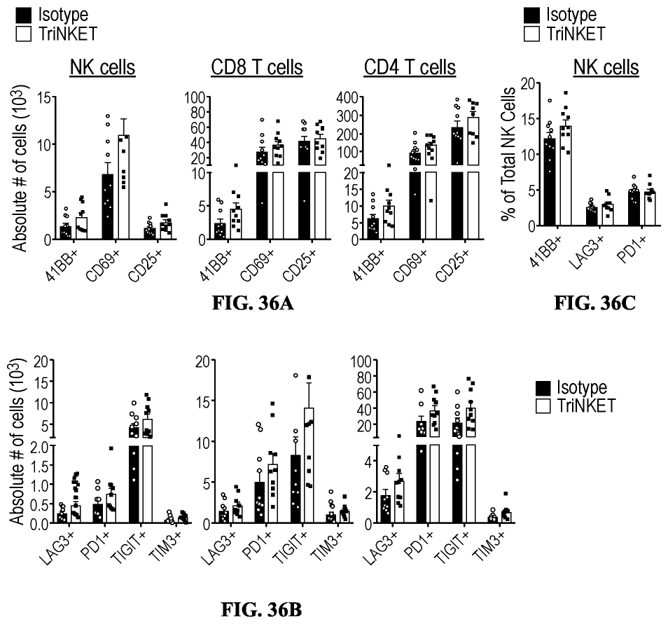

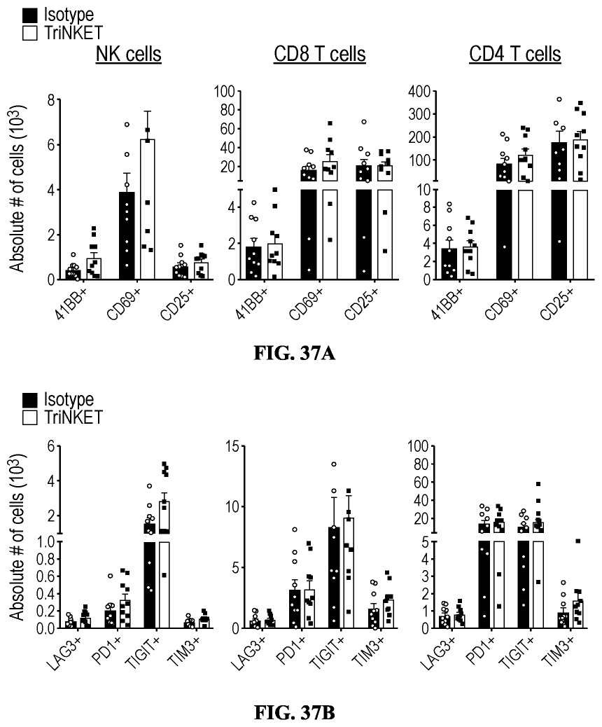

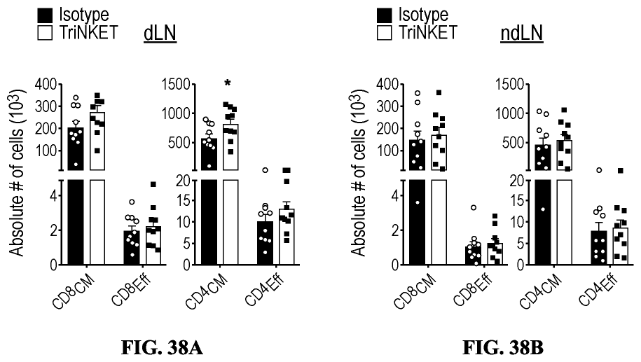

We also examined the immune cell changes in the CT26-Tyrp1 tumor dLN and ndLN. TriNKETs significantly elevated the number of NK cells in the dLN, and a trend of higher CD8+ T cells and CD4+ T cells was also observed (FIG. 35A). Unlike what was observed in the B16F10 model, TriNKETs did not affect the absolute number of NK cells (FIG. 35B) or their frequency within the total cells analyzed (FIG. 35C) in the ndLN. However, TriNKETs induced a trend of elevated numbers of activated and immune checkpoint expressing NK and T cells (FIGS. 36A-36B) in the dLN. No significant difference was observed in the frequency of NK cells expressing checkpoint markers (FIG. 36C). No significant differences were observed in the number of activated/checkpoint expressing T cell in the ndLN, however TriNKETs stimulated a trend of more activated NK cells (FIGS. 37A-37B). Interestingly, TriNKETs induced a significant increase in the number of central memory CD4+ T cells in the tumor dLN, but no differences were evident in the CD8+ CTLs memory responses in the dLN and ndLN (FIGS. 38A-38B).

In this example, TriNKETs were shown to have significant efficacy as monotherapy against established B16F10 melanoma tumors and CT26-Tyrp1 tumors in vivo. These data highlight a promising strategy to engage multiple anti-tumor immune cells, including NK cell and CD8+ T cells, with a single-agent. TriNKETs were able to turn “cold” B16F10 tumors to highly infiltrated “hot” tumors with expanded NK cells, CD8+ T cells and CD4+ T cells in the TME. TriNKETs also expanded NK and T cells that are highly active and express immune-checkpoint markers including PD1, LAGS, TIM3, and TIGIT. These immune changes can possibly overcome resistance to checkpoint immunotherapy in melanoma models, supporting the concept of using TriNKETs to overcome resistance to checkpoint immunotherapies.

Intriguingly, an overall expansion in the number of TAMs and MDSCs with TriNKET treatment in B16F10 and CT26-Tyrp1 tumors was also observed. When analyzed within the total CD45+ leukocyte population, the frequency of MDSCs was reduced, whereas NK cell and CD8+ T cells frequencies were elevated. This suggests that, although TriNKETs induce the infiltration of all immune cells into the newly “inflamed” tumor microenvironment, it preferentially expands cytotoxic NK cells and CD8+ T cells, tipping the balance towards cells promoting robust anti-tumor responses. Preliminary in-depth phenotyping data also indicated that majority of the expanded TAMs were M1-TAMs that are known to promote anti-tumor responses. It was also shown that TriNKETs expanded both memory CD8+ CTL and CD4+ T cells in the TME and tumor dLN that can potentially support long-lasting memory responses. Data utilizing a surrogate Tyrp1 targeting TriNKET suggest that successful reprograming of both innate and adaptive immune cells in the TME by TriNKETs simultaneously targeting NK cells, CD8+ T cells, and tumor cells can promote anti-tumor responses and render tumors more sensitive to combination checkpoint therapy in humans.

### INCORPORATION BY REFERENCE

The entire disclosure of each of the patent documents and scientific articles referred to herein is incorporated by reference for all purposes.

### EQUIVALENTS

The disclosure may be embodied in other specific forms without departing from the spirit or essential characteristics thereof. The foregoing embodiments are therefore to be considered in all respects illustrative rather than limiting the disclosure described herein. Various structural elements of the different embodiments and various disclosed method steps may be utilized in various combinations and permutations, and all such variants are to be considered forms of the disclosure. Scope of the disclosure is thus indicated by the appended claims rather than by the foregoing description, and all changes that come within the meaning and range of equivalency of the claims are intended to be embraced therein.

1-102. (canceled)

103. A method of treating a non-small-cell lung cancer (NSCLC) in a subject in need thereof, the method comprising administering to the subject an effective amount of a multi-specific binding protein comprising:
(a) a first antigen-binding site that binds NKG2D;
(b) a second antigen-binding site that binds HER2; and
(c) an antibody Fc domain or a portion thereof sufficient to bind CD16, or a third antigen-binding site that binds CD16,
wherein the NSCLC has erbb2 gene amplification and/or an erbb2 activating mutation, and/or is a HER2-overexpressing cancer.

104. (canceled)

105. The method of claim 103, wherein the NSCLC is stage IIIB, stage IV, or recurrent.

106. The method of claim 103, wherein the NSCLC has a HER2 level scored 2+ or higher by immunohistochemistry.

107-111. (canceled)

112. The method of claim 103, wherein the multi-specific binding protein is provided as a monotherapy.

113. The method of claim 103, further comprising administering a PD-1 inhibitor to the subject.

114. The method of claim 113, wherein the PD-1 inhibitor comprises an anti-PD-1 antibody, optionally nivolumab.

115-136. (canceled)

137. The method of claim 103, wherein the multi-specific binding protein is administered on Day 1, Day 8, and Day 15, but not on Day 22, of an initial four-week treatment cycle.

138. (canceled)

139. The method of claim 137, wherein after the initial treatment cycle, the multi-specific binding protein is administered on Day 1 and Day 15 of one or more subsequent four-week treatment cycles.

140. The method of claim 103, wherein the multi-specific binding protein is administered, in each dose, at an amount selected from the group consisting of 5.2×10⁻⁵ mg/kg, 1.6×10⁻⁴ mg/kg, 5.2×10⁻⁴ mg/kg, 1.6×10⁻³ mg/kg, 5.2×10⁻³ mg/kg, 1.6×10⁻² mg/kg, 5.2×10⁻² mg/kg, 1.6×10⁻¹ mg/kg, 0.52 mg/kg, 1.0 mg/kg, 1.6 mg/kg, 5.2 mg/kg, 10 mg/kg, 15 mg/kg, 20 mg/kg, and 50 mg/kg.

141. The method of claim 137, wherein the multi-specific binding protein is administered, on Day 1 of the initial treatment cycle, at the amount of 5 mg/kg.

142. The method of claim 141, wherein the multi-specific binding protein is administered, in each subsequent dose, at the amount of 15 mg/kg, 20 mg/kg, 10 mg/kg, or 5 mg/kg.

143-145. (canceled)

146. The method of claim 103, wherein the multi-specific binding protein is administered by intravenous infusion.

147-153. (canceled)

154. The method of claim 103, further comprising administering a cytoskeletal-disrupting chemotherapeutic agent to the subject, optionally nab-paclitaxel.

155-159. (canceled)

160. The method of claim 103, wherein the first antigen-binding site comprises a Fab comprising a heavy chain variable domain (VH) and a light chain variable domain (VL), and wherein
(a) the VH of the Fab comprises complementarity-determining region 1 (CDR1), complementarity-determining region 2 (CDR2), and complementarity-determining region 3 (CDR3) sequences represented by the amino acid sequences of SEQ ID NOs: 168, 96, and 188, respectively; and
(b) the VL of the Fab comprises CDR1, CDR2, and CDR3 sequences represented by the amino acid sequences of SEQ ID NOs: 99, 100, and 101, respectively.

161. The method of claim 160, wherein
(a) the VH of the Fab comprises CDR1, CDR2, and CDR3 sequences represented by the amino acid sequences of SEQ ID NOs: 168, 96, and 169, respectively; and
(b) the VL of the Fab comprises CDR1, CDR2, and CDR3 sequences represented by the amino acid sequences of SEQ ID NOs: 99, 100, and 101, respectively.

162. The method of claim 160, wherein the VH of the Fab comprises an amino acid sequence at least 90% identical to SEQ ID NO:94, and the VL of the Fab comprises an amino acid sequence at least 90% identical to SEQ ID NO:98.

163. (canceled)

164. The method of claim 160, wherein the second antigen-binding site comprises a single chain variable fragment (scFv) comprising a VH and a VL, and wherein
(a) the VH of the scFv comprises CDR1, CDR2, and CDR3 sequences represented by the amino acid sequences of SEQ ID NOs: 115, 116, and 117, respectively; and
(b) the VL of the scFv comprises CDR1, CDR2, and CDR3 sequences represented by the amino acid sequences of SEQ ID NOs: 119, 120, and 121, respectively.

165. The method of claim 164, wherein the VH of the scFv comprises an amino acid sequence at least 90% identical to SEQ ID NO:195, and the VL of the scFv comprises an amino acid sequence at least 90% identical to SEQ ID NO:196.

166-173. (canceled)

174. The method of claim 160, wherein the antibody Fc domain comprises a first antibody Fc sequence linked to a C-terminus of a heavy chain portion of the Fab and a second antibody Fc sequence linked to a C-terminus of the scFv via a hinge comprising Ala-Ser, and wherein the first and second antibody Fc sequences comprise different mutations that promote heterodimerization.

175-179. (canceled)

180. The method of claim 174, wherein the first antibody Fc sequence is a human IgG1 Fc sequence comprising K360E and K409W substitutions, and the second antibody Fc sequence is a human IgG1 Fc sequence comprising Q347R, D399V, and F405T substitutions.

181. (canceled)

182. The method of claim 160, wherein the multi-specific binding protein comprises:
(a) a first polypeptide comprising the amino acid sequence of SEQ ID NO:141;
(b) a second polypeptide comprising the amino acid sequence of SEQ ID NO:140; and
(c) a third polypeptide comprising the amino acid sequence of SEQ ID NO:142.

183-202. (canceled)
